(19) United States
(12) Patent Application Publication
(10) Pub. No.: US 2022/0090124 A1
(43) Pub. Date: Mar. 24, 2022

(54) HUMANIZED VIPR2 COPY NUMBER VARIANT TRANSGENIC MOUSE MODEL FOR ANTIPSYCHOTIC DRUG AND GENE THERAPY DISCOVERY FOR SCHIZOPHRENIA

(71) Applicant: Board of Supervisors of Louisiana State University and Agricultural and Mechanical College, Baton Rouge, LA (US)

(72) Inventors: Xiaohong LU, Shreveport, LA (US); Xinli TIAN, Shreveport, LA (US)

(21) Appl. No.: 17/001,590

(22) Filed: Aug. 24, 2020

Related U.S. Application Data
(60) Provisional application No. 62/890,430, filed on Aug. 22, 2019.

(51) Int. Cl. C12N 15/85; A01K 67/027

(52) U.S. Cl. CPC C12N 15/8509; A01K 2227/105; A01K 2267/0306; A01K 67/0278

(57) ABSTRACT

The disclosed invention relates to methods and transgenic non-human mammals comprising a full length human VIPR2 genomic region integrated into a genome of the mammal. According to a further embodiment the mammal is a mouse. The disclosed invention further relates to transgenic cells from the transgenic non-human mammal. The disclosed invention further relates to therapeutics and methods of treating Schizophrenia in a human comprising administering a therapeutic, where the therapeutic contains one of a pharmacologically effective amount of a hVIPR2 antagonist, and a CRISPR/Cas9 formulation. The disclosed invention further relates to materials and methods of determining efficacy of an antipsychotic therapeutic in treating a condition comprising administering to the transgenic non-human mammal.

### CROSS REFERENCE TO RELATED APPLICATIONS/PRIORITY

The present invention claims priority to United States Provisional Patent Application No. 62/890,430 filed Aug. 22, 2019, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### REFERENCE TO A SEQUENCE LISTING SUBMITTED ELECTRONICALLY VIA EFS-WEB

The content of the electronically submitted sequence listing (Name: Sequences_ST25.txt; Size: (278,501 bytes; and Date of Creation: Oct. 18, 2021) is herein incorporated by reference in its entirety.

### BACKGROUND

Schizophrenia is a chronic and often disabling neuropsychiatric disorder that affects 1% of the population, with a lifetime prevalence of 4.0 per 1000 individuals. The economic burden of Schizophrenia to the United States was estimated at $155.7 billion for 2013, with substantial non-health care and indirect costs. The symptoms of Schizophrenia fall into three symptomatic clusters: positive symptoms (delusion, hallucination, disorganized speech, and behavior, etc.), negative symptoms (anhedonia and affective flattening, etc.), and cognitive symptoms (working memory deficits, executive functioning, etc.). Current pharmacologic agents primarily manage the positive symptoms (responsive only in a small percentage of patients), while cognitive and negative symptoms are largely refractory. Therefore, ameliorating cognitive and negative symptoms to help functioning in people with Schizophrenia has become a primary therapeutic effort. Current antipsychotic drug markets represent approximately 5 million people and generated revenues of about $18 billion annually. However, since the discovery of clozapine in the late 1950s, the progress in antipsychotic drug discovery has remained stagnant, with no fundamental innovation. The situation is further exacerbated by the recent withdrawal of research effort in the filed by the major pharmaceutical companies. To the inventors' knowledge, all antipsychotic medications that have been developed over the past six decades are based on the D2 receptors targeting the 5-HT2A antagonism of Second Generations Antipsychotics (SGAs), and that there is a lack of effective disease-modifying therapeutics for Schizophrenia to prevent its onset and slow/stop the disease process. There is a pressing need for novel therapeutics that rationally target cellular and molecular targets, rather than just the D2 receptor to be developed. However, a lack of understanding of the pathogenesis/genetics of Schizophrenia and the absence of credible animal models are the biggest hurdle for the development of next-generation of antipsychotic therapeutics targeting disease process.

Current pharmacologic agents primarily manage the positive symptoms (in about 30% of patients), while negative and cognitive symptoms are refractory. A key barrier to resolving poor patient response to current antipsychotics is the scarcity of animal models for drug discovery that can model the underlying causal of the disease.

Current available animal models of Schizophrenia fit into four different induction categories: developmental, drug-induced, lesion, and genetic animal models. The developmental, drug-induced and lesion animal models can recapitulate certain symptomatic groups of patients, but they are primarily based on one aspect of the hypothetic pathogenic mechanisms of Schizophrenia that has yet to be proven. Furthermore, they are primarily acute or subacute models, where the onset and development of symptoms depend on the time points of induction. Therefore, the inventors note that the current animal models pose significant limits to being able to be used to develop disease-modifying therapies to prevent or slow/stop the development of the underlying causal of the disease.

There are a few genetic animal models that were established to introduce the human genetic variants into mouse. However, all the current genetic animal models have significant limitations: First, a lack of consistent verification by independent studies of genetic variants, and in many times, such variants cannot be repeated in the following large-scale human genetic studies. Second, the current genetic animal models were generated using traditional gene targeting technology to “knock out” a gene, which is good to study recessive loss-of-function mutation. But such Knockout (KO) models cannot model copy number variant or genetic polymorphism that is often seen in Schizophrenia patients. Finally, most of the current animal models can only manifest limited symptomatic groups of patients, most positive symptoms. None of the animal models the inventors are aware of can demonstrate a full spectrum of negative and cognitive symptoms, which severely limited the current models' usage for antipsychotic drug discovery.

### SUMMARY

The present invention is directed to methods, organisms, and materials, some embodiments of which satisfy some or all of the above shortcomings and drawbacks.

The disclosed invention describes a VIPR2 Copy Number Variant Bacterial Artificial Chromosome transgenic mouse model of Schizophrenia comprising the integration of multiple copies human VIPR2 BAC into the genome of mouse with null mouse VIPR2 that manifest behavioral deficits associated with Schizophrenia. Such genetic Schizophrenia mouse model can be used to study VIPR2 receptor antagonist as an antipsychotic drug to relieve positive, negative and cognitive symptoms and for disease-modifying efficacy to prevent or slow/stop disease progression. The invention also facilitates the development of a therapeutic CRISPR/Cas9 mediated gene editing method to delete/inactivate extra copies of hVIPR2, or downregulation of human VIPR2 overexpression as gene therapy for Schizophrenia. The invention provides a preclinical animal model of Schizophrenia to examine/screen efficacy of the next generation of antipsychotic drugs/therapies.

The disclosed invention relates to methods and transgenic non-human mammals comprising a full length human VIPR2 genomic region integrated into a genome of the mammal. According to a further embodiment the mammal is a mouse. According to a further embodiment the VIPR2 genomic region is within a Bacterial Artificial Chromosome. According to a further embodiment the Bacterial Artificial Chromosome and VIPR2 genomic region have at least 90% sequence identity to SEQ ID NO: 1. According to a further embodiment wherein the mammal manifests Schizophrenia-associated behavioral deficits. According to a further embodiment a single copy of the full length human VIPR2 genomic region is integrated into the mammal genome. According to a further embodiment multiple copies of the full length human VIPR2 genomic region is integrated into the mammal genome. According to a further embodiment a number of copies of the full length human VIPR2 genomic region integrated into the mammal genome is between 1 and 4.

The disclosed invention further relates to transgenic cells from the transgenic non-human mammal.

The disclosed invention further relates to therapeutics and methods of treating Schizophrenia in a human comprising administering a therapeutic, where the therapeutic contains one of a pharmacologically effective amount of a hVIPR2 antagonist, and a CRISPR/Cas9 formulation. According to a further embodiment the hVIPR2 antagonist is a small-molecule hVIPR2 antagonist. According to a further embodiment the hVIPR2 antagonist (2R,4S)-2-benzyl-4-hydroxy-N-((1S,2R)-2-hydroxy-2,3-dihydro-1H-inden-1-yl)-5-(4-nitrophenylsulfonamido) pentanamide. According to a further embodiment the CRISPR/Cas9 formulation therapeutic mediates genome editing to one of delete and inactivate extra copies of hVIPR2, or downregulate hVIPR2 overexpression. According to a further embodiment the therapeutic treats both cognitive and social deficits of Schizophrenia.

The disclosed invention further relates to materials and methods of determining efficacy of an antipsychotic therapeutic in treating a condition comprising administering to the transgenic non-human mammal of claim 1 the antipsychotic therapeutic, and measuring symptoms of the condition to determine an effectiveness of the therapeutic. According to a further embodiment wherein the condition is Schizophrenia. According to a further embodiment the symptoms are one of positive, negative and cognitive symptoms of Schizophrenia. According to a further embodiment the symptoms are each of positive, negative and cognitive symptoms of Schizophrenia. According to a further embodiment the method of further comprises measuring disease-modifying efficacy to one of prevent disease development, slow disease progression, and stop disease progression. According to a further embodiment the non-human mammal is a mouse. The present invention relates to generating a novel genetic animal model for antipsychotic drug discovery that faithfully reconstructs human Schizophrenia genetics in the mouse genome to elicit behavioral and pathologic deficits recapitulating cognitive and negative symptoms of Schizophrenia patients.

The invention relates a genetic Schizophrenia mouse model to study VIPR2 receptor antagonist as an antipsychotic drug for disease-modifying efficacy to prevent or slow/stop disease progression.

The invention relates a CRISPR/Cas9 mediated gene therapy to delete/inactivate extra copies of hVIPR2, or downregulation of hVIPR2 overexpression as gene therapy for Schizophrenia.

The invention relates to a preclinical animal model of Schizophrenia to examine/screen efficacy of the next generation of antipsychotic drugs.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 shows a schematic design of conditional VIPR2 BAC transgenic mice.

FIG. 2 is a chart showing primers specific for both the human and mouse VIPR2 gene were used to quantify transgene copy numbers in VIPR2 CNV mice. Mouse wildtype genomic DNA was used as a two copy per diploid genome control. Line A contains one human copy of the VIPR2 genomic locus sequences (i.e., BAC transgene copies), and line F contains four human transgene copies.

FIG. 3 is a chart showing relative expression of human VIPR2 mRNA in different brain regions of VIPR2 CNV mice (Line A) determined by qPCR analysis. FC: Frontal cortex; STR: Striatum; HIP: hippocampus; OB: Olfactory bulb; MID: Midbrain.

FIG. 4 is a chart showing relative expression of human VIPR2 gene and endogenous mouse Vipr2 gene in cortex and striatum of VIPR2 CNV mice (Line A) determined by qPCR.

FIG. 5 is a chart showing endogenous murine Vipr2 and human VIPR2 transgene expression in mouse Vipr2 Knockout (KO), fully humanized (Hu), VIPR2 CNV (Line A), and wildtype mice (WT) determined by qPCR.

FIGS. 6 and 7 are a Western blot and chart, respectively, of the VPAC2 protein in the striatum of VIPR2 CNV mice (Line A) and quantifications (n=4-6, t-test, **p<0.01).

FIGS. 8 and 9 are micrographs showing mouse Vipr2 expression as shown from the image of in situ hybridization (ISH) in the mouse Vipr2-BAC Cre mice crossing with reporter mice (Allen Brain Atlas) (FIG. 8) and immunohistochemistry staining (FIG. 9) to determine the human VIPR2 expression in the mouse Vipr2 null background.

FIGS. 10-13 are micrographs that show the expression of human VIPR2 was confirmed in the cortex (FIG. 10), striatum (FIG. 11), hippocampus (FIG. 12), and the suprachiasmatic nucleus (SCN, FIG. 13). FIGS. 9 and 10: Scale bar=25 μm; FIG. 11: Scale bar=10 μm; FIG. 13: Scale bar=50 μm.

FIGS. 14-16 hVIPR2 is expressed in dSPNs in the striatum as demonstrated by the double fluorescence staining of GFP (green) and VPAC2 (red) in Drd1a-GFP/VIPR2 CNV double transgenic mice (Line A). Scale bar=25 μm.

FIG. 17 is a schematic drawing showing spatial delayed non-match-to-sample T-maze task.

FIG. 18 is a line chart displaying the spatial delayed non-match-to-sample T-maze task data of the percentage of correct responses, represented as means with standard errors, and were analyzed by a two-way repeated-measures ANOVA (Interaction effect: *, p<0.05 (Line A); #, p<0.05 (Line F).

FIG. 19 is a graph showing acquisition (days to criterion) as a function of genotype and treatment is shown. (*, p<0.05 (Line A), # p<0.05 (Line F). One-Way ANOVA, with Turkey's post hoc test.

FIG. 20 is a graph showing spontaneous alternation task in T-maze. VIPR2 CNV mice lines (A and F) showed significantly less spontaneous alterations than control mice, and a significantly higher percent of incorrect altercations (** p<0.01 (Line A), ## p<0.01 (Line F), one-way ANOVA, with Turkey's post hoc test.

FIGS. 21 and 22 are graphs showing pre-pulse inhibition (PPI) of the acoustic startle response deficits in VIPR2 CNV mice. PPI Data (mean±SEM) shows the percent of pre-pulse inhibition of the startle response following the presentation of pre-pulse—plus—pulse acoustic stimuli. Two different inter-stimulus Interval (ISIs) (30 and 100 ms) and two different pre-pulse intensities (75 and 85 dB) were measured. In FIG. 21, VIPR2 CNV (Line A) mice showed a significant PPI deficiency when presented with an 85-dB prepulse with 30 or 100 ms ISI (t-test; * p<0.05. ## p<0.01. In FIG. 22, line F mice are shown to have a significant PPI deficiency when presented with an 85 dB pre-pulse with 30 ms ISI (t-test; * p<0.05).

FIGS. 23 and 24 are graphs showing social approach and social recognition deficits, respectively, in VIPR2 CNV mice. In the social interaction test (FIG. 23) Line A showed significance, but not Line F, of both VIPR2 CNV mice spending less time in the chamber containing the social partner (Stranger 1), and more time in the chamber containing the empty wire cage when compared to controls (**p<0.01, one-way ANOVA, with Turkey's post hoc test). In the social recognition test (FIG. 24), where the mice had a free choice between the first, already-investigated mouse (Stranger 1), and a novel unfamiliar mouse (Stranger 2), both lines of VIPR2 CNV mice do not display a preference for the novel social partner (Stranger 2) at a level of significance. (**p<0.01 (Line A), ##p<0.01 (Line F), one-way ANOVA, with Turkey's post hoc test).

FIGS. 25-26 are example micrographs showing that compared to WT (FIG. 25), both founder lines of VIPR2 CNV mice (shown image of Line F in FIG. 26) at 3 months of age had an increased D2 receptor immunostaining in dorsomedial (DMS, associative, A), dorsolateral (DLS, sensorimotor, S), and dorsal striatum (D), but not in the ventral striatum (V). Scale bar=500 μm.

FIG. 27 is a graph showing quantification of the D2r immunostaining intensity in different subregions of the striatum, exemplarily show in FIGS. 25 and 26, revealed that VIPR2 CNV mice (line F) have a significant increase of D2r in the DMS and whole dorsal striatum (both rostral and caudal levels of coronal sections were used for quantification. The data were expressed as the average of both left and right hemisphere immunostaining intensity. n=4 mice per genotype, t-test, *, p<0.05).

FIG. 28 is a graph showing Dorsostriatal Dopamine (DA) content was significantly increased in the VIPR2 CNV mice (Line A) at 3 months of age compared to the WT mice as measured by HPLC (*, p<0.05, t-test, n=6 per genotype).

FIGS. 29-31 are two micrographs and a graph showing VIPR2 CNV mice (Line A) had an increase of TH immunostaining in the dorsal striatum (*, p<0.05, n=4 per genotype, Scale bar=500 μm) compared with WT, with FIG. 29 being a WT micrograph, FIG. 30 being a CNV micrograph, and FIG. 31 being a graph displaying the difference in TH immunointensity.

FIG. 32 is a graph showing VIPR2 CNV mice (Line A, Postnatal day 18, P18) showed an increased striatal cAMP level (pmol cAMP/mg protein expressed as a percentage of control mice) in the striatum. No significant difference of cAMP levels was observed in the olfactory bulb (Ofb), hippocampus (Hip) and Midbrain (Mid) (one sample t-test; * p<0.05, n=8 per genotype).

FIG. 33 is a graph showing adult VIPR2 CNV mice (Line A, 3-5 months old) have significantly elevated cAMP accumulation in both cortex and striatum 1 hour after intraperitoneal injection (i.p.) of a selective VIPR2 agonist, BAY 55-9837 (0.25 ug/g) (** p<0.01, n=4 mice per genotype/treatment).

FIG. 34 is a graph showing the accumulation of cAMP in the humanized mouse model and Vipr2 knockout mouse model after BAY 55-9837 (0.25 ug/g) i.p. (* p<0.05, n=4 mice per genotype/treatment, 3-5 months).

FIG. 35 is a western blot and graph displaying the results thereof showing p(Ser/Thr) PKA substrates levels of the striatum from WT and VIPR2 CNV mice at P18.

FIG. 36 is a western blot and graph displaying the results thereof showing pPKAcThr197 from protein extracts obtained from the striatum of WT and VIPR2 CNV mice (P18). For p(Thr) PKA substrates, all the bands were used for quantification (t-test; * p<0.05, n=4 or 5 mice per genotype).

FIG. 37 is a western blot and graph displaying the results thereof showing PKA-dependent phosphorylation of CREBser133 from protein extracts obtained from the striatum of WT and VIPR2 CNV mice (P18). For p(Ser/Thr) PKA substrates, all the bands were used for quantification (t-test; * p<0.05, n=4 or 5 mice per genotype).

FIGS. 38 and 39 are micrographs of WT and VIPR2 CNV immature striatum showing at P8, vGlut1 immunostaining delineates a patch-like ‘afferent islands’ in immature striatum. Scale bar=100 μm.

FIGS. 40 and 41 are higher magnification of striatum in wildtype (FIG. 40) and VIPR2 CNV mice (FIG. 41, Line A) are shown. Scale bar=25 μm.

FIGS. 42 and 43 are a western blot and graph displaying the results thereof showing vGlut-1 protein levels in the striatum in VIPR2 CNV mice (Line A) are significantly increased in comparison to that of the wildtype mice in (n=3 per genotype, t-test, *: p 0.05).

FIG. 44 is three micrographs of the Drd1a-GFP BAC mice at P8, where the localization of dSPNs patches (GFP staining, green) corresponded to striosomes identified by mu opioid receptor (Mu-OR) staining (blue). Scale bar=100 μm.

FIGS. 45 and 46 are micrographs showing dSPN patches colocalize with the intense VGIut1 immunoreactivities as revealed by double immunofluorescence labeling for vGlut1 (red) and dSPNs (green) at P8. Scale bar=50 μm.

FIGS. 47 and 48 are exemplary micrographs of Golgi stains. Multiple brains from wild type littermates and VIPR2 CNV mice at P18 were subjected to Golgi staining. Representative traces of SPNs in wild type littermates (FIG. 47) and VIPR2 CNV mice (FIG. 48) are shown.

FIG. 49 is a graph showing that a Sholl analysis of intersection of SPNs in VIPR2 CNV mice and wild type littermates (30 neurons from 6 mice per genotype) identified a significant genotype—distance interaction (p<0.01, repeated measure two-way ANOVA).

FIGS. 50 and 51 are representative high magnification images of dendritic spines of SPNs from wild type littermates (FIG. 50) and VIPR2 CNV mice (FIG. 51). Dendritic spines of SPNs were categorized in immature (thin and filopodia-like) or mature spines (mushroom, stubby, and multiple spine post-synapses).

FIG. 52 is a graph showing that the average number of mature spines (mushroom) per 10 μm dendritic length in SPNs from VIPR2 CNV mice (Line A) is significantly lower than that of wild-type SPNs (t-test; * p<0.05).

FIG. 53 is a graph showing that the average number of immature (thin) spines per 10 μm dendritic length in SPNs from VIPR2 CNV mice is significantly higher than that of wild-type SPNs (t-test; * p<0.05).

FIG. 54 is a graph showing that the diameter of the dendrites in SPNs from VIPR2 CNV mice is significantly lower than that of wild-type SPNs (t-test; ** p<0.01).

FIG. 55 is a graph showing that the average length of spines in SPNs from VIPR2 CNV mice is significantly longer than that of wild-type SPNs (t-test; * p<0.05).

FIG. 56 is schematic representation of a strategy to for a CRISPR/Cas9 mediated in vivo correction of VIPR2 duplication in VIPR2 CNV mice. Exons I and II of a specific gene are separated by a duplicated VIPR2 gene. Paired guide RNAs (VIPR2 sg-5 and sg-3) are designed that recognize PAM on either side of human VIPR2 genomic regions. Following Cas9-mediated double strand break (DSB) and non-homologous end-joining (NHEJ), the aberrant duplicated VIPR2 genomic DNAs are removed. In the same cell where VIPR2 is deleted, paired gRNAs (reporter sg-5 and sg-3) guide saCas9 to delete the STOP cassette in the genomic regions of the reporter mice. Red fluorescence protein (tdTomato) may be expressed to genetically label the cells with Cas9 activity and correct deletion of the genomic regions.

FIG. 57 is a small molecule hVIPR2 antagonist, (2R,4S)-2-benzyl-4-hydroxy-N-((1S,2R)-2-hydroxy-2,3-dihydro-1H-inden-1-yl)-5-(4-nitrophenylsulfonamido) pentanamide to treat Schizophrenia.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

The terms “agonist” and “agonistic” as used herein refer to or describe an agent that is capable of, directly or indirectly, substantially inducing, activating, promoting, increasing, or enhancing the biological activity of a target and/or a pathway. The term “agonist” is used herein to include any agent that partially or fully induces, activates, promotes, increases, or enhances the activity of a protein.

The terms “antagonist” and “antagonistic” as used herein refer to or describe an agent that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein.

The terms “selectively binds” or “specifically binds” mean that an agent interacts more frequently, more rapidly, with greater duration, with greater affinity, or with some combination of the above to the epitope, protein, or target molecule than with alternative substances, including related and unrelated proteins. In certain embodiments “specifically binds” means, for instance, that an agent binds a protein or target with a KD of about 0.1 mM or less, but more usually less than about 1 μM. In certain embodiments, “specifically binds” means that an agent binds a target with a KD of at least about 0.1 μM or less, at least about 0.01 μM or less, or at least about 1 nM or less. Because of the sequence identity between homologous proteins in different species, specific binding can include an agent that recognizes a protein or target in more than one species (e.g., mouse VPIR2 and human VPIR2). Likewise, because of homology within certain regions of polypeptide sequences of different proteins, specific binding can include an agent that recognizes more than one protein or target. It is understood that, in certain embodiments, an agent that specifically binds a first target may or may not specifically bind a second target. As such, “specific binding” does not necessarily require (although it can include) exclusive binding, i.e. binding to a single target. Thus, an agent may, in certain embodiments, specifically bind more than one target. In certain embodiments, multiple targets may be bound by the same antigen-binding site on the agent. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or other members of the immunoglobulin superfamily, in certain embodiments, a “polypeptide” can occur as a single chain or as two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, the coding region of a nucleotide sequence.

A “conservative amino acid substitution” is one in which one amino acid residue is replaced with another amino acid residue having a similar side chain Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies of the invention do not abrogate the binding of the polypeptide or antibody containing the amino acid sequence, to the target binding site. Methods of identifying nucleotide and amino acid conservative substitutions which do not eliminate binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like, which is to be the recipient of a particular treatment. Typically, the terms “subject” and “patient” are used interchangeably herein in reference to a human subject.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “effective amount” or “therapeutically effective amount” or “therapeutic effect” refer to an amount of an agent described herein, an antibody, a polypeptide, a polynucleotide, a small organic molecule, or other drug effective to “treat” a disease or disorder in a subject such as, a mammal.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus, those in need of treatment include those already with the disorder; those prone to have the disorder; and those in whom the disorder is to be prevented.

The present invention relates to pharmaceutical compositions of a therapeutic (e.g., an hVIPR2 antagonist), or a pharmaceutically acceptable salt, solvate, ester, amide, clathrate, stereoisomer, enantiomer, prodrug or analogs thereof, and use of these compositions for the treatment of a disease such as Schizophrenia.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is Schizophrenia.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the Schizophrenia.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®, hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., an hVIPR2 antagonist, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, cam phorsulfonate, citrate, cyclopentanepropionate, digluconate, dodecylsulfate, ethanesulfonate, fumarate, glucoheptonate, glycerophosphate, hem isulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., Schizophrenia). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8th Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg; from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palm itostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes

The present methods for treating Schizophrenia are carried out by administering a therapeutic for a time and in an amount sufficient to result in decreased positive Schizophrenia symptom, and/or decreased negative Schizophrenia symptom, and/or decreased cognitive Schizophrenia symptom.

The amount and frequency of administration of the compositions can vary depending on, for example, what is being administered, the state of the patient, and the manner of administration. In therapeutic applications, compositions can be administered to a patient suffering from Schizophrenia in an amount sufficient to relieve or least partially relieve the symptoms of the Schizophrenia and its complications. The dosage is likely to depend on such variables as the type and extent of progression of the Schizophrenia, the severity of the Schizophrenia, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the Schizophrenia or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 pmol of therapeutic (e.g., 0.1-25 pmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of Schizophrenia.

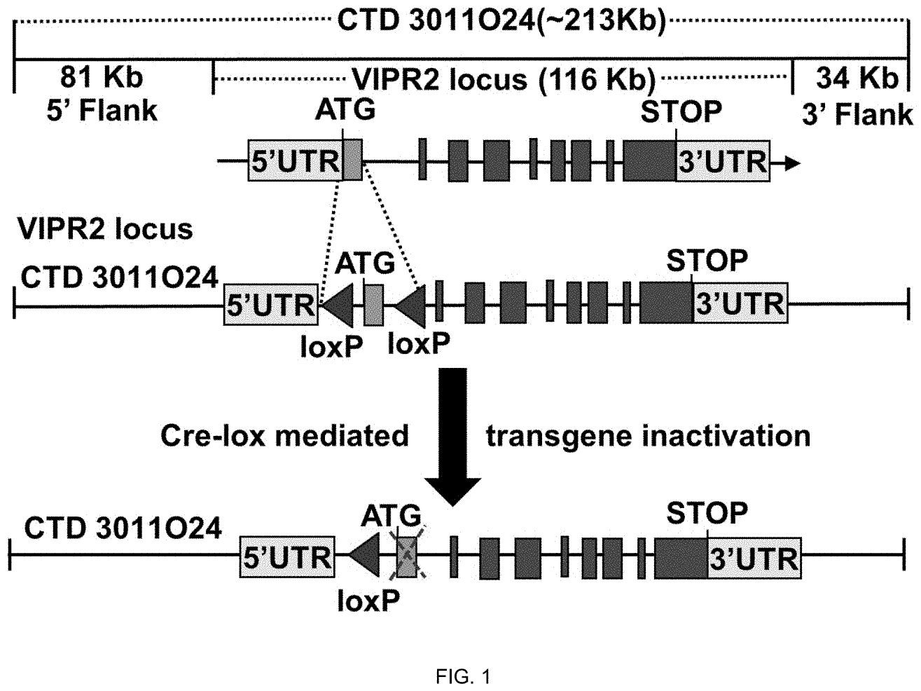

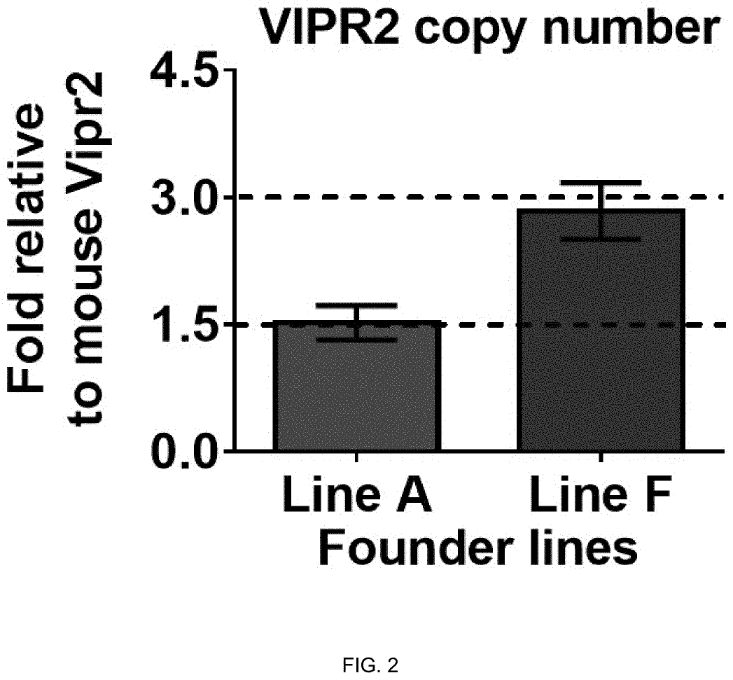

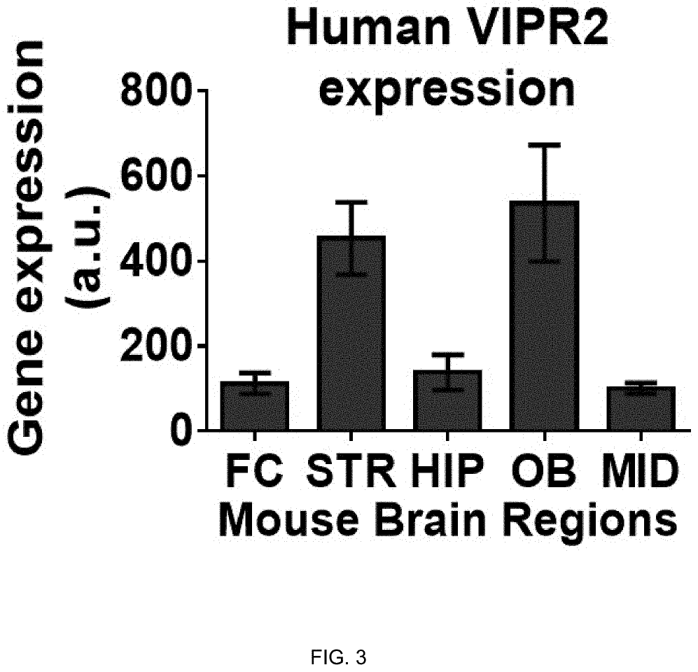

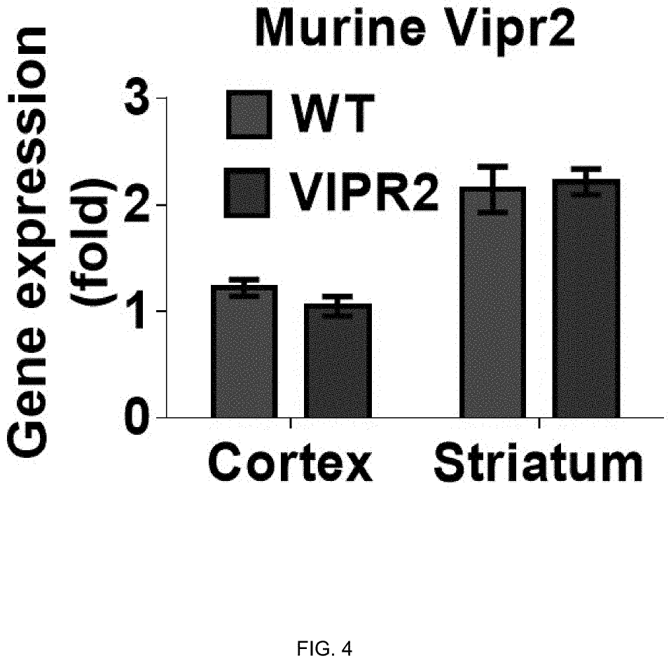

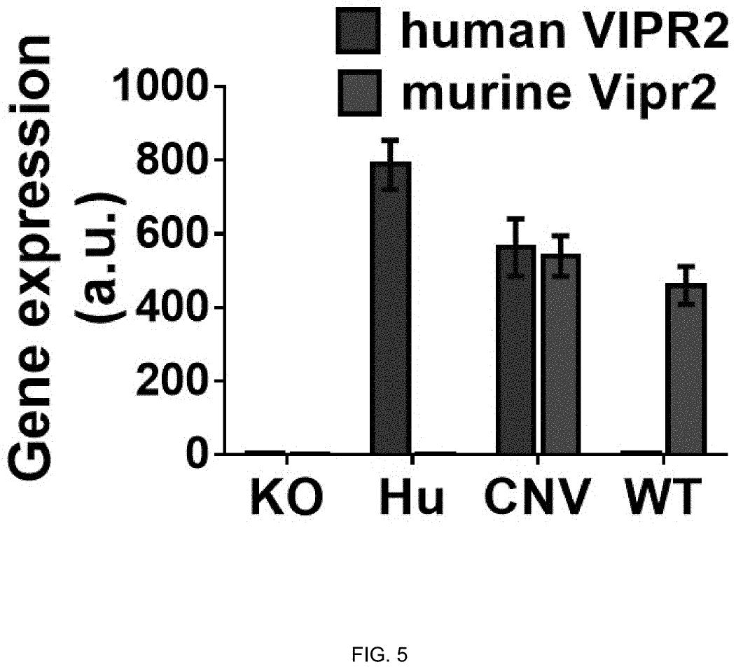

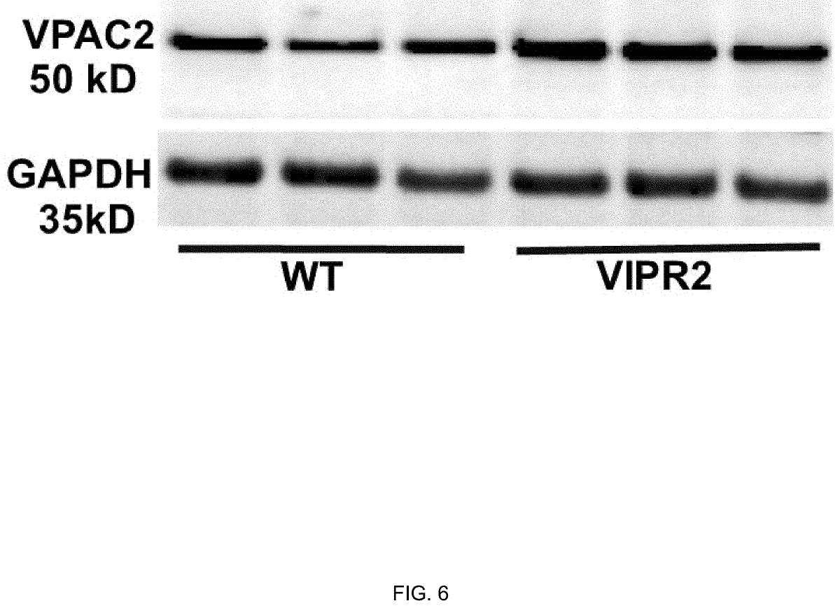

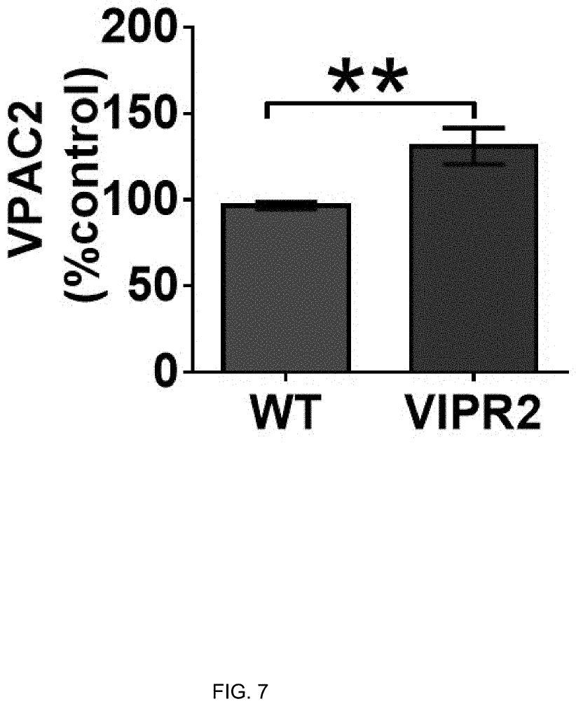

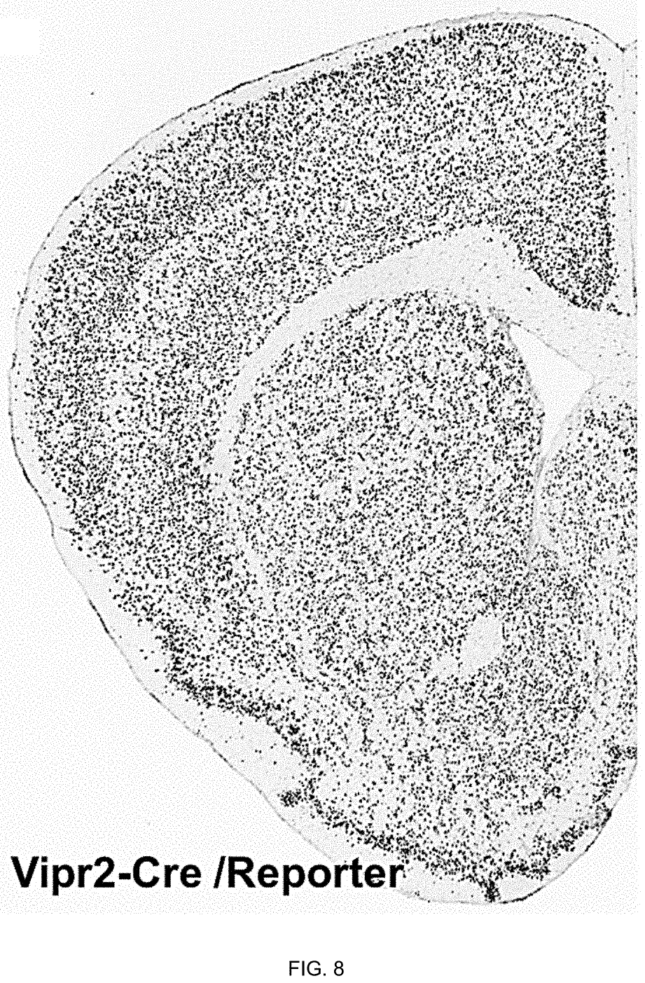

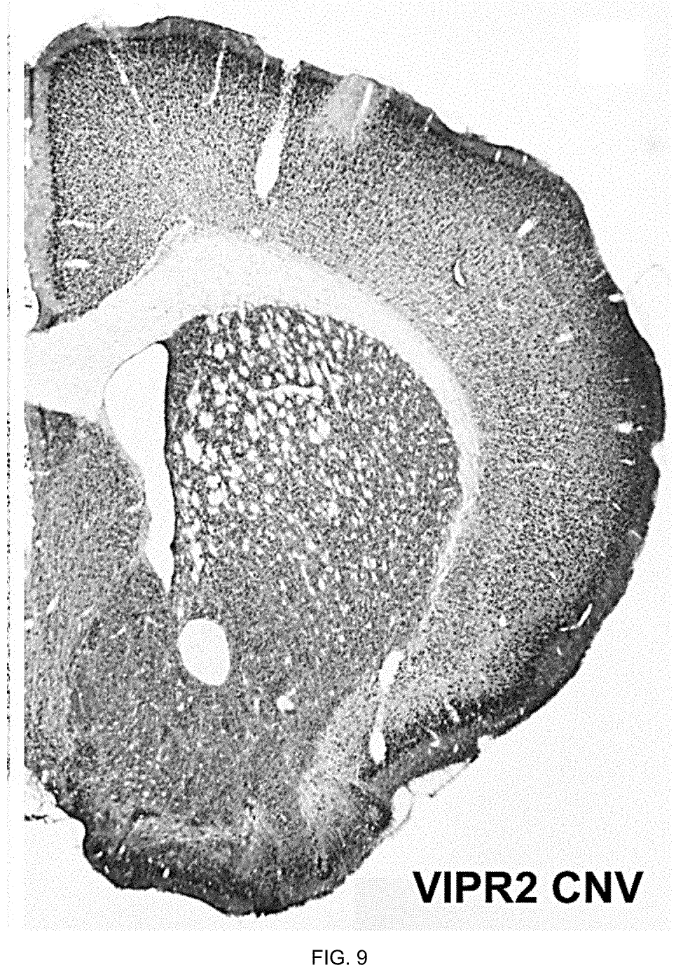

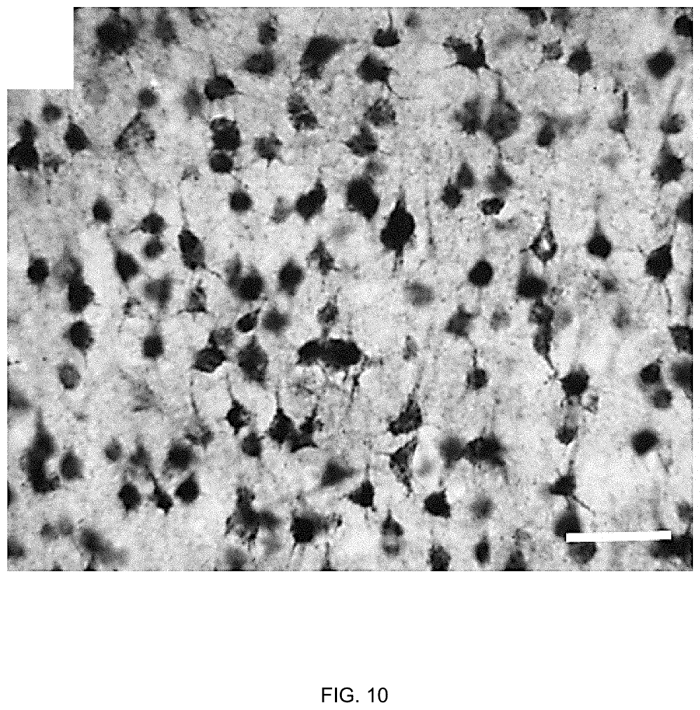

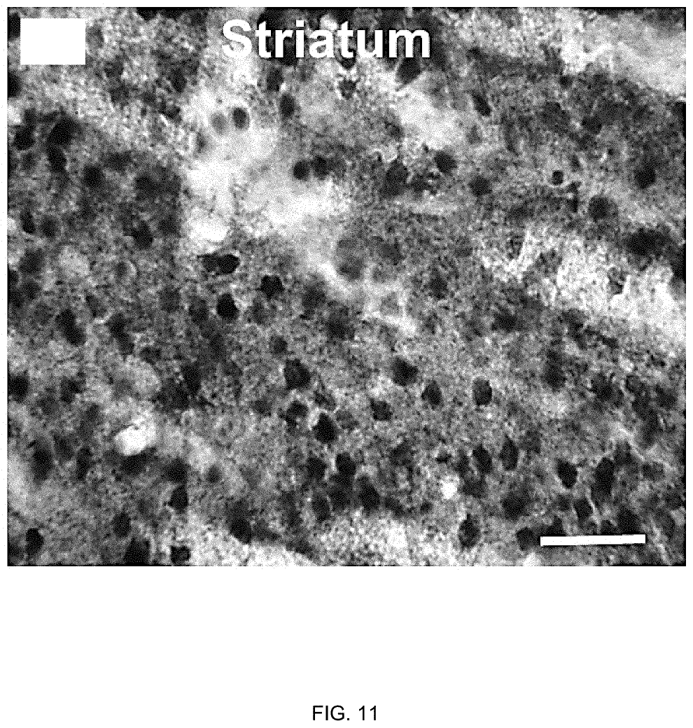

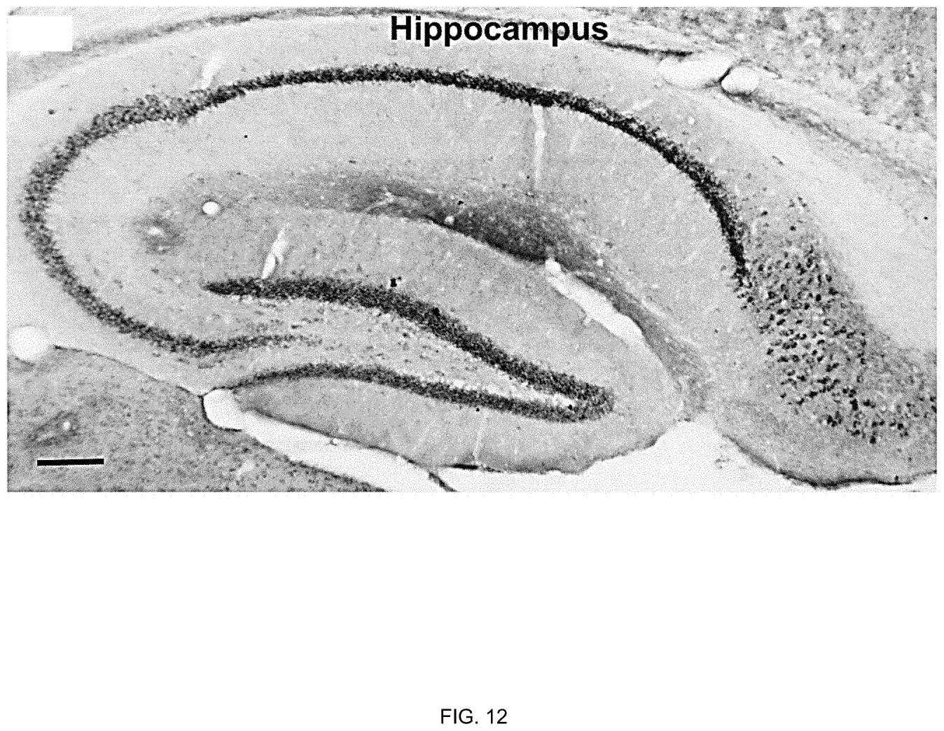

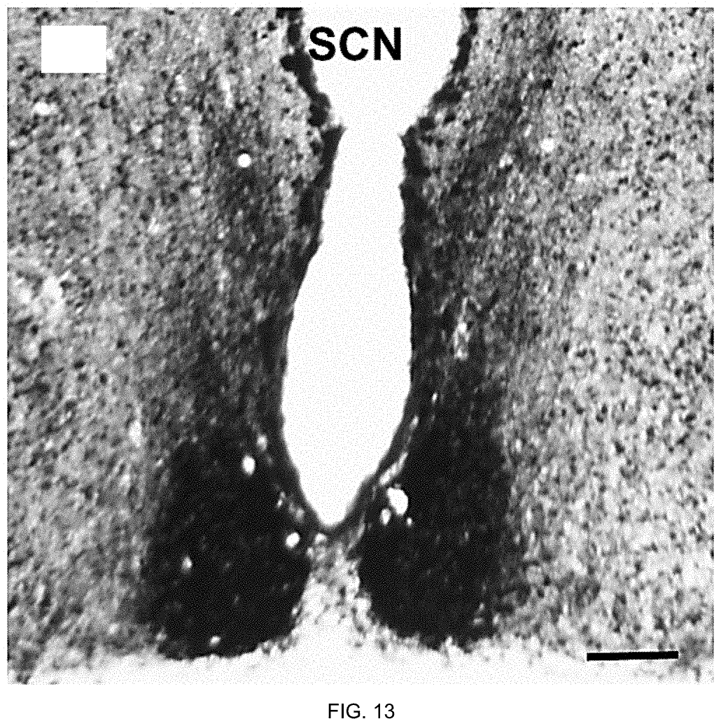

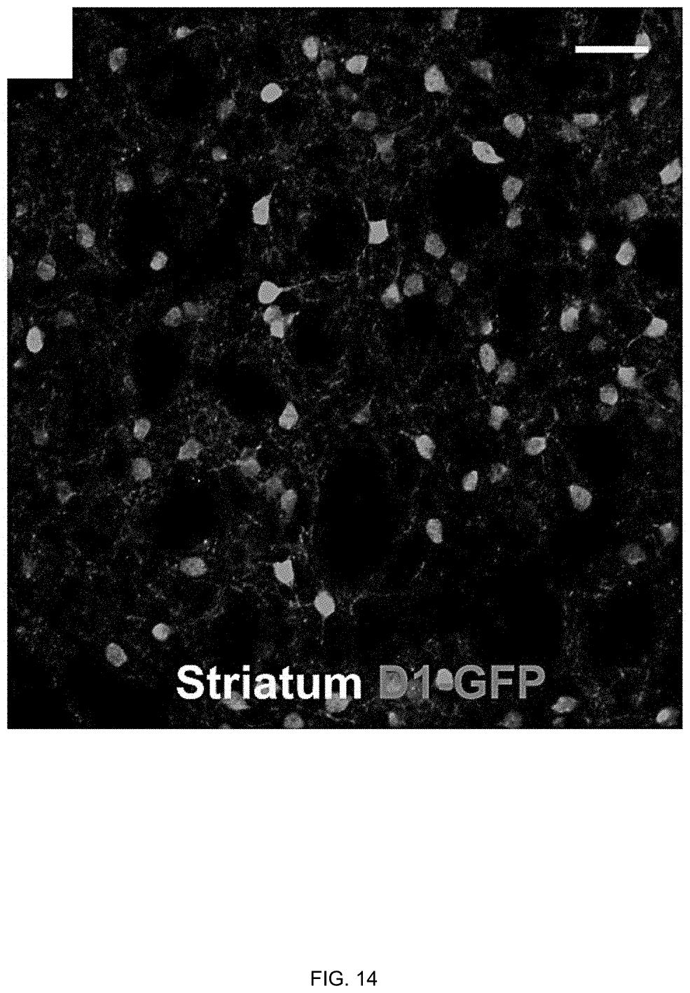

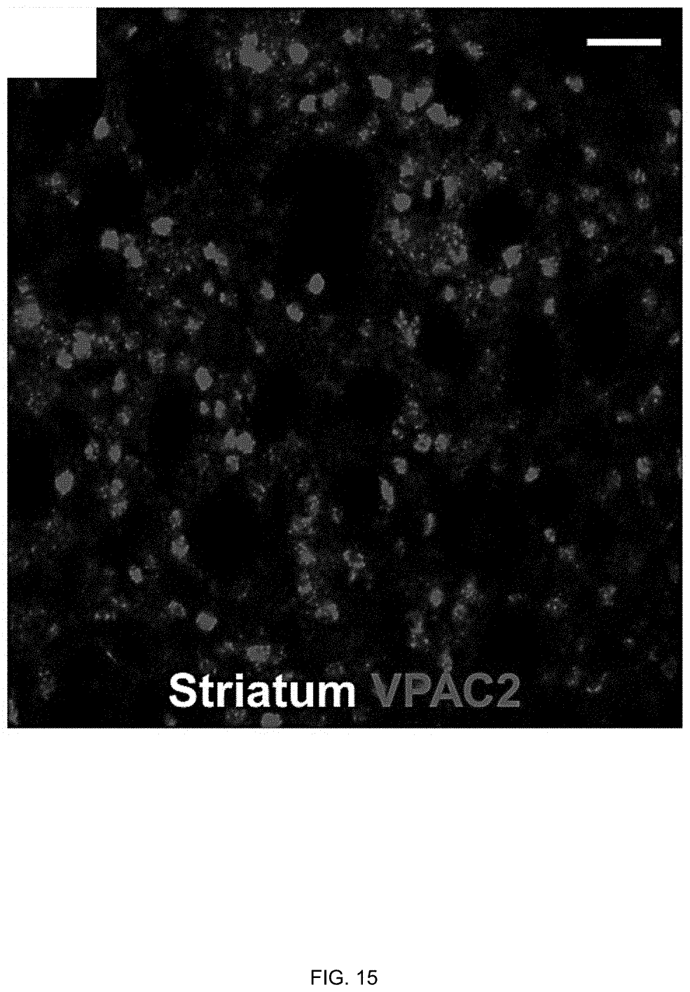

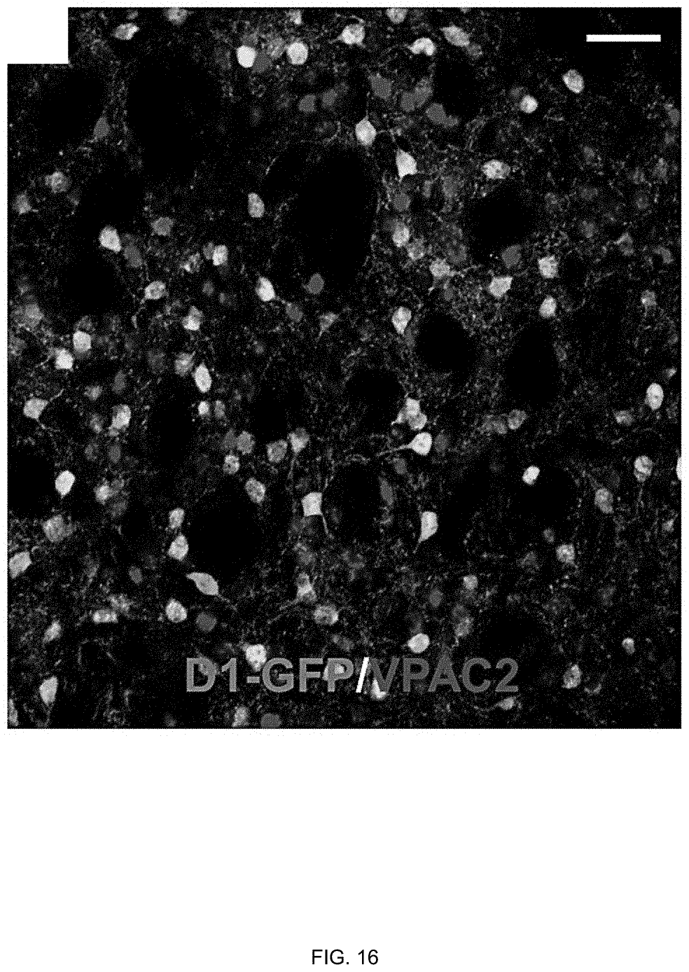

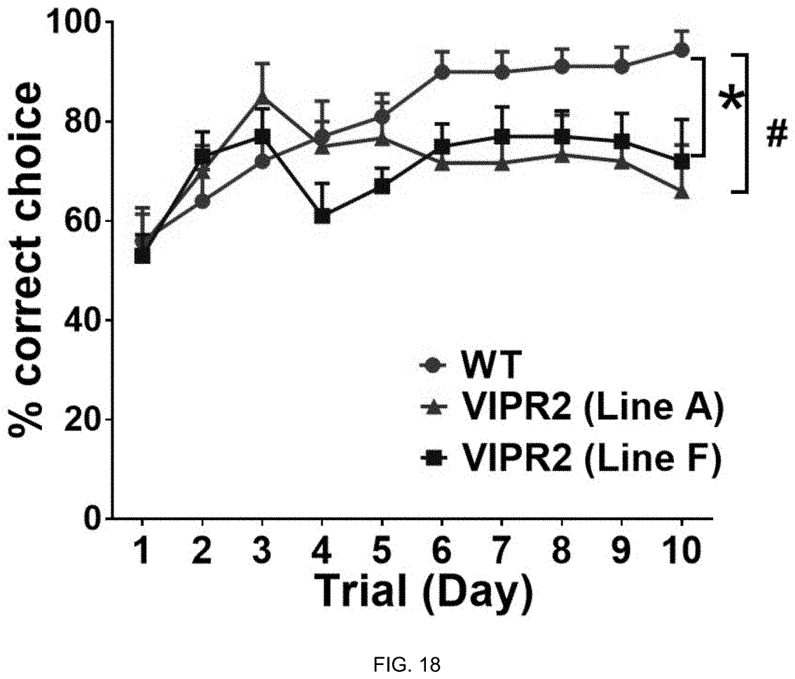

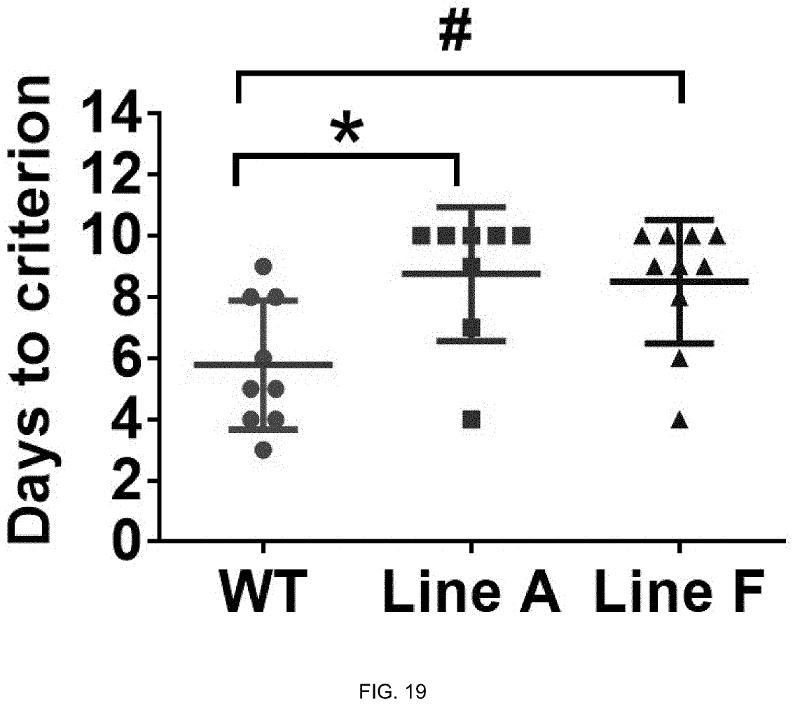

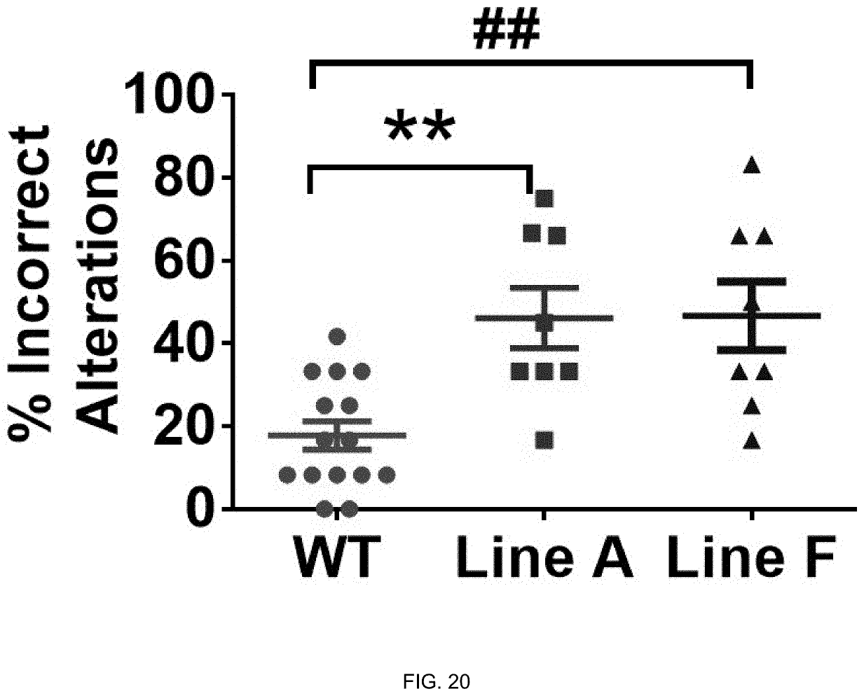

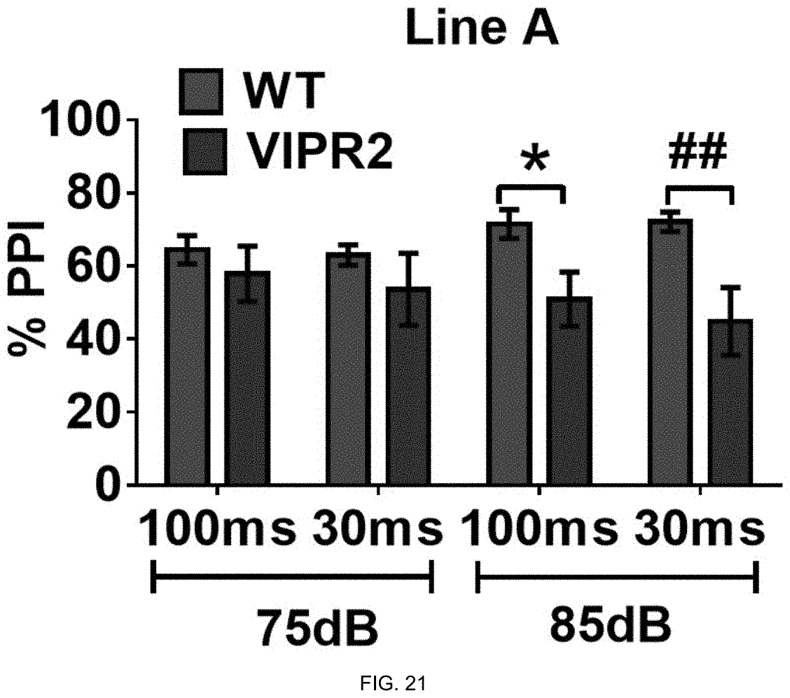

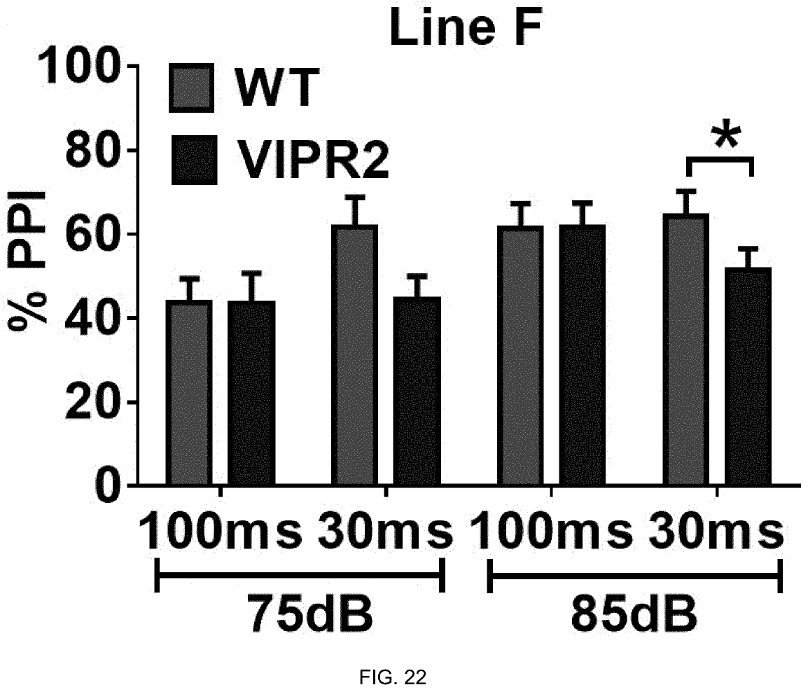

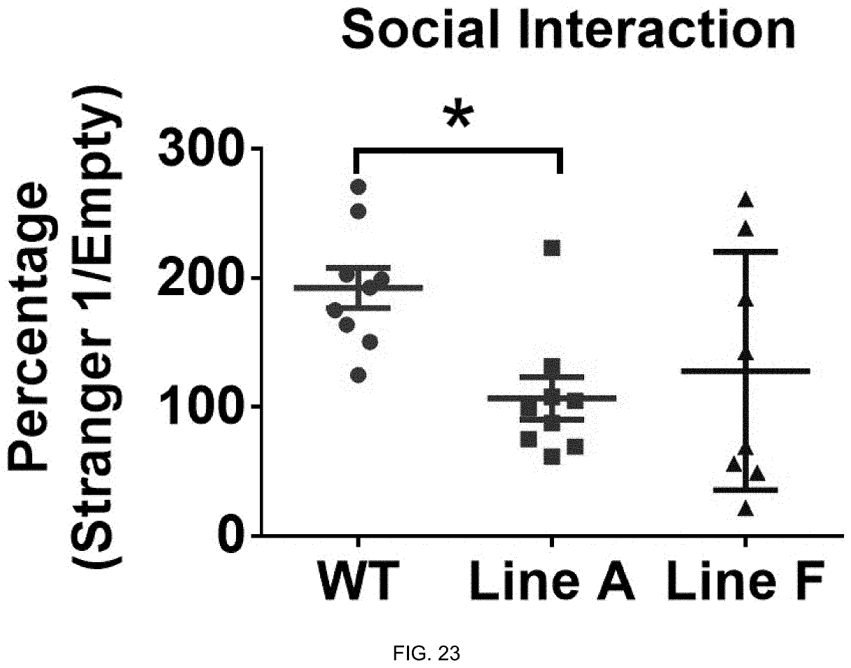

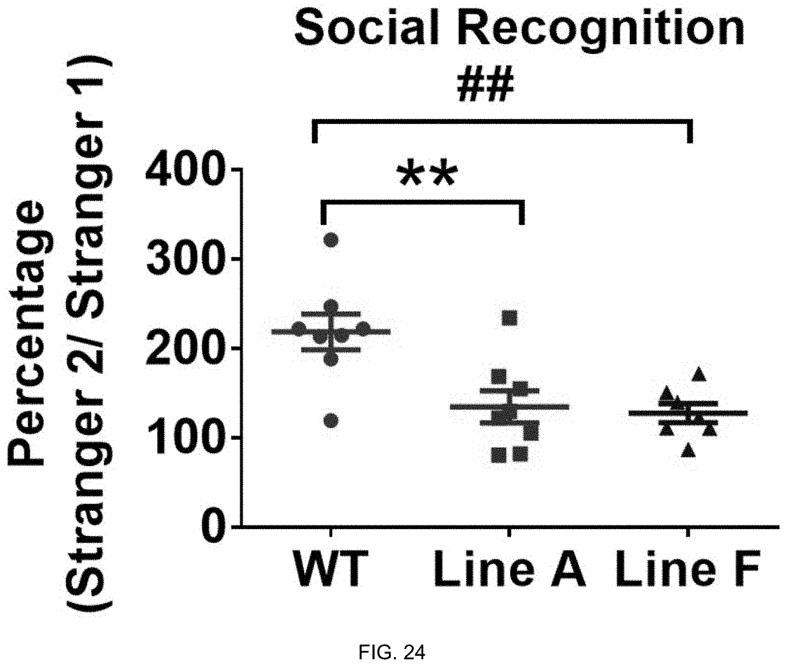

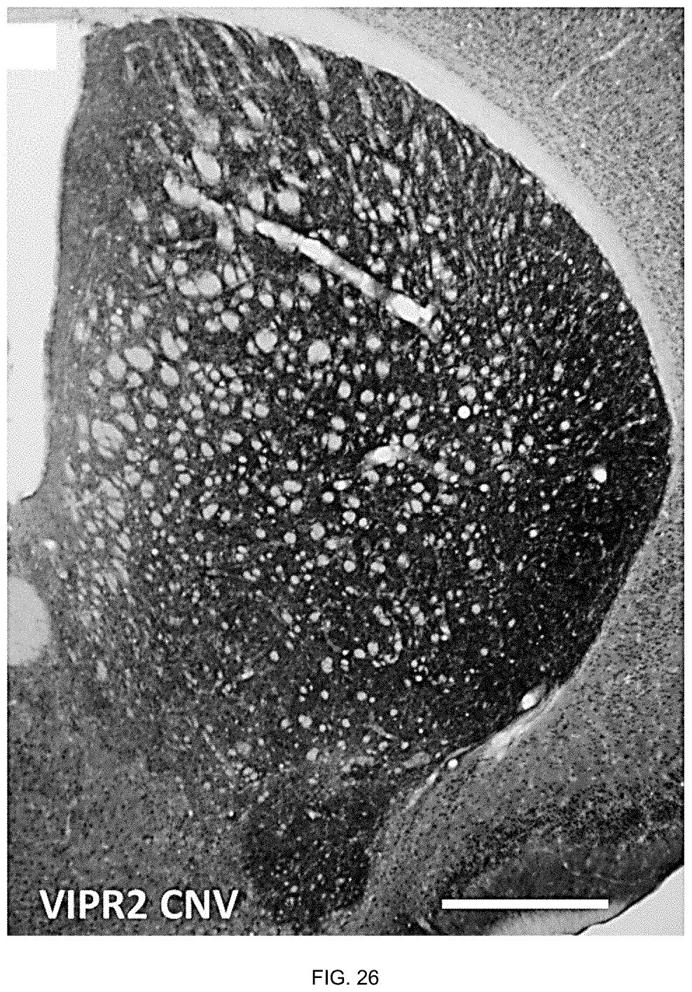

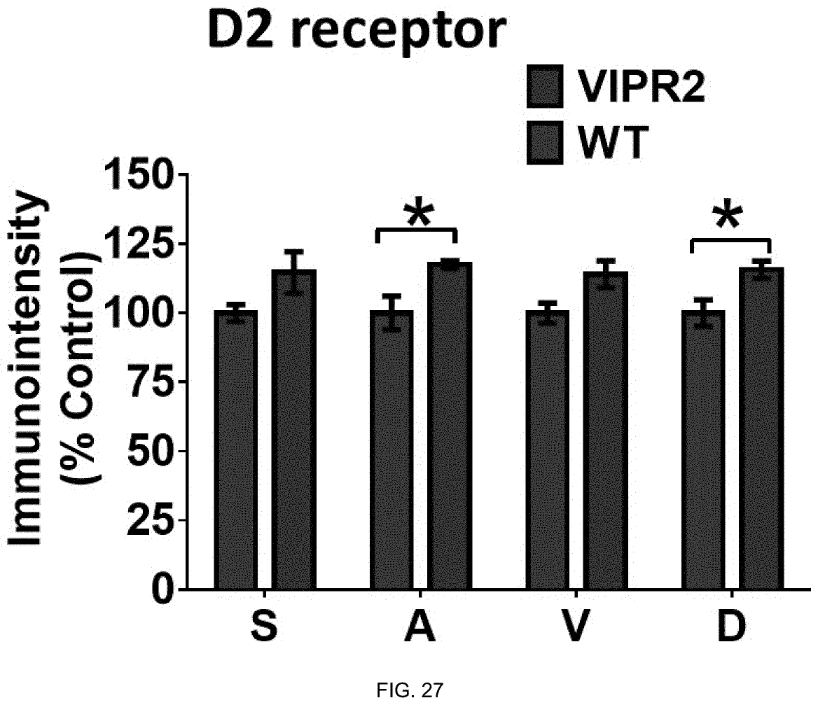

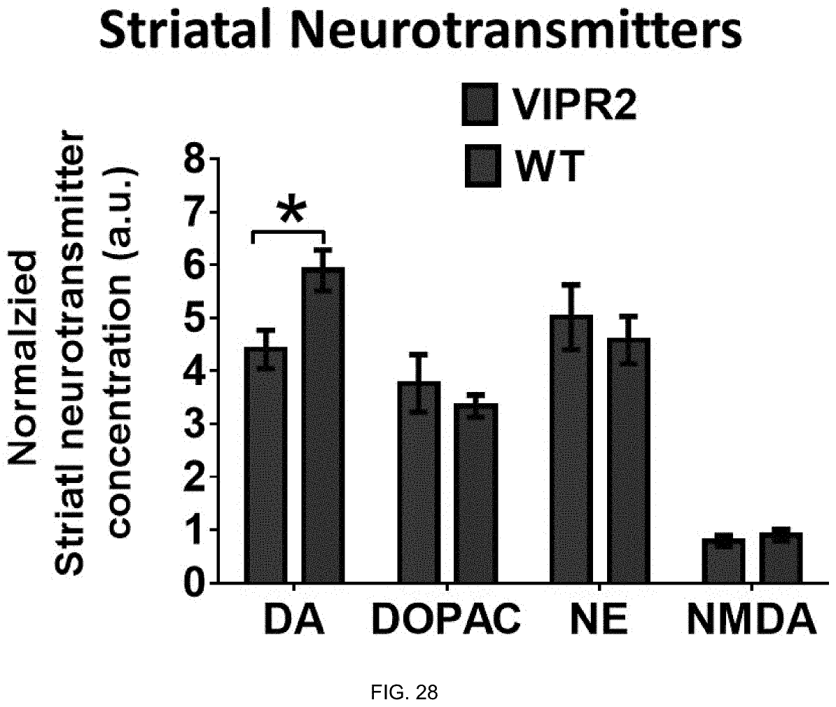

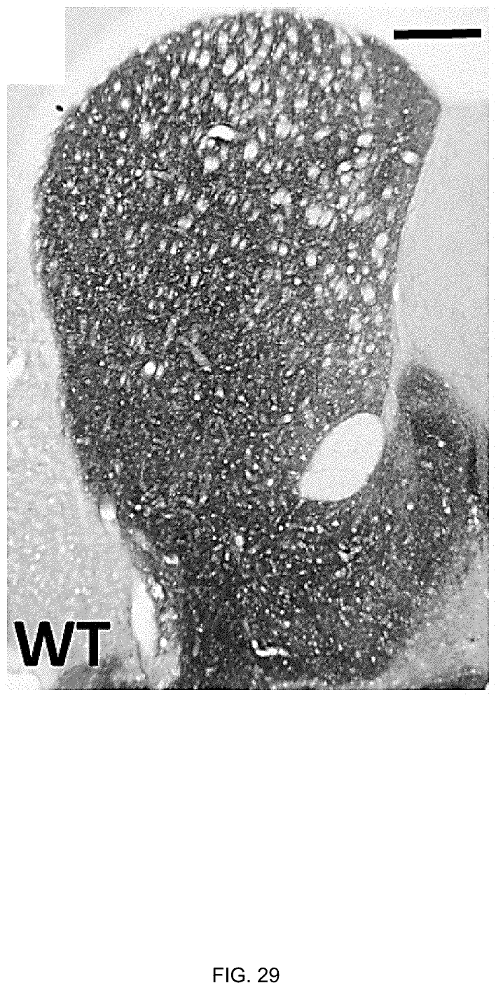

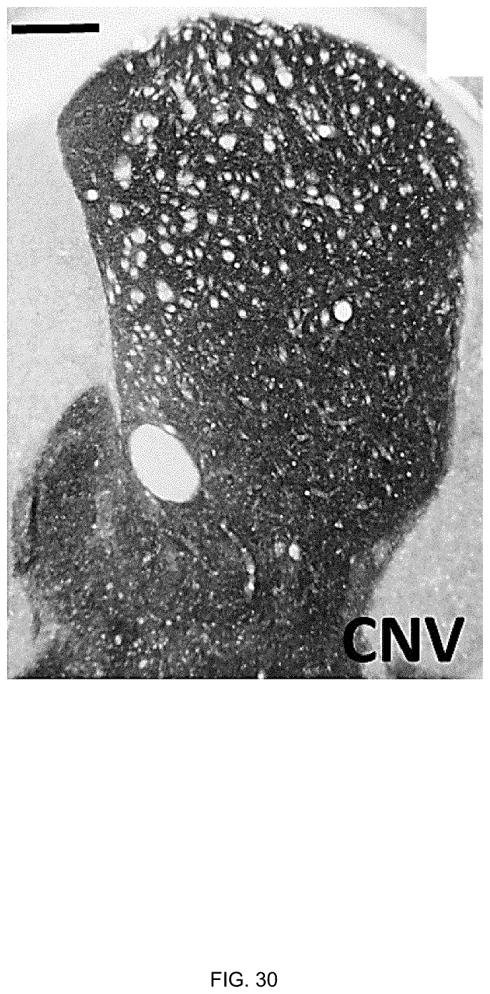

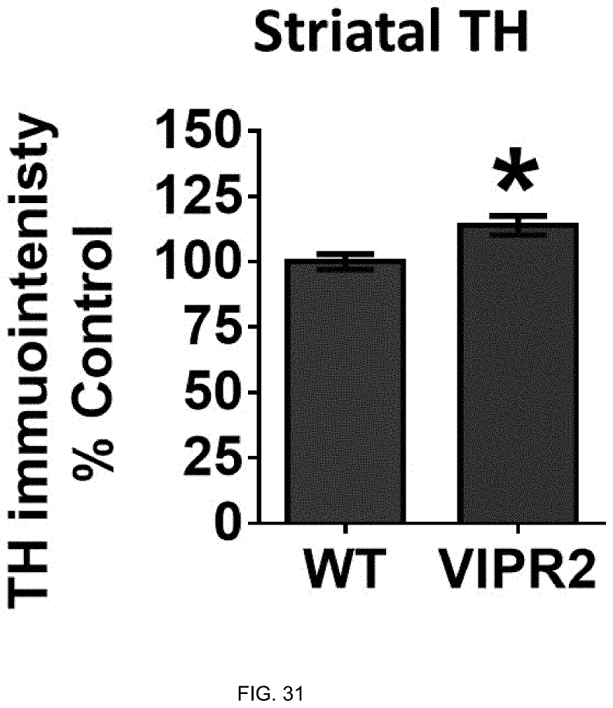

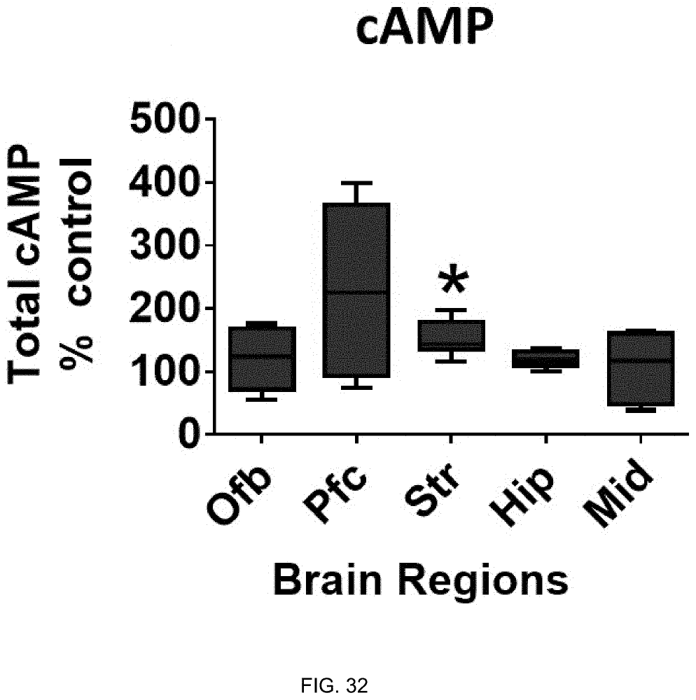

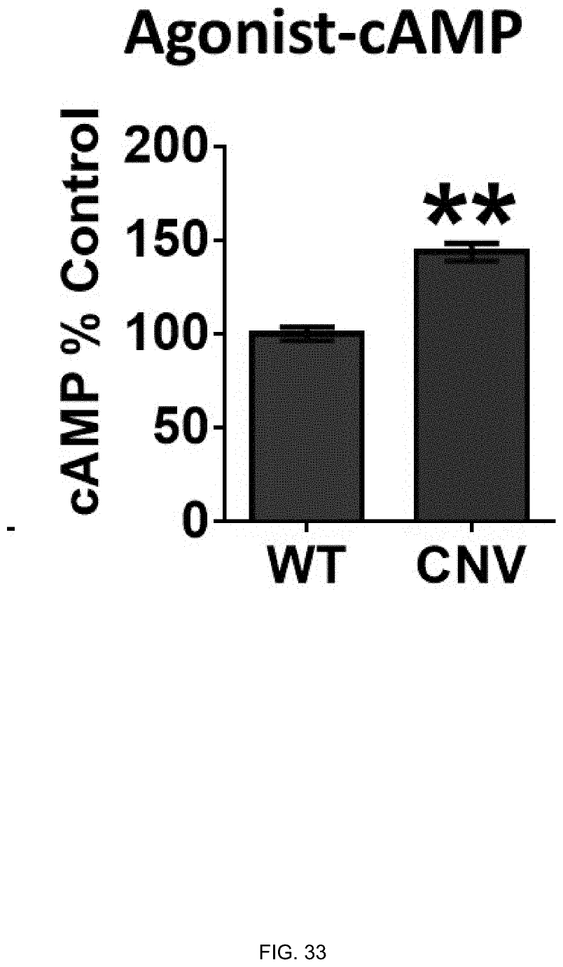

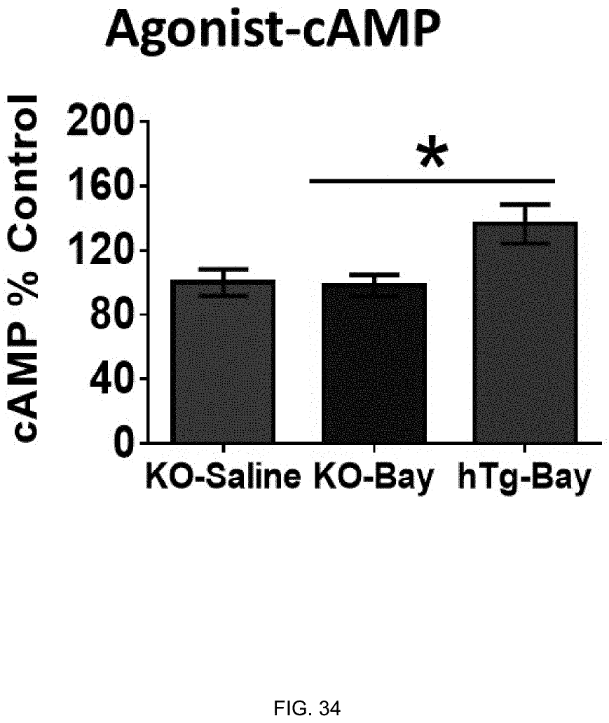

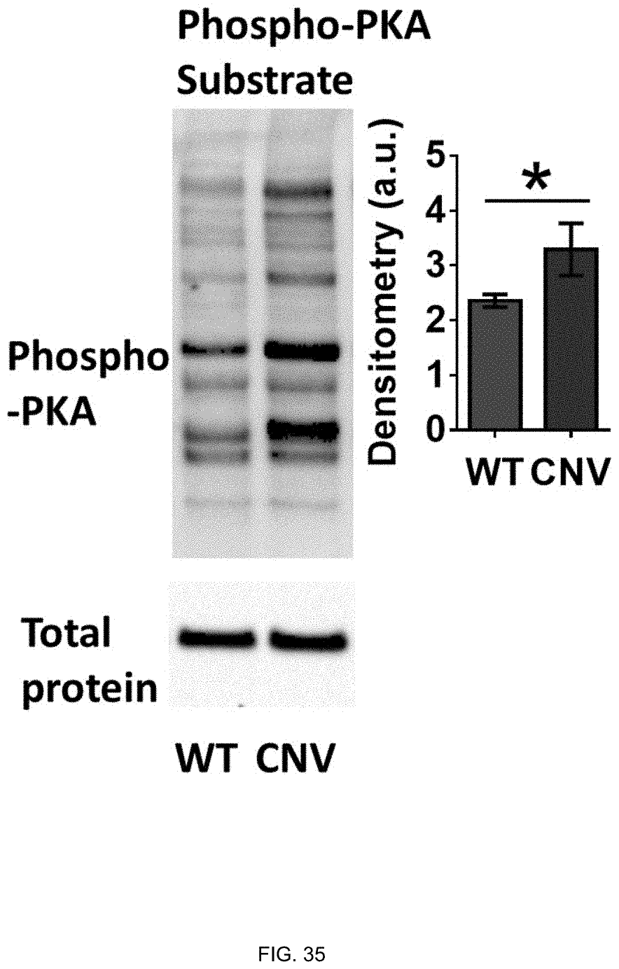

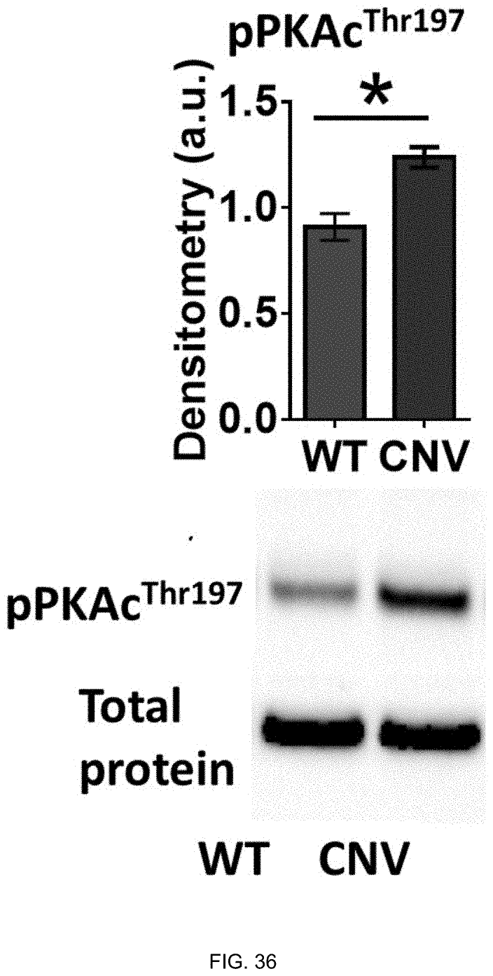

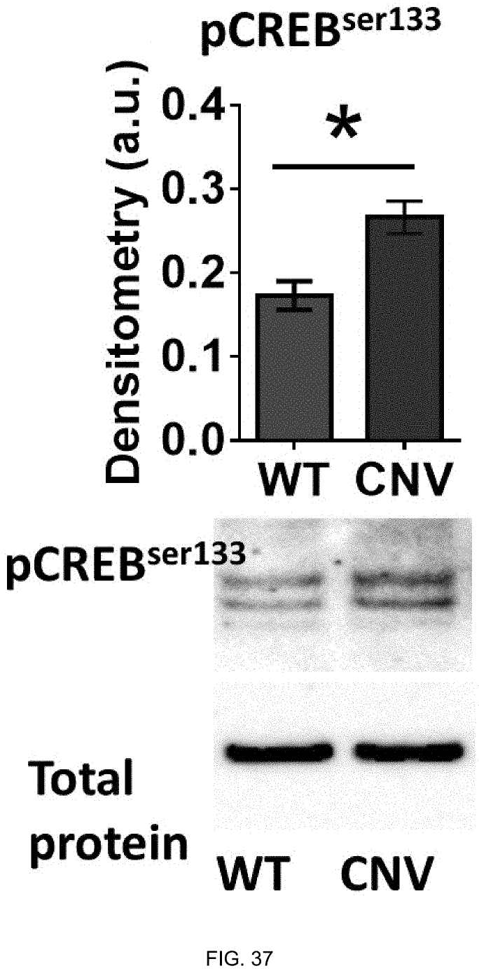

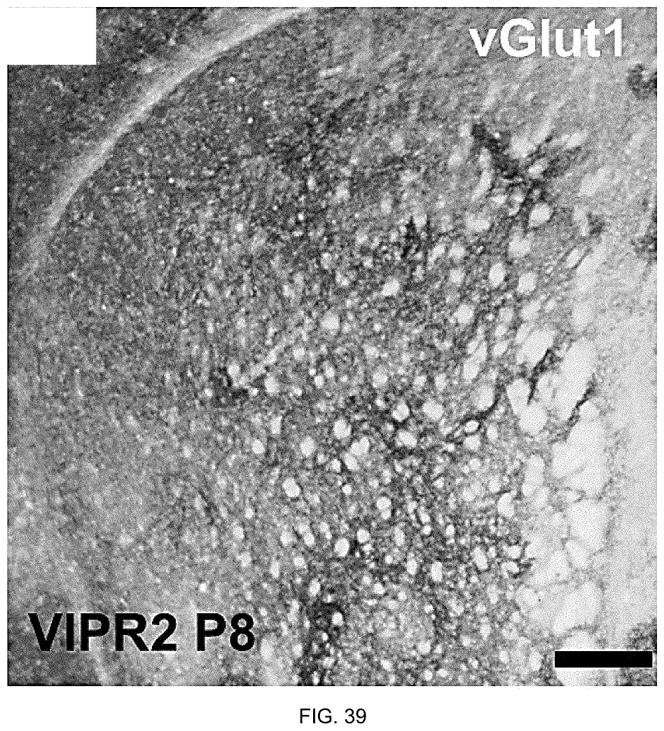

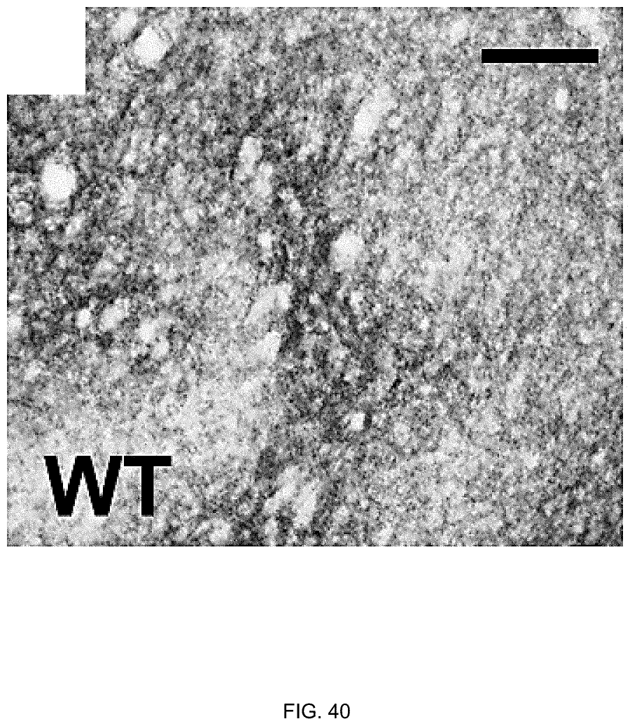

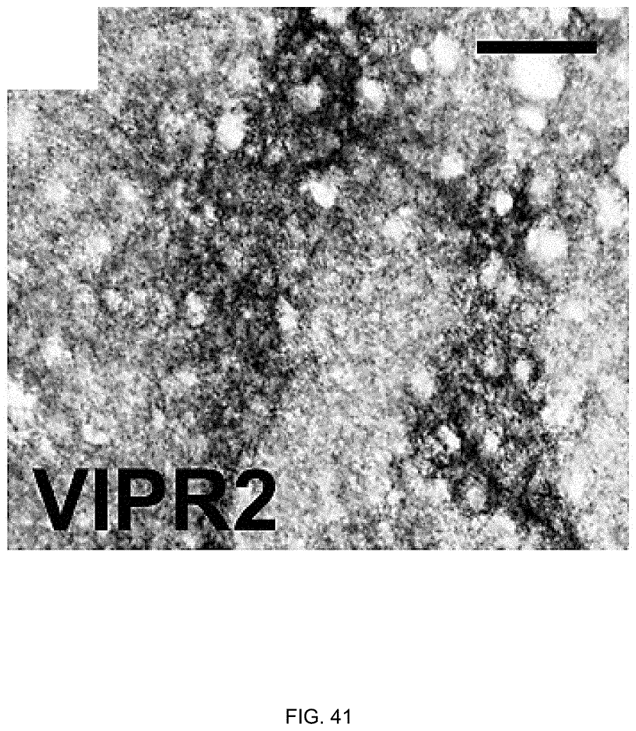

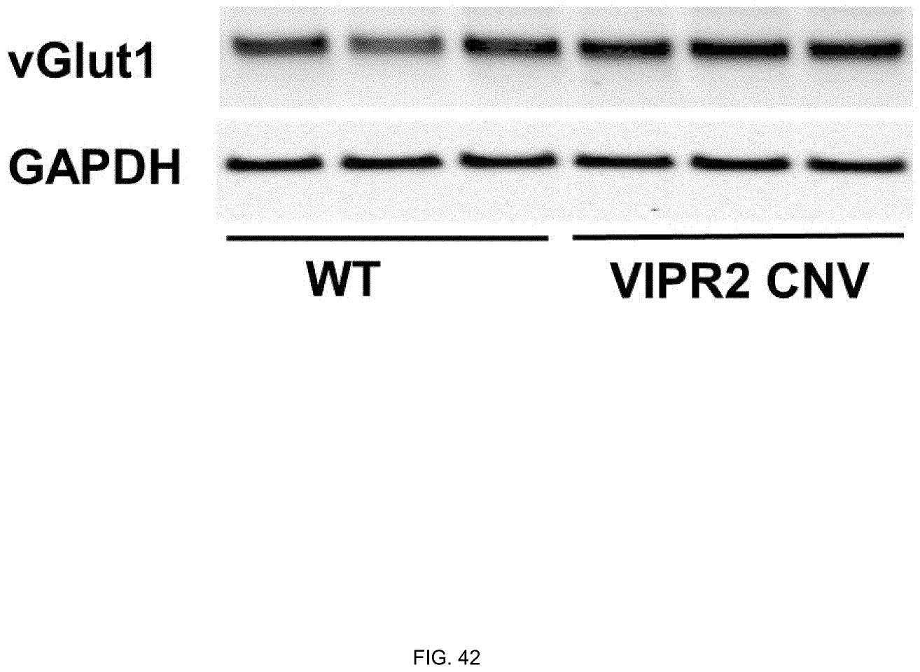

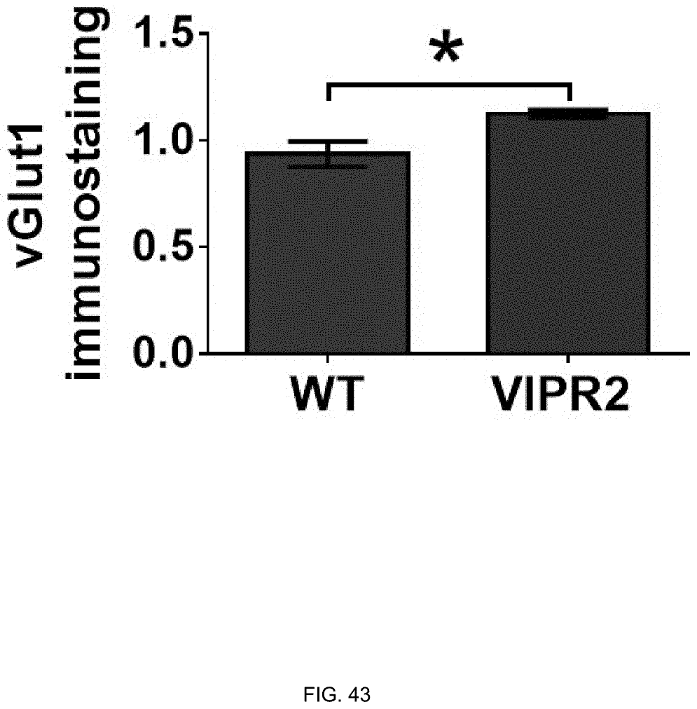

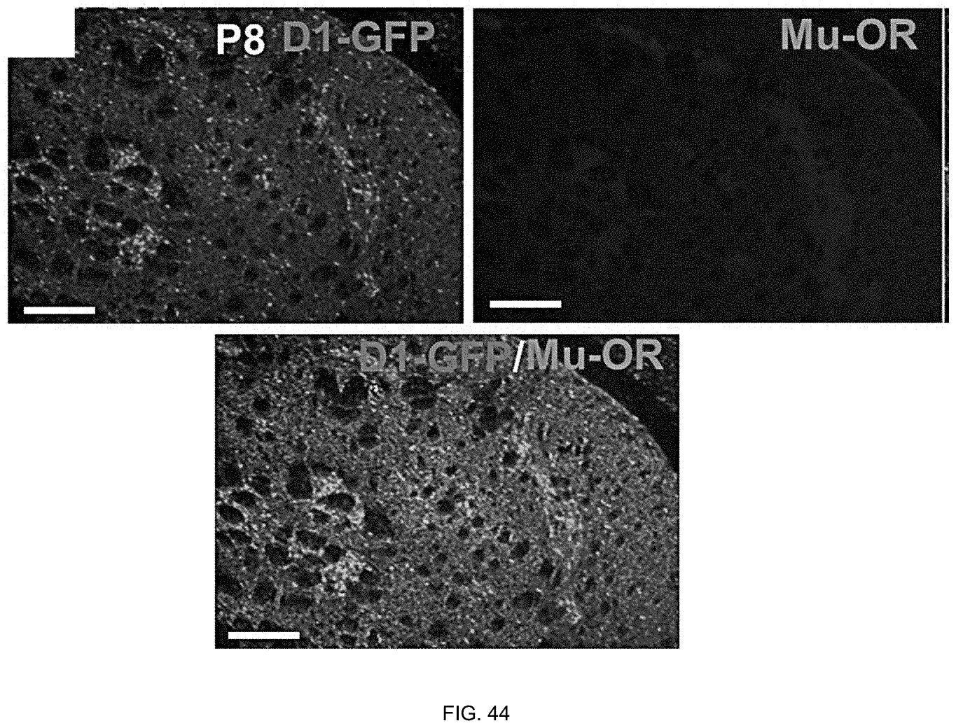

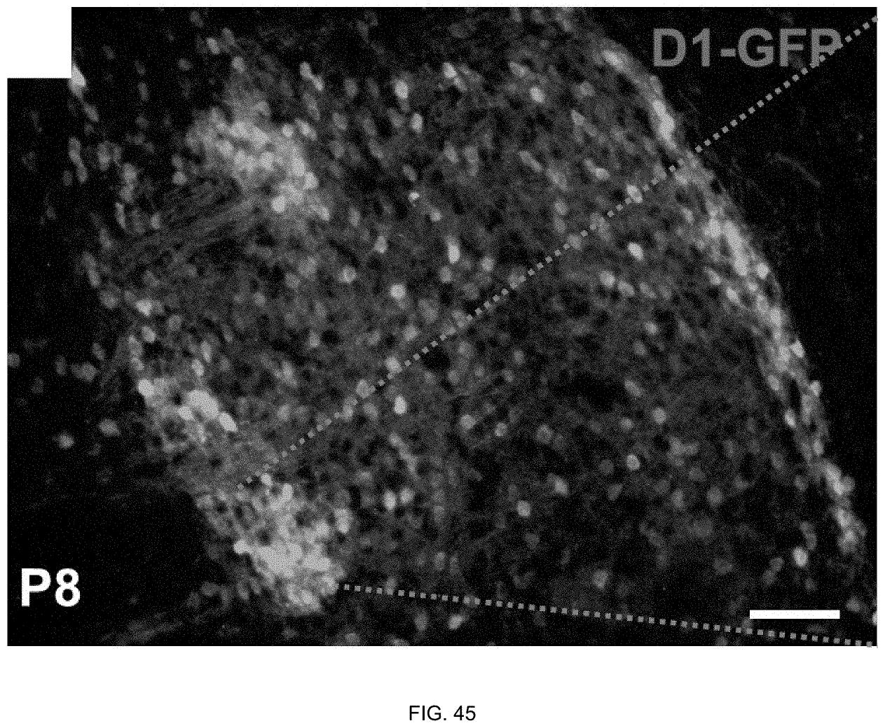

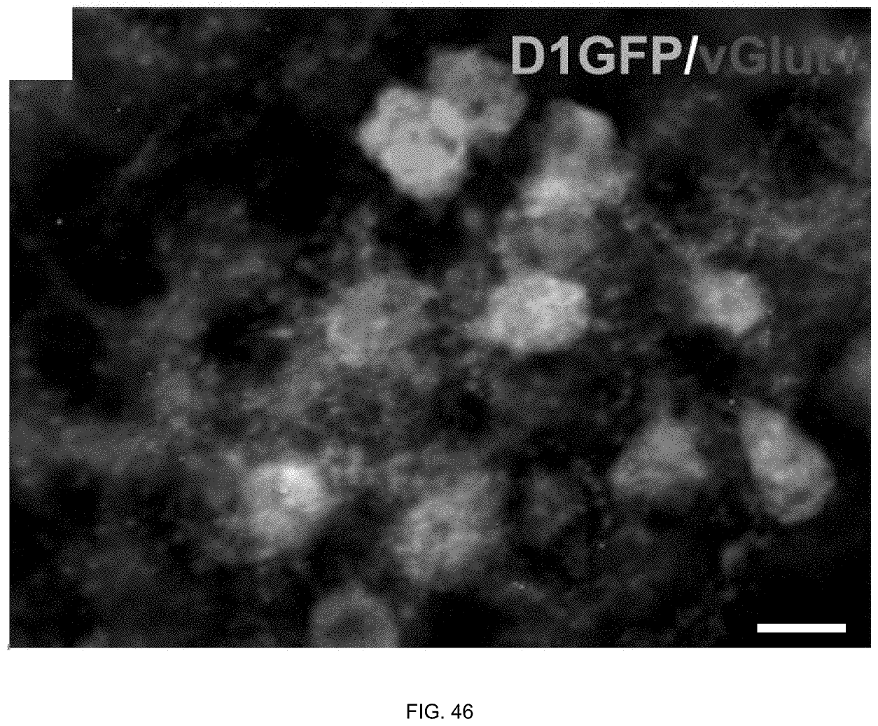

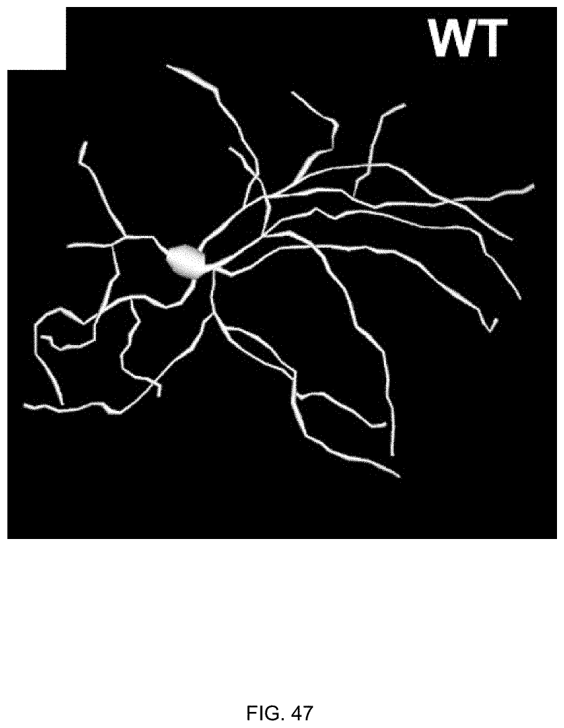

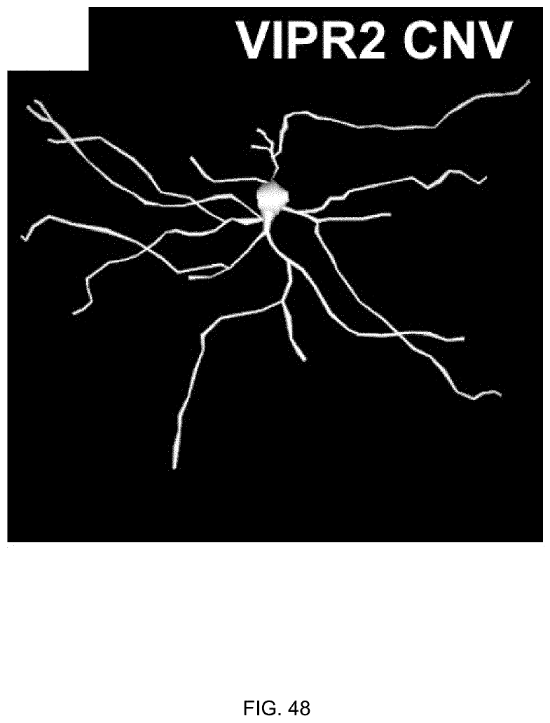

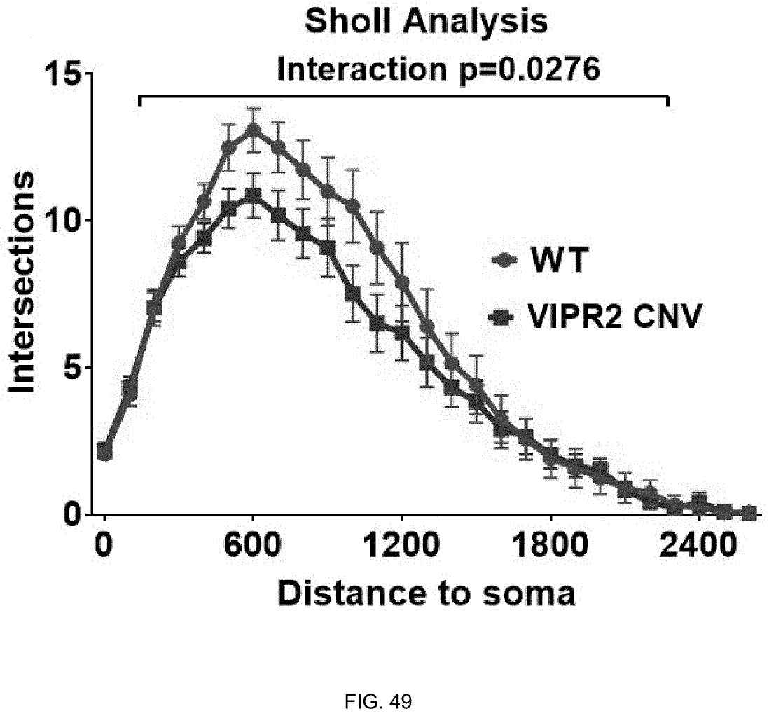

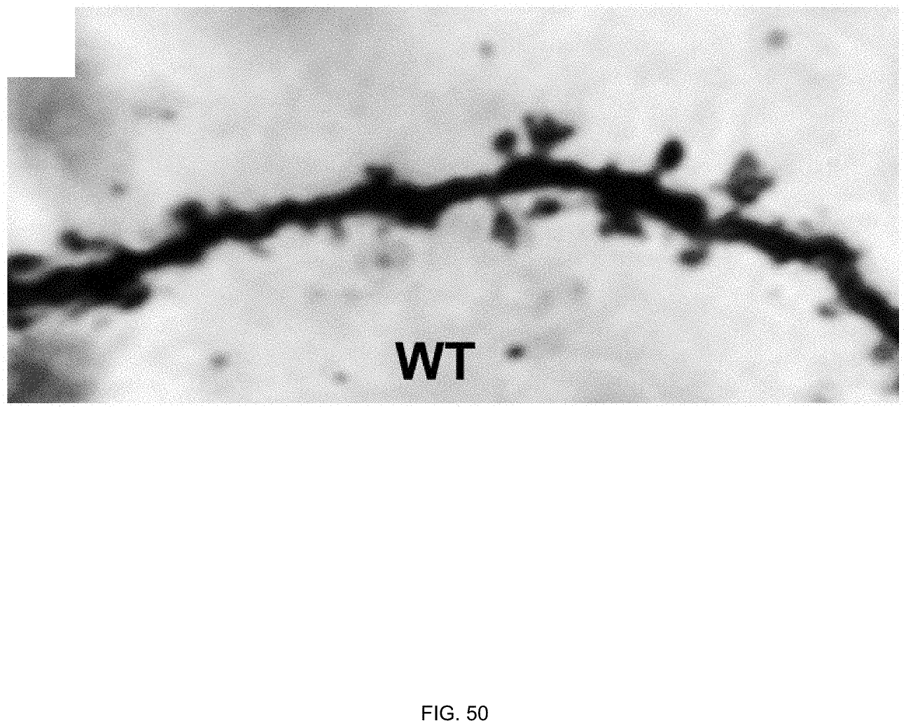

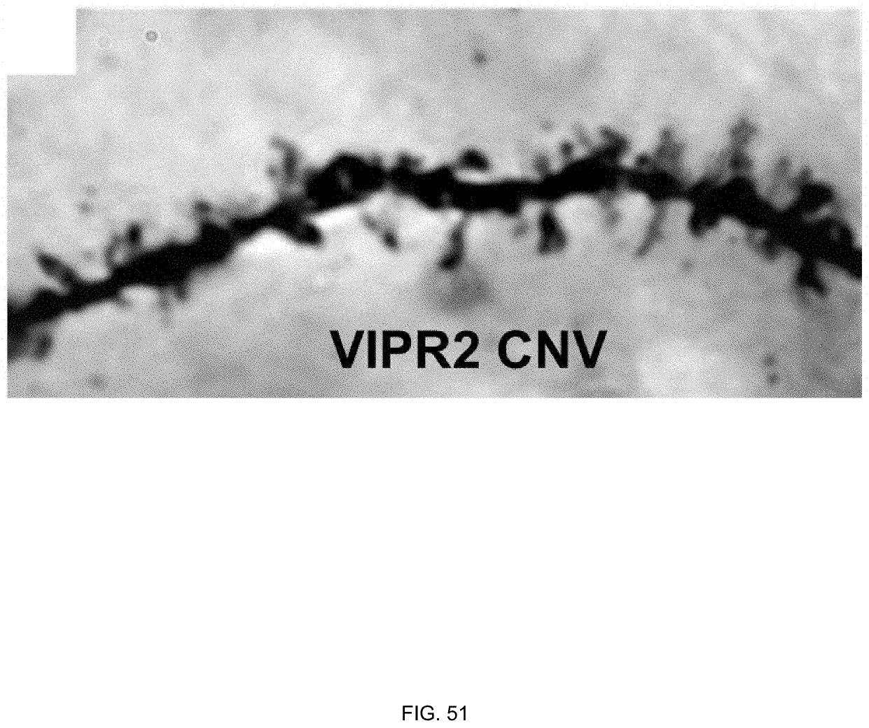

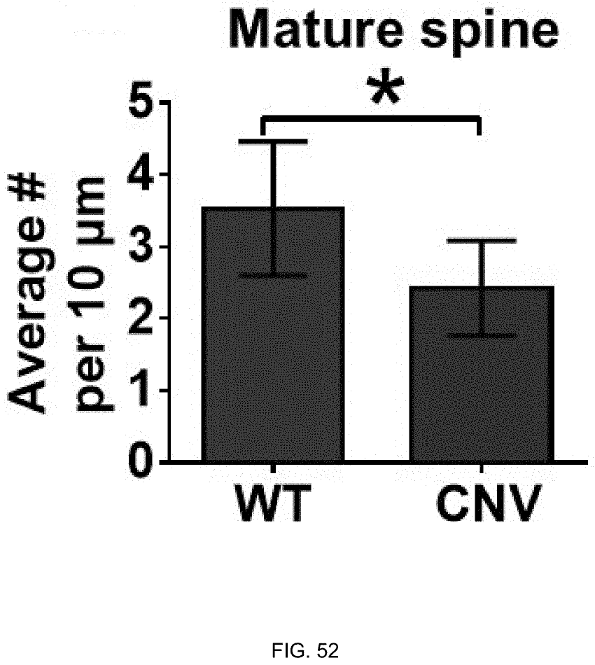

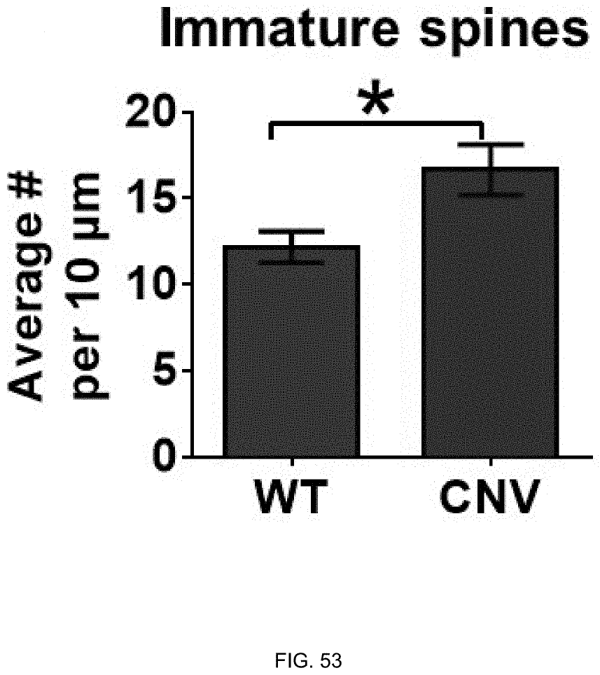

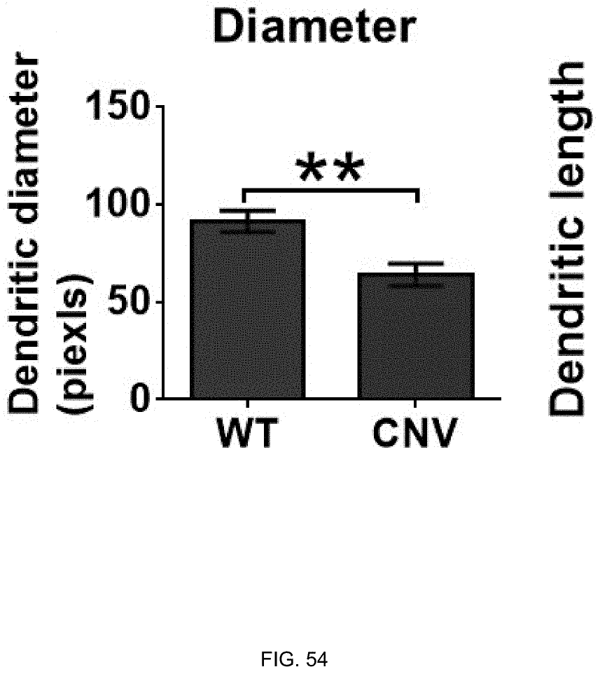

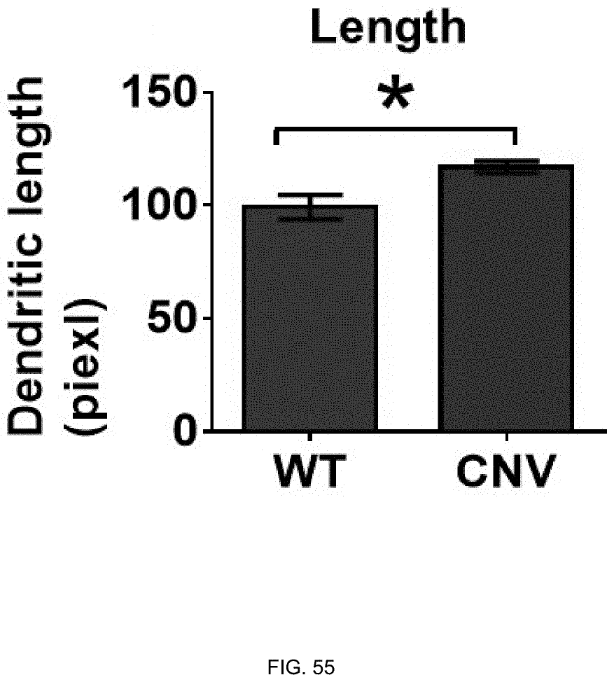

Turning now to FIGS. 1-57, a brief description concerning the various components of the present invention will now be briefly discussed.

Recently, genome-wide association studies (GWAS) have identified multiple disease-associated, and evidence of causative, chromosome structural mutations, or copy number variants (CNVs), which have been convincingly shown to increase the risk of Schizophrenia. In particular, two large-scale GWAS studies pinpointed a CNV at the chromosomal locus 7q36.6 in Schizophrenia patients at a rate 14 times higher than in healthy individuals, with all of the microduplications and triplications occurring within a single gene: Vasoactive intestinal peptide receptor 2 (VIPR2, also known as VPAC2). In the latest and the largest genome-wide searches of CNVs for Schizophrenia by the working psychiatric genomics consortium, Schizophrenia patients were reported to carry a mean of 11° A more CNVs than controls and 7q36.6 (which is on the long (q) arm of chromosome 7 at position 36.6) was again listed as a candidate susceptibility loci. VIPR2 gene encodes the VPAC2 receptor, whose ligands are vasoactive intestinal peptide (VIP) and pituitary adenylate cyclase-activating polypeptide (PACAP). VIPR2 is a stimulatory canonical G-Protein Coupled Receptor Pathway (GPCR) that activates adenylyl cyclase (AC)-cyclic adenosine 3′,5′-mono-phosphate (cAMP)- protein kinase A (PKA), and is, therefore, a druggable target. In the lymphocytes of Schizophrenia patients with VIPR2 duplication, cAMP signaling was significantly increased, implicating the VIPR2 receptor antagonist as a potential novel antipsychotic agent. The implicated causative role of VIPR2 duplication allows the generation of an etiologically relevant animal model and the opportunities to validate drug targets in the etiologically relevant animal model that are integrated with translationally realistic endpoint assessments. Such a strategy will arguably reduce the current attrition rate in Schizophrenia drug discovery and ultimately lead to therapies that tackle the disease process.

The inventors have conceived and reduced to practice the disclosed invention. Turning now to FIGS. 1-16, a brief description design and genetic characterization of the generation of VIPR2 BAC transgenic mice is performed A large insert cloning system, such as bacterial artificial chromosome (BAC) mediated transgenesis offers the potential for accurate reproduction of VIPR2 gene duplication and is likely to confer endogenous-like, dosage-dependent transgene expression. BACs are F-element based circular plasmids that propagate up to 300 kb of genomic DNA in E. coli. In BAC transgenic mice, multiple copies of BACs are randomly integrated into the genome, often in tandem repeats, thus recapitulating the microduplication of chromosomes as seen in human CNVs. Importantly, for most mammalian genes, BACs are likely to contain all the regulatory elements (promoter, enhancer, silencer, splicing enhancer, etc.), and therefore can confer endogenous-like, gene dosage-dependent transgene expression in vivo.

The inventors have successfully engineered a human VIPR2 BAC (CDT-3011024, SEQ ID NO: 1). Importantly, the first exon of VIPR2, which contains the endogenous translation initiation codon, was floxed by two loxP sites, The LoxP sites are located in the 5′ untranslated region of VIPR2 and in intron 1 flanking VIPR2-exon 1. Thus, these LoxP sites do not interfere with the expression of VIPR2 but do allow for Cre-mediated excision of hVIPR2 exon I. As a result, the VIPR2 CNV model is designed to be a conditional inactivation model in which Cre can switch off transgene in desired temporal and spatial patterns controlled by crossing with mice expressing Cre recombinase. The inventors confirmed that the VIPR2 BAC was correctly modified by using two sets of primers that flank the modification regions. Maxiprep DNA was prepared from the modified VIPR2 BACs and purified through cesium prep. The purified DNA was separated on the pulsed-field gel to verify the integrity of the BAC. The intact BAC DNA fraction was selected and microinjected into 200 fertilized FVB mouse zygotes to generate BAC transgenic mice.

Out of 54 pups born, the inventors have identified six positive transgenic founders using three different pairs of primers that are specific for the modification region. The positive transgenic founders are bred with C57BL/6J wildtype mice to maintain the lines. Further characterization of the animal model using quantitative PCR has identified multiple copies of human VIPR2 genes integrated into the mouse genome, ranging from two to eight copies in different transgenic founder lines. Quantitative PCR analyses of the genomic DNA revealed that VIPR2 CNV mice have tandem integrates of approximately one and four copies of the BAC transgene. Line A was found to have one extra human copy of VIPR2, and Line F has four extra copies of human VIPR2 genomic sequences.

Human VIPR2 transgene expression in mice. The inventors have used qPCR, western blot and immunostaining to define the expression pattern of human VIPR2 in more detail. The inventors have confirmed that similar to the mouse Vipr2 gene expression pattern, human VIPR2 transgene in the inventors' model has a modest level of expression in the frontal cortex, striatum, and hippocampus and a high expression level in the olfactory bulb and suprachiasmatic nucleus. The inventors have found transgene expression in multiple brain regions including: the cortex, striatum, hippocampus, thalamus, and suprachriasmatic nucleus (SCN). hVIPR2 is expressed in direct or indirect pathway spiny projection neurons (dSPNs or iSPNs) in striatum as demonstrated by the double fluorescence staining in Drd1a-GFP BAC and Drd2-GFP BAC transgenic mice.

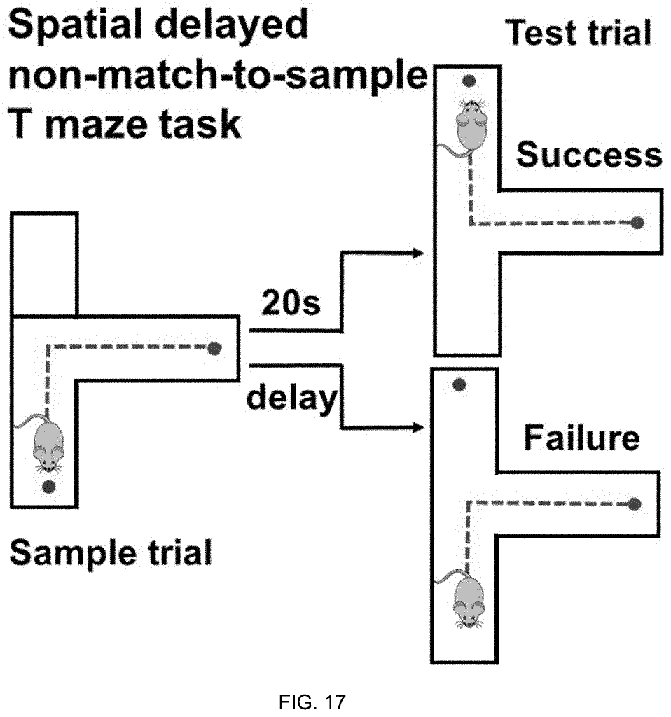

Turning next to FIGS. 17-24, a description of Schizophrenia-like cognitive and social behavioral deficits in VIPR2 CNV mice. Adult VIPR2 CNV BAC transgenic mice manifest cognitive, sensorimotor, and social deficits. Delayed non-matched-to-place (DNMTP) task in T-maze for spatial working memory. The inventors measured spatial working memory using a DNMTP task in T-maze (FIG. 17) in both founder lines of the mice. Performance over training, as measured by an increase in the number of correct choices made, were analyzed by two-way repeated measure ANOVA. With repeated trials (10 trials per day), the wildtype mice showed less of a tendency to enter a previously visited arm. However, both founder lines of the VIPR2 CNV mice took significantly longer time to reach criterion (70% of correct choice for three consecutive days) than the wild type mice, indicating a spatial working memory deficit.

Novel object recognition test. Recognition memory was also examined in the VIPR2 CNV mice using a novel object recognition test. Mice were presented with two identical objects during the first session, and then one of the two objects was replaced by a novel object during a second session. The amount of time taken to explore the new object provides an index of recognition memory. While the control mice showed a trend to interact more with the novel object, VIPR2 CNV mice founder line A showed a statistical significance to interact less with the novel object, suggesting recognition memory deficits.

Impaired sensorimotor gating in Prepulse Inhibition (PPI) test. Prepulse Inhibition (PPI) is a “cross-species” neurological phenomenon of sensorimotor gating. The reduced PPI has been proposed as a biomarker of Schizophrenia. An acoustic startle reflex measurement system was used (Med Associates, USA) to measure PPI in the VIPR2 CNV mice and wildtype littermates at 3-5 months of age. Each test session consisted of four types of prepulse-pulse trials that included two different prepulse intensities (75 and 85 dB) and two different interstimulus intervals (30 ms and 100 ms), plus the startle pulse alone trials that were pseudorandomized, with the background noise level in each chamber at 65 dB. For wildtype littermates, a weaker acoustic prestimulus (prepulse) inhibited the reaction to a subsequent stronger startle stimulus (pulse). PPI was indexed by percent inhibition and defined as the percent reduction in reactivity in prepulse-plus-pulse trials relative to pulse-alone trials. Increasing prepulse intensity led to an increased magnitude of PPI. However, the PPI was significantly reduced in both VIPR2 CNV transgenic lines as shown for 85 dB prepulse with 30 ms interstimulus intervals in line F and 85 dB prepulse with 30 ms or 100 ms intervals in line A.

Impaired social interaction and recognition in a three-chambered social interaction task. The inventors next tested the social function of the VIPR2 CNV mice in a three-chambered apparatus following the standard protocol 40. The three-chambered task consisted of two trials: social interaction (trial 1) and social recognition (trial 2). In trial 1, the inventors measured the social approach of a mouse toward a stranger mouse trapped in a wire cage versus the approach of an empty wire cage. Next, the inventors evaluated social recognition by allowing mice to have a free choice between the first, already-investigated, familiar mouse (Stranger 1), and a novel unfamiliar mouse (Stranger 2). VIPR2 CNV mice showed significantly reduced preference for exploring a stranger mouse relative to empty cage compared to wild-type mice as determined by the amount of time spent in each chamber or sniffing cages and the preference index derived from these parameters. In the social recognition test, both lines of VIPR2 CNV mutants showed a significantly impaired preference for the chamber containing a newly introduced mouse (Stranger 2) over a chamber containing a now-familiar mouse.

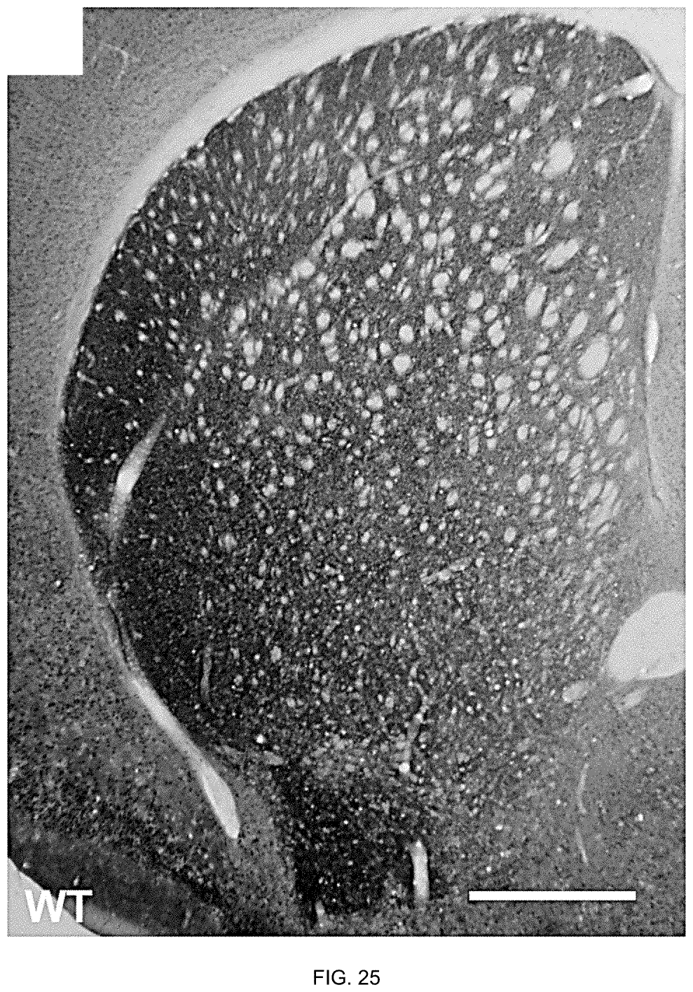

As can be seen in FIGS. 25-37, VIPR2 CNV elicited striatal dopamine neural transmission and cAMP/PKA signaling deficits in mice. Prominent dorsostriatal DA neurotransmission abnormalities in VIPR2 CNV mice. It has been suggested that excess dopamine neural transmission through DA (dopamine) type 2 receptors (D2r) in the striatum is an underlying mechanism of Schizophrenia pathogenesis. D2r immunostaining was performed in the VIPR2 CNV mice and wildtype littermates at 3-5 months of age using an antibody widely used and validated in the KO mice (Millipore AB5084P). The immunostaining of the D2rs was consistently found increased in the dorsal subregions of the striatum, but not in the ventral subregions of the striatum in both founder lines of the VIPR2 CNV mice compared to WT mice. The inventors also observed an increase of the dopaminergic terminal input to dorsal striatum as immunostained by Tyrosine Hydroxylase (TH) antibody. Finally, tissue monoamine levels were measured in the dorsal striatum of the VIPR2 CNV mice by High-Performance Liquid Chromatography (HPLC). The main finding was a significant increase of DA, but not 3,4-dihydroxyphenylacetic acid (DOPAC), 5-hydroxytryptamine (5HT) and Norepinephrine (NE) levels in the dorsal striatum. All these results collectively suggest that VIPR2 CNV mice have a prominent dorsostriatal dopamine neurotransmission abnormality.

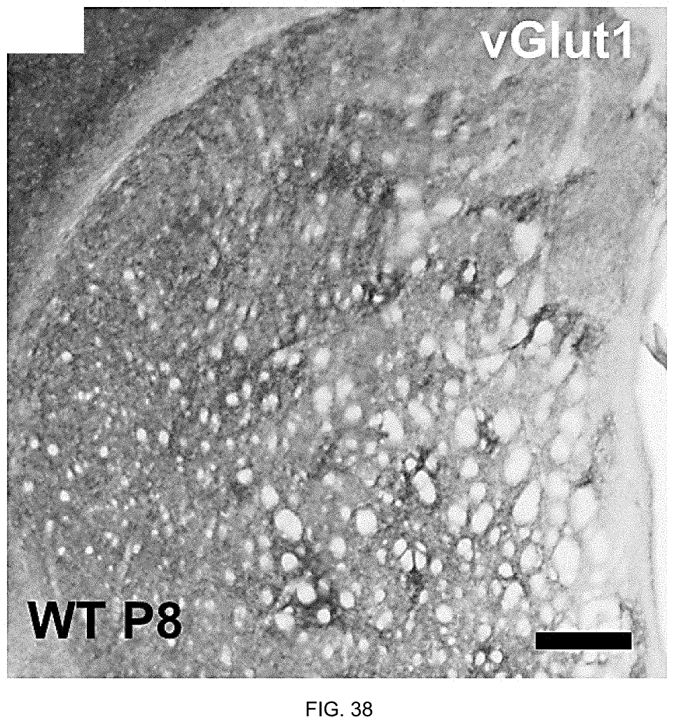

Turning next to FIGS. 38-55, the early postnatal striatal developmental pathology in the VIPR2 CNV mice is described. (A-B). Striatal developmental deficits in VIPR2 CNV mice. In VIPR2 CNV mice (line A), the inventors observed a significant increase in the intensity of vGLUT1 immunostaining) and vesicular glutamate transporter 1 (vGLUT1) protein levels in the striatum of the VIPR2 CNV mice in comparison to the wildtype mice. At P8 striatum, VIPR2 CNV mice also have significantly increased vGlut2 immunostaining, which is a marker for thalamic excitatory inputs. The inventors found the presynaptic marker synaptophysin level was also significantly increased in the striatum. All these results collectively indicate that striatal SPNs in VIPR2 CNV mice received abnormally increase excitatory inputs in comparison to wildtype littermates during early postnatal development. The inventors performed Golgi staining in wild type and VIPR2 CNV mice (Line A) at P18, using the FD Rapid GolgiStain™ Kit (FD NeuroTechnologies, Columbia, Md.). The inventors traced Golgi-stained striatal SPNs and their dendrites to investigate the cellular morphology and complexity of these cells. Sholl analysis revealed dendritic hypertrophy as measured by a significant decrease in the complexity of dendritic arborization in VIPR2 CNV SPNs. The inventors next categorized the dendritic spines based on their morphologies and found that there was a significant alteration between the genotypes. In VIPR2 mice an increased number of immature spines (thin and filopodia-like) and spine length, but significantly reduced mature spines, and dendritic diameters were seen. Thus, morphologic and behavioral changes were seen based in the VIPR2 CVN mice indicative of Schizophrenia. In summary, these results suggest that striatal SPNs received abnormal glutamatergic innervation and the early postnatal striatal dendritic development was disrupted in the VIPR2 CNV mice.

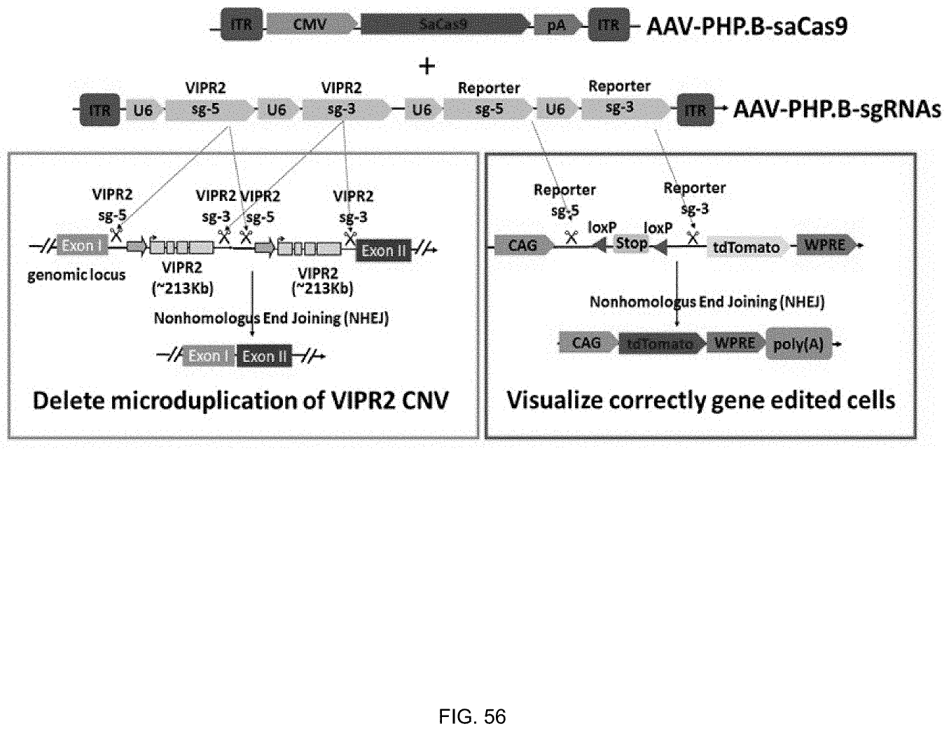

Turning next to FIG. 56, a schematic illustrating a strategy of a CRISPR/Cas9 mediated in vivo correction of VIPR2 duplication in VIPR2 CNV mice is shown A further object of the invention is to develop CRISPR/Cas9 mediated gene therapy to delete/inactivate extra copies of hVIPR2, or downregulation of hVIPR2 overexpression as novel gene therapy for Schizophrenia. To explore a therapeutic strategy that can provide long-term remediation of CNV related developmental deficits on cognitive circuits, as a proof of principle, this proposal attempts to use Cas9 guided by sgRNA to delete the whole microduplicated human VIPR2 genomic regions in VIPR2 CNV mice delivered via the latest highly brain-penetrant Adeno Associated Virus (AAV), and then to evaluate the therapeutic efficacy on molecular, cellular, and cognitive circuit-level deficits, as well as to evaluate the safety of the approach. Compared with traditional rational drug design, mRNA lowering, and Cre-Lox strategies, the inventors' innovative highly brain penetrant AAV mediated gene editing strategy offers the following advantages to target human chromosome microduplication related neurodevelopmental disorders: 1. Permanent correction of CNV on host genome offers lone-term therapeutic efficacy; 2 Can be delivered at the neonatal stage after prenatal diagnosis; 3. Allele-selective CRISPR/Cas9 strategy based on Protospacer Adjacent Motif (PAM)-altering Single Nucleotide Polymorphism (SNPs) can be designed to target patient-specific CRISPR/Cas9 sites, therefore convey personalized and precision medicine. 4 The translational significance of the approach is tremendous, not limited to Schizophrenia, but affords new strategies for the treatment or prevention of broad gain-of-function mutations related neurodevelopmental disorders, such as birth defects, learning deficits, intellectual disability (ID), and epilepsy, as well as psychiatric disorders such as autism spectrum disorder (ASD), bipolar disorder (BD), attention deficit disorder (ADD), and obsessive-compulsive disorder (OCD). The inventors have the sequences for VIPR2 deletion.

The gRNA sequences to delete VIPR2 CNV are Target 1: gRNA: cctggagtctgaaaggactg and Target 2: gRNA: acagacccatcgatggccaa (SEQ ID Nos 2 and 3) Based on the inventors' experimental results (not included) VIPR2 CRISPR/Cas9 improves the cognitive and negative symptoms of Schizophrenia in VIPR2 CNV mice.

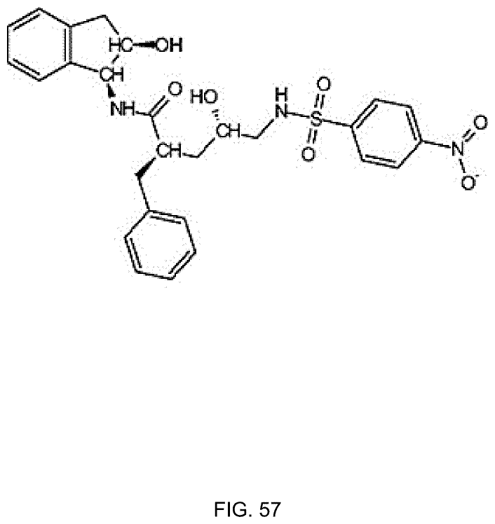

Turning finally to FIG. 57, a small molecule VIPR2 antagonist, (2R,4S)-2-benzyl-4-hydroxy-N-((1S,2R)-2-hydroxy-2,3-dihydro-1H-inden-1-yl)-5-(4-nitrophenylsulfonamido) pentanamide, is shown. Another object of the present invention is to use the VIRP2 BAC Schizophrenia mouse model to identify VIPR2 receptor antagonist as an antipsychotic drug targeting cognitive and social deficits, or as disease-modifying therapy to prevent or slow/stop disease progression. Currently, the majority of VIPR2 agonist or antagonist are peptides. However, two properties of the peptide hormone are problematic from the perspective of therapeutic applications. First, they have a short half-life as a result of its rapid proteolysis. Second, most of these peptides are not specific and activate three broadly distributed receptors, VIPR1, VIPR2, and PAC1. A high-throughput screen on human VPAC2 receptor using a cell-based cAMP assay has identified a single confirmed antagonist hit from a 1.67 million-compound collection. This compound is the first specific small molecule antagonist for human VIPR2 receptor. The full name of the compound is (2R,4S)-2-benzyl-4-hydroxy-N-((1S,2R)-2-hydroxy-2,3-dihydro-1H-inden-1-yl)-5-(4-nitrophenylsulfonamido) pentanamide (FIG. 57). This compound is a selective human VIPR2 antagonist and does not antagonize hVPAC1 or hPAC1. This compound is also highly specific for human VIPR2 and completely lacks activity on the mouse Vipr2 receptor. The inventors experimented with this hVIPR2 antagonist in the cell line of VPIR2 CNV. The inventors have found that this hVIPR2 antagonist blocks the VIPR2 receptor and the downstream signaling in the cell line expressing the human VIPR2 receptor. Therefore, the inventors' human VIPR2 model is the sole existent animal model to study the in vivo efficacy on cognitive and social deficits of these VIPR2 small-molecule antagonists.

A still further object of the invention is to use the VIRP2 BAC Schizophrenia mouse model to examine preclinical efficacy of the next generation of antipsychotic drugs.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A transgenic non-human mammal comprising:
a full length human VIPR2 genomic region integrated into a genome of the mammal.

2. The transgenic non-human mammal of claim 1 where the mammal is a mouse.

3. The transgenic non-human mammal of claim 1 where the VIPR2 genomic region is within a Bacterial Artificial Chromosome.

4. The transgenic non-human mammal of claim 3 wherein the Bacterial Artificial Chromosome and VIPR2 genomic region have at least 90% sequence identity to SEQ ID NO: 1.

5. The transgenic non-human mammal of claim 1 wherein the mammal manifests Schizophrenia-associated behavioral deficits.

6. The transgenic non-human mammal of claim 1 wherein a single copy of the full length human VIPR2 genomic region is integrated into the mammal genome.

7. The transgenic non-human mammal of claim 1 wherein multiple copies of the full length human VIPR2 genomic region is integrated into the mammal genome.

8. The transgenic non-human mammal of claim 1 wherein a number of copies of the full length human VIPR2 genomic region integrated into the mammal genome is between 1 and 4.

9. A transgenic cell from the transgenic non-human mammal of claim 1.

10. A method of treating Schizophrenia in a human comprising:
administering a therapeutic;
where the therapeutic contains one of
a pharmacologically effective amount of a hVIPR2 antagonist, and
a CRISPR/Cas9 formulation.

11. The method of claim 10 wherein the hVIPR2 antagonist is a small-molecule hVIPR2 antagonist.

12. The method of claim 10 wherein the hVIPR2 antagonist (2R,4S)-2-benzyl-4-hydroxy-N-((1S,2R)-2-hydroxy-2,3-dihydro-1H-inden-1-yl)-5-(4-nitrophenylsulfonamido) pentanamide.

13. The method of treating Schizophrenia of claim 10 wherein the CRISPR/Cas9 formulation therapeutic mediates genome editing to one of delete and inactivate extra copies of hVIPR2, or downregulate hVIPR2 overexpression.

14. The method of claim 10 wherein the therapeutic treats both cognitive and social deficits of Schizophrenia.

15. A method of determining efficacy of an antipsychotic therapeutic in treating a condition comprising:
administering to the transgenic non-human mammal of claim 1 the antipsychotic therapeutic, and
measuring symptoms of the condition to determine an effectiveness of the therapeutic.

16. The method of claim 15 wherein the condition is Schizophrenia.

17. The method of claim 16 wherein the symptoms are one of positive, negative and cognitive symptoms of Schizophrenia.

18. The method of claim 16 wherein the symptoms are each of positive, negative and cognitive symptoms of Schizophrenia.

19. The method of claim 15 further comprising measuring disease-modifying efficacy to one of prevent disease development, slow disease progression, and stop disease progression.

20. The method of claim 15 wherein the non-human mammal is a mouse.


SEQUENCE LISTING (3 sequences)

SEQ ID NO: 1 - DNA, 213535 bp, Artificial Sequence
  bacterial artificial chromosome containing Homo sapian DNA inside of Escherichia coli plasmid
cacatcctgctccccaggccttcaatgcccaggcaaccttggacaacaatggctttgagacctcaccttgcagtgcaaccgtggacaatgcgtcctccacaggaccctccctcccagcgactgtgtgggtcgtggcagctgagagcagcgctgcactgttgggtcaccctctcagcttgctgagacctgttaaagtaaaaatttgggccttggtttacatttttaacagctaaaagttttaaaaatgaatttccttactcaatgtcatcctggaaagaagctgacaccataatggtcctaatcggatcgctgagttccaatgggtgagggtttggtaaaaatggcacaggatgggctgggctggttgagaatgtaccatgttggcctctctcttgctctgctgcttgctagctatgcagcttggtgaggtcctggaactttctgcatgcagtgtctccaattttcaaaagtccacagctcatgtttatagtgctgtctttccagatggaactgggtctccaggggcaaaagggcagtgtgtcatcttctgttctcatgctgcccaatggcttttcagaaaacaacatcaatggacatttccacagcactctgctctccaacccagcaccaagcacatttggttttagagtcaggcataacagacgttatctccattgaaaaggaaggagaagtgagaacaggttcctggccaggtggagctgaagggtcttcctcccagagtgggggtgaaacctcccaggaaacggagctgccacatcctctctttctctcgcccgcccaccccgcctgctgtggcagatggcattttccagcatctcccaccccacattctcctctgcagtcttctctggccactccctccggagaggctcttgtttcttcaccccttaacctgggcaggacttgcggttcagactgagggtcggaggcttgaagctgctggctacaggcatagccctgacctggcctgcggccgatgtcacctgccttcaggaagccactccatgtgagaagcaccacgctgactccacacacgtggcagccccagccacacagagagcccacgtggagagacagagagaggccaggcacgctcagaaaagccaaatgagaggtgggtccttcagcctcagctgtgccagcacccggtaagaggggtccctcggcctcagccgcaccagcacccggtgggaggtggtccttcagcctcagcagtgccagaacccggaggatcagaggatcccttgtccatggaatcgtgtgcaaaatcaaacaggtgtttgaagccactaacgtttgtgagagtttgttatacagcagtagacaatcaacacgcgcaccacggagcagggagggcgcccactctgtgcttttctaggctaagaaagcagcatcgagaacgccccatcacaacgccatcaagcttcaccctagaaaagtcgctaagatttcccgtctaagggtccagctgagagtgcggctgctggaccagacgggctggggcaagtcctgccccacggagccgcacagctttgcttcacctcattcctgcactcgtgactctgcatctcgaagtgaagaaaaagtggcattcgcctcccgtgggggctaaaagcattcacacccagacacagtggcacacctagaacaggtgcacccacacttactgttcccaggctgcatctggaaacaggctaacatgaaacctgcaaggaaggtgttagactttgtgacccgagttcacctagaggctctcagcatttgtgactgtggccagggctggcgtcaccttaggaccccatttcatcgggacgctttccggcctgagagtcagctgagaaaggtgcaggatggatggtgccctctgcacagctgcaagatgtttgacatcatccgggacctgcttcagctgctgctgctgtggaacaaacactccaggatttagcggcttcaaacatgaattttattttgcctatacacaaaatgttgtgtgtcagaaacttgggaagggttcagctgggcagttcccacgtggggccattcatgtcgccgtggccagccccaggctggacgccacacggctcaggcacacacacgcctggccaggggttcacgtctctgctcagcccctcagcatggcaggctcaggccaggcttctcactcgcctccccagtgagggtcccaagtgaattgggtggagggcccacgacagcctcacttttaataagtgaaaaacttacggaattgatttttaaggatgaattagcagacaatttaaaaataaagctaataaaataatttaaaaataaaaatcagttcatgaacttcaacttcaaaaataaataacaggctgggcacggtggctcacgcctgtaatcccagcactttgggaggccgaggcaggtggatcacctgaggtcaggagttcgataacagcctggccaacatggtgagacccatctctactaaaaatacaaaaattagccgagcgtggtggcgcatgcctgtaatcccagctactcgggaggctgaggcaggagaatcacttgaacttgggaggcagaggttgcagtgagccgagatcgtgccattgcacttcagcctgggcaacagagtgaggctttgtttcaaaaaataaataaataaaataaatgaaataacaatttcactggttttgtgaaatttcccacgacatttcttcttccactgaaacccccgttacatgggtcacttcactgttacaatgttacaagtgttcagcgggtggggaagggaacagaagcgattcatttgggtgggtttggtcgtggaaggggtgatttgtggtatcgctaaggagagacgggcctgccaagaaagatggggaagaatcccacggtgggaaacgcctcccgaggagtcactgtggggagtgagccgttcttacttggtgtgagcagtgtctaggttggatgttgacaaaaatgagcaatttagtggagacgtcaggaagagtctatgtcaccggcaagaggcctgttcctgcaccaagaggtgcggctctgggaagcctcttagagggacaccaagcatagcttcccgagggtccccaagccaccacccacagcaacgccccggagcacagcactgtgatctctggctggtgactcagagcacttcatttttccattttcttaggaattaaattcctagattgcattaaactagtattttcaaaggagcatttcataaacagaactgaagggaaattggtatattaaaaggaatgttctcttattcaaagcaacatgtgaggctgtgaagccgcagaaccgccagtcatgtttcaacgggcgccgccagtctccagtctggaatcctgctcagcagctgcgacgccccattgggaaaccaggggtttgctccacaccggcttgtccttttgggtttctgtacacttcagtaataaaatcaatgatttttttttaaatagtccttttcttccattaaaaaaaaaatgggtccatagtatctgcagcagtggctgggctgaactttgtctccaagtgagtaaatgacattttgttacaagtataacagttgtctccggtaactgatgttttcttctagggataagaagggtctcggagtcacagagtctgggggacctactcctccacagcaggcgggagcaccaggcctgttgggtgcaggtgccaggctgggcaccatcagccgctccttccctgctcattcacaagcgtttacccgaagctgctgcgacggtcggctcctctctgggatccagagcttcatgccggccagtgagagggcagagacacaccgacgcaacttgaaatcacaggacctgcctctgtgcagaccagaaaggcaagtgccttagagtgagaaggagggagcctcagaagacgttaaggttatgggccctaaaatgagaccaccaggactccctaccaggctttaccaccacaatctcagtttccttgtcactaaaacaggaatacaaataacatttactccatagcaatgagcgggattgaatcaaatattcatgtgaagtgcctgccgtaaagtaagccacagccttctcaggatgaaggaaggcagggcctgctctgatggaggtggtggacaggcagcttctctccacagggactagcccctggctaccctgtgccagctggaatagaaggcctggactcaaggctacagcaatgaaaattcagcacacataacagaaattggtaattcagtgttgtcctggatgtggcctacagatctatccaacaaagcattccaagccctgggctggctgctgaggcactgagagaggcctgaccgttgtcatggcacctcacactgggacagaggagcagaagcatcctgcatagacacagcagccggccctggcttctccagttgactgcgtggccttcagcaggcgtgttgtcacctgcacacgggggcgtcatggtccccagggtccatggactctgaactccacgcaactgtcaacatgccggtggtgcttatgaaaacactgctgccaccaggttccagggaacaagtcacgacatggaccaacctcacataacccgcacacaccacccgggcaacaaacaaagcacctttcccagcacactcggctccatacgatggagtagggaaatcaacacagggtaaatctaatcgtcgtttctgcatcttgcttttgagtatcctcttcattttcctcacattctttgtcacttccttaccacaacatcttttacggtctggagggagagtatactgcaagaaggaaattaaagtagaatatgaccgctggaggagcgaaacactttaacacagctcaagtgccctaattataagaacattcttcaggagaaatgtgggttgatttgtgggcttaacagatcttcattttctgccgttacaatgtgtccaagttgatgtgtttagctgttttgttgactcacaaaggaattttagttaaagctcaatacttatttattttctgtctatctaaattccatctgtgtggtcagttgagttgattagaacaaagtactaaagaggacatggtccgtggtctcactgctgtttctcctcagatgccaagcaaagcaccttgcacatcctgggtcctaagtaaattgtcgataattgattaaccagttgggagtttgttttctgtcttgtgagaccatctgaagacgactggggcgtggatggaatgttgcaagttctatgctggcaaacagcattgtgtggaaagcacctccttaggcaccactggggacaagagacgcaggtccgaggccctgcactccgcaagtctgcaacctagctgagacgtggaaacacaaaacagtcagaggtcacacaaatcagcgtgtggttacccatcagaatgagcagcagcagcaccagtcatagtcctgggccgggtcacccattgccgacgttccgacaccgtcgcccacattccgacaccatcgcccatgttctgacactgccttggttgcagttttgaaatgaaaggcacggctgagccaggcagcggaggtggcatcgtggccaggaggtggagcaagggagcccctgtgtcctccgatgttccgatcttcattcgaggagtggttccacaagtgtaggccactgaaaaacttgctggtcgcccacttaggaactttgccctttactgtgtgtaacttatgcctcaacttaaaaagcaaagaggaaaatgattttaactacagccaggcacacatttagttgagtctggaaacttttgaatttttggcacaccaagcgtcatctggcacgtagaatgattgtggactggagaagacagcgaaagaaagaagattcttgaaagtgcatcaccatgtgcttcttgatggaaaagatggaacagcatatacagtggaccttgaacgaagagggggtttggggcccaaagcctcccaccccctgcacagctgaaaatccacaaagaacttttcactccccaaaacacttagcttctcatagccttctgttgacaggagccttaccgctgacatgaataggtaaataacacgtattttgtatgctgcatgtattacatactgtatttttacaataaaatcagataaagaaaatggtattaagaaaaccataagagagactaactttactgttcacaaagtggaagtgtgcctcgtgaaagccttgcctcctggtgtgacggggagcggtgactaggacggcagcactgcaaaccgcctccaagcttgccttctgttctccagagctacaggccttcatcctcctcgtccccacctggaggggctgaggaggaggggaaaggggagggcttggtcctgctgtctcagggtggcagaggccagagaggcgcagcacaggggaaggagggggcagaggcaggctcacccaggacggtgtttcttgaaaacaacccacacttgccatgaagttcgatcgcacgcatttgttttctcctatccatttatttgtgtagaaaatttttcacctgatattttaaaatccaaaacttgtcacgaatggagtagttaatatttggaaaatgagttttgccctccaggctctaatgcacagtttagtgagttttgccctccaggctgtaatgcacagtttctaacccgcttccgggaaaagcagcaagtccctggtgtctcacacagttcgaggctctaagaggagcgttgtgcccgtgggaaatgtcatatcctagagtcctccacaaatctcagcaaaatacgggggatagaatctttcctcaaggaggcgcggtacacatgtgcgtgtctgatggttaattttaggcatctacttggctggattcgggacatctgtaaagctggtgaagcatgatttctgggtgtctctggggagatcagcgcatgggtcggaggacggagtgggacagatctgctctcactgtgggctggcactgcccaatcggctggggcccagaggaaacaaaaaggtagagggaaggagaattctctcccttccagagccgggacactgcttctcctgcctgtggactttggagctccaggctcctcggcttttggaccccaggacttggggatttaccttggcagaccccggactcgcaagccctcagcccaggactgagaattacaccatgggacccctggacggagaattacaccatgggacccctggacggagaattacaccatgggacccctggacggagaatttcaccatgggacccctggacggagaattacaccatgggacccctggacggagaattacaccatgggaccgctcgatggagaattacaccatgggacccctggacggagaattacaccatgggacccctggacggagaattacaccatgggacccctggacggagaattacaccatgggacccctggacggagaattacaccatgggacccctggacggagaattacaccatgggacccctggacggagaattacaccatgggacccctggacggagaatttcaccatgggacccctggacggagaattacaccatgggacccctggacggagaatttcaccatgggacccctggacggagaattacaccatgggaccgctcgatggagaattataccatgggacccctggacggagaattacaccatgggacccctggacggagaatttcaccatgggacccctggacggagaattacaccatgggaccgctggatggagaattacaccatgggaccccaggacggagaattacaccatgggacccctggacggagaattacaccatgggacccctggctctcgggcctttggactgaattgcaccaccagcctccctggtgctccagcttgcagagggcacatcaccggacatcacagtctccgtaattctgtgagccggtttaggcccccaataatccccctctcacatacccatatctacatcatgtcctgttggttctgtctctctggagaactctgactaatatagggtgtgtggcttttcctaaaaggttctgctagtccccaagtgtcagtccctacgagacaggaagttattcagagttcctagtacctggcttggggcttggcacattgcacatgctctaaaataggtagctatttaacacatttactataaatagagattatagaaaaagtggaaagaaaatgtatttccacaggtatgcatcgttctgttaaagacttcagaagtaacatcagctttcacaaaattgagcttcattgaaagtaaagtcacagaaaaattctgaggttgacaaatttaccctgagaatctctatccccggccccccttttttgtggctcattcacccctcatttcctcacactcctgtcctctgctctccaatgtaaagagttcccctccgcagtggcatctgtgcttaaacgctgaccgcagccctaggctagaagctccaactgtgattatggaattccagacacatccctggagtctgaaaggactgagggttgggggtgagcagggccagccatgaggtcagctgggcccacagcctttggaagagacagggtccccactgccagaggtggctgggctcagggtctaagccagacctcagctctgcgtggcggagcattcacggcctccctgtcagagagcagtttcctcctggctttcctagaaccgggctattacaaatgtccctggtcttggcaattttcagacagcgactttcctggaaaagtcatcttttgatgagtgagctgtcactgtaatacctttttccttcagatgaggagctgatccttggagtaaggacagaatcccacggcacagccgagacacagctgtggccacaggagtgctcctgatcccaggtgtgccaacattccagggcttctgtcttccaaagatgaaaaagaagaaactataaggggttttatttccggttttgagagcataggaggcaaaacgcctcataatgtcagtttattttgttaaagactccaagtgaatatgaaaatgaagctaaattcagtttttaaatgtcaccctagtttgcctttgcgggttctgggggtcactgaaatagtctaccagcccttcaactggttccccttacaaatgaggagatttgtgtccggagagatcagaaaatgcttccaggtagagccggcacacctgggcctctgggtgcccagggctcttgattgccacagggttgaaaataaaagagaagaccctgctcctcaggacgccagcaagggagacgttaatcagccactgtcagcgttatgccttgggaaagcatggcttagtaattggcagcaaataacactttgaaaactcttatattgagggaatatctccatcccgtcaccctcatctgtctgtccccatcctcttccaaagggcattcagaactggggagaggacctagcaaaataggttagaaaatgggaaaaaagattaagaacaaaaaatcagtattttaatgaaaatacgaggacaaagttcttgatattgtaaaaactctggttttgtgtgtaaaaatagtaactgagaacaattaggcatagaaaatgcctcactttcatgtgatgctttaattactcccaggagaacattattaattatttaaaggaccacttgagtcattaattatccttccctattcatttctaagcaaatttttaaatttagtctcattcaccaatttactccccaatgagatttctatttctgcttcacacgcagctaaaaatagttccctcttttgttcctttaatgttgtttgttttcttctcttctccctaaaaagccacttggaaaggtggcatggccgtcaaaccagaccctgcttctctctgaactccaggaccctctggcctggaatcgctggcctcccggcctgcagggtcccccgtcactcccaggaccctcctttcaccccctctccccttctccatgctccctggatcccccacaccctcggaattttggacaagtgataaatgcatgaccgatgcctaccgatgatgaccagggaggctcagctgacagctttctccctcctcccaccaggccccacccgggcctcatctctcaaaaaggaaagtcctggtttaccaattcacccacaaatgttagcgatttagcacctcttgcttctcctttgaaagggaatcagagaggagagcataacccagaaagtgaggacgccctggcgaggggaagggtctggagggcagccccactgacctgctccctgcctgtggagccgaagcgtgggctctgggctgggcttgagggcgagtgctcagggtggggccctgctgtcctggcagccgctggctgaaccctccagactggagtgagtggaaccaggagtaactcatacatctgcccacttgctcatcaacaaacagggaacacctgctgtacaaacccacaatgctgggcattcactggaggggcagacatcaaataatctctctaaatattgtatcctatcgcatcatattatattaagctaagaatagttctccaagaaagtggcaacacctccttgtaaagggaagtttaaagaactctgagcctggttggtctgagaaattggaggatggcaacagtcagggccccaggagaggagaaagagacacctcctggaagaactggtcagaagcagaagggagaggctggtggcctgtctctgtgagccccacctgcctggggtgggagggggcccgggcagggctatggcatgcgctggtgtgtgcctgcctggggtgggagggggacccgggcaggctgcggcacgcactggtgtgtgcccgtctcttggacaaaccatctgagatgttcttccagctgctctgcctccatcgctgagcctcctgctgagctgagtagggtccccggctggcctggcactaaactctgcttagatatgtccaagggtctctgcttttgccccagcgaggaaaggcaggatgcttctccagactgggagggctttcctaaggcagacacctgagccaagacaggtggaagctgggaggcatgtgcctggggtggagggtgcctggggcggagcgtgcctggggcttccagagatcagcaaggctgaggcatgagaggcagagtggaggctgcgagacgggtccaggaggagactcaggcccagatcaggtgtggcctcgcatgtagggtgaccatgtgcttcacataatatgtccaaaccaggacatttccaagaatagggggacacgagtaacggttacgctggggcaatgggcaaagccaggccaggcccagcatggcaggtggttgcctgttcaaaggtgtaaaccaaaatgtatttgaggcaggtctcaatcaacttagaggttgatttttgccaaggttaaggacatggcccagggcacagcctcaggaggtcccaagaacacacacccaaggtgacagggtatagcttggttttatacattttagggagactgaagttacaagaaaagacataaagcaatatctggaaggtatacattgatttggcccagaaaggtgggacatcttgaagcggggtgtggggccttccaggtcacaggtggattacaggatttcctgattggccgttgggtaaaacagagttaagatctgcctgaagacttgatatcagcttgagtgaaaataaagggggttgtggaagccaaggttcttgtggatgaagcctccggctagcaggcttcagagagagtgaatgtgaatgtctactatcagggccttgaaaggtgtcagactctccagaaagacctagtgacggacagggattctccacagagtgcaagattccccaacagacagctttgctgggccatttcaaaatctgtcaaagaaatatatttcttcagggcctgctggccgtcatgtgatgctctactagagtctggtgggaatttgctatcttattgctatgaacagtctgtttggtcagccttaggagctctgtttttatgttaatgttggtcatgaactccaggaaaggagggtgtagtgaggcctgcccaaccccctctccctgtcatggcctgaactcgtttttcaggtttctttgggatccctttggccaagaggggagtccattaagttggttggggggcttagaattttatcttttgtttacaaaggcatattgagaactttggtcatttttctaagagcatcaggtaaccgcagggtttggggtgttcagcaaagcccgtgattacacacacactggaccttcagagaagtgtgccatctagctacgaccacagccatttctcctggtctaccccgtccatcgggacgcactgctgcaggggaagactcacccatcatgcaacccctcactgcaccctggacagtcacttctaaaatgcaggccctcaacaaggtattgacgtgtgcattaatcaccttgattattacagagcccctctgcggtggaagggcaggtgctgtcattcctacaaatgcatagagctcaaagttcatcgctcacccaggtggaaccgggagaaaaagatgcgtttgtcgaaggcacccagcgccacgagctttcttcacaagccaagcagcccgcctaaggcccaggtctccctctcctcgtctcccactagatacacatggatgaagccagatagataacaggtagaggatagatgacaggcagatgacagataggcagatgatagatgatcgatagatgacaggcagatgatggatgacaggcagatcacagatatgatagatcacagatagatgacaaataggcagatgatagatgacaggcagatgacagataggcagatgatagatgactgatagatgacaggcagatcacagatatgatagatcacagatagatgacaaataggcagatgatagatgacaggcagatgacagataggcagatgatagatgactgatagatgacaggcagatcacagatatgatagatcacagatagatgacaaataggcagatgatagatgactgatagatgacaggcagatcacagatatgatagatcacagatagatgataaataggcagatgatagatgacaggcagatgacagataggcagatgatagactgatagatgacaggcagatgacagataggcagatgatagattgatagatgacaggcagatgacagataggcagatgataggtgattgatagatgccagccagatgacagataggcagatgataggtgattgatagatgacagccaggtagatcacagatatgccggatcacagatgatggataggcagatgatagatgacagataatagatgatagattacagatagacgatcgataggaagataatagatgataggtacatgacagatcatagataaatgacagagaggtagatgatagatgataaataggtagatggtaggtaatagataggtaatagatcgcagagagatgataggtagatgatagctgattgatacatgatacagagatgataggtacatgatagatggatagatcataaatgacagcagatgatagatgataaataggtagatgacaggttatagataggtaaattacagatgaaagataggcaaatgatagatgataggtacataatagatggatagatcatagataatagatgacaggtaggtgatagaggaaaggtggatgatagatgataggcgcatgatgatagatacctgacagatggatagatgaaaggtagatgatggacaggtagatgatagatgacagataatagatgacagattacagatagatgatcgataggaagatgatagatgataggtacatgacagatcgatagatgatacgtagatgatggataggtagatgatagataatgtggatgataaacatcagatgatagaaaaatcgatatctgtgaatacggggggtgggggatcagagagaagcaaatgataagcccaatgaggtaaatgttaatgaccgaatccacaaaaaggataaaaaggagttatttgtaatattcttattcttctaatttttgtgtaagtttgaaattatttccaaataaaaaattaaaacaacacgaacttaacttgtatctctcattccagtgctttccacttgctggggaacgccgagctctcctgggttggtcacgcgggcgccttgggaggccgcgcagtcccgcgtggggcgcggcggggcggccccagtggcaccgcgtgagtccccgcccagcgttccccacccgccgccgcgtttgcggggagagaatgacccccgttttgcaaacgcaggacacaaaacccgccacccagctgaggctgggacccacctatacccccgtctgcgtttttctccagctttctgttcgacctgatgtcctctttgaggctgcggggatccccaccaaaaagcactctgattttctccattttcagatgccccaacctagccccactggctacaaagacggcgcgcccaccccggacgggtctctggccgctttcttgggcttccgcagttgtttagaaaggcaggtacaggcggccgcaaaggcatccgccccttccgagcgcacacctggcccggtcctcctctcccggccccgcgccggccgctctcccccagttctctctactcggagccgggcgcgcactgagcaccccgcttcccagcccctccgcccacagatcggggaggcgcggccgggaggggggcgtgggccagggaagaggggaggagaacggagagagaggctgggggggtcgaggagaccagagggagcgcgcgcggcggggacagagagcccaggggcgaggagagggcgcggggcgcagcggaaggggaagtggggggcggtgaggaggggcgcggagcgggggttctcgggggaggagggagacgaggattggggcagcgcggggcggggacaggggcggggggcggagcggcggggcgcgtgggggacgggcgtgcattgagcgcgctccagctgccgggacggagggggcggcccccgcgctcgggcgctcggctacagctgcggggcccgaggtctccgcgcactcgctcccggcccatgctggaggcggcggaaccgcggggacctaggacggaggcggcgggcgctgggcggcccccggcacgctgagctcgggatgcggacgctgctgcctcccgcgctgctgacctgctggctgctcgcccccgtgagtgcgcccgcgacccccgccccacggcgcctcggacccggttctccccttccgagaagagcgctcctcccgccggtcttcgccttcctcccgggtgctggagcgcgggcggggtccgggagagggagcggggtcgcccggggtccggagcttcctcccggagagcgtgaagcgctgagctccggtcccgccgggttgcgggactcgggttgggaggctgcctgcgcccttccccgcgccccaccgtccggggtttgctggaaacgggatcgtttcttcctggacgcgtcaacgatgagctcgttcggggccgtcccgggagctgggagctgcgggcgcctgcgcgggctgcgcgtttcacggggagatcggggttggcgttggccgcagatgcctctcggtccctccctgtacttactggttagttttagtgtcggggggacgtggcgtcggttaaataaagtttccttttggaaatagttgctttttcccaagggcactggaggaactaactgctcataagtttgttgattaagtgccttcgcacatggagttctgaattattccctctgtgaaccttaaaaaaaaaggaaagaaaaaagaaagaaaaaaacccgagccttgtttcttgctttcctcttggagactgaagcgggtttgcggtgcgcgcggttttcgtgcgcgtcgcagccgaggcaggtctggatcgatccgcggcccctccgcagctgggaggctgcgctgctgacccgacagtgtcattgattaaccggcgacgcccggtctgcagcggggctcgagccacactccctggccttgcgcggaggcgcagctcggctttcctccctcgagggtgggcgctttgcggagcgggtccttggaaagtaacgggttttatcacggttaatgctgctttcattgctaaaatgtcctaaaagaagtcattgctgtgcatattccaggaacgtcaggtattattaatgtcttttacactttaacaaagtttttctttttacatcaatataatttccttaattctgaaatttaagccacttacttgtcctgcatgaaaacagaggacttggagcccaaagtcagtgcctggtgctggttcagtcacgagcaagaagcaagctcggggctcttttaaatggggctgaaggtggcagaagttccagcattgttttccccttaattctgttaggatattgtgttcctggttggaactctcattgaccatttcaatagtaagataactaaacatagatggccatagtacgcccaacagtatgattctctttagttactactttagtacaagtagaattttggtcggtggtgggggagggggaagatcaagaccaaaaggatgggtggggggagccctccttaagcccccaagggccgcttggcaggcctgtgctgacttcgcggagggcgtgaagaacaaggtagaaacggtgccttctgagaggaagcattgtgtagttctgccttcttctcacattagtagactgttaattactatctttgtggcacctttcgctcccctgatggatcctgactggaaatcgccccctcttctctgtggggccacaatggtgtgattcccccacctctgcctccctcctcatcttgctgatttctttgaatatctcttccaccccaaattctagtcaaaacgattgcagttttatctgtagttagagggttaagctttgaacatgtaccaacacctttaaaaataaagcccttctatcacggctctttcccgggtggaggccgtcctcggaggaagcggcacagtccacaggcatccgcttaaaccaaaccaaagggtgtgaaacggaggagccgggtatggagagatgaatggagtaaagagctctgttgcctccaaattagtaccgatgtattctgttagtaagttcattttcctgctcagaaaaaaaattctattagattttgtgtttgttttataatacctttacaaagtaaatagtgaagtttttaaaaaatgccagaaaaatcaaggtgattggttttaaatgcattttggtttggaagcagtttaaagctgtgaggtttggggttgtttttgtagaatttttgcttgcttttcttcttcttgtggaatttttagttattaatatttcttttaaccgtgaacaggtgaacagcattcacccagaatgccgatttcatctggaaatacaggaggaagaaacaaaatgtgcagagcttctgaggtctcaaacagaaaaacacaaaggtaaggcagaggcttggtgagtaagaactcccgtgacattctcacccgggaatgcgcctcactcagctgtggttcagggtcaccacctgcatcttaaaaaaaaaaaaagaaaaagaaaggccacttcccattgtttgttaatgaactactggtaaaactcagagagagttggataggtagggtcttcacacacacctttaaaaagataaccttaatccttagcagtaacagtttccatggtttgagatgatagcaacagttactaaaacaatttcatgggctttgttatgctttgcaaaagcagcttcagttttccaggggataagcatttatctgaattcttgcattttaaattcttagctataggacagatgattttgatctggttaaggatggtgggtacttggcagaaagcaaacaggaaaatgggactttggttgtgctgaagacgggcctgcggtgaacactgtgaacgtgcagatcgccttctccggtttgacagaggcatgctgggccatcatgctccaacagggcctgctatcaacctgtttatagactctgccctgggatagcacccggcctataaatagtagcaaggcctttgtccctcagctttctctgctctttgtcatgattttctcctcttcaagaaagaaattaaatctgtgttaaaacagtctgttgtcctgtagagggcctcagatggtgggctttattcaagaatagccctgcccagcagcaacagaatgcaagccacattacaatgagtaaaaagaagcatgagagattaattttaatagtatcctttatttacctgaataaatctgaatcgttaacatttcaacgtataatcaacctgaagttattaattttgcattcttatttgatgctaagttgtagaagtccagtgtgttttttgtgcttacagcacgacacagtttgggccccatttcaaggtccagtagccacaggcggccagcggtctccacgctggacaatgggctgtagattgtcactaaaacaggtgtccctgcccgttcccatccccctctgagccaccgtttcttggtctgaaacgtgcaggctctgcctccgaggcttcttccggctgtaaagatctgcgattccgcggtgcacgcatggttctggcccatcttccagattgtctggttgaactctgtccagagaaaggcaaataaagtaacttggctgaagccatgctgagagtctgtcctgggaaatgctgatgcggcccaggagatgtggatggaggaggcagccagtctgccacggcagaagcagcagcgtggccatgggaggtccttcttccagtgaaagttctgagttaggaaaggtagtcctgaggagagacaggcttttagggttgccctcctcacctccaagttccaaattgtccagcttcctgttttcatgggatattagaaatgcagatttatataaaatattccactttcttaaaagtttgcaaacaatacattaaaaaaaaaagattttgagggagcctggagccatctgatggctctctgcacggtagctggaccctagcttaacctgagattggcgacgcctcctggaacaccctcacccaaaggcaggtcagcacatttacactattcacaggggctccccctcagtgggcagtccgtgtgtgagtcatatcgggcgctaggggccgcaggtcccaggcagtcactcagccccacgccagcttgagcgggttcatatgcaggcccgagagcccctcgcaggcagcgttggttgggaaacttgtcactcggctttcttgcagcgacaaggtgtctacctggacatctcatattgaaggctcttatttccagtgtcagctctgtaggaaaaatatctgtatctgatctgttttacctttttgacttgcacgaagtacttggtaacaagacactgcccttcaggagagaagcagcagcatgccggggcctcagcctggccactcttttgtttttctgtactctttgaaatatgcgtggctcaaatgccagggatgtaaatagaagtaagttaccagaataaagatgctatcagcgatgtgaaccggaatctacaggctgaggctgaggcaggcgtctgattagatgggctgagtgactccacgagacaacagtgtgcatttgcacctgacgccaggtgagatgagatactgagggtctgttacagccacttttaaatccatacgattaagaaatttgtgtgaaagagtgccagaaatgaggtgaaaaaaatctagcttccaattccaatgaggtaatttgtgtaagtctgatttggaaatgtttctctttattacatgttgaatctaaaaaaaaaacaaaaacaaaaaaacacgtaaatccagacagaaaccaatttcaattctaagctctcttacgggattctctttgtaagcttaggagttatttggagctgtatgacttaacatcgatgcatcactaagacaagctttctcttgccgatatgtgtatgaggcactcccggcctcagagccgagatccagccctgtttccatggcccatgccaactcgcaggtggaaggcacaggtccctgagggggctttgtccaggcttcctgcctaggcacaggctggataggaactttcgtgtgactttatatcattcttacacccgcgtctggaggggtgtgacctcctctggggagaagcagaggtggtctctatgtgttctgggagctcactgttctgagacatccttcagaggcgcatggctgaaatggatctgactgtgaatgtcagcttagaccaggccatggaaagtgctaggcttaaaaactaacttttctttgattttttttaaaacaatggatgctgaactaatttcgatctacatgtcaggaactatggagggtttgaggtcattccccacttgcaggctaacaacgtcgcctgccacagttttgcagatgctgcagaagacgtgagatgctgggccagagacaagagacttgctgttcacagcacagcaggcatcatgagctgcgtgtgcgcacgggttccctctgcttctcaagccccccaggagcagtgggagcggccccctccgatgcagtgcatttgtgttgtagctgaggagaactggcttaggaaacccccagtgtcaaaaggaggctgctggcaaatccccagcctttgttacagagagagatggtattgtaattatcttaattatcgaactctcctaggttgtttgctatacaaacatcctcagaaagaaggatgacttatgtcagagaaacatattctctatcatacttgggggaatgaactcctaaagcaaggactggtggctgctgagtaggatggaaatgtgcttcctttcctgtcccacagcatcctgtgactttcgtgatcataactcactgtgtgatttcttggtccttctcctccagtgcccagaggagatggcatcatgtttctgctgcttctcccaagtctcacacagaacttagcatctagagagatattttaaaagcttttgctgaacaaattaatgaaaattctcacagtagaaattaagtgatatacatatatatatctaatatgcacatagaaaaattctgttcaaaaatgtatataatgggaaatgtctctacagacttcactgctgccagccccaagttgcccctggagagttggggtgcgcacacgtccacacgtgggtagaccccgcttttccttccagacacgtggaagctggcatactcttcacacatgtgctcggcatcttgcttttcttttctgttttgtatttcagtgtaacaatgtatttggcaactgttttgcatttgttcttatattctctttaagagctgctatagtattctaatgtgtttctttagtctctcccaggtccatggacttttaggtagccgacagtattttgctactacaaacaatgttctagtgaccgttttgttggtgggaataaaatatttcaccagcatttgagggttaacttcaagccactcatataaaatgggaatcaccatctcctatgaccaaatgacactattttagcccactttctcattcctgtgtcggttgtgtcatgtgggatatggttaatcacaacccattttacacctaaccccttcttgcttgtttctctttgtcagaggaaaccagacagtgtccacaaatgggttcttgctcacagcgtcatgtgggcactgaccctcctcggagggtccaaggccctgtgtggctgcagcattccacctcgctctgtggtctcggctcagctgccctgcgatgctgccctctgctgcgggagctggagctctggtgggagctcgctgttcgtggtcctcagcagatattcaaggcagacctgttctaagaaactaaatgaataatcactgtgttatgctccagaaggtctgaaccgagcagttttggcatccacggcagagagcagagaagtaggttcctctgatgtcaatattgatggtgtagacgtcagttgtatcttaaaaggtgcctatatctttccttttcattttctttctgatataagtaggtccttgttctagtccaaataactttttggactgatgagataaacagcctggtcacttttttcttttggatctttttgactagatttcttccattttctaaactctttaaggtcatttcctcgtcttggatatttaaataattaataactgtttattggttgggacagtcttaaagtaatgtctttatgcctggtgcaatgggctcatgcctgtaaccccagcactttgggagatcgaggcaggagggtaacttgaaggcaggagttcaagacaagcctgattaacatagcaagacaccaaaaaaactttttaacttaacctggtgtggtgacacacacctatagtcccagcttctcaggaggctgaggcaggaggattgcttgagcccagttcaaggctgcagtgagccatgatggcaccactgcactccagcctggacaacacagacaacagagcaagaagctgtctctatttttttttaaagtaatgtctttatatttactttttataggaaacaaatctcactgaattaatttttaccctgaataaaatgcttaagaatcataaagggaattatggttgagaagtcagtttctggcaggtgaaggagtgggccactcagaagtgcactaatggcttctcctgtctgggaagaacggagctgttgcctgggtgaccacagtgggtcacaggaagccattcagggtgggcggccggccccttgaggtgagctgctgctgatggggtcagagtcagcttcctccccacccctcctgctgaggtcagggctcagtctggtgctggtgaccccagacacagcaagctcccaccagtcgatggcctctcctggtgcatacctctctgccttctccactcccctcccttgatctctgtctcctccatccttgggctctcacacctgcctcctcctccccaggtatgcctgggtgtcctcagaacctggctgcttcctgtcctgctggtgggggttggtgacgagtggcggctgcttctccacatggccacagcagtaacaattcctgctggcctcattttttatgattttatatttatatacccatttcacattctttacttatgagccatggtttttacagcatgttgtctgtcggggcctcctccttttcctgggctcagcctgttcttccacaaggactgtgttgacgcgggggttcttgagaggctcagagacctgggtcagtggaaacgaaacccacagccagcatgcagctcaccacacacacagctcctcagtgtgtggccagtgacctctcagcttcttccggggcagcagtggttgtcttgggtacatggccactgtggatggacactaatgtgcgctttcatgcaacctgtcagattcttggtggcttcgactctcacatgtttttgatttgctatggtttgaatatttatccccttcaatacttaggttaaaacaatcctcaatgtagtagtattgagagacgggccttgaagaggtgattgggtcatgaaagctctgccctcgtaagtgggttaatccgttaatagattagtgggttttcaagggataggttttataagaagaagagagacctgggccaacacgctcagccccctcactacacgatggcctgtgccacctgggaaccctgaaaagtccccgccagcaagaaagccattgtggccactcgaccctggatttatccagcctcataactgtgagaaataaatgcctttcctttataatttacccagtttccggtatgttgttataagcaacagaggcaaacttagacacatgagcagactgcagctcttcctgaaggggacttgggactcgaacctctttggtcacaaagctttgtagagttttctgggtcagcacccagtcctgtgattccctgaaggcttcatggccaggacctgtgtgtttgagggttgcctcgtgtcgtgggatgagtggaaattaaaccgtccggctgacatttgctgatgcgttggctgatcctctcatttatttattttttctgagatggagtttcactctgtcgcccaggctggagtgcaatagcatgatctcaactcactgcaacctccgcctcctgggttcaagcagttctcttgcctcagcctcctgagtagctgggattacaggcgcctgtcaccacgcccagctaatttttgtatttttagtagagatggggtttcaccatgttggccaggctggtctcgaactcctgacctcaggtgatccacccgtcttggcctcccaaagtgctgggattacaggcgtgagccactgtgcccggcctcctcccatttattaacatttttcagcaggtttttatggaacagataccagattgtaataaagaactttcctttttaaatattatttttattttatttattttttgaaacggagcctcacgcactctgtcacccagattggagtgcaggggtgcgatctgggctcactgcaacctttgcctcctgggttgaagcaattctcctgcctcagcctcccgagtagctgggattactggcatgtgccaccatgcccaactaattttttggatttttggtagagacagggtttcactatgttggccaggctggtcttgaactcctcaagtgatccgcctgccttggcctcccacagtgctgagattacaggcgcgagcccctgtagccggcccttttttaatgttaaagactcacttttcttaactaatcaaagactagagacagcatgaagcttaataagttatcctgataaaacacaaaatcattgttttctaggccaattacttaaaatgttaaaaaaaaaaaaaaaaaaaaaaaaaacttttgtaattaagagaagaccaatattcaagaaaatattgtcctattggccaggcgcggtgactcattcctgtaatcccagcactttgggaggctgaggcaggcagatcatgaggtcaggagatcaagaccatcctggctaacacggtgagaccccgtctctactaaatatacaaaaaattagccgggcgtggtggcgggcgcctctagtcccagctactcaggaggctgaggcgggagaatggcgtgaacccaggaggcagagcttgcagtgagccgagatggccccactgtactccagcctgggtgacagagcaagactccgtctcaaaaaaaaaaaagaaagaaaatattgcactattaatagaaaacaaactcactttgtgtcagtacttgaagctttttttaacaaaaacttataaataaaatcattctaatgttagtagtttgaccacacataaaatttcttttgtcagactttctacaactgtgtatacccactcagtttttttcttactcattttcttcttttttattctggaacaatcagtcatcttgctttaagttaaaagtactctctttttccttaaacacagacctcatatacttttccttattaaaaacacatcatactttctctgtttttttcatatacagttgtttgctttcatctttatttccagtgattttaattacacatgttaattcaaattcttaatccttagtgacactgatatgcactgtgaattaggaagtacatgatatcatagttattaaatggcatgtactttctcagcacaggacacacttactggcagacctaggtagctccagttcctctagaataagccaaaaatacttgagcttaaacttctttttaggaattagtgttttagtagtttatcttatttgggcatgatctagatatgtagtaaatatcatttaatttaacttagtataatttcagagtttcaagttttcaaaaatattttggaaactgtttttaaacagacggataatatcaaactacctgtcatctcaggttatttttcttgttgacaaattttttaacaggaataatgagcttatttgactagctcagctaggtagaatgtccatttctactacatcatcgacattcttatttattaaagattactcaagttagatgaacttgaaaaccattgggttagttccttttttctcacaaaatatttaatttttacaagcacctaattttctttaggcaaataaaatagatctcgtttatatgttatctctaataataacatcagaaggtagggtaatatgagatataaataacatacatacatatagacgtacataaccaaacagacagaagcagagattcctggcctctgttctcaaattgtagccctgtgccaggaacgataatataaaactcactggtcggtccaaattgtgttcttggcagataaagttacctgttcggatggccaaagtcttttgttaatacatatggaaaggactttacgattttccatttgccagtttttttctacagtgttcccattttttctcccttctgatgcgtcatttcccagatgcctgcattttaaagatttcggtatctctaaggcagggagaaaggacattaagttttctccagaaaggagctgtgggcatagttgcctgttgtccggggtcagctttattgggaccgtgggccgtgtttagggggtggccaggagacactggtgtgttgacgagtccttctcctcttcagcctcaggtggctgcttttgtaatagctcagggttgtgtgcaagaggccaaaatctgagttagttgttgcagatgccccaggctcagtggaggatttaatttgtaaggaagttggttttcagaatcttcttatggaaaaggcaagaaatcaaaatagcgttttgtttgagtgttgtagagattcctttagggaggctgcatttttggaatcattctgtctgaaatcaatctgtatgccaatcattctgtctaaaatcaatcggtatgccaatcattctgtctgaaatcaatctgtatgccaatcattctgtctgaaatcaatctgtatgccaatcattctgtctgaaatcaatctgtatgccaatcattctgtctaaaatcaatcggtatgccaatcattctgtctcaaatcaatcggtatgccaatcattctgtctgaaatcagtctgtatgccaatcattctgtctgaaatcagtctgtatgccaatcaaaaaatatattgcgtggtttgcagatactcacgattccttgttcttgaatgagcccgtccgtgaatgatttcatgtggaataaacttcccactgtggccactgtgactcggagtcatccccctgagctgcacccatttctccagaaaggcacgggctctgggtctgtagtttctgaacatataattagctggagttccagatggggtgtcccagactccatggattctgatgaacccataattccctgtttttctcacatttttcttacctgaggcctccgactggatccaggccagttcctgtagaacacccagtttcactctggatcccagtccaggttccagtgcagtccctgtcagacccaggctgagctctggaataaaaactgctcccatggagtcggagagctcagggtgcaagttgtggagctcgacctcctagaggggcttcccctgtggccaccaaatgcagcaagacagcagtgggtgcagtggcccgggggctgcccgcgcctggttactcgctgctcctggggatctcacttctgacgagactgtcaaaaaggtaaactgaggcacagtgctgtgtttaaagagttcatttgagcaagcaacaattcatgaaccaggcagctccaaaccagaggtggttcaggaactcccctgaaggaatgtgagggggaggcttttatagacgacacagaagtaaagcaaagaatgttggcttggttacacttgcacagctgccttatttgttctgtcccattggaaagtccctcgttgatacgattattaaagtttgttggctacatctggttgttgagcttaaatcctactcttctttaaaatgggaatttacaagaaatggctcaggttaagtgtcgcttatgtctgcaaatcagtcaaggttgaggtaatttgagacctgactggttgtgtctgctcaggaatttttcaggtgttgtctccattttaactttgagaagcttaagccacctctggtgataaccaaaatcagtgacttaggcatggatttcaatgaaggtttcaagaggtttattaagccagtcttacagcagattcagaaaaaatgtgagtcacagacacacctgtgactatcttttctgaagaggtcttcaggagggttagtgtttatacttgcccttaaagcagcaaagtcctgtggggagaggggcgaggaggtagtaaggcaaatggccacatgctgagactttagttggtacccagtaaatctgcgttttacatcagaccagatgaacttttgaagaaagaaaggggataaaggaagcatcagttttgcaggtatttctgggtaggtagaggaaatggctgatctcatcttgtcattctgcacctgggaagacaagcttgtaattgacatgatcagtgtgaaatgaactgggcctccatccttaacttttcctttggcataaagagtttgggggtcctgagagttttctgtttgcctttacacaggagataaggttttccatttgttcaccatccactttgtgaaatcagcattttctcttctctggtctgtcgattctttgaagtttgacagcagggccccgtgttcttctttctaagatttgatgacccctcctcgttccactggctagagtctcccctatcacacccctggcacagaccaccccattatctacgtttctgcatgtagatgaactgggcggaggcagcgcattctgtctggaagaggagactgccatgctgggccttggatgccatgtccagagcacgctcagatgtctgcagctgcttgagtatcctgccgtttgtagtgcttctgtggctcagaattaacagttaatttcaaggcaaccttcctcccatcccaggagattattaacagagcttttcaaaggatctgtaacttacctgtaactctttccttgccagtgaagaatgtaaccacctgatgggttcttcctgcccatggcacaaacgaaatcaatacacagcatttcagtaaagaaagaatttaattgacgtgatgccggccacgccatgtgggagaccgagttgttactcaaatcacctcatcaaaggctcagaggctagggtttttcaaaggtagtttggggattggggggtgctaggcagtagtgcttgctgctgattggctggggtgccgtcacaggggtgtgggaaatggtcctcagtgggctgaatcacttctgggtggggctacaggagtggttggccggtccaggtggagccatggtgtcagacatgcagaaccctgaaaagatatctcaaaaggccaatcttaggttccataatagtgatgtcatctgcaggggtaactggggaagttgcatatcttgtgacttccagaataatggctgccaatcatttatatctgtgccttagcagaattcagcctcctctcctctccctagcccggtggtctctcattagctttacaaagatggctgagtttgggggaagggctcttatcacttaaaccataacctaaaagtctctcgaagttagcttgacctaagcccaggaataattgagggcatctaaagtcaagatggggtatggttaaatcaggtctctttcacagccatcattttctcactgttataatttttgtgaaggtagtttcaagaggggaggacagaggccaatttgaggggacagagtgtgaagctggtcacctggccctgtcccccccagaggccagtttgagggaacagagtgtgaaactggtcacctggccctgtccccgccagaggccagtttgagggaacagagtgtgaagctggtcacctggccctgtccccccagccctgtcccccccagaggccagtttgagggaacagagtgtgaagctggtcacctgaccctgtccccctcagaggccagtttgagggaacagagtgtgaagctggtcacctggccctgtcccccccagaggccagtttgagggaacagagtgtgaagctggtcacctggccctgtccccgccagaggccagtttgagggaacagagtgtgaagctggtcacctgaccctgtcccccccagaggccagtttgagggaacagagtgtgaagctggtcacctggccctgtcccccccagaggccagtttgagagaacagagtgtgaagctggtcacctgaccctgtcccccccagaggccagtttgagggaacagagtgtgaagctggtcacctggccctgtccccgccagagtccagtttgagggaacagagtgtgaagctggtcacctggccctgtccccgccagaggccagtttgagggaacagagtgtgaagctggtcacctggccctgtccccgccagaggccagtttgagggagcagagtgtgaagctggtcacctggccctgtccccaccagagtccagtttgagggaacagagtgtgaagctggtcacctggccctgtccccccagccctgtcccccccagaggccagtttgagggaacagagtgtgaagctggtcacctggccctgtccccaccagagtccagtttgagggaacagagtgtgaagctggtcacctggccctgtccccccagccctgtcccccccagaggccagtttgagggaacagagtgtgaagctgctcacctggccctgaccccccaggcgggtggtgacgggttcttcttacagctgcagacgtgcgtgccattgcccccaagaccacgtctcctggcaccaaaatgtgctgtgggagctcacaggagggagggggtgcatttacactggcgctcgggctggctttgccctggagcttgaagggtggtgggctttggtgtgcagcagatggaagaagcctgtgtcaggactgagctcagccgcagggagggaaggagggagcagttgagggagaagagcagttagcgtgcccaacctgcagagctgagaccactgaaggctcctgagaagtggggttcgcatgtgagctgtgtttcaggaaggtccatggagccgtcatttctggaaggaattagggcattcctggaaatgaggaaactcattgatcgagttcctccaggtgggagatggagatgccctcgtggtaagtgtgggaagggcaggacccaggcttcaggatggcagtggatgctcctcctgaaatttcatggagagcaaagaaggcagggtcagaggcgaggaaagtgcctctggtagggactgccaaagctgctggagggagggtgaaggggtcccagcacacaggtgggaggcccaaggcaggaggggagtggccaggggggcccagaggtttttacacacagttgttaggacctcagtcatctgaggggcataaggtgtgtcctggtgccagcaggcacctgggggcacaaggagaccactgcaggctcagaactgctccagggtggtcaggggttggaggaggatggggctgtgagatcatgagagctgggtcccaaggtatggatggggcagacatcccagggcccagcagctgttccacaccatggccggccgttctgtggaccctgggcacagctgctggccctgactaagctggggtggtgtccccccacccccgtcacatccctcaggggcagaggcagcaggagcctgaaggagagaccactaccaggtgtcctcacctggctccattcctgccgagacccacggtcaggccctgctgcaaggtggggggagcggtggtttgtcctggcgctgtggatgtatgttctgggatgatccaggaagagacacaagtcccaatcataaatctatggaagattgtccttggaatagcacggcaggactccccgtgccttgaggccttgaatgttcaccctctccaaggcctcgcgtggccccagccctaagcccctgcggccccagcagcatccgagccgtggcaggagtttgggaaaagggtttgagaatatttgacgtggaaggaagcactggctgtccatgaaggtaataacagcaaacatctggaagaggaagagtcaggtattttgcagatgaattagcacacatccattttcctgatgcccacatgcgtttacatgccaaagctgagggccccgaggaccctgcaggtgtggaagagactcccacctctgaggcatttgcaatgatgccttctctttaggcagagatggctggtcagggatcccaaccacatgtattggactgactcaggctcaatttggagcatacttggatgtttgggttgttttggattcagcacattcttaaaaattcataggttctatctggacctataaacgttactttgaaaaatcctcttgttcatttaatagttttggttatttctggatttggagttgtgggtttaatgtttgttcaggtaccagaatgtggcccccgcctgtggaatccacctgaaccccaacccagaatcaaaccgctgtttctgaaattcagaattctgctgtgatccagtttccaggtgcctcctaagggcggtgtcccaggagatggcgtttcccaacagtccgtagttcaccatcaggctgttgagggtgggatgcatgcagcgcactaattagacagaatgtcagcgtatctgagtcatctgaagaaaatccggcttgctgtaaagtatcctctgcagcattctaaacacgtttggagaccgcgctctgctgtggggcattgtctcctcctgtcttagctgtttctcttagggaccaggaggggactcttgccagccagggggatgttaggagccaatctccacctcaaggctgtgtttgcagggttaagctttgctgtcctttctggcaagaaaagccctggtcaggtagccctggacagtggcaggtgatcctgcccatagtacagtgcgtggcagtgcaggagatgctcagtaaacgtttgctggagaaacgaatgctcattactgccaagtagaagaaacttgtctgaaaaccatgctgaatcatcaagagcagcctgatggttggacggtgtcttgcctggtttctgcctctgtggacagtagggtcactgtggcgcccagagccccttctctgattatctctgcagttagtccctcagctctcggctgcagctaaggcccttcccagcatgtggccgtctcacacctccgcctgttgtctcctgagaagagcacattctcatttctgtactccgtgcattccagggcatgctccttctattagttttgacaggtaattgctttgtgtatttccctccgaaggctctttcctctctgtttctgccaaaagcaattattgtcatcgttcttaattgcagcatttctaaaatggcatttggcatatcatacccagcttggcgaatgcatagatgtagctctcctcatatcacttgcaaatattttaagaacaaatgatgtaaacatgcatcctcagaacaaaaagaagacagaagccttcatttggtagatttgtataatcactgtttgtgttagaattactgagattctgtaggagtgagagtaacaagatattttgtaattggccacctgaaaaggcagcattcaaaaatgtcactgacagggagatttcaggagtctggcagtgtgggccaccgtttcagcatctccagatgcagggaaggtttgtttacgagagcccccaagttcacattccacagggttgtgaaatctggacctggatggtgtcgcatccagcttcctgcattgtttcatcagcttcattctggagctttgtttaaccataaacactaagacaagaaggtcctggaatgcagctactagaacaccgagggtctctgtacgactgctccctgaaggggtaggcagggacgctggcagtggcttcgaggaaatgtccacccacctctcaactatgagcccctgagctctggagatggacaccgtggggcccttgctctgcatggtcagctgggtggctgggaaatgatggccctgagtgtccaggctgacaccaggtgtccaggaatggtgttattttgaagggtctctgagcagaaagtcaaagtaaggcagggatggaaatcatgctaaactgctgccaatcataactgcagcagccccttgaaggctccagaaagcagaactagggctgggggtagaaatcacaagtagccagattctggccctaggacataccggagaattttctggtgaactaaggtggacagctgctgcaggaggtgctgacttcccagactctgagagtaaccagagggttgggctgccctggaccagctctgtggcctttccagtcctgggcacccactggtccagtctaaagtgaggaaggaaccacagttccttgtgtcctcctcacccagggaccctgtggactggccaagaacatcttacactgcaggaaagactgtgtcttccctataggcatggacgccaactggatttgcaaggtcagcaagtaggggtcactttgtctggtctgggatgattcaggtcagagaaaatgtccgtggttcactgagaacaaagcagttttaaaaaatgattttagatgccctctctcaccactcctattcaacatgctattggaagtcctagccagggcagtcaggcaagagaaaaaaataaagcgcatccaaataggaagagaggaagtcaacttaccccttttgcagatgacatgattctatatctagaaaaccctatagtctcagccgaaaagctccttcagctgataaacaacttcagcaaagttttaggatacaaaatccacgaacagaaaacactagcatttctatataccaacaacagccaaactaggagccaaatcaggaaggcagccccattcacaattgccacaaaaagaataaaatactttgaaatacagctaaccagggaggtgaaagatctctacagtgagaattataaaacactgctcaaagaaatcagaggtgacacaaacaaatggaaaaacatcccctgctcatggataggaagaatcaatatcattaaaatggccatactgccatatagattcaatgctattcctatcaaactactaacagtattcttcacagaactagaaaaaaactattttaaaattcatatggaaccaagaaagagcccacatagctaagacaatcctaagcaaaaagaagaaagctggaggcatcgcattaccgtacctgaaacaatactacggggctaccataaccagaacagcatggtagtggtacaaaaacaggcacatagaccaatggagaatagagaacccagaaataaggctgcacacctattatcatctgatcttcaacaaagctgacaaaaataagcaatagggaaaagactccctattcaattaaatggtgctgggataactggctagtcatatgtgggagatcgaagctggactctttccttacacataaacaaaaatcaactcaagatggttaaagacttaaatgtaaaaccccaaactataaaagccctggaagacaacctaggcaatagcatcctgaacacaggaatggaaaaagatttcataacaaagacaccaaaagcaattgcaacaaaagcaaaaattgacaaatggagtctaattaaactaaagagcttctgcacagcaaaagaaactatcaacagagtaaacagacaacctacataatgggagaaaacatttgcaactatgcacctgacaaaggtctaatatccagcatctataaggaactcaaacgaatttacaagagaaaaacaacctattaaagagtaggcagaggacatgaacagacatttttcaaaagaaaacatacatgcaaccaacaagcatatggaaaaaaggtcaacatcactgaacattagagaaatgcaaatccataccattatgagataccatctcacaccagtcagaatggctcttgttaaaaagtaaaaaaataacagatgctggcaagattacagagaaaagggaatacatatacactgttggagggagtgtaaattagttcaaccattgtggaagactggcaattcctcaaagacataaaaacagaactaccatttgacccagcaatcccattactggatatataccaagaggaatataaatcattctaccataaagacacatgcacatgaatgttcattgcagctccattcacaatagcaaagacatagaatcaaccttaatgcctatcagtaacagactggataaagaaaatgtggtacatatacaccatggaatactatgcagccataaaaaagaaccaggtcatgtctttggtgggaatgtggatggagctagagacagtttccttaacaaactaatgcaggaacagaaaaccaaatactacacattctcacttgtaagtgggagctaaattatgagaacttatgaacacaaagaaggaaacagacactgaggtttttttttctttctttttttttttttttttttttttttttacagagtcttgctctgtcaccaggctggagtgcagtggggcgaccttggctcactgcaacctctgcctcctgggttgaagcgattctcctgcctcagcctccctagtagctgggaccacaggtgcacgccaccacgcccagctaatttttgtatgtttagcagagacagagtttcaccatgttagccaggatggtctcaatctcttgacctcgtgatctgcccgcctcggcctcccaaagtactgggattgcaagcgtgagctactgcgcccggccgacactgggtcttcccccagttggagggagaggagcagaaaagataactactgaatactgggtttaatacctgggcaatgaaatagtctgcacaacaagctcccatgacatgagtttacctatataacaaatctgcacatgtaccccagaacctaaaataaaagttaaaaaaaagattttcccacctcaactggtgaatcattccatcaagctgttagtcctttaaattgataataggcatagaggatcaatggctttgatggccaaaatgaagataattagtttttctacaaactctgttacatctgcattttagagctcagatggaaacctccttgctcagcagctgtctttccagagaagacacaaagactcacaggtgtgccctgcacctgtatgtgacctgtgctttcacattagaggcagggactcagaaacacagtcagccagcaccctccggacccacccagtggcagaaagtgaggtgagagcgttcactctcctttgttgccaggcctgtttttatccagttcagtctcttcctgggagaccaggtgagctccgcagaagcaagagagcagtggctcaggagatgacgtccagaccccaaagagagattccgatgggagcacacttgcccctggagcctcgtctctgccaccagccaggcaccacctcagttctgtttgtgtagcctttggtgcatgacctcggggttcctttgaactagaatttgatgctaaaggcttgattagattcgggtccaaggcttttgggaggaagacgtctggggacactgtgagcctgctggtgcatttcccagtgcatgatcctgccacccgtcatgtcccttgacctcctattatgctttttgtcacctggttaggaagtgacctctagaactccacgcataaaagcgtctctttcccttcccagggagttaaagcggggctggagaatcacctgtgcggttgctagcacagtggtgtaagctctggttcaacaacttttcacgtcttggcttttgtatctgtcaatgattcgtaccaatcagttgtctttgaaggttaaaccatgttgacttctccaattctaatcattctccacacattattaatgagcatttgcagtaaagaagggcttttcttcattgactgggaaagaaccacaattccttttaaaaggacaggataaaagtccaagcattttcggcctctattaaatactgaataaccagatttcctatcagacaattatttcattgccatttgtttatatcataaatactgtggatattgaagattacaaatggggcatttaactgtgttatgattgtactgtggaatattatacagccattaaaaatgatggtgttctacatgaataacatgaaaaatatgcatgatatattaagtgggaaagcaaggaagatttccatatgtataatatcatatttttgtaaaaagaaatcatacgtaacttctgtgtatcccacgcacgggtacctaagcatgtgtaacagtgtgtcaaaatgaactcctggggtctcagattcattatggccctaagcagggaggtcctcttcccaagcaagtccctgacgttgctggactcagaagtggtgtcctgatagttacctctgcccttggaatgtcatgtctctcccccatcccctgtgaagggagaggactggaaggaaaacctcgcctagtaaatggagccctgggagccgactcgtgcagagggagctggcctggggctgtgttagagtctgccccgcacgacgaggaaggaggaatcagaacagcagcatggagggagggagggagggagggaggtgaccacccaggtctcctgagcaggtcaggagcagtctcccaccctcccctccagctcctcacgccctgggcatgggagggagacgcctcccctcttcccatgcaggtttctgccccaaaggttatttaatatgtttagctccaaactttaatgtttaagtccaagcttaaaaagcaggcaagggtgctttttgcagagggtcagccaggaggcggcaaagtagggctggacggcccctgtttaagtccaagcttaaatgttaaagtttggagccaaacatattaaataaggaaaccaaatcataccttagactaggatggttagaaaatgcattttcaaagagtggacagaaatactagggacagaggcacctgctcacagaggtgagaagcgacaaggcccagcctcttacccgtcataaatagggtgcggtgggacacagagatggagcccgcacagctgcacctgccacccaggctgcacactagcctgttactatgcagtggtgccaacgtgaggccttcactcttctgcctgtgctgtgttgtgtcggaacatgttcccagcgggcagagattactttcctaatttgaaaatttacacctggaaaaatgcttggaatcttgaagacatactgaagtgaggcatgtgtattgtcaccttagatatgaaaagttctaggctgtttcccataaaagaatcctggtggcccagggcagcctggtgtgatgagacttggtggtgccccctctgtccctgccttcatgacctgggaggggtccaggtgtggtgggcagcccctccttggtgagcactccaagggggaccatccggccctgctttgctgcctcctggacgaccctctgcaccaggcatccttgcctgctcttctccctggtagaaggctgctccttatttaagagacaggtgaggagaatgacaaatacagtgagaaaaacacgaaatttttttcccattcagagatgtttagtatgagtctgaactgaaccaaaatacagaacacaggcacggctgtgtgggccgcgaggaccctcagcaggtgcagaaagctcaggaggtgcccctgcagaggtgaggggcccacgttgggagatgccaccagggacaagctcagagctgagggtggccccagcacactgtcccctccccaagggggacaagcatttcttccgaccccacctaccaggctttgtgcatcaaggggacaagcatttcttccgaccccacctaccaggctttgtgcatcaaggggacaggcgtttcttccaaccccacctaccaggcttcgtgcatcaagggggacaggtgtttcttccgaccccacctaccaggctttgtgcatccctggacagctctgggtaggaaggccttgcaaggctgcacttcccaggctgggccctcttggccttcagcccacctgaccccaccctggaggatgctgtgtggaggctggggagcagaggttagggcatggctgggcacccactggctgcttcacctcgggcactacacacatgggagatgggactggtggtgcctgcccttgggcttctggtgatgaatgagctcccatgtcagagcgttggggttgtgccctaggaggggtttgcctcggcctctgcctcgcaccgctgcctccactcccccgtgcagcaggctggtctgggcatctgcgtctgggctcctacaggtgcagggggaacagcaagcatgagtccgagtctggagacgcaggccacgtgttgtcctgcaggagctctgagtgccgccaccctgcagtcgaggccccgtgcacccagctctaccttgcggtgcttgtgatcacttcctacctggtgtcatctctgttctgcagaggagcaaactgaggcctagggttagcaacatgctcaggggcagccaggagctgtggggcgggacccctgcatgcctgggccccatccttcctatctcctgtgtccacccggggagctgcggcccattgtcagaatcgccagggcctgtttaaatggatggctagggaccaccctacatggtgaccccattccagcctctgcattccaaggctgggcctcagccctgggtacacttggaaccacgaagaatcctgaaacaataaaaacatcctccaggcaaattaaaccagaacccctggggtgtgggtgtttatagcagtttcctctgaaggaaattatagcgaattttcctctgaaggtgtggtctgtatggaggtgtggtctgtaggagtgcacaaagcgctgtgccgcacacccaggcctcggttctgtgcactcggaacagcagagcccatgctggtgacagcatccaggagcagctctgtgcacccgcgccaagtgaccgtgggagccacgttgttctggtcacagcagattttatacaaagaatcaccacggtacaagaagtgcagtttttctggtttctcttaaggtaagagaaaaagtcactttctataatgaaagaacttggacctgatcatgcatctctgtctgggctggaagagaggggctagtggagctgggctcatgcctgccattgaatggcggctacgtttccatttctctcttccttcccctccgtctcattcaccgtaaacacccactgaacctggaagcactgcacaggaagaggcaggatcaggaggcaggggaggtgggatgctgagcacgctgttctcccgccttggtcttgctcatcaggacctgtgttttaagctcgttccctgagatggaccaggctgactgctttcacatgcagctccttgagacacagaaaatggtctccagcagcacactccatcacaaccaggcaatcggaattcctcctgcaggtcttttcctcgtggctatctccaggaagaatcctgttaccgtttggggcagaagcggcgaggattgcagagaactcaaccactcagacatgcggctgggaaggtatttaaacacccaccttcggaaggagtggtcacaggggcttcagcagccccccagcaggaagagagtggctcatccttcagcccgttatttgctgtgcaaggggagtattcgatgactgggacgcccccggcctctctgagctccccgtgagaaccagctgggacttcctgctctgcccagattctggaggaagctttgttgttgtcacttgagacataagtcaagctttcttgagtggaggatgcgattggaaatatttgcttctaaatgcgttggaggcaagctgtcatccgagccgatggccagtctcaggaggctttgcttctcagctttccttttcccactcgctggttcctagagctgttccccagccctgtctggatgaagcagcttcagccctcatgcccctcctccaagtcctgctgacacttggtcactgatgacagcagcagggtggcttcccatgactcgggcctcctgggccacccaccctcagctgccactctctgtggagttgcatgtggggtaggggcaggtgactctgaagatggaaggagggtgtgggggaaccatgggaagatctagggcaccacctcccagagctgcccattgtccaaagagagcatgagctgcgtggcaagcatggtgcaggtgacaactggagttgcacccatctgtgtccatgactcaggtcagcaggtgcagccccacctctgagatgacgtcctgcattgcctttgtgtggtgcggcactatgtttcatgtgctcactgttatcctgtctgttcacaggccatacaaagggattcatcctcattcacgccggaggtttaaagaggatcctggacccacatacctacccattggctccatgaccagtgcttttataattacaaaatcattgagaaattaagtcacaggaagcctcgtgtgtggtatacactggtgataaagcaagtcccatgcagtctgagatggcaagggaccttctagggaaagggcagtgacccagaagcccgactgctcggctggctcccgcactccgtcctgtggtcagagtgtgtttcctcccctctgtgaacctggtctcctccccagtgtgaggacaggggctctcgccttcttcagggagttgctgggaggacaaaatggagtatatgtaaaagtcatttcctagataagggggagcatgtcgtaagagtctcttgaaatttaattgatgtttatgtcattgacattgaaataatgagaaaataaatccaaatccagagctcaaagtaacacctataacgtcatctgtaagagccaggacttcctagggagtctctggacagaaggcagcgtcttttatacggtaggattgcaggttcctgtaggttcctccccactctgtgcgcactcccctttggctggaaggtgtcacataattgagttactgctgtgttcctcagaattccacctggaggaattcccagcataccggtaagaacttgttcaagcagcagcctttcccaaaccctgtgtattccgttgaaaatgctgtgaagcctcagaaatgtcggaaggctgcaccatcacagggtccgtttttctgaacgacttagtctacagtgtctttttcaggagactaagctgattttcaagcaaaaattaggaagccaccactgccagtctacattgaaaagtcactcattgcctggaagcttccagccccctgaccagcccctgtgactgaccaccggtcactgcccactctgcccacttgctttcccaaatgcactgaccatgcgggcactgaccatgccggtgccaacccactgcagtgccctctggagggcccatgtgactctgctctgccagctgtttgcttcccaggaagatttgctcctggttatgtgtggctatggcacaaatctcctgaaacttggcttcaaaattagtagttggctcctcccaaggctctgaggttggcccaaaaggttctgcccacgtcacccaagctgttcctttcctacattcagcaggtgcctaagccttctccctgcagcctttccgaccagggctcccaccaactgctccagcccttccgtgatgaccaccttccagaaggaagagcctgtcagtccgaggggtcagcacaggtcacggggccacatagcagggtggggacaagaccccatgtccaatgggtggagctcacagcagccctgggagaccttgctgttcacaaacaaatcgatgcctcacacctgctgcctgactacttagctaaacgcagcctaagctggtatatgaaaggaagcacgtggccccaggaagctgacttgagtgcttaggaaagactagaagcagcaacggaatcggaggtgagaagggcagctagagggcgtggggcagctcggacaccagctttgccagcctgagttcctccattcccagcaccctgaccttggaggctgcaggcagcacgtcaggggcgtgatctgaaaaggccaagggacgacccagccagcaagcctgagctcaggggctttggtgacacattctaagcactagtggtgacacaccttgctgtgttctaattaaggtgagacgtccaaagcagacagattgaagtcctgtttcagccaggcttgcctgccaagccctgaccatgactttgggtgagaggctgctgagatcagaggaggctccttctctacccctctgccaagttttgaaagaggaagattggggtgataagtgaaacaactttatcatttatattgattcaaaggtattttggattatttacatgggacattttagccgaagaacaatgacccagagaagcggctcctctgagcttctgcctcggtgtgcgagccaaagggtttcattgctcttttctctctctttttgcttcttgatttgtgagacaggtttgctgggtttatgttgcatttgtctaactctgaccctagacagtgctgtcgagtagaatttcccatggtagtggaaatgctggtgtgatggctaccagccaagttccactgcggagcattcaaatgcggcggctgcaactgaggaaccaaacctttaactgcatctccttttaattagctttcatttaaatggccatgtgaggctagtggttgccatatagaaagtacagttctagggttttttctctactttttaccaatttatacctcttagttcttctttcatctgctgttttagctgcaacttatgacacattgttgaataataatgaattgcaggtacccaacctggctcctcagttgtacaattctgtttaaacatgtctaatacttgctctttctctttttcttccctccccgctccctccctccctcccttcttccttcctttctctctccctctctctctttctattctttctttcctcttttttttctttgagacagggcctcactcagtcacccgggcttgactcaagctcctgggctcaagcaatcctcctacctcagcctcctgggtggctgggactgcaggtacttaccacagtgcctggcttaatgcttgctatttttaacaaatattctctgtgatgttaagggagtctcattattttactattttatttagggttttaaaaaattagaaatggccactgaattttaccaagtaacttttcagagtctattgatacattttccacttttcagagcgacctttgattttatgatcaccatctggaaaacctaggttgccaccagagcttaacaagtcccatttccctgactgccttatccgagcacttttgtttgtttctcactccatctgcaaatcctttgtttcatgaaatctcagctgcccactgctttccaaacagacaggcgtctccaagtggctgtgcttctctcgggggtggccctgcctgcctccccttctcctcccatgccgtcctgtgcctcggcgcacactcctacagaaggacttgctgcctctcccaaattcgctgcctctcccaaagggttcactgtgccaaatttgaatcacttatgtccaacactgaactgatgacctattcatgcactattttgctttggcctgcacaatgttccttaaaaatgtaaacatttaaaaatgggtatcttaatataatgatacactttaaatgctagttttcaacagagtgcatttataaaattgtaaaaggcaaatgatactacaaggctgtatgaagataatagtctccggcccctcgtcatccccagttctcagccctatggcagcatctctttcatctcttctctggtatttattttatttctaaacagcatacttatgctcctgtttcttttctttcttcctcccaatttacatcccagtgtccattagatttctaacaagaataaagatctatctctcctgtgccattcccaccacccaagtataatttatcatcctgccagcctcctgatagactcacttcacattgctggtgtacatcattgccatcacagttgagccacataatatccagcatttcctttcttcataactttatgcttttcctcgtttttctgttgctgggtttttcacctacctctcaataattaggcatcaaacctcaggccagaacactctcaagacactgcactgaggtggtctcagggttccattttttctcttgtagcaggacgagctgcagacaaaacccctcagacaccgagatggtgaagggagtggctttaatcagctggaagcattggcagactagcgtcttaaaatccgagcttgtcaggtgcacaatttttgtcccttttaagggctcacaacgctaagggtttcacatgaaagggtcatgattgattgagcaagccagggggtatgtgacaggggctgcatgcacgggtggtcagagtgaaacagaacagaccgggaagtttcacagtgtctttctatacaatgtctgcaatctatagataacatcagttgctaggtcaggggtcaaattttaactaccaggcttaggtcaggcaggtccaggcctggtgtcgggtctggttcctaggcgctgggctacctgcctttagtttcgcttctgtttccttttctgagtataaaacaatctaaaacaatgtgagagggtctgtctctcttctctcactcctagagccactccttctggattccccctccttgccaagtcgggatccttgagctggggtctggccccaggcaggcattgtgagcctcctgctcacacagtcggggtcctcggttggggatctggccccaggaaggcgccatgagcctcctcctcacggtcaggatccggtcccaagtaggtcccgtgagtctcctcctcacagtcagggtcggtcccaggtaggcgccgtgagcctcctcttcacagtcggggctctcagccgggtcccgtcccaggcaggcgccatgagcctccttctcacagtcagggtcggtcccaggtaggcgccgtgagtctcctcctcacagtcagggtcggtcccaggtaggcgccgtgagccgcctcctcacagtcggggctctcggccgggtcccgtcggttccctgcagcgtcctccacccttgccggtgccgggggtaccgatggcctgtctctcgcttctccccctcctctgcctgcctatttaacacagttgcttctgtctgcaaggctgtagtgacatctcttggtgctgccctttcaaatttttggctgtcctttgcgtgaggttgagaagctgagagagaaactcctgtgttcccgcctggcagctgttgccaggaggttgcagccagtgccacgtgtgcgaccggcactgtggcagcgcttccggctgcgccttcggctgcacttccggctgctacctcagcctcggcgccctgacgccctcgagtccgcttcctcacatcctgacagcgtctagctctgctccctcacgtgcggtccaataaaggaacaaatgaagagaagactttccctggccattgaagggatcagatgattagctctagatgtttaaatcaaatcctactgtaatgcaaagactgtttgatgtggtatgaatttagtatctctgttgaaatttaaagcagcaattagtattatacagtatataattattgcattataatttgtattatgtattagttatcttactatacattatcttatttattgtttatttattgagacgaggtctcgctctgtcgcccgggctggagtgcggtgccatgatctttactaactgcaacctcgacctcccaggctcaagtgatcctcccacctcagcctccctagtagctgggactataggcatgctctgttacctccagcgaatttttaaatattttctgtagagatggggtctcgctatgttgcccacgctggtcttgaagtcttgggctcaagggattatcccgcctcagcctgccaaagtgttgagattacaaacatgagtcactacacccagcccattattttaaaataacaatattatataattattgctttttttttttgagacggaatcttgctctgtcacccaggctggaatgcagtgatgcgatctcggctcactgcaacctccaccttctgggttcaagcaattctcctcccttagcctcctgagtagctgggattacaggtgtgtaccaccacacccggctaatttttgtatttttagtagagacggggtttcaccatgttggccaggctgttctcacactcctgacctcaaatgatccaccaacctcatcctcccaaagcgctgggattacaggtgtgagccaccacacccagccattactattatcaaggtagaaatttgtcattttgtgataaaaagctgaaccttcaaaataaccagacacgaaactgccagagcttgctgttttataggagatggacagaactttgctgatcacatgcagagtcttcacttggaggcctagacatcttcactttcctctccatcttgttcactcttggctaggaaccccatcctgacacctctccaccacctgccctacacaccagagttagaagtgcttggtttacgaacttagcatttaaataactctactacttagactctaagcagccttacacgggggctcacgcctggagtttctagtgatgcatttacatttgatcacttgttttgaacattttaaaagaaaaccaacattttctttaaatatgttaacattaatgtaatatgaatttatgtacttattcaagtattggtatgtaattttattttataataaatatattttcacttttattgaaagtatttagaaactattttcatctgcaaaacttagcttatggccagcttttcagagacatttctgtattcttgaaagtaaaacactcctttgttcacaccaaagctgtgtgttctctatggatgtattcctgcaattattgcacaggtctgggtggcatgtgctgaaaggaaaactgattgtttgtttgtttgtttgtttgtttgtttgttttaccgaccaaggctagcacattgcttttctgatactgaggtttaagtaaaaggtaaaattttagaggtctcaaatatagtataaataccatggacaagtaggaaagtcctgggtttaacatgcattccatcactgttgtgtgatattgagaattcattaaaagatttgaaccttagtgcttttcatctggaagataattaatgcaacatttttatatctaaccttcaccaggtggttattaagaccaaatagtaggtctaaaaatgtttaagctatgcattatgcaaattatttgacaactccatgaaggtaacctaagattttgttgctaataaggcatcttgacagtgaaaaatcaaacaaatattatcaaatacttttatttgcaaataatcattgaagtcagattccacagaagcacaagcacactttccctgggcgtctccctccccgacggggccttacactaatacccagtggtgcccatttcatcacactaggaaatgctgatcacaagaagtaagtaatgattctcttaaaattcagaaggtacatggtatacattgaagtactcaaatgctttctaccaccatatggttattcctccaaagctaattgagatgaacccacaagctaactatagtctcaataattgtgttggtgcaagtgtgtttcacaggaaaggagtttgcgaggcagttatctcattaatggggtttctatttgtgacacttctgtcaccttgcacctgcctcactctgcttctctccttccagcctgcagtggcgtctgggacaacatcacgtgctggcggcctgccaatgtgggagagaccgtcacggtgccctgcccaaaagtcttcagcaatttttacagcaaagcaggtactgtccgtcggttgagttcctgagctcctccagggtgtttgcatctgagcgtccattttttccatccaagaagcaggaacatcactttccttcaggcatctcctgctaccctgggggtcagggaacagctgtggcatattggggtgagtgcccgtcttagggagcagaagggcatcacctgccttcagccaaaccccagagagcagggcaactacagggaaagaaccaggggagacaggagccaaatgggaaccaaagcacgggaaacgtgcatgagaggggaggggaggcaagggggccctaaaggaggctcccctcctcccactgagtcggcctgtgagccccagagagcacactctggaagtttctgggaagtgggaagtgtggcacccagagcctccatgctcagggcagaggccccaggacacacctagtttatcgaacccactcccccaacctccctgccgcctcacaggtttgatgctgcctggaatttgcagtttctctgggcttccaggctcctcctctgcacccagggcatgctccgtatgcggtgctgccaatatctcttggctgaatcctgagagaaaggttttgtttgtttttgtgagtgattcagattttcatagtgtgaagaaatctattcagaaaaatagaaaacataggaaacatctggaaacagaaagcataatactttggagcactatggagaatttacttttatcttggacagaggttaaagggtaaagactttgggaagtaataatgctgtagtgttttaaagcagagttttggaaaagagattgggacagaggagaaggagattccgaaaggaagattttgactttctcaccccgagttgccagcaaagcagcctgaaggtagggaccatgcttgcattaccatcactgcccacccctgagttagaacctaagtcactttagagaacctgaactttaagaactgttccctccaatcagtagcatttctactgagcagcttgggctgcatccattctcctggagatctggagggaattggcatggactctggcttttcctagtcgatggcatggttctgaggcttccagaaagcacccagcagcctggtagtttgcacattagcaagcaccagaatccagtccagcctctgccagacagaagccatctgggcaccaggcgcccaggtcagtgaggctctggccctgcctggaagcatcctgctgctgcctgtgagaatgaaaactcccagttggctttgggttaaatgcttattccacctttttcccaagcagggagcatgacaccaccttgttattgtgaggaaacttgtggtgtttccccaagtctgggcacttgagtagtcgcagctagatgatctgaaatgcaggggaaatatgcttctgctttttcaactgatcacaaaaacgtgagctatgcgaggacatgcacaaactgtggacagatggtccccatggaatgtccgcatggaattcatctttggggcattaaatgggcatcctttgcaaatacttagagagatgtggatgcgactgggggccgagcagggctggggtgaagccacctggcatcgtaccacagaagctgaggcttcagagactaaatttggtgggataactgtagagtcttagaaagaaatgaaagtcaggcttgcgtctgcatggtgctctgtggtctacagaacactctacacatgtgatggctgctcctcccatgacctgtaaggagagctgggtcatagaacacacgtggaatcatttctgcccacaaacccaggtgttccagaaataagtcaccattctaatgaaacattgtgttctcttttgatttctcatccttccagcaggtgggaggtttgggggctgtctgtcctatgctaggcaggtggcctctgggcatcatcacgtgtgtccggcactgtgcatcatcacagtgaccagcacactggcgccctgtgtctctgtgccaggcgggaccctgggccctgtccagaagcagtgggcagctccttctccaggtcctaacccctgccctgcagcagctccagaagcagtgggcagctccttccccgggtcctaaccctgccctgcagcagttccagaagcagcgggcagctccttcaggtcctaaccctgccctgcagcggttccagaagcagcgggcagctccttccccaggtcctaaccctgccctgcagcagtgggacccgggatgcaagtcctcttcccacccgggctggacttcaccactgctgcttggggaaagctgtgaggattaggtgcttcagacatttcctgcccccatcccctccaggaggcaatgagccaactgtctgcatagaggggaacaaaggaagcagggagccagggtgagaggcacgaggactcatcactgggaggacagccttctgtgagcagctgctgttcatgccccatgctcaggggccaggaggcagctccgacagctgtgtgctgacctttctcccttgacttgctttctgtgtttccagttgctcctggtgcccacctgccaattctgcatggcactgttttagccagacccagggcagcagctcagccgcccatccagcacaggtgtagggccaggtgtggttctgatcctttaaaggggcatcagcacatcccccagtggaggatgatgtaggctcatggggcttggaatagtcgctgagaagcactgaagccctaagggaggccttcaagtattcgtcatcctgcaagtgggcgagagtgtgctcccaccccccacaacctttctctgcacctccctggcctctctgcccctggatggcctctctctgcacctccctggcctctctctgtacctccctgatctctctgcacctccctggcctctttctgcacctccctggcctctttctgcacctccctgatctctctgcccctccctggcctctgtgtgcctccctggcctctctctgcatctccccggcctctctctgtacctccccggcctctctctctacctccccggcctctctgcccctacccagcctctctctgcccctccctggcctctctctgcccttccctggcctctctgcccctccctggcctctctgcccctccctggcctctctgcccctccctggcctctctctgcccctccctggcctttctgccccttcccggcctctctcagcacctacctggcctctctgtgcccctccctggcctctctctgcccctccctggcctctctctgcccctccttggcctctctctgcccctccttggcctctctctgcccctccccggcctctctctgccccttcctggcctctctctgcccctccctggcctctctgcccctccctggcctctctctgcccctccctggtctctctgtacctccctgatctctctgcaggttgccacctcagtcatttcctgtccttccacatctgccccatgtgtctcaggagtgagaagtcagaaaggataaagaacctgtgagggaatctgcacttcatagaatgactttctgacttagtttgtggagttttatttcagcatcttctttggattggtaaacactgatctcccgtaggaaggagaaaaggctgcacctcgcagaggatgagtgacaggctcagctcccaccgaggtcctggcttccaggctcaggataggggcctgcaggtgccctgctcaggtgagagccaggctgtcaggtgtggcgcttggcccatcgctcaccagtgtgactttgggcaagtgacacgacttcctcagccttggccgtctcatctctgagattaattggagagctgcctcctgtggtgtcaggattaactgggttgatgcaagcacagagcccggcattcctgagggctcagtgagtcgtagtggccactaccacatgactagtattattataataactattatgagtgctgttcctgagtctgtgatgggcacattatttatctttgtggacctgtctccttgtctgcacaatggggccgtgggaccaggtctctaggaggcttgcctgctttagggctctgttttgcctctaccgtgtatggggtctctgcagctaagaggcccccagaccctgctggggctccctgtgtgaggtgtgggccttgtagtcagagcccctgattggtgatggtgtcggggtgccagtgggcagggccagccctgaggccttgctgagaccagtctcccaccccactcctccccagtacagccagagcctggcccggccctctctctgcctctcatcactgcaggacaagaatcatgcacgcaccccactcaaactactgagtcttcaaagccttgttttccccttctgtaaaatgggaacagcagcgcccaccaagtctgtcttgcagggttgtcttgaaattaaaaagtcacgggagtgaaaatatttgctaaatccaaagccccagacccttccagtcacccaggcagggagacaggggctccgctctggctgtgccaggagtgcatggaggggtccggcctcctgggctctgctgtgggggtgaccctaacaccatgcagccatgctcggggcaaggctggcacggcgttcagtgcctggttttattcttacggctcggacaccttcgagagctttatcacttactgttctgtgtggccatctgctaaggacacacacaaatacaaggaacattgaatttctgagcttacatatgattttagttccgtaggcgcaaaagatgagattgtaagggaacaactgccttggtttcctacaattaaactgtaattccgccatcctttcttccaacaccagtggccaaatggagcctgccctactgggtcacggttggtgatgagggcctggccaggaggagggaaggtgctgtcaggaactggggcaggccaggcaccaggtcaccgttggtggtgagggcctggccaggaggagggaggatactgtcaggaactggggcaggccgggcacccggtcaccatcggtggcaagggtgctggctgggaggagagagggtgctgtcaggaactgggtggggcacccagtcaccgtcggtggtgagggcctggctggaaggagggagggtgctgtcaggaggagggagggtgttgctgggaggagggagggtgctgtcgggaggagggagagtgctgccaggaactggagcaggtggggtacccggtcaccatcggtggtgagggcctggctgggaggagggagggcattgctgggaggagggagggtgctgccaggaggaggtagggtgctgtcaggaactggactgggcacctggtcaccatcagtggcaagggtgctggccaggaggagggagggtgctgtcgggaactggagcaggcgggacacctggtcactgtcgtgctgttgggaactggagcagtggggcatctggtcaccgtcagtggcgagggtgctggccgggaggagggagggtgctgcccagaactggagcaggcggggtacccggtcactgtcggtggtgagggcctggccgggaggagggagggtgctgtcaggaactggggcaggctgggctcctggctccaggatggccagggcttggctataaatatcacttatggtggcagaacctaagagtctgttaagaattttcattctgccccaaaagaactacacttacagttgcggtaattatgacttatgacacagtgcgttcactcacatctcaggacccaccggccccgagaggtgacctgctgtccaggtcacatggactaggagggcaaggaaggttttttgagtggcagtcctgtctcctagctgacccctgggtgtgggaagcattttaatagagcacgggctcagcacggactaaggtcccttctaaggccagttttcatggacttgcagataaaatatctgaacttcactttccaggaaacataagcaaaaactgtacgagtgacggatggtcagagacgttcccagatttcgtcgatgcctgtggctacagcgacccggaggatgagagcaaggtaggctcctgcgtggtgctacggtggtgaggttccacttcctaacactgcaccagaagccccctcctgccctgcaagaattatgtagatttttcccctaaataatcctctgagtaccaatgctactttctgctttccaagttaagcgaagtccctgtttccgttctgctgctggcacttgggattgtttgattgttccgtcattttgcaggcagggaaaggccggttttcttgacctgctgtgtatgtgtctgggagatggtgaggagaacctgagagtggccccgcctgcccgaaagttacttttcagatgagtcctggagagcatgccctttctgcatttggatttcttgtcccatttaagattttgccacattctggggcattgagaagtcagcgccatggtttcctgagccctgagcctgcagcaggagatgtgatcacaattagggtgtcttttcaactaagagacccagcttgtcctccgccaagaggtgtctgggagcaggggtcccaggatgagttggggaccccccaatccgggctgcacaaacttgctgcagtgatctggagctcttgggtgttatgtccactgtgaacccttctgcagggctgtgatctttcagtaatgaggatgtttctcccgagtgtgggatgtgtggccttaagctctgctctggtccatccgtgcaaacttccccagctctgcctcagacagctgcttctcctgagggctcactctgtgctacgtctgcctgtggtttatcaccagacagacctccatccttgccggcagccctggcagcaccgtgtgggtggcgggcaccatagcaacctgcagccagaggtgtgtgcagagtgtgctccctgcacagggcaaggcaggaggcaggggatacattggaggctgctctaaggagccctgggaagtatcgcgctccccacatgcaaatgttctcctacaagcccacagcattccgggcaaaaccatcagcttgtgggccgatcacctcctgccctgaccctgctccgcctgcccctcctggaggtgtggcttctgccctctgggcccccaggggatggtggcaatgctgcctggctgcctcttgggagaatgagcccaactttcttacagaggcctcctttgctcacttctctaagattcacaactctaagccagttctttctccgccaaattattaaaccctaggaaaggaagtatcttatacagtaaccaatatcccaacgattggaaggtaaaactcctgacccatggtatatgggtcattatcacactgctgataaagacgtatctgagattgggtaatttataagaaacagagctttgatggactcacagttccgtgtggctgaggaggcctcaaaatcatggcagaaggtgcaaggcacatcctacatggcagcaggtgaaagagagaatcagagtcaagcgaaaagggaaaccccttatcaaaccatcagatctcgtgagacccattcactatcacaggaacagtcggaaccgcctcatcggatcttctgagacccgttcactatcacaggaacagttggaaccgcctcgtcggatctcgtgagacccgttcactaccacaggaacagtcgggggaacggccttgtcggatctcgtgagaccagttcactaccacaggaacggtcgggggaatggcctcgtcggatctcgtgagacccgttcactatcacaggaacagtcggaactgcctcgtcggatctcgtgagactcattcactaccacaggaacagtcggaaccacctcgtcggatctcgtgagactcattcactaccacaggaacagttggaaccgcctcgtcggatctcgtgagacccgttcactaccacaggaacagtgggaaccgcctcgtcggatctcgtgagacccgttcactaccacaggaacggtcgggggaacggcctcgtcggatctcgtgagacccgttcactgtcacaggaacagtcggaaccgcctcgtcggatctcgtgagactcattcactaccacaggaacagtcggaaccgcctcgtcggatctcgtgagacccgttcactaccacaggaacagtcgggggaacggcctcgtcggatctcgtgagacccgttcactaccacaggaacggtcggaaccgcctcgtcggatctcgtgagacccgttcactaccacaggaacagtcaggggaaccgcctcgtcggatctcgtgagacccgttcactaccacaggaacggtcgggggaacggccttgtcggatctcgtgagactcattcactaccacaggaacagtcggaaccgcctcgtcggatctcgtgagacccgttcactaccacaggaacggtcggaaccgcctcgtcggatctcgtgagacccgttcactaccacaggaacagtcaggggaaccgcctcgtcggatctcgtgagacccgttcactatcacaggaacagtcgggggaaccgcctcatcggatctcgtgagacccgttcactatcacaggaacagtcgggggaacggcctcgtcggatctcgtgagacccgttcactatcacaggaacagtcgggggaacggcctcgtcggatctcatgagactcattcactaccacaggaacagtcggaaccgcctcgtcggatctcgtgagacccgttcactaccacaggaacagtcgggggaacggccttgtcggatctcgtgagaccagttcactaccacgggaacagtcagaaccgcctcgtcggatctcgtgagacccgttcactatcacaggaacagtcggaaccgcctcatcggatctcgtgagacccgttcactaccacaggaacagtcgggggaacggccttgtcggatctcgtgagacccattcactatcacaggaacagtcgggggaacagcctcgtcagatctcgtgagacccgttcactatcacaggaacagttgggggaaccgcctcgtcggatctgagacccgttcactatcacaggaacagtcggaaccgcctcatcggatctcgtgagacccgttcactaccacaagaacagtcaggggaacggcctgcatgattcagttacctcccactgggtccctcccacatgtcggaattatgggagctacaattcaatatgagatttgggcggggacacaggaaaaccatatcacgtggctagtggataaggtggtgctggctggcattggcaggtgttactgtgaacccactgtcattgtgttcagcagataaggtggtgctggctggcatgggcaggtgttactgtgagcccactgtcactgtgttcagtggataaggtggtgctggctggcattggcgggtgttactgtgaacccactgtcattgtgttcagtggataaggtggtgctggctggcattggcaggtgttactgtgagcccactgtcactgtgttactttagttaggcttgaaaacaactcactgcatggatggagaagctgaggcttaggggagtggtgaggtgtgctccccactggtgagcagccacagccaggccctggtagtctgattcccatgcagtctgcttgttgccatctggtcgggacagctcctggccccaccgccctccctgagtgggttgaaggcacctgcaggagtcgcctggctggggaccacattgacctgtgggcccgtctaagatgcgctgtcccccatggcatccagtggagttgagagaagactgagttgagtgttgggggcccagctccagcacgcatggctgtgagcccttgaggaaggtcctcagggaggatcctggggcgcatggctgcacccagctcagccagggagagggggaacattgcggcctctgtcatcacacaccatggctgacagcggctctggtgtgtgagatcacagcagtgataataccatctgccccaggcttccataggaaagggagcagccaggggtctcaggggcttctgtggcctgggggggatttcaaggtccaggtgggatccccggtgtgttggaggcaccacctctgagtcttcatcttcgtctgcacctagccggcaactgtgcctgggagacattaggacagaggtgctggtgaagagcagagaccaaggcccccagcagcgtcacccagagccccgtatgtgcaggctccgtcgttcattctctccttcctttattcaataagcattccctgggaacctcctttctgagccccagcctgggttataaagagtgatgtggatctgggaggcagtgacagccgccgcctgctgtggcaatgtcagcccacagagtcatgcccaagctccttatctcctgtctcctcctgggcacaccctgtgaggcatcccagggacacgtggcaagctcttctgggtccactggtccttcatcgaggcctgtaagtaaaccctgcctgcgtgagcatagtggctctgtgatcccctgatgctttgctgtttagtctgaagaactatttttcttcttaattaaacatttcagagattctgcaaatattataagtgcatattcactgctaagggattagaaagtcaaaatattttaaagtgagaaagcaaagaaaaggatggcacaaaaccaaaatgaataatttttcttcctgttgcagtttctgctcgttgaatactttgaatacttggttggtttagggactcgcttccaagacgaagcaacagcacatagcacaggcggctctggaggctgcaagcaccagaacaggccccaggcagccacggggtggacatgcacggtcctgggccggctctgcccaccggcgagcgcagggctgcctgggagctgaactgaacagagtttctcctgcccggagctcacgcttcgtcccatcaggcccacttgcttcccagattcacatttttagttgtgtttcctgcttgcacagggccaaagtctgccctgggtgttgtgtgtgcaatcattattgtttattgttttcgaaattaattaaattcgagaaaaaaaattagcctccacagaagccacagacttggctacatgtgtgagggaatcagcagagatgtgggtgtgggagcccctgggaggagaagggctctgcggaccagagggctctagggcagccctgggcactgctgagcagctgcatctcatctggacctggagcccaaggggccgcaccagccgtggccatggtctgggcctttatttggggtctcacctgctgcagtcagatcctgaagcagataagccagccagggatcagtctccctgtccaaggtcttcccgtctgtgtcctcttcctgccaagaccagctttcaggcctgggggagtcttaggtcttcactgagtaaagggtttcatccacagtgtatcaaggagcaatggccccagtccttgccctccacagctgctcctctccgcttgcccacccgcactgtaagccacagttctcgagcaccaccaccagcagactgagggctcgggatcaccaagtgtgggaggctccccgagccgtgtgctggggcccgaccacccggccctgccctgccctgcccttcatcccatcacaactcttctgtgctgatggatgggggccggggttcccactgcgtggaaaacagaacagctcaggcagaggacgggagcggcagtggccgggctggtgtgaggctggggattccgcttgccccggcagggctcaggcttccaagaggccacggaggagctcctgggactgatcgtggtgcgggctctggcccagtgggtctgtgtgggggctgagaccctgcctttccagcacgttcccagctggtgccagttggagtcagagcctggaatccttacacgagaggccagaagccttggcctcagcagtccaggcctgagaacccacccagctgtcactcaccgatggctgagtgactttgcacaaagccctgttgcccgccctatcccactcctgggatcagacccagaaatcttggtctgtgtgtcccaactggctacaattgctgctcagcttctgaaaggtgcctcttcaagcctgtttcttgaaacaaggcactgaaaagcatacgttgtgtcaaaagcccaaaaggagtgggaattctccttggtgatttggatttcatcttgggcactgatgaaagagcacagagtgggggcctccctccttgagtcagggtcaggcaggggcaaggcagtagaatctctaacaccctgggcactcacagccctgccattgcttctgtggtggaggtggccagggcggagtgtggcgttcccgggaatggcctcagcagcacaccgtggtgggcacgcgagggggccggctcccgtaagtcttctgtgttacatactggttaactccgttcttacataaagagcacgtctgctaaatttagccaagagaactgtttggcatttcggcctccccatgcttcaagtgcacacatttcttccgtgttacgccacatccctaaaaataagcctctgccctcctccagcactcaggccaccgcagtgactcgttcccactgatggcaaacttcactccccagggctcccatccctggtgacaaagatcacagcagctggcactgcagatgcctttgtgggaggcaccatctgatcatcccgccaaccctcctgcaaccctgcaaggacacaccagcctcccagggtgccaggaggagcctgccagagacccccagagcggcctgccccaaatcacctctgagagcagcagcttttctgaaggaagcctcccctcatggtcctcaggcccagcaggtgccaagctcaatgccagccacgagggcattggctcatcgagtgatgggaacggggacagcaaggcggccacagagagggtggtctcagccatggacacagtacggcggaagcatcccgaggtgggccgggacaagagctgggcctgtggctgctgcgggggccacgagactgacctcaggcaggggcagaaatctggccacaggcagaaatcattcagctgtcacctggcacaaccagacaggattatgagcacttttctgctgtttagttttaacctgtgcatgtgatgcttaatactttccaaataaatggatgagtcttttcagggccaaaggagatacgggaccagggaaggaggttaagaaggaaatgcacagatacgctggtgcctggggatggcgattgggtctccaagttctttaagttctaaaggaatccgctagggaagattctcctaatgggagggagatgaaggtgaaggaattagaggtccacatgatcaggaaggtggaaggatggaggagcacaccaggatgatgtgcagcacagggcagcggacctgtaggaaacacagcggtgcgccaggcgcttctgggcatggagctggccgatgccggaggaggggaggtacgtgcctgacccaggatggaccatccaaggcagcagggtctgcacaccaggctctgcatgaaggagtgtgtggccccaggcaggggttgaaactctccgggcctcgatggataggaggcctgtctttgggcttgggcagccttgggtgggacctccggcattgtgctctggggaagggtccagcctccctgctgctccccttaaggggaggtctgtatgtgggcggcagatgttcagcttgggcacccagcagctggctcttgggctccacaggtgggtggggcaggggcgctggttctgagaaggtgtgcgtcctgcagcttgttgccaggagcccccggggagcctccagatcatacaccacacaccacactggcccctccaggacacacagcacatgcttcaggactcagcatttatggtcctcagaaacaactgtattgaagaaagtgtggaaacccaaatttcagggttctagggaggtctgttaaagacttaaaaaaaaaagagagaaggaagagttttgaagggattcacctactgcctgtcaggtcggcaccaactgtcatgtcgatgtgatttggagcctggacaggagcaggggctcgtggagaccaccgtgggctggataacggccaagggggcagaacagcagggccttgagtctggggcttccggtgacactcctgttctccctcttaatagtccttttgctctattttctgctttttcaaaaataagaatatattggttttatatttacaaaaatcaaattttaagtactaaaaaaaccacttaaatttatacgtgctttgcctttgaaccccagactttgtcagtacaaagaggaaactcagttgtcacattctctctgctgtatctttcttagcagctgttttgaagttagttgcaagaacagggccaggttccccgactgtgagccctgtcaccggctgtgagcatttgcccggccctcacactgggtgtgtgcagcacacagtcacttgcacacacgccttttcaccccttaaatgttaggaatcccctaaaagcagggctggtctcttaaatctcttcagtagtgtcatcaaatttaggaaatgtcccattttcactaatgctgtcatatgatttgctgtctcggctccgatttcatagactccaggccaggatagtgcgctgtgtgtggttgtggtggctccccggctttaatctggaactctccctagcccctctccatctttgtggccgtgatgccctcagagacctcagccctctcatttcacgaagtggctttgctggggtctatctgatgtctccttgggactaggttcaggagatctgccaggcacatgttttaaagcaaagtacaaatatttgctagcaaagcaaaggcccccacttcctaggtcagttgccttttttaatcctaaactctgtgcatgaactaggtctcaatgggataaaaagctccaaatgtctctgtttctatacttcagaaatgtcatgtttggattttaaaagaaaataagttctccctctaacttggacataccataaaggaaacacgtggcttaaaataaggtgtgtggcacggcatggagtcgcactgattacgggctgtctgcctcacagccgtgtacacgcaggatcttcgtctttaaaacgtcaccaccagagccggatgccaccctgcaaagaagctgcaaaatcacactgcaattatcaccgcaggctcacttggggtaacagcagtgcacactcttctctgggaaaccagcccttattcagaggaggaacaggaggccacccaaggatgtctccggtgcctgtgcacgtggccacgacctgggcctcccagcagctctggttcctgagtgccagccctggctgacgggtcaggggctccaagggctcctgagggctgagcttagctagtggctgatgtcagaagcagccatgggctcaactttccttgggccttgggcactctctggcctgtgtctaaatgatgtccccattcctcagacagcaagatttaaggggcaagattccacgtgctcatcagagaccccgtggcctcttcctctgctgagtctggaagtgaagccacaggtttcacatcccagatggcagaggcgaggccctggctcccatggggatggagcagaggggaggagcctcccctgtggagaatgagaccagggctgcagtgagagaccatcccctcctctgcatctctcctcttggggcctcttaaatgatttggaaatcattcccctaaaaattgagcctttccttgagaggtcaaggcagactttcccatcacagatgtgagcctgttccccatcctacccttcagcttcctgctccctttccatcacgcagacatgaccaccagccctgaaagagggagtcacgaggggcgcccgtcagcactgggcatggggtggccgctgtccaggagactcagggacactgcggggcctctcccatgtccggacccagggacgctgcagggcctctcccctgtccggacccagggacgctgcggggtctcccatctccggacccagggacgctgcggggtctcccatgtccggacccagggacgctgcggggtctcccatctccgaacccagggacgctgtggggtcttccatgtctggacccagggacgctgtgggcacctgcttgttgtgggtggaacagctcctcgctcccatctcggatcctgagcccagcgctgctctgcactgacagactccaggggagttttcagcccagggaagtggttatttttgtgagaaataagactatttttaaaaatctcccctgagtgagttggagaggctcctcacttccggcagtgagagtgagctccagccacacccgccagcaccctcgaaagtaagcgtggagccgcggtgcctgccctgacttctactaaaggacgcttcttaaaacacggttggtctgagtttcccatttccctgaaggatgacctctgagaagatggacgagaccagcctcctgtgttgacgttttcagtctcaacctaaggaaaagtaaatgagcacacaaagttctcaaagtctacagtgtttttaaaaatataatgacgtttacctctcaccttcctccagtaactgccagtgtcttgaggaaatgtcttctgcccccaccagcccccgcaggaccacatctctcagcttgggagcagcatctccgctgctagacgggttctctgagtggacggcaccagtgcagaagtggatggcttgttccacctgccttgactcccccaggtcacccactctccccggagaggctgcacactcacatgcgtgtgtgggtaacaacagtcacagccttgttcccaaggcctgagtcttgagagcagaagggaggctgctcttgctggcagctccacgctgctgcccagcaccaggcccatgagacacccaaggcccagaactctgagacacggagccacagtccttccaagcagaaagccacgttgcaaacaccgaccacctccctctggcagacacacaaatctctgagaacagccgaacccgggaggcggagcttgcagcgagccaagattgcaccactgcactccagcctgggcaacacagcgagactcagtctcaaaaaaaaaaaaaaaaaagaaaagaaaagaaaacagcctcctgccaaaaagcatcccgaaattgcccatggaaggaggttattttggtgatctggttcaagttattttgatgccagaggctgtacaatttggaatgagtcaagactcttcagtttaaaatgggaaaggaaatgaatatttgaagataaatatctcaaagggcagtgccagaccagaacccagaacccctcctcaccctgtcatcttgaggagaaaatcccagaggagaggataaatggggtactcaggtcgctgtcccctcctcctgacctcagattagaacatggtggctgggcccatctggaggggacagggtaagacccgctcctgtcacacctggtgctctcggcttcatgagtgtcagattcgccaatttcgctctgggccccagccgtccattgctgtgtccttttgcttaaaacaggctcagcctgcagagtccacagctcaggcggaggtgggcagtctgtgcaggtgggaagccctcacgtcagcctctgactctgtcttcacaggaaagccctgggaggttctcacctccgttttctgagggactaaccgctgcccaggagttcagcggcataatcttggctcactgcaacctccgcctcccaggttcaagcgattctcgtgcctcagcctccagagtagctgggactacagacgtgcgccaccagcctggccaatttttgtatttttagtagagactcctggcctcaggcgatctgcccgcctcagcctcccaaagtgttgggattacaggcatgagcccctgcccctggcccaggtggcttttctaactgcggttgctcaggcccgtggaaggaggacaggtccaggcccagctgactaggaagcagccttcagggccaggcaggctcccagccgcagctctgagtattccagcctcctcctgtggcaccgaccagtgctcgcgtcccgggaaccacaggaggaggccaggcaggtctcatggcccctcgttcccccactgctattgctgatgggggaaccggactgtgtgaggagtgacgtgcagggcggggctttccctgggccccctgcgaggcaggcatgtgcaaagcaggcaaggagggctgcagttgtgggattacacaacctttcacatcccttccgttataaaaattcctttcatcatcatatcacaccaaatagcataaattattgagcgttcacagagcacctggcaccatgctgaactctgcatggactagctcattcagtcccccagccatccatgggataaggcgctgcatgaccacactttaccatacagggacacggctcagaaggggcagccgcagactcatccatgagcaggggattcccacgagcagtcgagacatcagcgcttatcttctgcttcatgcgcaaagccctctattaacctcacttgtgttgctccagttccgtaaaccacaattcgatgcttctacggagctcctggggcaaagtaggttcccaaggaatccaggctttaccggtttggtaaaatggtcacaggaagtggcctctgtgtgtgatcacagcgtgtctgtgctgaggtcaccagagattgtgatggtcacaagaggtggcccctgtgcgtggtcccagcgtgtctgtgctgaggtcaccggggattgcaatggtcgcaggaggtggcccctgtgcgtgatcccagcgtgtctgtgctgaggtcaccggggattgtgatagtcacaataggtggcccctgtgcgtgatcccagtgtgtctctgctgaggtcaccggggattgtgatggtcacaagaggtggcccctgtgcgtgggtcccagcgtgtctgtgccgaggtcactggggattgcagtggtcgcaggaggtggcccctgtgcgtggtcccagcatgtctgtgctgaggtcactggggattgtgatagtcacaagaggtggcccctgtgcgtgatcccagtgtgtctctgctgagggcactggggattgtgatggtcataagaggtggcctctgtgtgtggtcccagcgtgtctgtgctgaggtcaccggggattgtgatggtcacaagaggtggcccctgtgcgtggtcccagcgtgtctgtgctgaggtcaccggggattgcaatggtcgcaggaggtggcccctgtgcgtgatcccagcgtgtctgtgctgaggtcaccggggattgtgatagtcacaataggtggcccctgtgcgtgatcccagtgtgtctctgctgaggtcaccggggattgtgatggtcacaagaggtggcccctgtgcgtgggtcccagcgtgtctgtgccgaggtcactggggattgcagtggtcgcaggaggtggcccctgtgcgtggtcccagcatgtctgtgctgaggtcactggggattgtgatagtcacaagaggtggcccctgtgcgtgatcccagtgtgtctctgctgagggcactggggattgtgatggtcataagaggtggcccctgtgtgtggtcccagcgtgtctgtgctgaggtcaccggggattgtgatggtcacaagaggtggcccctgtgtgtggtcccagcgtgtctgtgctgaggtcaccggggattgtgatggtcacaagaggtggcccctgtgcgtgatcccagcatgtctgtgctaagtgtctcctttggtgcctgccttgcttttgatttgccatcccctttcatccacagtaattttttaagtcctgatttgctcttcttttctctccacgtgtaacttctcccttccccacagtgatccttgctaatcacagcatcttctagatgtggaatgccttgttctcacagaaatgctgcctcctcctgccatcctgtatgcagtgtacagccagtgaggtattagttgttagggtcccgtgcactttttgtgacattgggacaggtttttgctgttgttgttgttgttgttttacaagtggagaaacctgagttgtaaagcatgtaaagacttgtctcaggtttccttgctgacagaaatcactaatatgtattgaacatctagcatgtgctaataatattagttataatgataataatagttgttgttaagggcttactgtgctctaagcactttacatgaaattctaaatttaatgcgcaacactgccagacaagaactattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccacagatgaggagccgaggcctgaggctattcccattccccagatgaggagctgaggcctgaggctattcccattccccagatgaggaaccgaggcctgaggcactgggattgttctcccagcagcgtttccaggctgtgaccagagtagctgaatggactccggagctcactgctttcactccgtggtgcaggccgtgctgccaccgctgtgtgattccctctccagctcagacagtggcttctccaagaaatcctactcaggtcagggcttagtccaacttgtgagacactgtcctgtgcacttgctcgctgtgatgtgaataacgtggtgggactgtcctgactccttgcaggtgtataggtgtgctgcagcaggtgtgcaggtgtacagctgtgctgcagcttgtgtgtggtatacgggtgtgctgcggtgggtgtgcaggtgttcaggtgtactgccgcaggtgtacaggtgtgctgcagcatgtgtgcggatgaacagctgtgctgcagcttgtgtgtggtgtacaggtatgctacagcaggtgtgcagatatacaggtgtgctgcagcatgattgtggtgtacaggtgtgctgcagtgggtatgcgggtgttcaggtgtactgtcgcaggtgcgtggctgtgctgcagcatgtgtgtggatgtaccagtgtgctgctgcagatgctggctcctctgtaggttctctcaggattgggcaacagtcaaggctgatggatgctgtgctctagtaggtagaggaagaaccattcttgactgtcagaagaccctaaggctcattgccggcttcccctgtaaaagaccctcaggctcagtgccttcaggggtcgggtgggcaccgattggggcccccagccagccacaggacttcctgcagcctggattatgggggcagcagggagaggatagggagtgcccccacttgggtattgtttgtttgggaagcagtatctcactatcaggatcctgtgtgttgtttgctggggaagccgtatctcactatccgagtcctggatattgtttgttggggaagctgtatctcactatctgagtcctgggtattgtttgttggggaagccgtatctcactatccaagtcctgggtattgtttgttggggaagccgtatctcactatcagagtcctggcagagtgtggttattgtttgttggggaagtcgtatctcactatgaggatcctgtgtattgtttgttggggaagctgtatctcactatcagagtcctgggtattgtttgttggagaagctgtatctcactatcagaatcctggcagagtgtggttattgtttgttgggaagccgtatctcactatccgagtcctgggtattgtttgttggggaagctgtatctcactatctgagtcctgggtattgtttgttggggaagccgtatctcactatccaagtcctgggtattgtttgttggggaagccgtatctcactatcagagtcctggcagagtgtggttattgtttgttggggaagtcgtatctcactatgaggatcctgtgtattgtttgttggggaagctgtatctcactatcagagtcctgggtattgtttgttggagaagctgtatctcactatcagaatcctggcagagtgtggttattgtttgttgggaagccgtatctcactatccgagtcctgggtattgtttgttggggaagccatatctcactatccgagtcctgggtattgtttgttgggaagccatatctcactatccgagtcctgggtattatttgttgggaagccgtatctcactatcagagtcctggcagagtgtggttattgtttgttgggaagccatatctcactatcaggatcctcggtattgttggttgggaagacgtatctcactatggggatcctggcagagtgtggttttcttcacgtgaggacatgtgagtctactctagaccatttgaggtaagagctgatgttttattcacagaacctgaagacaacctgctggtgtcacaaccagcctccaagatgcacttttgctgcttcagagaattgtgtcacttccatgtttattacatgggtatcagctactcactgaggagtcatggctttgtgccatcctacggggtgggaagcaagcaccgtgagccaagggtcctgggcaggcgctcggaggcaggcagaactcagacccagcaggaccagctcctgggcacaacacctggccatggagaatcagcgcagctgtcacacccaggtggcccatgtaaagaggaagagcatccctggaaagaaacttcacatttgacatcccccgggttgagagcttgtctctcaccttcctgaacactcagcagaaccatgcaggccccagcctcagcccttcctgggggcaaacccatgtggcaggtctcagccatgcagccatggtgcaggatccaggctgtcacagcttttgctttgggaaggccttttcagcttacctcagattccgggatgttacagtcccccgcgtgggtcagaacagggcacctccccacaacccagctcagcacggtctgattttcataggactccatgggcaaaacaagtcaagatcatatctgtgctttaccttgctgttggaaaaacagttctcactggaagacacattcttaagatggaaccaccccaggcatccctgagttctccactagacttagcagctcctgatttttcaagttaatcgactttttagagcagttttatggtgacataaaaattaagcagaaagtacaggggattctcgtgcagtccgcactgcccagtgcctcttattaacatgttttgttaatgtggtgcacttgttacaactgatggctttggtataatattgaccccttgttgttaactgaagtccctggtttacttagggctcactcctggtggtttgcattctgtgggtttggacaaatgcctaatgacaaggacccgccatcacagcatcgcgcgggtcgtcctcgctgacctgaaactcccctgtgctccgcctcttcgtccctccttccccagcccctggcaactgccgatctttgccctggctccatagtcctgcctttccagaatgtcttgggttggaatcctgcagcatgtgggccttttcagatggaatcaccagttactcctaacgaaaaaaaaaaactgaaacaaaagagtaaccctggattcttgtggtgggagacttcctccagtgagacggtgtcatgggattcgatcctggcgtcagcttcagcgtgacagccgtgtccttaaactcaccatgaggccgggcgcggtggcccacgcctgtaatcccagcactttggaggccaaggcgggtggatcacctgaggtcaggagttcgagaccagcctgaccaacatggtgcaaccctgtctctactaaaaatacaaaaattagccgggcgtggtggcacgtgcctgtagttccagctactcaggaggctgaggcaaaagaatcacttgaacccgggaggcagaggttgcagtaaactgagatctcaccactgcactccagcctgggtgacagagcaaggctccgtctcaaaaaacaaaacaaaaccctccacccgcctcatccgtgttgtgggaacggagcagtgagactcaatctggtgtcttcacagggttgttgccataactgcatgaattcgttttgtaaattgcaaaatgctgcaccatcatgacgatggtcctgacagtctcctaagtggagtgacttgtagaatctgatgcttcacaccaggatatttgccacaggatagatcctcaaagtaaatgtcctcaaaggatattttgccagtaacttgaaagaagaaaaattcaacttgctaatgcttttacacagtgcagtgttctcatcctgaatcttgaaacagcacctccgtttttagaagaagcttccatgggccatgatgcccttggaaaagattgcagtggcatcttggagaacatttcccacaaaaataggttatggtttctgacggtgtctaaaacatttgagaagcaaaaggaagctccagccttcgacttctaaggcaaaaacaggcacctcctacctctgagactggcaactggcggcctccagtgaggggtggagtccatgccccgaaatcctccctgtaccccactccgcgccagcaccaggcagaggccccccaacaggcccacatgctccggggacccataagaggtgcagacaaaccctgcactttctgcagcgtgaggccacgccgccaggggccctgcagacactgaccagggactgaccttgcctgccattgcacaggcatagtgccctgaatcccgtgcccggcgccctctctgcaggagcctgaagcctggcagagggaacaggggacacattgtgccactctgggaggaagaggccaagggagggtgctgtcactcggcatgaaatgctgcgccaggggcccccacatcctgtctcctgcctcacgctgttcctgtctcacctggcggcactgccctccagcgtgcccagccgcctgccccatcactcgctatttcccttcacccactaagtgctcccagagttcctgccccgagcactctcatcctcccgcccacacctcgtgcggcctgccaagagtccactgcctgcctgcagaactcagaggcatggcctttgcccctctggcctggtgcgcgttggtcttgtttactaaggaccggacagaagctgtctgagtactgatgcggctttgctttctgcaccccacagcaccctgtgcaaagtggcattggttaagacgttcatcattacacaaagggtctggaagaagcagtttcccagacggagatgtgtgtgatgggggctgaagtgggctgggaagctgagactttgtcagagcccatccagcaagggcacatctgctaaggggtaaagaaggcttctatctgagggtttcctgagaatgttcacatgcaccagcccagacactgcctgcagcagacgcagagtcaggagccccctggcctagcccaaagcttgcttcaagtttgcctgaggcctgcagttagggacacagttcacactgtgatgcagtgcagacacctcacaaaaccacaggttctcttccaaatgttgcttgtcaccccccaaactgcatacctcctggatgggttatgaatggactgacagtaaacgtgtccaccagacctggcctctctcgagagaacggggctgtgcatgattgcccgggacagcaggtgagaagcaggcctggcccggctagtgggtactggctgccctggctgtggctctggtggctcgctgcctggaaagctgttgaacccgcgtgctgtgtgaaggactctgctatggtcactccagggcagatatttatggaaaggtggagctggaagggacttagacacctccttgaccaacacactcatctagggatgaggaaactgctcccagaaggggaactacctgaggtcacggctttagggaagaaagtgggctgggcgcccattgagattcctcgcaaatgggactggaacagaagcactttctccatcatggccataaggttccggatgaagctgggggagactccctcacccagacaaagatgaagggaagaggctctgcagagcagtggggcaggcttgggatccttgcccagtgagtgtcaaggctgctccggggtggacaatggaattagtgtgtgtgacaggcaagtggagtcaggacaggggcaggtaggacaggtgggaccagggacaggcagggatagttggttcaggaagacttgggggtgtggccaccaagatccagtattagaacactgaagctcttgcccttctgctctattcttgaagccaaccacaactgaaacaaatataacaccgtgtgtaattacatgaggatgatggttagcctcatttgcatgtaattaggaaggatctaatcattgttcatcacacattcggtgacccattgatacactgggaatggtcttgagccacccttcatgatagctgcctctctgtccatctgggagcaggtctatctgtctgtttgtctgtctgcctgtctgcaagtctgtctggctttctgcctatctgcctatctgtctgcaggtctatctgtctgtctgtctgttttcagcacagcctattctactaccttagaaacccaaaggaaatgggcgaacaggtaattaggatgaattcagaatgctgctctctcatagaaacattcttttttttattatactttaagttttagggtacatgtgcacaacgtgcaggtttgttacatatgtatacatgtgccatgttggtgtgctgcacccattaactcatcacttagcatttggtatatctcctaatgctatccctcccccacgaccccaccaccccacaacagtccccggtgtgtgatgttccccttcctgtgtccatgtgttctcattgttcaattcccacctatgagtgagaacatgcggtgtttggttttttctccttgcgatagtttgctgagaatgatggtttccagcttcatccgtgtccctacaaaggacatgaactcatcattttttatggctgcatagtattccatggtgtatgtgtgccacattttcttaatccagtctatcattgttggacatttgggttagttccaagtctttgctattgtgaatagtgccacagtaaacatacgtgtgcatgtgtctttatagcagcatgatttataatcctttgggtatatacccagtaatgggatggctgggtcaaatggtatttctagttgtagatccctgaggaatcgccacaccgacttccacaatggttgaactagtttacagtcccaccaacagtgtaaaagtgttcctatttctccacatcctctccagcacctgttgtttcctgactttttaatgatcgccattctaattggtgtgatatggtatctcattgcggttttgatttgcatttctctgatggccagtgatgatgagtattttttcatgtgttttttggctgcataaatgtcttcttttgagaagtgtctgttcatatccttcgctcactttttgatggggttgtttttttcttgtaaatttgttagagttcattgtagattctggatattagccctttgtcagatgagtagattgcaaaaattttctcccattctgtaggttgcctgttcactctgatggtggtttcttttgctgtgcagaagctctttagtttaattagatcccatttgtcaattttggcttttgttgccattgcttttggtgttttagacatgaagtccttgcccatgcctatgtcctcaatggtattgcctaggttttcttctagcatttttatggttttaggtctaacatgtaagtctttaatccatcttgaattaatttttgtataagatgtaaggaagggatccagtttcagctttctacatatggctagccagttttcccagcaccatttattaaatagggaatcctttcccaattgcttgtttttgtcaggtttgtcaaagatcagatagttgtagatatgtggcattatttctgagggctctgttatgttccattggtctatctctctgttttggtaccagtaccatgctgttttggttactgtagccttgtaatatagtttgaagtcaggtagtgtgatgcctccagctttgttcttttggcttaggattgacttggcaatgcaggctcttttttggttccatatgaactttaaagtagttttttccaattctgtgaagaaagtcattggtagcttgatggggatggcattgaatcaataaattaccttgagcagtatggccattttcacggtattgattattcctacccatgagcatggaatgttctttcatttgtttgtatcctcttttatttcattgagcagtggtttgtagttctccttgaagaggtccttcacatcccttgtaagttggattcctaggtattttattctctttgaagcaattgtgactgggagttcactcatgatttggctctctgtttgtctgttattggtgtataagaatgcttgtgatttttgcacattgattttgtatcctgagactttgctgaagttgcttatcagcttaaggagattttgggctgagacaatggggttttctagatatacaatcatgtcatctgcaaacagggacaatttgacttcctcttttcctaattgatgccctttatttccttctcctgcctgattgccctggccagaacttccaacactatgttgaataggagtggtgagagagggcatccctgtcttgtgccagttttcaaagggaatgcttccagtttttgtccattcagtatgatatcggctgtgggtttgtcatagatagctcttattattttgagatacatcccatcaatacctaatttattgagagtttttaacatgaagggttgttgaattttgtcaaaggccttttctgcatctattgagataatcatgtggtttttgtctttggttctgtttatatgctggattacgtttattgattttcgtatgttgaaccagccttgcatcccggggatgaagcccactttatcatggtggataagctttttgatgtgttgttggattcggtttgccagtattttattgaggatttttgcatcaatgttcatcaaggatattggtctaaaattctctttttttgttgtgtctctgccaggctttggtatcaggatgatgctggcctcataaaatgagctagggaggattccctttttttccgctgattggaatagtttcagaaggaatggtatcagctcctccttgtacctctggtagaatttggctgtgaatccatctggtcctggactttttttggttggtaagctattaattattccctcaatttcagagcctgttgttggcctattcagagattcaacttcttcctggtttagtcttgggagagtgtatgtgttgaggaatttgtccatttcttctagattttctagtttatttgcgtagcggtgtttatagtattctctgatggtagtttgtatttctgtgggattggtggtgatagcccctttgtcattttttattgtgtctatttgattcttctctcttttcttctttattagtcttgctagcggtctatcaattttgttgatcttttcaaaaaaccagctcctggattcattgattttttgaagggttttttgtgtctctatttccttcagttctgctctgatcttagttatttcttgccttctgctagcttttgaatgtgtttgctcttgcttctctagttcttttgtgatgttagggtgtcaattttagatctttcctgctttctcttgtgggcatttagtgctataaatttccctctccacactgctttgaatgtgtcccagagattctggtatgttgtgtctttgttctcgttggtttcaaagaacatctttatttctgccttcatttcgttatgtacccagtagtcattcaggagcaggttgttcagtttccatgtagttgagtggttttgagtgagtttcttaatcctgtgttctagtttgattgcagtgtggtctgagagacagtttgttataatttctgttcttttacatttgctgaggagtgctttacttccaactatgtggtcaattttggaatctcactgagcgggtggagccgtggttggggcgaaggtcagtccctacctgcatttctgtgtttatttgacagttcttcccttgtggtctttctctgagtggagctgagagtgcggccattcagcattcctgtcaccacccatccttcctccatggacactgaaggcttcaaagtgggatctcagtgggccctccaggttgttgcttggttctcacagagaccatttgctgagtcagtgggcactggagcggatacaacacccgagagaggtggtggatgttactctcaggaggataacacctagcgtccaaaaatgccacctgcccagatttaaccagctcaaaagataaaatgagtcacagtctgattcaggttgtatgtttccaagcccacctgcattttcctggataacgtgtaattgggtttacatttttcccgagttacttatgtaggcacaacgcgatgcaacaacgtgctcctcctcagccatgaatagcccaaggctgtgtggttgcctaatgtaaatctggctcaacagtggatagatctttgctataactgtaaatattttgcagtaaaaaagaccccttccaacaagactcaaatgcagactttcgttggcatcttttccaataaactaagtctccaagttctaatgtatggggcacattgccttctttaggaagcagatcatagaaaacttcttagatctggtggaaatcagcttttgaatattgcattagtgccttacagtgttgaaccgtgtcatgattacctaagacaagataaacatggggatgtctcatttcaggaagcagtgctacaggaaagcttcctcctaaattaggcttctacgtggtgtgagcaatcaggtatttgttagatatttctatggaaacaaaaaaaaaggttactagttggaacagattataaactcagtctctcagttcagagggaagccagttgagatttaatagatttgagctggaattatctttagatggtaaaatgagggggggaagtttcaatgtgaacaattttcctgatttgcagtttgaatgtcccaggcaatatcaccaggtgttccaatgagctctctaagtggcccacacaggaacaggcatgaatgttgtccatgcatgaactgtggcagcgatttctctgaagtttatatcaaattttccagtttcaacttgcagggagtgagggaaagggcagttttatttcttgtgattccaggtcagaagagtgagggaaaattggaaatgttcatttcgaaagtcacagccagacattggaggaaaaaggaaaaactcaagttgcagtccaggttagaggtacacagcaaacctcaaagacattgaacagggctagaatctgataacaggtacaccttagtacctcaaagacattgaacagggctagaatctgataacaggcacactgtagtacctcaaagacattgaacagggctagaatctgataacaggcatactataatacctcaaagacattgaacagggctagaatctgataacaggcacactgtagtacctcaaagacattgaacagggctagaatctgataacaggtacactgtagtacctcaaagacattgaacagggctagaatctgataacaggcacactgtagtacctcaaagacattgaacagggctagaatctgataacagttacactataatacctcaaagacattgaacagggctagaatctgataacaggcatactataatacctcaaagacattgaacagggctaaaatctgataacaggcacactgtagtacctcaaagacattgaacagggctagaatctgataacaggtacactataatacctcaaagacattgaacagggctagaatctgataacaggcacactgtagtacctcaaagacattgaacagggctagaatctgataacaggtacactgtagttttctaccagaacacaacttttgtctcttcagtcaccccaattcctaccaaagataatcatactaagactaatctgtttgcaaaatcagtctaatctcattaaacttggactgatcatttatgtaactgcatcaaggatagtggttcaatgcacattctcaaatatgacattccagtcaaagttttggtgataaccaaaggggttattagctccataaagtcaatctctactcattgaaactgtctggtaataaggagtctcagattagacttttaaaagccactccaggctaagcaaccaagccatggacttgcacaattttgtaaaatggagataacttaactagtaataaaagttgtatgtctgtattataactaatattgacaactctgaaggtatatccttttctttttttaaaaaaactaaaccaacgatattcttactcaccaaagattatccgtgtcacatgaacttaaaaaacatttgagtaaattcctgtttttctgaaagttttagaattacttatacaagtgcttatttttctttgagccaattaaatagagctattttatacattaattttggaaatgccatccagaggttaaaaaaaaatacacatgtcataaagatagagatagacagagatcttacagcttccatttagaaatttcagtcatgtgtctggtacaataatacaaaacttgctagtttataaaagaacagctggatccaaattgtgtttctggcagacaaaataatttgctcaaatggctaaagacttttttactaaagatttttcatttgtgcactgtaaggatacttttggagaatcagtttgtcttttagaagcttctgcataacaaagaaggcatttcactgtctctgaaaagatgaagatcctctgtcttcaagggcacccctaagctgatctatcttacttatggacttacacgatctacaagcaacatttttgtttttttcttttctaatatgtaaatgttaaaggttccccaaagtggccatggcagttccaaattatccttttttaagtttacccatttttccaaaaacgcacaagatcctggcccacggaggattttggaccaggtggaacccatctttgccttaaattacccagacatgaacagctgttccaaaacagttcagctgtttcttccacttctgtacagaacagggtgacccaccaagaaggttcaacaaagattactcaagaaaatgggcaagctcaacacaaagggtgtggagattggatcctagagacttaccctggacttccacggcccgtgagaaagcagtaaatgcagtaggtccttgggtgccggccctgggctactcgctgatgcagcgggtccatgggtgccggccctgggctactcactgatgcagcgggtccgtgggtgccggccctgggctactcactgatgcagcgggtccgtgggtgccggccctgggctactcactgatgcagtgggtccgtgggtgctggccctgggctactcattgatgcagtaggtctgtggtgccagccctgggctactccctgatgtggcaggtccttgggtgctggccctgggctactccctgatgtggcaggtccgtgggtgccagccctgggctactccctgatgcagtgggtccgtgggtgccagccctgggctcctcactggcctcaaccctcatcaggggtcttctttggttcccactcctgacactaaaaatatgatatgaagattttattgagtgagaaacaaactgtttgcaaatagggagacttcaaagccaaaactggtaagaagcttgtctcatggttacagcaaggtttatatgctatgaaagaggaagttcataagcttaattctcattggttaggttgagtaaattacctgggtcactaattggctacaatgtgctgaggtgtttcaacaaagaaataaagctcaaaattggtttacagtttaagtgggtaaatggacatctgtagattttgagctgagctgagggtaggccattgactcacaggcaaaggtcagttctagcctccatttttattcttatttaacagtggtaatgggaggatcctaaacttttcaaaagcatctgcaaaggccacatgtagtcatgtagtctaaggctaacagaacaagaggtttcttagcctgtctttacaccaaaagccaagccaagctatttaaatatcagctaaataaggtattcattgagttcacttttcatagacatagagacactcctgttccaagttacctgtctcatggcacctcgagcataatgggtacccagtttttcctacatgttctttggtctctttctattggcttcagaaaaacgagttctttttttatctgggtttttgtttgtttgtttgttcattgtgacagagtctcactgtgttgaccaggctggtctcaaactcctagcctcaagcaatcctcccacctcagcctcccaaagtgttgggattacaggcatgagccactgctcccagccagaaaaacaagttgttatagatttctagtcataagaacaatttttaatagtatattatcttagtctattcacgttgcagtaaccaaatatcataggctgggtgacttatgaaaaatggaaatttatttttcaagctttggaggctacaaagtcccagatcaaggtgcagccgattccttgtctggtaaggccacatttcctctcacgatgctcccacacagtggagggacaaggggctctaaccctgttcatgagggctccaccctcacaaactgatcaccttccagaggccccacctcttgataccatcaccaaaaagttaagtttcaacataggaaatggcgtcctgagcaagctgagtctttcacacagaaatgggtctgtctcccttttatttggatttctcttctgttgcttggggaagtgcttcagtttcctcactttaggcccagaacattttccgctcagtttattcctagatattttacactttgtgcagctcttttgaatgaggcctgattaaatttacttgatagttttactgattatttctgttgacttctcttgatttttctgagtatggacttacatgatccacaagcaacagttttgtttttttcttttctaatgtgtaaaaaatttcattttgcatgttttattttattgattaaaatgtgaaaaacattttgtggtagaagcaggagtggacattgctaatgttaatgagaaatttctagtcagggctctgattccatcagcccccagaattgtgtgcacagttgtctgtctgtgcactcatgcctgtgtgcattttcttcctgtagaaaaaaaggttccacgtctcccttagatggacagacagctccacagcccagaagaggtgtagagttctgccctggggctcccccactccatgtggcactgcctgtgcctgaaaggaattgttctaatgagacctaagaaattataattctattcaattaaaaaaaaaaggataggctgagcacggtggctcacgcctgtaatcatagcattttgggaggccaaggcaggcagattgcctgagctcagaagttcaagaccagcctgggtaacatgatgtaaccctgtctctacaaaaattagccaggtggggtggcacacacctgtaatcccagctactcgggaggctaaggcatgagaatcacttgaacccgggaggcagaggctgcagtgagccgagatggggccacttcactccaacctgggtgacagagcaagactcttgtctccaatcaataaataaataaacaaggacaagtgaatatcaactgggataagcatttttttcattgacatatggatgcaatatattgtactaattgcaagcatgtgtcatctttgcatcctggggaaaatctacttgattgaggtgtgtgtatttagtatactcctaaactgatcttcttttatccttgatattttgagatttttccttttgtatttgtaagtgaaattattctgtagttattactatttatttcagtatattgtctgcttttgacatcagacttatgctagctttaaaaataagatgttgtgctttttctttatttgttactatgttctggaacaatttttatatcctaggaataatgtgtaccttagaagatgaaaagaactcacctgtgaaaactcctgggttcactaacatttttacaacttatatttcacagactttaaaaaaatagctcttctggttattggtgtttttaggttttccttaaatctgttctggaaatgtttacagccccagaatgttaccatttcgttgaagttttgactttgttagcacagagctgtgcatcacactttaattaaaatgtgtaaaataatgttcctcattatttttcttgttttgatatttcataaataatctactctattaggctataccaaaaaagtgggttattgtaaatctgaccattttttaaaacttactctccttaataaatatttcctgagtattcctgtgtgccaaccactgttctgggcattttggatatacatcaataattacaaaaccaaggcccagggcctcatgggcctgtcgtccaggaaggaggtaactttctgtgtctttcgtttttgtgctcttgttattataatttccttcttcttgctttccttgacttgttttatcatttttcaggcttttttagttgaatgctttattttcttattaagaaaggtatttaaggctctaattatgccactgaccataattttgacaatgttttgaaatgttaccattttctctttttcagatgtttactgtttttctagttttaattttattttttgagtttatttggaaaataagttgactgttaattttcttgctctttaaaattttatagtcataagtgtcattctgtaagctttagagacatgggactctggattttgttgctagtctagcgatgtgtgttttcttacctgccggtaccatcgtgccttcatccagcaggtgccgcagggttcccggcctagagacgtgggactctttatttttgttgccagtctagtgatgtatgttttcttacctgccggtaccatcgtgccttcatccagcaggtgctgcagggttcccggcctagagacatgggattctggatttttgttgctagtctagcgatgtgtgttttcttacctgccggtaccatcgtgccttcatccagcaggtgctgcagggttcccagcctagagacatgggactctggattttgttgctagtctagcgatgtgtgttttcttacctgccagtaccatcgtgccttcatccagcaggtgctgcagggttcccagcctagagacatgggactctggattttgttgctagtctagcaatgtgtgttttcttacctgccggtaccatcgtgccttcatccagcaggtgctgcagggttcccggccatgctgtgtgcggtgtgactgcacttttcatggctcctttgtatattatgaattctagtttaccagaagtctcctctggatctaaagttaaagagaaaactctttgcacctgttctcctgtgaacagtctttctgatatggacgggttcactcattaccgctgaattgaactcctgttctggacaaggagggggcctgggccttgggatgaggctggtggggttgtgggtccgagctccaggcacaactgaggaaccatgaagaacagaacgcgatgcccatggaaaagccagtgccagggagatgctcagagccaagattctctctgaaggccccaaatggggacaggcactctctgagtggtccaaggccaagggtctgcctggggacccaggaggaaggaagccaagatgggaggctgagccggggaaggttctggagtccagagggagaggcctcctaggaagagaccctctgtgcagttcttgaaactgctgagctggcttggccacctctaatgaagataaacttcacctcacagtgaagatggaaaagtgtgcacagttcctcatgaccatgttctaaacattggtgcttattacaggaggttgaatgaatgtctgtgcaacccagaaaaaccctggaagttgctcgacccacctcctgcaacacagtagcctggcggttttcttgtgagttgcctcatgtttttaaaaactgactacaggaaggatttcttgccggtaatgacctgcgtggtgaagccacagtcagccagtctgtgggcgtgccgtttcgtgtgctgctgggccacgctgctgaggttctgatgggaccttcccacctgcgtgtgcttcagcactcaccgcacagtctgctttgcttccccaagaccttaatggaggcatttttgcaagaaaccaaataagccttcttaaatgcttgaaagcacgggatgtgtaaaatcaaaaccagctttaagcaaactaatataatttgcaatgtagccctttctattcttctagtgaagacaaacacaaattttaattcatattcatctcactttctagctcatttattgtcttctgggacagttcccaaaacaaacttcctgtggtgaagtacaggccgagtgacttgtggggcccttctgtcagtagcggccgtgcttcccacaacgcctgacgtgctgtctgcatcctattaagcgtccatgaatgtcctggatagaatgaattaaactaaccaaggataccaaacacattcagcaagaaacacaaatcttacatgagaaactgggcataaacaactcccaccacaaatcatcccaaaatgctaaatcaaacataacaataatcctttaaaatgtgtagctgggatttcaagaaagtaagaaaatctaaaaaaaaaaagagctgagaaccagagtgaccgcagtggcagcaccgggccctgggtcgtgtgcggagtcctccgtgcagccctggatgcaggggcggctgcttggaatctgaccccgggacagcaggaaaaattgccttatatgcctttgctttccacaaagaagatttttaaagtccttcccaggaatctcacgatgaatatgccctcctaaagggcaagttcctttttaccctcggtacagaagccctgagctgagactttggcgttggctgttgctgagtgggcagccctggaggtaccggacaaagcaaacaggttctgtctggagggaaatgtccaccaggcctcacaaaattcccatgggtaaactccaaacatcagctcacattccgggttgacaaactacagaaggaaaaagcaccatgaacaagaatcagccaacgacagacagtaaaattacacccaatcttcagctcaggaaatcatcaggcaccaaaaggaaataaatgcatttaaaatgttttttaaaagggggaactgggggtgaaagtgtgaaccagaaacaaggtactctaaaaagtatgggttacattagtttcccttatttggggggaaaaatggagcagatgcattaaacagcatattagacatgatgcaaagagggttctggaccaggaaggatccgtacaaagaaaccagcccaggtgcaggcagagagagggacagaaaacagggttggagaccaggattgaccaaggccagtcacaggcttagtagaggttttccagagtttttgtgaggctccaatcctcagattcaaaaagcctgaagaccctaaatacagtgaataaaaggaaataactggccagacaattgtggtgggtctggaaagttctggagacaagagatggattccgcacacgtgaggaggagtgcagcaggtgcattgttgtctggggcctacagccgttcgttcccaggagtgcatacccagccagactgcacgtgggttccagaaagcagggactggcagggtccccagccagcaaccctcactcggagcgcctggagaaggtagttcaggggaaactggacacaggaggaagaactgacaggaagaaaaagtgagcagggagaaaggcccccatgagccctgagcacagttgctgaggcggtagtgaaactggagacaggtggagtggacaccgcacagccaggagcctgtggggagatggaagccctgtgaggagtacctgcagattaaggctgcccagtgaggtaccagaaccagaacggttcacgcggctcagtgaagcctaaggccgtagtactgcacagcccagagacaggggcctgccagcacccactgagattttattcctgtgcagttctctttctgctgtttgttagtagctgcgtgtcttccttatattgttttctatgcttttggtaggtttgaaacactgttttctgactgtttagtctgaactctgatcacaagataattggtgaagagcagccattccactctgctccccatgggaatgtctcagaaaggaaattggaaaggagacagtggcctcagtcacagctgtggagacgccttcagaaccctcctcagaccctgccttctttctgaggccgtgctgttgcacccgccgtccagaagcgccactcctacaacaaacgtcacagcagccaacagcgggatctgctttctccctcacactcaggcctgtcctggccccggcaggctttgtcgtgggtgccagggctgtctgcgctgacttcagaattgaacaagagaacagacaacccacttctaaacacacacgctgtggaacacagaaggctggccgtgtgggaaccctcttcctgcattgagccctcgctgttggctaattagaaatgggtcacggctgctgatggtgttctaggggaaagagcactgggtttggggtcagatggctttgccattaatgctgagtgtgcttgatttctctgacagattttaattagttctattccccttaaaggaaaaacatgtgaaaagagaggggctgtttttttaaaaggccttgagtcccaaaggcaagaaccctggggctgggctgactctcccctcacggaagtgaggacctgctgctttgggtcttggcacactgtctacacactgttggggcagggttagtaatactcgtatctaaggccaggttctaggacgtgcttgatattttcagtaaacacaccactggcctcagtgtttttaaatttctgagccggttgctgttcacttctggagttgggtatgggcagctccccagcggttagcatgcaggagccgcagcctggattgctcaagaaggacctctgcagagaggggctagcggcccccgcagcacctgcccttcctgccctgacagtggctctgcccagccacaaaccattgcaagtgacagcagcaaagtacctgcttcctttaggcaagcccaagtgatagccttgaacctatgagattatctttcacccttctccttggcctcgacaggatcttggttccggggaaagaacttgaaggtctttcaaatccttctttccaacatcagtgctggaattaacttctgctgtgaccgagccactccctggaattgcagctgttagtttaggccctttctgatcatgaacacttcctgctggattattctttctttctagtaggaaatcatggtgtttttagaatcctgtaatgatcagagctattgtacattcttcagatttaatatacccacattgagcagagcattttcttgttgcgaggaaatctgaaagcattgagctcatgggatgagtgtagcttccaggtgtcaggccggcgccagctcctcatgcacgctgggttctgggccgttggtggaaaggttagtgctgttcaaatagatcatgctttataagttctgggcattgcccatggacctgcccccacacacctggaatccagagctctgtgttgggaataaatcctatagctgcacagcagaggtgatgactttcaaatgggtcagagcatccgtttggctgccctttatgtgagactggaaaatacacacatgcaaatgttgattaacagagggaaggtgctcgtgtgggtgaatggtggtcgtatggtctctgtgccaacagaactgtctacgctgatgggaacgttctttatctgagctttgtcgagtgtggcagccactggccacatgtgaccgtgagcaccaaaatatggctggtgcaactgagtcactggcgtgtcaattttacgtaagtttaattaacttcgatctcagtatccgcacatggcaggcacgtgctggagagcgccagtgcccactcacaggagagttccactcagtgtggatcttggttttcctactgaggtttaacaaggcaaaaaaatcccccttctaaaactgtattaatacattagtttcccagatcaaaattgatgtcaaccttgattacctgaactatttgaggagttatactaacaaatttagttttcttgctaagtatttatcagtattgtctttctaaaatgtgttagcccaccactgaggctttgtaagtagtaatgagcatagggaaacttgccttgaaatatcatcactgagctctagttcttgcaccacaaagatgcagggagtggaagcaattcagacaaatctgcttggtccaccaagccatcccagagcagaggctgtccaactaggccaagtggcctcccagaatgccatggaagggttcaggagcaattatcctggaatccacgggatggtgccctgaatcccacagaatgctgcaggagcaggcatcctggaatcccacagatggtgcgggaacaggcatcctggaatcccatggacgctgcaggatcaggcatcctggaatcccttggacgctgcaggagcaggcatcctagaatcccacggatggtgtgggaacaggcatcctggaatcccacagacgttgtggaagcaggcatcctggaatccacggacgccgcgggagcaggcatcctggaatcccacggacagtgaaggaacaggcatcctggaatcccaccgacggtgtggaagcaggcatcctgcaatcccatggacggtgcgggagcaggcatcctggaatcccacggacagtgtgggagcaggcatcctggaatcccatggacaacgtgggatcaggcatcctggaatcccacggacgctgcaggaacaggcatcctggaatcccgcggacgctgcggagcaggcatcgtggaatcccatggacagtgcgggagcaggcccctaaaggaaaacctaccccagcagccctcttttaatctctaacacatgttgggttaaatgaatttgcttcacctgcgttttctgtctcaggggaggtcaaacctcacagctctgtgctctgtgtggcttctttcattccctaaacgcaatgtgtgggtcagccctgagcactctgtagtggacactgtggtcgagaacgtaactacacgtgggccacgggccacagaaaaccagggtactttctgggcggagggtttctgagaccggaggagcaaagcactttcaataactgtgtccctgatgtcttctcgtttcctgtgcgatctcaagttctaaaaccttagtgttgaaaaattttcctggtcaatctgatggtctttttatggggatttgagttcataaaaaatgttattgtacatgcaaggaacagtgtgtcgattcagggttcaaacaggacactgctggcacacgccaaggataaaactgaaaagagcttaaacgcagagaatccttacagaggtctgggcagtcacggagccctgtgagaactgtgcagcctccactctccagccacgggaagccggaaccctggccagaggggcagggaagggacccgagacaccagggcttgacgtatttgagcagccacggagggccagagcctggggtggccgccctcctgcccccgtctcctgcgggctcctcctgtagcagccactgcaggggaggggagggagggagatgggcgggcagagggaacagcagcatgacgctgtccaaggctttctaaatccaggggttgagatcctagaacaactcagaaacacagacacatccagacacttctctctcagctctcagatcttccactccccagggatcaagcctttaggagagccaagaaagccttagtccaggaggaatcgtgtggggttccaacaccgtgttgtgatttccgccctcccagctttcagagagcggtgcagtgggcccgccatggctccaagtcccacggagttcccaccgcctgcttctggaaggtgggcaggttctcggtagaggtggcgatgcagagtctacgtggggaacatcaggcccctgcaaagctgagacaggagcttcctccacacttcatcaaagcctgtcttgccaggcatggaaccccgggcctctaggcgaggcctctgcagggactgaaggagggagggtcggtgccccagcacacagctgtgcacaggcggccccgggtcatgtggtgccagagctggggcgaaggccgggccagcagacggccctggatgcgcctcaggactggacgctgacctggcaggactcagtcagcaccgactgctcggcctcgagagagcagcctggccggcctctgtgccactgcacccaggacccaaacccaggccctgtactcctgctgctgacaacctcgaagccccacccgacagcgggtggctgctgccaccttcagcccagcacccagctggccgccccgacttgagaccacagttctggtctcgtccattttcaccccgccatcatcctcactcacaacgaacccggacactgctgcctcaggagccgccaattccactccgtctccatcccttcctgcatctctccccgccacggtcccgtcccggagggttctgcacagaggggcagccgggggtcctctgtggacccgctcgagcccacggggcccaggtgcagggtcgcctcagccaggccccagtggctccggggcaggggaccggggctcccgtgaaatgcgggaagaaggagccctcctggcccggcgcgtggggccgcccctctcccaccccagcagccgcatcgcctcctttctgctccctccgcgcccacccacgccgggcgcggcttccccatccagaatcacatgagaggcgtttttgtcattgaatttcttggtgaacgccccaggcctcagtgctgcctgagcctgaggaagtctcaggggacgcctctggcctagaccttcctcttgaaccccagcccggtgtgagcaccgggtgctccacgacccccggaccccgccaggccccccagatccaaccaggaccccacatccctccaggaaccccagatccccccaggacccccagaccccaccaggacccccggaacccaccaggccccccagaccccaccaggacccccggaccccaccaggacccccggaccccaccaggacccccagaccccacctggactcccagaccccgccaggtcccccagatcccgccaggccctccgacccgccaggaaccccagatccccccaagacccctggaccccactgagaggtgacagcatgctggcagccctcgctcgctcttggcgcctcctctgcctcggtgcccactctggccgcgcttgaggagcgcttcagcccgccgctgcactgtgggagcccgtttctgggatggccaaagccagagccggctccctcagcttgcagggaggtgtggagggagaggcgcgggcgggaaccggggctgtgcgcggcccttgcgggccagcgcaagttcgtgggctcggtaggccccgcacttggagcggccggccggcgctgccggccccgggcagtgaggggcttagcacccgggccagcagctgtggagggtgcaccgggttcccccagcagtgccggcctgctggcagtgtgctcgaattctcgcggggcctcagctgcctccccgcagggcagggctcgggacctgcagcccaccatgcccgagtctccccccaccccgccgtggactcctgcaccgtctagcctcccttacaagtgccgccccctgctccgcggcacctggtcccatggaccacccaagggctgcgggcacgcagcgcaaaactgggggcagctccgcctgcggcccggtgcaggatacactaggtgaagccagctgggctcctgagtccagtggggacgtggggaacctttatgtctagctaagagattgtaaatacaccagtcaacaccctgtgtctagctcaagatttgtaaatgcaccaatcagtgttctgtgtctagctaatctagtgttgacttggagaacttttgtgtctggctcagggattgtaaacgcaccaatcagtaccctgtcaaaacggaccaatcagctctcagtaaaacagaccaatcagctctctgtaaaatggaccaatcagtaggatgagggtggggccagataagggaataaaagcaggcggccggagcctgtagagacaattcactgggtctccttatgtgctgtggcagtcgtgttcttttgccctttgtaataaatcttgctgcagtaaatcttgctgctgcttgttctttgggtcagcactgcgcttatgagctgtaatactcactaggaatgtctgcggcttcactcctaagaccagcgagaccacaaacccaccgggaggaataagtaactctgggcgggaggaatgaacaactccagagaggggccgccttaagagctgtaacactcaccgtgaaggtctgcagcttcactcttgaagccagcgagaccgcgaatccagcagaaggaacaaactctaaacatgtccgaacatcagaaggaacaaactggggacacattacctttaagaactgtaacactcactgtgagggtccgtgtcttagttcttcaagtcagtgagaccaaaaacccacgaattacgaacacctcgccaggacccccagacccgagcccgaggctggcgctgctctctcacctgcgggctgctgtggctgtgacttgccggggctcaggctgaacccttcggggtgtccttgccctgtttttctcccgtctccgtcgggaaggcccgcgggccccccatcaagtccctgttgggatccggccagttctcgtctgcttcacacccgccggcgccccatggccacaccgccgcctgaagaccccacacctcgccctctgcagggccctcgggatggggctccgcaagaccaggcttctcaggacggggctcctctagaggcttcaggctttccgccctcccgccatggactccagacgcggccgccgctcccctcccaccgggatcccagagcggcaggcttctcccgctagcagtgcgtccgagccggcctggcttccccgcacagccgcgcaccgagcttgatcccggtactccgccgcggctcgccctcccgctgagcgtgagatccagaaagccgtctttcctgtctgtcggcttctcccgctttctcaaccctggtgggcgcccagtaaatacttgctgaatgagtaagggtggaagtatggattatttttcactttttttcttttttttaacttttttttttgcgacaggtcctcgctctgtcacccaggctggagagcagtggtgtgatcacagcttaggcagcctccaccttcctggcacaggtgatcctcctacctcagtctccccagtagctgtggaccacaggcacacacagccacacccggctaattatttttccattttttttatggagacgggggtgtcactctgttgcccaggctggtctcaaattcctgggagcaagcagtcctcccacctcggcttcccaaagtgctgggattttgcaggtgtgagccaccaagcccagccttttttttttttttcaacttttattttagactcagggggcacatgggcaggcttgttacctggatatatttcatgatgataagtttggggtctgactgatcccgccacccagctacagagcatagtacccaacagtttttcaacccttgcccctccgtttctccctccatttctcctccctgagtaccgtgttttgctcccacttacgagaacacgtggtatttggttttctgttgctgtgctaattggcttaggatgatggcctccagctgcctccatggtgcttcaaaggacgtgatttcagtctttgtggctgcgtaatattccatgctctgtatgtaccacatgttcttcatctagtccacctttgatggagacgtattgattccatgtctttgctttgtgaatagggctgttgatgggcatgtctttttgctagaatgatttctgtgcctttgggtttatagccagtaatggtattgccaggttgaatggtagttctaagttctttgagaaatcagcaaactgctttccaccacaaagtgaaggtggtggttacgtgatgtgagggtggctgttaggtgaggtgagggtgggttaggtaaggtgcaggctgtggttagttgaagtgaatataggttagatgagttcagagtggtggttaggtgaagtgaggttagtggttaggtgaggtgaagatgggttccatgaggtgagtcaggtggtgggtaagtgaagggagtatgtgttggatgaggtgagggtggtggctagataaagtgagtaaagtggttaggtggagatgaaggtggttgttagatgatgtgagggtggcagttaggtgaggtaagagttatggataagtgaggtgaatatggtgggtaagcgaagtgagtatgggttagatgaattgagagtggtggttaggtgagaagagtggttaggtgaagtgaagttgataggtgaagtgagatggtggttaggtgaaggagatggttagatgaattgagaatggtggttggatgaggtgagggtcatggttaggtgaagtatgggttagatgaggcttgaggtgagggtggtggttgggtgaagtgatggttaggcgaggtgagtgggggtggttaggtgaagtgagtacagattaggtaagttgagagtggtgattaggtgaggtgaagttagtggttaggtgagtgaaggtggtgattaggtgaggtgagggtgatggttaggtgaggtgaaggtggtgattaggtgaggtgaggttagtggttaggtgaggtgaagatgggttccgtgaggtgagggtggtgggtaagtgaagtgaggatgggttggatgagttgatagtggtggttagataaggtgaggagagtggttaggtgaagtgaaggtgactgttaggtgaagtgagatggtggttaggtgaaggggatggttagatgagttgagaatggaggttagatgaggtgagggtcatggttaggtggagtatgggttagatgagacttgtggttaggtgaggtgagggtggtagttgggtgaagtgatgtgatggttaggtgaggtgagggtggtagttgaagtgagtacgggttagataagttgagagtggtggttaggtgaggtgagggtggtggttaatgacatattctcgtgagtggagtcgggaaggatgaggaatagcaaggagaagggatctggccaggactactctgtgctcttctctgagctgtcctaagtaagcagggccgaggttcccaagaagagattcctgcaatgcaaagtaaatgggttaatatgttctaaatgattaaacagtaaagttatacttaagagccaaactaataaacaagagcataactttctgagttttgccttaaattttgtattgcaaaataacagtgaggtttggttgtacttatacggacttagggcctcttgacaaaaataaaattcatcacataagcacacgtggattgtttccctctttattttataggaattagctttatcaagaaaatgcagaccaggaagcggcctttgggagtggcgagtgatgatgatgccctgcaggtgttgctggtttttatcgcaacaggcactggttttccttctagaggacccagttctttttgtgtaggattccctgttgtcatagaactccctgtgattctgccaccatgatgatcatgaatccctcctgtacctgatgatttcgtggagattaaaagtatcaagccatggaccttgctaaaccaagaaatgttaaggaaataaaattcccaagatactgcagctcagcagctggctctggcttaggcaagtacctctgtctggaacacctcaccctgtgcctgtaggtaatagtgtttaacacactcagacctgtgtgtccacctgagctcatccaggagtgtcagaaagctggatgtgctgcctcggagacagactgagctctctttctgaacttagctcagcgcttccttcctttgctttccctgatggtggcgggtcgtgttccctccagacagaggtgctttacaggaaccatccaaagtcattcatgatgaattataagaaaaaagtggaggttccaaattgtgggtgtcattttttggttcaaaatggaggtggaattataaagcgagactatctcgtgccttccaggcaggcaagagtccttcatggccagagcacagggctggatcctggaaccattcctgttgtctggtgttgctggtcaccctcagcttacagatctgttttctttctttgtagatcacgttttatattctggtgaaggccatttatacactgggctacagtgtctctctgatgtctcttgcaacaggaagcataattctgtgcctcttcaggtaaggagcagaaattagtaattctgcaaagaatacaagacaagcaaagtttccatttaggccttctaggcgccacccagcctggagccctctgtgtgctaagccaataagactcccatggagaatgctgccattaaatatgatgttaacatttacatttttatttaaatactctgcatttattatgtaaagagataatgctgaccttggataggaactcagctcagctatcatttattaaatacttagccattatgcaaatgcgtggaaaatacagaaaaaataggatctcacccttgccatccagaagctcctgtgttttggctgtaggtgctctattagagagatgctacgacgtgtttaagagcatgaaggtggagtcaggctgtctgggttcaaatcctgggtcagtctcttagcagctgtgtgaccttggacaaagtacttaacttgtctgtgcttctgtaatgtgtggagactaagaagcccccttcagagggctgttgtgagaacttgatgtgtcggtagatgtgaagggcttagcatggcacgtgaggttgtgatgagggcttattaaatgctgcccactcttccctggcagtgaggtgagatggcccctgcacacctgcatctttagtgggtctcttggtaactaatgatgacatagtgcagatgatttgaccaatcaaatcattgttcatttgggttgggggtagaggttttggtcccagaacggcagtttctctataggaagagctctggtgaattaggattaaatctgtcttacgtatcattagcactaaataaaatggaggctattcagcttattctatgtatcaatcactacatattttatttctctttgctaaaatgtaatcgaaatgtttgccagtatgtcagataaaaatcaatgataaatggaatatttctagaccctaacaacagtccattaggagatccatacttggaagtgtaaaataaacaagttaggagtggttgagaatccagctaagaaagccagcaaatgtaattgagctatgctttttattgttcaggcatctcaactatggttggtaattctcagaaagaataagtctacctttgatacagggctgcactttgaaaagtgaggaaggcctatcatattctagttatttttaaaatctggtacaatattaatatttgttctattatacttatattttatagagtataatttactatatatgtttactattttactgtatatgtttaaatttttaagttgctaaagagaaagaggaaaactttaaggaagtaagctgacccagggaaattgaatctgaaaattttatcttattttcctatattgaacaatccctattttttacacacatctgtttccttcacatatgtacagcatgacatatttaattaggcaacccaagagcatcaggaattatcccgcctatgaacactgatcctttagggcacagaacacacccctccatctgtaggcatcattcgtcacctgcctgtggcacttctgagctgaaaaaagtgcatgtctgtgttggatgtcattctgctggtgaccacatagtgtccagagctggtgtgcagctctaggcttggggctcatcccacgtccttctgtcagaagcagggtccagtgccacttcctgagagctgtgagagtctgtcaattctaaagtccgaattataaaaataacgtgctccattgtctcagtaattatacccaggaagcagaatgctttgcgggcattgaacatttcttctccaaaatagcccaataagccaagaaagccttcagtagccatgttcttattttatacacgggaaatggagctacttacttcttagaacacatcccaaagtgtgcagtgttgacgctcacaacaaatacaggaccagctttgtaggggagtcatcctatggtgacagctgcccctgtgacccagggactgacagagacggttccagtccatgccagtccagcagggtccacagcacggttgctcctcagacaacacgggtttgaactgcatggtccacttctctgagaatgtttttcaatacagtcagccctctgtattgatggataccacagatcagaagtatggcctattctcaggatgagaagcctgcagatacagaggaccagcttcatatatccacgggtcctgaaggggcaaatgtgggacttgcacattcatggactttggcatggggtaaggggggcaagattgggggagggggattggacccaatcccctgtggataccaagggacagctgtaactgctaatatgcaccgtgtcaagtactttacagatgttaacatcatcaggtaagtactgccactgacccagtttctccaggtggagagtgcagccccaggaaggtcgctggacattgcagggttgctgagctggtgcacgcagagccctgatttcaacccaggctctcatcactgcagcctgcacctctgtcactgtcagatggacgtggcctccaaaatcctttcccaccattgctaatgagcagactctggtccccctttccggaagccacactttctactacctccattgcccattggcagatccatacgtggaagtgaaaatttaaaacgagcagtgagtaatgaagaatatcactgagaaacccagcaaacatgtatttggatggcttgctaactggaccaaaagcatgctcatctctgagcaatggctgtaagtgccaaccccactccacagaaacacagggggtggaagagagagggaaggagaaaaaataagagagggaaaaaggtgatttggagagatgaatgcagccaaggagagaatcagtgggaagcaaaacggcaccggtgggggacatttgccttctgaggaaggggtgttgaggagaagccagagggagcggcactggtgtgtgagcctggcgcagtggccacacagctttggaattgctcccaagacaccgagaaaattcagagcatatttacaattctttgcttaccaaggctctgtggcagtgttacatatgctaaacgtttgctaatgcagtatatgttcagaatgcatgtttaccaggcgaacacagcaccagatcgagatgtgggatacaactctcaccagggaccctgcagccacccggtgtctaccccacctctctcctgaaaagcagtgttattcagagttctggcaccagagacaacttttctctttttgaaaacgttacctcggaatcatgcaaaaggaactcctggtctctgggttctttcactcagcattttgcaaggaggttcatccatgttgcatggagttaccatgttttattttcttgctgtatagtattccactcaaaaaaatatgatgtatttatccaatcaactgttcatggacatatgggttactcagtttgaggcatagcaaataatgctgctgttatattcttagtcatcatctcatgcacagatgcacatgttactttaggtatgtgtgtatatgtacacacatttatatttacatttgtgtttatcttatatttactggtcataggctatacatagagttagtcttcctaaatactgccagtagatatgctaataccctgatgggatcatcataccacatagatatgtatcaaagcatcaaagtataccccataaatatgtacagttaccgtgtgtccattaaaaataaatttattaaaaaatgaatgctgccaagtaatttccaaagtggttaccagtgtacacacccactagcagggcaggaggcttccattcacaccacaccttgcctccattcggtattgtccatttttaagccttcagccattcttgtgcgtctgtaataacatagaacggtgttttaatttacatttccctgatttccctggtggaaaatgagttcatacattttaaaatcttgctttttttgaagtgccatttcaaaatgagtttgctcatttttctgtttggtctctccctctatttaagtcaatgtttaggagtcttcgagcctttcattacatacgagtattacggattttgtctccagctctgtgactgagtttaagctctcttcgtggtctttggataaacagaagctcttaactttaatgaagcttatttgccatttgtttctttatgtcattgtttcttgagtcctcttaagaaatctttgccatattcttttttttatcatctagagatgttattattttaacttttcatgtttctgtccataatccacctgtgagttatttctgctgtggtgtgaagcagggtaaagtttctttagggctttcagtgtgtgtatcccgttgtccgggagcgggtacaggaaggagcttctatggcctgcaatgcttctcttcacagagcaggcagtggcatgtgtgggggcctcgagtcccggttctgctcctttggccttctgccactgcactgtttctgtttattataattaccctgcctttaataagtctttattcctaggagtccaagtccatcaacccttttttattttttaattaggaagagcttggccatttgtacttacatgtgaattttaaaaatcaactcttaaacttttaatgtcaattgccgttttaaaaaatcaggctggttgcattggctcacgcctgtgatcccagcactttgggaggccaagacagaatgattgcttgtgctcaggagttcaagacaagccagggcaacatagggagaccccatctctacagtctctacaacaaaatttaaaattagctggccgtggtggcacatgcctgcagttccagctacttaaggggctggggtggaagaatcacttgagcccaggttgaggctgcagtgagctgtgatcacaccactgcactccagcctgggcaacagagcaagaccatttctctcaaaaaaacaaaaaaaacaaaaattctattgcaatttcatttggtattgcattgtatctatagatcagcttggatagacaggacatcgttctaattatgaatcatccaacctgtgatcatagtagatccttccatttgttttggttttctttaatttctcagtgttttatgattttccgagtggaagtcttacacataatttaatagtcattcctagatatttgtttatggtattataaatattttgttaaattccatttctaattgtttcttgaaaaaatttattttagtatattaatattgcttcaaaggccaggtacagtagctcatgcctataatcccagcactttgggtcaaggcaggaggattgcttgagcccaggagtttgagaccagcctgggcgacagagtgagacaccctgtcttagaaggaaaaagaaaaaaaaaagacaaacctcttgcttcagcaacctcactaaatccatttatggattctttattccataaattcacttggatttttctatgtgtaattcatgctgtttgtgaagtacaatgttgaatagaagtggtgatagcaaacaacattttcttatttctgaactcagagtgaaagctttccatacttcactataagcatgatgtttgctgtagatattttgaagataccttttaccaaattaagtttctctcaattcccagttttctgagactttttagtcatgaatggcttttgaattttatcatgatttttatgtacatacatatggtttttaacatttgttcatttaatttgaaatattacatggatagattttctaatgttaaatccacctttcattcctaaattaaccctacatggttttgatatattatgtttttaatatatagctgaactcagcttgataatattttgttgaggatttttcatctacattcatgaaattgatctattgttttcctttcccatgagtcaatctggtaaggtttggtgttggcccataaagtgaagaggaaagtgttttctcttttctattttttggagtagtttaggttagataagatgttatatcttctttaaatgtttggaagaagtcaccagaagagccaacagggcagagagttttttttgtgagcaggttttatttggctgtccaaaatttctaaaccagatataggactattcagactctcggtttctttgtatgttagttctgctaagttgtacttttcaaggaatctgtccatttcatcaatattttcaatgttctgttacatttttaatgtatgtaagatctatacagatctctgctccttcagtcctggtattgtcaatttgtgttttctttctttttatcttgatgagtcttactgaataaagtgttaccaattttatcagtgttttccaagaaccaactttcagtcctctgaagcttgttgagacttgctttatgatctggcatgccatctgttttggtaaaagtttcattttcgtttgaaaataatgtaaattatgcagttcttggttatgtgctacataggtcattcacatcgtttttcaatgttatttaattattccaaatctttactgattgttttaactgctttttctaccagaactgaaagacttacatttttctaccagtactgaaagacatgtattaaaatctccaactataattgtgggcttgtctatttgtccttttaattctttcactgtttaccatctatattttaaggctatgtcattaggagtatatcagtttaaactgccaatgtgtagtaatgtctcctaccttaaaaatctgctttgtctgatatttaagataattttattagctttattttgcattttcaaggtatacggttttctgtgctttttattttctacttccctgtgttttatattaaatgtagatattttataagctccatttaattggaatttgctttttgtctacaatctttatttttaaatgcagcattttcatacgcacagtctacttatgacaactgtgagttctaatctacctttcatttctttgctttctaattgtctcaactgttctttgttggttttgcacatattcttgtttcttttgaattgatcaaatatttttattcttctggttttctcctcttttggattgttactattcttctagttgtcaccccagattcacaataggcatccttttctcatgagttaacctccagtaactacttttaccactttccagtcaattcaagagttttgcaacattttagctccattttacctctcatgtgttttgtgtttttgtcatgtattttaattccatatataaatactattgttgttgtgttaaaggtcgcatttacttagcattccctcaacatttctttctggtgctcttcattctcctgcattttgtgattccatctggaatcattttcctactgcaggaagatttccctgtagtgtttctctcagcacagtcccaccagcagtgaattcttatgagttttggttgcttgaaatcattttaattttgccttcatttttgaggctattttggagaggattgaattctgttggcagttattttctctcaccacattaaagatgtacgtcgtgacctcttctgaaaagtcagctgccagtgcaccattgttcctttgaggcagtgtgtcttttttcctctgaaagctttgaagacttttttcagtttttagcagttacactctcataggacttaaggtggttttcttcacatttctccttcttggggtttgtagagtttctttaatctgttactgtcttttgtcagtttcaaaaaaccttgtggctagcatctcttcaaatactgctccttcctaattctcttctcctaaaactacatttcccatgtattatattactattttttcctatatttttcatatttttggtttctactttaatctagacatactactgatccaattcatggtactcgcatctgctgtgttaaattaatgttagtcccctctatttaactcttaatttcaaggttttttaagttttaaattttctatttgattgtcttaattttattatctaataaaattctccatttctttatctgttttcttaaatatattaattttatagtccatgtcttaaaacactagcatctgaatcgctattgtctgggtttttttgtttttttttgtttttttttttgtttttttgtgtttttttgagatggagtatgtctctgtcacccaggctggagtgcagtggcatgatctccgcttactgcaacctctgcctcccgggttcaagcgatcctcctgcctcagtctctcaagtagctgggattacaagcgtgcaccaccatgcccagctaatttttgtatttttagtggagacggggtttcaccatgtgggccaggctggtctcaagctcctgatctcaagtgatccacccaccttggcctcccaaagtgctaggattacacgcgtgagccaccgtgcctggccagtgttttttctttttaacttggcttttgattacttagtcctgtttctggcatacctagtaattgttactgaaattacaaatacttaaattgcagaggcttcagatgattatcttgctgcagaggatttgaatttttcatggtaacaagggtagaccagcctgggccgtcagggccatgtgggtttcgggctgcatttcaggtttcaagcagccaggtgctctctggtttgttgttactctgggccacagctattcgggactcccccatgaagggaaccctgccaccctcaggccctgaatcctggtgcttgccacccggcacccatgaagctgcaggcatttcccccaagtactcgcccttggccactgctttctggtttatttctcagcctctcacccaacacacagccatctcagagaccaaaaggatttggatgcagcctctatttcctttcctgcagagtcgttgctccccgagggtcacccaacagcccttatctcccgtttctgctgtggcgggtgaggctgccctcgctatggccctggctctgggcttgtcctttctgaacaccctctgtgcccggagagctaaagcagcagatacccacggaggccctggagggcccaggcgctccccacagtgcccttgccttctccccagagtccagccctttggtccccacagctgcggctgctccccaaagcccttgggctgctttttaggatcaccctgatcctttcctgaggtatcctccttcacggccacaagtggaggtcctctttagggtgtgttccaattaagtcttctctgaagcagaatttaagtgttctaagagagttgtaaacaaaatgttctgaatactttttagaaaagcaagatgtcacataaagttaagccccaagaaaggttcatgttgacatttgatgagggtcttgctggaagaagagaattccaagagagaaagttttatgggagtgcgtgcttggcagaggaacagggacgctcagagtccacagagggtgcagtaatgggggtggcatgttgatagaaaaaataaaaataaggttcaagaaactgaaggctaagaatgaggaatcttttgagtgggagagaagggtcctccatgccttgaacaagtggacccagcagaggtttgtggaagaactagagaaagcccctggagctgtgcagaacatcagtaagggctgagctgagtgcacgctggctgtggaggagtttgacaggtgccagcccaaagaggagctgaaaaagcacaatccaatagctgttgagatcagcagtgtccaacagaaacataaggcaagccactagcataattgtaagcattctagtagcctcattttaacattttcttttaaaaataaaaagaaaccagtgaaattaataatacattaaagttgtcagatccaaaatactacttcaatatgtaatcagtataaaaatattaatgagctatttacattcttgtgttcagagtaagtcttcaaaatccagtttgtagttcacacagtatagttcaatttggtcactgatttgtattggaaatacttgatctgcctttagagtttttaaaatttacagttgaaaaagttgttcaaacatacctagctgtccaaacatacataaaagctctccagcaagtcactctggtaccaggagtcatttccctttactattcatatccacattaacaaaactaggttaccatttttagaaagagttgatctaactttgaagcaaaagcacctttgtttgcaaattatgtcttctcaagttaaataagcttacccttgtatcagctcagtataactaatgtcaaattccaaggggcaatgcataaattggcaaacaactctaaatccattgattagaaaaagtgtttctcaaatttatatggtatttttctgccagtttaaatggttctcttgattgtaattaaaatattctgcatatttattcatgttagaaaaatgctaaatcattagttatgatttatattatgggaagtttttatttccacataatttttcatacctagctatgtcacaaataagctctcctttcttcaaggttccaaaaccaacaggatagcactgagaaaacataaattatattgtcattttgcttttgataattgaatatttggaaagcccttactgtttgagaaaaatcttgacttggagttaccaatacagtgaatattgtaaaattcttccatgactcacccaagcatttgcaaagaacactagatcattaagttcattgtcttgtttctttcaggtgttgtgtaaacgctcagcgattcaaagcatttgcacataaatactgaaagattttaacaacaacattcttgatggtttttctagagtctacttcataaaatggaacacaaatactttcaatagtattatacagtggaaagaagcaataatggaaatatctgtcccttgttttaaattccaaatgaattgcaataaattcagggttttgaccttgcagtagctggagcagtgtctgtcataatagaaactagttttttcatatataaccgaattctttgacaaatggaaactatgaaaaaatatttacaccataatttctaatttttataaacaattgacatttgtttataaatttggaattcttctgtaattaaagaattcaaaatattaattgtccagtgagtttctcttaaccacacaactcatcaaaaaccaaagaaaagtacttgcaacttttcaagttttaaattaaaggatcctttatagtttttttgtttgtttgtttgagacaaagtttcgcttgtgttgcccagcctggagcgtaatggcacaatctcggctcctggattcaatctcaacctctgcctcctgggttcaagcgattctcctgcctcagcctcctgagtagctgggattacaggcacccaccaccaagcccggctaatttttgtatttttagtagagacagggtttcaccttgttagtcaggctggtcttgaactcctgacctcaggtgatccacgtgcctcggcctcccaaagtactgggattacaggcatgagccaccgcgcccggcccaggatctttaataatgttagaaagggcttttactctgtgggcaatagtttggcttaaatgaaaattttcactttttgtaaaatatttttcttagtctgttctttatatttctctaacaaatgaaataaaaatttcttttaacatccttttggacaaaaattatttttcttttttatgcaagaatcgaaattgtttcagctggccataattgtttaaaaatttgagggggagggtggatttccaaaatttgtggtaaaatacccataacacaaaatttaccatttgagccatcttaaggtacacaattccttggcactaagcaccttcacagtgttgtgtaactatcaccattagctagttcccgaacttcctattgtcccaaaaggaagccttctatacatgaggcagtcactccccataccctccggctcctggaagctagaatctgccttctccatggaattgcctccactggatgcgtcatacaatatgtggccttttgtgactggtgtctttacttagcataatgttttcaagttcgatccacgtcagtacttcatgcctctttatggttgaatagtatttagttgtatggatatgccacatttagtttatccgttcatctgctcatggacatttgaactgtttcaccatttgcctgttgtgaatagagctgccatgaacattcatgtacacgttttgtttgaaaacttgtttccaactctttggagcgtatacctacaagtggaattctggggcatattgtaattctgtgttgagaaaccaccaaactgctttccacaatgactgtaccattttacatttccactagcaatatatgagggttccagtttcttcacacccatccaatactttttattttgttttcttaatttattattattattactattgccatctagtggtatgcaatgaagtcacattatggttttatgtgcttattgaccatttgtatatctttggagaaatagatagttgagtttttgcccattttctaattgggtttttgtcattgagttgtaagagttctttatatattttagatactaatccttatcagatatataatttgcagacattttctcccattctgtagattttctttgaactctcttagtaatgtcctttaatatgtaaaagtttttaatgttgatgaagtcaactttatctgttgtttttttgctgtgcttttggtatcatacataagaacataagaaatccaacagagcctattagacttgtggctgacaccacaagtacacaggaaatccaaaggtcttgaatatttgctcctatgttttttcagaagagttttatggttttagctcttatgtctaggtcattgattcattctgagtaatttttaatatgctgtgaggtaggggtccagctgtattattttacatatggacatctagttgtcacagcaccattcgttaaaaagactattctttccctattgaatgtccttggcacccttgtcaaaaatcaaatggccttagatgtgtaggtttatttctggactctcagttctatacagttgatctatatgcagtgccacactatttggtagtctgtagtaagtttgaaattgagatgatgagttcttcagctttgttctgtttaagattgttttggctgttcaggatccatcataattccacatgacttcaggatcatcattttggcactcccccatgcgtgtgctggtgcacttgatggaattccacaagtctcttaggctcttgggctctcttcattttccttcattctttgttttctttctgctcctcagactggataatttcatttgacctctctgcaagttcacagatgatttcttttgcctgctcaaacctaggccaggtgcggtggctcacacctgtaatcccagcactttgggaggccaaggcaggcggatcacatacggtcagaggtttgagatcagcctagccaacgttgcgaaaccccaccccatctctactaaaagtacaaaaattagctggtcatggtggcaggtgcctgtaatcccagctattcagaaggctgaggtaggagaatcacttgaatcagggaggcagaagttgcaatgagccaagattgcgccattgcactccagcctgggtgacagaatgagaattcatctccaaaaaaaaaaacctgctttggcctaccccagtgaatcttttagttcaattgtgcatttcagctccaaaatttctacatggttactttttctaatttctatctcttttatcaatattgtctatgtgttgagacattgttctcctggtttcctttatttgtctatggttttttgttttaggtttttttttttttgcactctttaagcatatttaagacactttatttaaagttgttgcctatgactttaagtcagaaacttaaggtaaatctatttcatgtgatgcctcaaggataatttttgttgattcattttatttttcctgtaaatgggccatattttcttattattattttttgctttatattttttgttgaaactgggcattttgaatattctgatgtgataactctggaatcacagtctcccacttctaggatttgttatttcttgctgccggttgtagctgtttatttagggattttctaaactatttttataaagactcttggtctttattggtcgctgaagactctatacttttagctcagtcatcagctagttgtttgatagagatttccacaaacacctggagtcaaagagataaaaaactgaaaaaaaaagaaaaagtgccctctaaaggcctttgtaggctggctctgtgttgggagcactccttcaatgcttaaccagactgtctgcagctctgccttagctctcgctgcctgcttgtgctaagcccaaagagcagcctgagataacagcttgtgggattctctgattctttctgccctgaacgtgcacctggatttctgggttccccacttcatgtgagagcttcttgaagcccttattctccaagattctcactccacagcttttcctctcaggctatcagaggacctgatgtttgcttcccctgccaagtgtctcctgaggtggcgggagcttattcattcagcttccagcatgtctgcaaaaccacttctcagcccgcggaatcttagtcttttgcccagaacccacagaaaggtcaaaacaaacacaactccttgggtttatgaaatttctccaggaccaagtacccacaataggaacatgagcttctgtcttcaaaatgaccaccatgttggggtggggtggaaaagggtgagctaaagacagcatgccctctgcctattcagggccttcccccggctaagcgttcacttggttggcataagcctttgagtatctttgttgttgttgttgttgtttcttgaatggtccctacaccaccattttcactgacctcctttgttgggattttaacagttttttaattgttgataggaaacttcttatcaaattcagtatgtatttgctaaaaacgtttcttcatctcatctactttattatctcaaaaaaattcttaacagagctttttcatttttctctgccacagcaaattgcagttgccattcatggtgaaatctgcttcatgccctcaactcttttatttttctgtgcttctctgatcatagtatgagtttctcatctctgtctggttctctctccctccctccccttccccctccaattcctctctctcccactcccccacctcaggaagctgcactgcaccaggaattacatccacctgaacctgttcctgtccttcatcctgagagccatctcagtgctggtcaaggacgacgttctctactccagctctggcacgttgcactgccctgaccagccatcctcctgggtaaggctgtccccaccacagggtcccactgtcttgtccctttatctctgttccctcttctcacgatgaggacgtctctgtgctggtgcagggttcctgtcagggcacagggtcatggctgagaaacagagagcctggcagagaggctggggccagaacaggcccagttggcagctgagacatgggggtggcactgaggcagggagggtggggttcccaggcatcatcagggctgggaagtgaaagggccctgagccctgacccccatggagctagactgaggaaaggcacacccttcctcacacacatctgggttgagaaatcttggcacagatgaacacacacaactctgttgctttctgtctgaatctcctagattctgagcagtaaaggcttccttccagattgctggaagaacttgatcttgtgccatgggtaaattccttgtaaagcctgattgtgaactcagttgcatatctatgaatatctgcgtccaactctttgatttcaggataatcttaattattagttcctacaaatgcagtacagggtgtttgactccacccacgcccttctccctcagctgtatttgtagtagttattggtggttatatagtacctaatcaagtcagtttaacatagcaggtgctgttgagcacctcctacatgcaggccgttctaccaagcacatccctgctggcccagagcactccacatgctcggcatctttgtgaaggattcgtgcccctggtacctcactctcaaccagagacccttgctaccaaggttcttcttaaggagccaggagctccagactctcctcccgcattccacgcagataggtccctctgctggcacagttgctcagtctaaacagatttgaattaacttagttttaagatagtaaagccgtttataaccacacagacgtcagctttttcatagcccatcatcgtcttcatgcaggaaatgtgcctctggggataagctggcgcgtgatgcctgtgaatcagatttgctgtgataacaaaattaaaaacaagatttttcacctggcaagccccgttccccaggtggagttgggcccctggagttcctgtcctagtaatagggtctccccacatgtgtaacagtggtcaacgcccataagaagtgtgaggatggtgtcggattctctctcaccgagatggtttcaggcttcactctgcagatcccctgcccttagctcttcatgaagaaagcagtcacccttgacagacacttgacagaccctcagaggaccactcggacccatggcagaccctcaacggacccatgacgtcaaaagcagtgacccatgaaagaccctcaggacccgttacagaccctcaggacccatgacggaccctcaggaccctgacggaccctcaacggacctgtgatgtagaaagcagtgacccatgacagaccctcaggacccatgacagaccctcaggaccctgacggaccctcaatggacctgtgatgtagaaagcagtggcccatgacagacccttgggacccgcgacatggaaagcagtgacccgcaacagacccttgacagaccgttaggaccctcgacagaccctcaggaccctcgacagaccctcagtggaccccagcggaccctggaggacctgcaacatggaaagtggtggccctcgggactgggtgctggcctggagcagcagctctctactctcccctattgcttcaacctgaaaggagtgcccagaacctccgaggcagagtggatctgagccagcgtggctgccccaccgggagggcctggcctcccacacactgcacagagcatcctgtaaatgccgggctgtttctgtggacactgggtgaacactcattcgccttcagaagagcagttgccccatttcacagacgaggaaactgggcagaggggaacccagcaactggcccggagtctcgggatccaaagcccaggtgcatgtggggccccacgatgcccagcagcacagagccagtgccccaagtcaagctctgtgaggacagagggagtcggctttgccccttgtgccagatgccccgtgactctagtaccagcaaggggaaagcagaggcaaagggaaaagcatgtgacagacagaggagggaagggacagaggacaggagggaggggagatgcctgaggccagagaggagccaagctgggtcttccctctcgctgtcctctaaagaacgctcaggctttgaataaacaagtcagccagaaatagtcagacctcagtataagaaacaggaaaaatagctttccttccagttgaatttttttgtatattttttgttcccagctgacttcaccaaaccaaggttattctcccaggcagaacacctggggcagcacccccaaaataggtgcagggtctttttctcatgatattaggatcagccaagaagatgaacagttctcttcttggccccttccatgcggcctagacagagacacagtttcctctggggccctgggctgtcccgggacacggagcaaacctgccctttattctcaccatcagccacagcaggttcccctgcggggtctgtgggactcccagaagcacacccccaagcttactaagctgagtcctgcctgcctcacgtgcacatgtgtgcgtactcacgtgctcacacagaggctccccgacaaggccacggggctgaggagggaccagctgttgactgggcaccgggccttggtgggtctcctggccggggttcctcacatcaaccctgtgggtggcaactggccctggagttgggcccctcctgtggtgctctggagcctgtgtaatgaggtttgaaggtcacactgtagaaaatgaagtcagtagaggccttgtcattccccagacctcccttcctcagtgtgcttgtttggtgaatgggcacatgggaaaaaatgcagagatgtttttgttcccatttaagaagaaaatggaatcgtcttcagcacactcttcctgcgtcctctttgctccttcagcagaatgtgccagggccccaccaggtcggcgcctgctttcaggcggcacacggtccctcatgtggcatggtgttctccgcgtagcccatcctctggtatggggcctgctctgcactttatttttctcttttgccgctgcaggcatcaccggacatgcgtctttatggactggtgttcttatttctgtcaggtggaactcgccagtgaggttgccaggtcagagggtgtctccatccacgtgtgctcctgtcgcagacatttacttctcagttctggaggcctgagaccgaggcaccaccagattcgggtctgacttctctgtgtcctcacgtggtggaaggggcgagggatgttgctggggtccctttacaggggcactaatcccatcgcaaagcgccacccactaacaccctcccacagcgggtggggcttcaacatgtgaattctggcagcataaagaagcctccagcccattgcaaagggcatgcatatttttattttttgttttgttttgttttgttttttgacacagactcactctgttttccaggctggagtgcagtggcaggatctcggctcactgcaacctccacctcccaggttcaggcgattctcatgtctcagccttctgagtagctgggatcacaggcaggtgtcaccacacccagctaattttgtgtgtgtgtgtgtgtgtgtgtatttttagtagagatggggtttcactatgttggccaggctggtctcgaacccctgacctcaagtgatccccccgccttggcctcccaaagtgctgggattacaggtgtgagccactgcacccggctgcatgtgtatttttaaattgtagtaaatctgttttgctagattgccttccaaaaggctgagacagctgacagctgcacgtgggatcacaccagagcccaaagacctgactggttttaccattttgcctgtctggtgggcgttggcactgcctctgtggctctcatgcacactccagggcagcgcacctgctgagaactggcatctttccacaagctgttccggccctctaaaacaccgcattcatctgtaatattggcgtcataaaaattaaacatttgtcatttctattgtaaatatgtttccagattcatgatttgtcaattgtatttttatggtatttttaccatctaaaagcttttaaagtttatataaccaaaattgctttccatccttttagcacttcttgttttcatttcagatttcccgtcttggttaagaggtttctattctgagattgttcatttgtgtcttggttcatctgcagtttactccttgtgtttaagatgcggtcttttaccgagagacaaaagaaggcacgttgcatcccgatgggtgactaactcccagaatctcacctcccagagattttaaaacttgatttatttcactttacttttcaaatgagtggcagtaagtcacaaattgacttactgtgactctcctgcatccatggctgtgttatcatgtgtgtctttccacataagaggctctgtcctgatgcccagggcagaacttgctgggatctgagtgtccattcaccaggcgttgacacacgagatccgcgtgcaggcactcgccttactctggtggcaaagatggacaataagggtaactctcacctgtgcagtgtctcagaaaatgaagcacccaccctgctatgcttgggaaaaaggaaaagcagaggaaggcaaggcagatccagagcactggggaggtcggggccgcctgcagggctcaggggaggtcggggcagcctgcagggctcaggggaggccagggacagcctgcagggctcagcggctctcaggaccccggaggccaaattgaaaggccaccatatgaaagtatatccacccatatataagtgtatccacccatatatataagtatacccacccatatataaaaaagtatattcacccgtatatatttatagggatagccatctgttatatgtaggtagtatatccacccatacatatgtaagtatatccacccacaaatatgtataggtgtatccatctgatatatgtatcagatatatatctgccatacgtatgtacacccacctacatatgtctatagatatgaacataaatacagaaataagaatggaaatgaaaatactattctttttcagcacactttatataagcttagtgattaaaagtagcagtttcatggttgctcatgaagctgaaatgcctggtacatttagttagatgaaggtctgagagattaaaatagatggattttatataataatgcaattctttcccagtgaaaatccttttaaaatgtcctcagaaaatattttatttgctagatttctcttgttttcccctttattttttcatgtttagttaagttacccaatattaaaaccagttttttacaattgaggaacttacatagttgtcaaaaatttaaaaaaaaagtctggggcctggtgtgggtccactgctttctcttcccaggcgctgctcacctgagctgctagtgagggcagctggcatgtgccgcgccctctacctgcactcgtgttctgttcacacaaagggacagaggccagagccctgggcccctgcagggcccctgccacccatacctcgccccacacatgacctcctgccggctcttctggttctttgctggccggcaggaccactgcgggggttcccgtgttgaagcaggtcagagaaggggaagtggagtagccgaggggaggagctgagaacgggagggccagggagctgtgcggcctgtctaggaccctccagcagtgagtgcgctcacctcaggcagtgctgttcacagttgcgtttgcagtgatcctcctctggagccgcggatacaaaggtcccagtgccaccagcaggcattcctggccagcgccctggcctctgtgttaatcactttccttcccaatggcaccggtgtctccttccttgcacagataccaagcgccctgtgtcctggagcggcactgcctgctggtagctgctgttaccggatgaggtcatgctttcctctcctctgtttgcggtttccaggtgggctgcaagctgagcctggtcttcctgcagtactgcatcatggccaacttcttctggctgctggtggaggggctctacctccacaccctcctggtggccatgctcccccctagaaggtgcttcctggcctacctcctgatcggatggggtaagtgtgccccttcccacccacaaaccctccatcccatcccatcggaccaaacaacgcaaacattttcagctcagaaaacactttgccgcccccgtagaatgcatgtcagggtcatatcatgaagagctcttccaggatgactggaggctcctacaaacaacacattaatatttctggtaaccaggaacctagtttaaaatactcccattcctggccccttgtacccccattcaaactgggctgttaacaagtaacaaaacggtttcctcctccgcctctacccacttcacctttcactgagggtggggatcagagaggagaggcacaccaggtagcggaagccttgccttgcccagggttggaaaggggtgcaaatttgggacatcctcatggacaggatgaggcaaacgtagccgatagctccgtgggtggtcgcaaaaatcagtgaacacatctccttctccgcacgttcaactccatggcctccagtaataacccttctcatgaggaatggcaagtctcagattctcagctctcaagcgtgtgtttaattatcttcactgcttactaaggcatgagcataaacttgtaaatatttacaaagactttaaacaagagattgcacttaatattcccgtgagaaagcgtgtccttgagggttttgctggtgaaggcacccacctgtgtgcgtgcacaccgctccgctccacctgtgtaaccaggcgtctctctgcaggcctccccaccgtctgcatcggtgcatggactgcggccaggctctacttagaagacaccgggtgagtccatgaccagactgcaacctcgaaaacgggcccggcaacatctggcaacatgaagtcctttggaagccagaaggttacctggttggaaggacagggagcgtttgcggccaaccgacagccacgcaggaagcatctgggcgattcatggggagaaatcctgtgaggttggagtgttggaggaagccccgtgcaggagacaggaagtgagccaggttttgcagcatagttgagattgggataagccaggcagagactggggcagcccctgcggtgggcacagcgatgtccacagaagaaagccaagtgccctgcatgttgtttacccggagcaaagtggaaggtcaggtgatggtggctgcagggccccccggcacttcctaagcactggtcttggctcatgcggcccagagcgcttttccactatgctctccctgagcgagaccacggcccagacatgagcacgcgtggttccagcacaaatatcttgccttttaaagggcctcagtgtgcatcagttacccactgctaagtaacaaatcactccaaaatttggtggtttaaaacacccaccgtctagttacggtgtgattctgcacctgatgacctgggaggactcagcagctttgaggactggttccccttggtccccttcagccagtctgagactgcaccagcctgaaggctcctgccctcggcccccgagggaaagcatggacccaagcctgcaatggagattctgctttatgggagggctgcagacttccagcgcagggtgtggaccctggggagaatcactgaggcgttttacaagccaggggtcacagcttcctgctggagacaccccatgcgctgaaaatcaaaacaaaatgctatgagctggagagctggtggctctaaggacgttttaaaccctttgccatatacatgcaaaccattcgggaggtgaggggaagctaattttcaggaggaaggttcttttctcctgatttcagctgaatggggctgacacacgttcatgctcagagtggagggcgtcagcagccctcctgcccttgtcctggcttctggggccgcacctccattcctcctccccacaagccgactattgttttgaaagcatgaaacaaaacacaatcactccctgggacagattcctttacgatctgtcagtgcagagaaattgtgcttccacggtgaggggagtgggcaagttgggaggggcaggcgctcattcttctgtacctgtcttgcgaaagtggttggtggtaacctgatttcccatctctctctgacagttgctgggatacaaacgaccacagtgtgccctggtgggtcatacgaataccgattttaatttccatcatcgtaagtagtccctgttgacatgtggattatctgtggaatcacacacttgggcagacagcaagcctgccatcctttattaaatagaactgaccccattccacgcatcactcagccttcccggaaaaggacctcttcaggcatgtggacccccagaggcagttcccagggaaggagggagggactgagggagcggaggggagggtcgaaatcaggtggacttcagggcaaattagggatccacgtgtctgtttcacaaacttattatctcttgtgcaggtcaattttgtccttttcattagtattatacgaattttgctgcagaagttaacatccccagatgtcggcggcaacgaccagtctcagtacaagtgagtgtgtgtcccggcctggccgtccctgatggggatgccgtgtggagacaggagacccccacgctgccaggaaggccctggaggtgcggcgactccgcctccgccactgtcacacctcgaggctgggccccagggctctgctcctgcccaaggctcagagaggccctggacagcatttccctggcgaagtaacaaatcactccaaaatttggtggtttaaaacacccgccatctagatagggtgtgattctgcacctgatgacctgggaggactcagcaggggcacttctggccttggcaactgtggaagtcaggcaccatttcttttatttcaaggccagaagaaagtcctctccagtgaacttgcttgtttaagcatccgcccacttacatgttatacagcctgtctttttctccttgaccacacagtattttttattttacatttaattctgtctccccctcagacatggccatgtgtccatggccgtgtgtctactgccagccctaataccaggccttgtacttaataaaaattcagaaagcgtttcctgcccacgtgattacatgaagatggcattgtgatctcccttcagtagatgaagacagggcagacgttcagccagtcacccccagtcatccagctaattcatgatgatccggggaatctataaagtaatgcaaagactccataaatttatatcaagaatttatttttttttaaaaaggtaaaaaccaaaattcaagtataaactaaacactggcttggtgtggaagtggttggcccagcccagaaggtgcctcgggctcagactgaccgggggtggaagggcggctgtgcacagagctgtaccatggcaggaaccctgccgcccagcctaggagggtgcctggcctgcatttcccttttctcccaccttagaaaacagggatggatttggggaggggcggttagacaggagcatggccaacagccccaaagctgggtgcagataccggccagtgccctgagggaagtcactttgccactccatgcctcagcttccccatctgtaacatggaagtaataatgtcactaaggtcaggaggatcaatgcagtctatatttgccggatatttgctgcctggcaccagagtattgcaaaagagtctggtaaaccagagaatctaattctgctgtgtagtgtctaaatccagggagtatcccgtctctgcttgcccacacgagggatgggattccatgtcagaccccgcatggcagcatgaccttgtccctgctcaggggagtccggtggtgcctgtgtggcagagacccccgaataagatcgttcttggttgcctttcaggttgaacttggatttcctgtgaatcttgtttagtttctatgctgacctttgcattggttctttgggagtgtttgttaaattgaacttggagatggatgcttttatttggaaacaaaatctgttccctttccaggacaacagaaaaggccgagacacccttgaggccacacgaggtcctttctgtgtaactctggtcacagaagtagcaaaacctccctggataggggccaagagctcagccgcagataggcctcttttccagcaaacttcagatgcgttaatagagggaatcaggcaggtcctctgccgctgactgagggacctggctcccacggagcccagctgttacgagctgcaggtccgggagcaggatcggttctcatcatttaaacctgcgtgtggcagcggtggaggccagccttcttgtcccatgcctgcctcaggatgggccgggctcccaggcggggcctgtgtaggtttggggtcactcacagggaatcttgcccctgccagccatgaaccccaggacacagcgtgtgtccttgtgagggaatttgggagaaacggacacttccggaatgcacgttcccaggccctgagtgctagatatgcacaggggcctcagacataaacgtaaagaaacagtggtttttcttgttgggcggggactccagcagtggagaggaggtgtggagcccctcccacacccagctgccccctcctgagaaggacccagccaggcttgcagagggtccgagggtccagcagctgagctggctcatctccttctgtgcaggaggctggccaagtccacgctcctgcttatcccgctgttcggcgtccactacatggtgtttgccgtgtttcccatcagcatctcctccaaataccagatactgtttgagctgtgcctcgggtcgttccaggtaggtgtgcggaggcggccgccctcccccgagcgcgggtcctgcctgaccatctcttctcattgcagggcctggtggtggccgtcctctactgtttcctgaacagtgaggtaagttcatggccgccttggaaccaagcacaggtactcacttgcttcctgagatgctgcccagcctggtgccgcagcccccacacatgcctgtcttccgcgggctcgagttactgcccgggaccccacagttctgtgtccccatcagagaacccacaacgacacaggcaaacccactcttctgggcccagcttccaaaccccgccgtccttccgtccaccctaagtagcatcgactgccgtccgtctcggagccctctcgggaaagctcccggccgggtctagacagaatctgtttttccccatggtcggcgtctcgctcctgaccatagcttgtgagtcccagaaagccgggcctgggttggggggtggggctgcacagcgtcctgtgagtccctggggaggaagcccggagccgagctctgccctgcgttcattcccagccagggaggctgtctccgggctgctccaacggcctggccccgtcccctgacccctgaccctcccgccccacgctgcggttgctggcgctttctttgcttgacatgcagctttcaccagcacggcttctgcaaagctgagcccgagtagtgccacttgtcctcctccctcctctgccgtccgggcacccctcactcatccccaccccctcccagggctaacattccctgcccgctgggaacagggaaggagacggaggcccagcaccgtcacaaccccggctgctaaggaaaatcttttcagagaaatattaactatctgagactgtgggactgcgcttaccgcggcagtggcgatccccgaaccacccacgcacagtcttagagtctggcagcgtcccccgccctcctgtggccgtccagttcccgctccctccagcctctgcccccaaggccgtttctgtttctgtccatctgctggcaacacagatacgggagcaggcgggagggagagctggaggctcgcgggccgcgcttcccatagcagccgcccgcacctgcccaggctcacgggcgttcccgctgccctcacctcccaggtgcagtgcgagctgaagcgaaaatggcgaagccggtgcccgaccccgtccgcgagccgggattacagggtctgcggttcctccttctcccgcaacggctcggagggcgccctgcagttccaccgcggctcccgcgcccagtccttcctgcaaacggagacctcggtcatctagccccacccctgcctgtcggacgcggcgggaggcccacggttcggggcttctgcggggctgagacgccggcttcctccttccagatgcccgagcaccgtgtcgggcaggtcagcgcggtcctgactccgtcaagctggttgtccactaaaccccatacctggaattggagtcgtgttgtcattgactcgatttaaactccagcatttagataatcttgtgcaaaatgtgtttcagccgtatagtggatccacttttttttttttttttttttttgagacggagtctcgctctgtcgcccaggctggactgcagtggcctgatctctgctccctgcaagctccgcctcccgggttcacgccattctcctgcctcagcctcccatagctgggactacaggcgcccgccaacacgcctggctaattttttgtatttttagtagagacagggtttcaccatgttagccaggatggtctcgatctcctgacctcgtgatgggcccgcctcggcctcccaaagtgctgggattaaggcgtgagccactgcgcccggcccaagagaataggggagccaaggaggaaatgtggaaacgcagttgtgtggcccagcacgagcctgggcgaccaccgggtgacatccgtcccacatcagggcggcctcccaggtcccataagggtagccccctcatctgcaggacagagggaagccagtcagggcccccctgacgttaggaccaggagaaatcaacaggagggcagcccgtcctctctcttggggcgcccacccggcccggctgagccctgccccacccaactccacagggctgttttgcctccccacggaaggcgggctgaggagacaaccagatcaggagagcaaggtcatgaaggaggggacctctccacacaggtgttccgtgggaccctcagcagctctggctctgcctcaggaggtcacctgccgccctgtgggagccgcagagcctgacgctcagccccaggccagctgcggccaggcctgcgggcccctggtgatggggttacgtggggtgcgggatacagctgagtgggaaccggaaacctattctctttttaacaaaaataatcttaggataagaattattttaacaacatataaaactgtttcaagccctcctccccagagctggcgctcagcagccctagcggctgctccttcaggcgaagggtggtttgcagatgtggggagggtgtctggggacgttgctgagccagctgcagaagggtggggatatcagggcacagtctccatgtgtgtgccaagccctggcccccacagcgctcgatggacctcagcaagctgcccagccctggcccaggtgccccgactgtgggactcagttgttctgagcacatttgactccacttttcccttaaaaatgaatgtcttgttcctgtgcattggtggcatcacagaccccagctggggcgcgatgtcaaaggtcgggacagctgtgccgggaggcagccacagggaagctcacacatcctgtcagtgtcaccttggtttgcaaaacccatatccccggtaaaatgaggccggacagaggggctgttaggacagcaaagcagcagtgtccagagacccctcaatccccaaaggtccgcaccctgtcctgcacaccctgggccacgccggccacacccctctgctgcaacaagctcatccctggacttctgggagaatgaacccgaggttggtttggggagacaggtgaggcggtcggatctacagaacaacccaccatttctgggggccgcagaggatccatcacagacggatactggggagtaaacggcccaggccaggtgcccaggaaaggacggctgagcatgtggagcgagagggaggcaggtggacgctgcagaccccaggttcagtgcggcccctcggctgttcctcccctgtagggtttggacagacccacccccagccttgcccagctttcaaaggacaaaagggagcatcccccacctactctcaggtttttgaggaaacaaagatttgtggtaactgaaggtgttgggtcagtggccaggtgccgacactgagctgtgacccagaggggacgctgaggaagtgggcgtgagtggacgtgtcaggtggttaccaggcactggttgttgatggtcggtggttgggtgtgggcagtcatcagtcatcaggtgtgctcaggggacaatctcccctcaaccgcacatgtgccactgttcagcggagctgactggtttctcctggtagaggggccggctgtttcctgacagatgcctggtgagcaggggaagcaggacccagtggtcagcaggtgtctttaactgtcattgtgtgtggaatgtcgcagactcctccacgtggcgggaatgagctgtgtaaatacttcaataaagcctgacttcacatctgcatgctgagtaagaacttgctctgaccagatctgaggctctggagcatggaggacacacttggcgtgttagtcgtggctgctgcattcagatgaaaacagagcttggacggggagggagagggtgggcagggtcctgccagccacagggcaccagggaggacattggaaacagccacctctccctgaggagagtcacggacccctgtcagagcgtcgccattggggagcctccgtctggcagctgtgacgcatgagcagtgccctgtggaggaggcgcctgcacgcccagccttgtcctgccgcacccaacacggcctctgtcagctctgcagagcccaagaacctgttaatagagcaggactgagtgcaggagtgtgtgtgtgcacgcgcgttcatgtgatgcacatgcgtgtgtgggtgtgtgcgtggggcgtgtgcacacatatgcgaatctccgtgtgtgctgagccttagccacatgtatgctacaagcaaataaaatgcatttccaatgaactccacgctccaccagatggctgtgttgtcaggagggatacagcacgctggtaaaagccacattcctaaggagtcaaacctagaatgagccacaccaaccagcctttggtgactttcctaagagcaaagcaggtgggaagggggagaaggggctgggcacacagccctccagggagtcactgttttgtctgactcaggagcttagtgtccatttgcctgaggtgggaaatattcagccgcctgaggagactcagccctgatgggaggcagaaggtgcagggccgggggccgatctgcagaagcccctcctccacccgtggcaatagcttcgttctttctcatcaccaatggcagctttttaattggctatttttttcaggctttcagatgacaaattatacaggacagttacaaaacgtcaaactctgtaggcaaagattacatagaaagtgaagtcctctgcctgcctcctcccctctctgggggaaactcgtgcccgactgaagcggggtcccagtgccttcctccgtcggtacaaacttgtctgccctttcttctgtttattcgtgggaatcgggctaaattgtgtgaagcttctgcagctgtctttctcccgctgagacaggtggtgtccatctttcccatctgaaggtgcgtatctcgtgacttttaaccgccgcctaacattccacagcctgggcctggttggtttgaccgactccctgctggcgaatgtgtgagttgtttttgcaggtgtttctgtgcttacccgagtattccctcactgtacactcctgaaggggaaaggtcaaaggtcacgcacatttggctctcgaaggatgtttgctctacggctccaccacccctcaccgagcatcctgccctgggtgcgtgggaaacacttccttttgcatgtcttgaatagtttcttcacaggtttttttctttaacactgacgtaaagctgaacctctgcctgacacactcacatcgcagctgcctgtgtctccaggggctgccccgcacacaagtctgcgtgccagtccacaaacttactcccgttcttgggacccgagacgaggacaagagtcggcctcttgtctgcaggcagcagtggccggcggcgttgctttgtgtcagcgcacgctgcttgtgggcggcatcacaaaggatttagggcaccttttatactgaaaagtcttctttggaagatggagagaaaaggggctctcggctcctttgtgttctctgaaggtttaatgaggagagaggggcaggcagcgagggcagtggggggcccacttgttccacttgtggaaacagtcccttcccaggacttcctgcaggccccgtgaagtgggcgccccagggctattaccggtaccgggatcttttgtaaagcttctattcagtacctggaactgaagaagcaagagggaaaataaactattaaaatataatacaaaataatgagacctgctgggaccaagctctccctttgagattcagggctctacatagcctgacacgtgaaggttccatgggataaagtctaggaaagccacgcatgaattcagtgcacctcagaagcacaagctatcatggtttaagacctatgactgtgaatccagatgttaaatgtgtgactttcaaatccttaaatcatgactgtcctttcggatttaaaagcacgcgatatcacaaagctccactgtcccatgactcagtcgtgcagattttcgcatggcttggatggatcacactctcccaaatccaccagcatgcagtggagacctgagtgtccattcctacctctggagaagctggagacgccggcataggctccatgcctccagtttccatttggaattagtattgtataattattacgaagagtctgagggaaggggacaagaaggaagccatgagaggacaaggacttccctcgtaaggggcaacgagggccaggttcagtacaacaggagggagtagcatggagctccaagtgggagagcaaagggcaaagaggaaaggtcaccaggaacatccaggacctgggcacggcctccaagctgtgcagaaaacacagggaaggggttggacttgaaatcactcttgatgtgttcattcttttgattgtaacaccaccctggaaatgaagccggcggcccatggctccaggggcacgtgtggtggtcagccacgcccagtctgcactgtgtctcctcatctatccatcatccggatgtctaactatgtgaccgcaagtgagcattgcagaaacagcacgaagcgttcggcaggcacgctgcctctcagcaagggagagatgaggaggaggggatgaatgcaagcaggtggtgtccacataagtccatgtgcagaactgtgcacagtgagtgctgtgttcccagcggcacgctcagttcccgtggggagaagcaggaactgcagtgggcacccgtgaggtcaccctggaagggcacccagcgccctgtcggaagaaaaatgagagctctgaaggctttgaagctggagaatgacatgatccaggcaatggattaacttggtggcatcttagcagttgggaagaggagatgccaatgggtgtggtctgcagagggtcacagagcagagacagttcctgaagagatgatttctggacaataaaggggtcccagggggcggaagttgcatagcacctgatcatagtgtgccctgacgctgggagaccgctgcgtgcccaccgactgtccacacacctgatcgcggtgcgccctgacgctgggagaccgctgcgtgcctgcaactgtccacacacctgatcgcggtgcgccctgctgctgcaggaccgcttcgtgcctgcgactgtccacacacctgatcgcggtgtgccctgatgctgggagaccgctgcgtgcctgcaactgtccacacacctgatcgcggtgcgccctgctgctgcaggaccgctgcgtgcctgcgactgtccacacacctgatcgcggtgtgccctgatgttgggagaccgctgcgtgcctgcgactgtccacacgcctgatcgatgccacagcgcacggtaaggtggactttttccttcacacggatgcttcacgatggcattggcagggcatttggacagtcggtggcacacagccatctcgcgtcggaggacgcagcagtgctttagggcactgggcagccatgacctcctcacagtggtgcagctcggggtgcctggcggcggcgaggacagccaccttcagggacagcacagctcaacctcgggcctggccgagctcctgggtgcccattgggcactctggcctggatcaagttaaacaggtcttatgtgagtcaaaccccacatccctgccagaattctatttggggcacttaaaggatatgcagtgctgtgagacgcctcataggggaggggtctgtcctcgagaatgaccctgaagggtcgatgacaaggttaaggtcacacgaccaggcccaggtcaggggcagcagagcagagtggaggacagggcagcatgggagtgagggtcagggtggggtggctgcagccacacagcagcacagcacggtggtctggggcccgcggagcagcaccgcagagagagggtgctgggtcagcagtgtggtcccataaaccaaacctgccacgtgctctgtgtggagagaaacagagaggccagtccccagtgcagcatgtgagaacctgtagctggtgatatcctccaggtgggactgtgggtgcagccaggggctggcgtggtgaggtgggccaggagcagtgtctgcatcgggtgggactgtgggcgcagccaggggcaggcgtggtgaggtgggccaggagcagcgtctgcatcgggtgggactgtgggtgcagccaggggtgggcgtggtgaggtgggtcagcagcagtgtctgcatcgggtgggactgtgggcgcagccaggggcgggcgtggtgaggtgggccgggagcagtgtctgcatcgggtgggactgtgggcgcagccaggggcgggcgtggtgaggtgggccgggagcagtgtctgcatcgggtgggactgtgggcgcagccagcggcgggcattgtgaggtgggtcagcagcagtgtctgcatcgggtgggactgtggcgcagccaggggcgggtgtggtgaggtgggccgggagcagtgtctgcatcgggtgggactgtgggcacagccgggggcgggtgtggtgaggtgggccgggagcagcgtctgcatggtgtgttgccccagctgggctctctctttggagctagcgtggattagtagggacagcaatgaagaccactcctccagaccccgttgggccaacactgggtccaactatactggaccttggcccagtggcctccccagcaagatcatatctgatgccagccttgcagcagcacggacgccttctgtcctcagcacagcccagaaagtgctcttcactcaggccataatgtgaatgtgcctgcaggtgggcctctgtcagggcccccaggcactttgtgccctaaagatggacaccatgggatcctgaccctagctgcccagggaccaccgtatgccctggaaatggacaccatgaagcatttgactattagttcatctgcacaaagttctccataggccaaaagccacccatcctccaccactctcctttaccttttaacaggatgagggtgcaggccgtgacagagtctgtcgaggtcccccgccccagctctcacacaggcctgtggaccccgctaagccggctggacgccaggccctggacagtagactgtgagctcccacccaaagtgtgagacccggtgtcatcaagagccgggtcctgcagctgcctagatcctccccaaagcctccttggctgttggccaagtctgtttcacagggaaccctccctgatgacgcagtctgcagtgtggccacatcctgactctagggcactgggaaatgcatatttatttgctcgaattttccatagaatttcacaaaaggggagataatacagagtttgcagttccaatttggccaaagattcaaccaaaacacttttttggcctaagttttttaaaggtccaggaatacttttcctactctataggcactcccagcacaggtgcaccatgccagacggggccccgcggtttacatgcatgacgggtttcggcctcccagcaccaccaggacagactgagtcagcagtgcctaggagtgaatgccaagccctgctccgcacacgggacctcagggcaagaccctccagtcgccatgctgtgaggatggatgggcagccccagagccaagcctggcgccaccagtgcccagcaggtctccgctattacttatttcttcttaggggaagaaattctaatctccctgcaggagcttaagtaaatacgaaaaataacactttgaacactggcaaaaggacaaaaacagataactgatgtacacaagactgtagaaagcaaaaagaataagggagaattacactctccaataagcaaaagaaaactaaagctgggggccataattggctataaaaatgacacacatcccaggatagggcggtgaggttacagttgtattatgtagtgtgatattataaatttggatttcatggttttactgtatcatagtcttataatacaatataaaactcacttatgtccaggcatgatggctcatgtctgtaatcccagcactttgagaggccaaggtgggcagattacctgaggtcaggagatcgagaccagcctggccaacatggtgaaacctcgtctctactgaaatacaaaaattagttgggcatggtggtgcatacctgtaatcccagctactcaggaggctgaggcagaagaattgcttgagcccaggagacagaggttgcagtgagccaagattgtgccattgcacattgcactccagcctggctgacaaaggaagactctgtcaaaaaaaaaaaaaaaaaaactaatatagcttattattatgccatatttatttactctataagctaaatatattataaacttagaatgttcattccattcaacccagtaatttcattgctaggaatttttccagttaaatattcagaattatgcacagtgatttgtgtatgagaatggtcatcatggtgttatttgtaatatcaaataattgaaaagcaaaatatctgattattaactgattaaataaatggtagttgggaatacagaggcagactggcttggaggggccctttaaaggaggatatgtcctggctgtcactgtgtacaaggtgcacccactcagcaagtgcagacaggaggcagacaggctggggtggaaaagggcaggtccccagtgaagccccaccttcaggctggggaaggcctgaagcctgggggccgggacgacagccccatggaccacagggggcgcttgttctttttcctgggcccgcccatggccacccatggaccaatcagcacacacttcctcccctctgaggcccataaacaccctggactcagccagactcagggagaggaccagaagaccggcctgcagagaggagttactgaacgaagtctgaatgatggggaagagtgaaccacaagcagaagaaaccatgaaatatgtggtgtgatttgagaggcatgtaccagcctcagaatagtgctcttggagcaggcaacttggaatccattgtgcattgcttgcagtaattcactctgccaagcaaaccattaataatgcagcacaaattatgttatctaattactcacctagcaaggatattgagtgatgaaatttaggacagccactttttgatcgtaagaactggggaaaagggaaagttcagctcatctaacatgagtggttttattataatggaaacaactgacatatcttgacttccagccgggtgtctctccttagaaatcttcccctggatatcccatcctgtatgcagcatcttcttccccttgcataggactcccagctgggcatctcttcttagaaatcttcccctggatatcccatcctgtatgcagcatcttcttccccttgcataggactcccagctgggcatctctccttagacgtcttcccctggatattccatcctgtatgcagcatcttcttccccttgcataatgaaagaaggtcagttcatcaggaaagcataacaatcccaaatgtgcatgcacctgataacagagcttcaagatacatgaagcaaaattaactgaaaagaaatagatccacatggttgaggttttaatgcacgtctctctgtgtcattgatacagcagactaagaaaatctgtaagaatatagaacattcaaacaataatattaactaacaatattaaccaacttgacctgatcagcacacagagaacatttcacccaacgactgcagaatccatatttttttcaagtgcacatgaacattcaccaaaatggaccatatactagaccataaatcaagtcctgataaattccagaggaatgaaacgatacagagtaggctctgaccataattaaattgcactagaaattaataacataaaaatgccaaatatttgaagttaagtaatgcatatctaaatatatcaaagaagaaatcacaagagaaatttcaaaatatgttgagctgaatgcgaagaaaaacacatcaaaatttgtaggatgcaactaaaggaatccttgcaagaaaatttgtagcttaaaatatagatattagaagggaaaaaagattagaaaaattagttcattttgttggaaatacatttgaaaatatttaattccatgagaaaaaagcaaagcctaatttttttttttacacgaagtctcactctgttgcccaggctggagtgcagtagcacaatcctggctcactgcagcctccccatcccgggttcaagagatttctcgtgcctctcagcctcccaagtaactgggattacaggcgcgtgtcaccacacctggctaattttagtattttttgtagagacagggtttcatgatgttggccaggctggtcatgaatgcctgacctcaagtgatccacccgcctcactctcccaaagtgctgggattacaggcatgagccaccacgcctggctgcaaaaccttatttaaagtgaagcaatgatctgttcgtcaatcaggtctctgggctcttcctgtcaggcatcacacaagatgcttgttgctgtagcaaccactagggaaggtgaaataataactgagggctcttccacagagaaaaattttcaggttttgtttttaggagctcttttcattcagatcattgtgttctcaaatggcacacagatctggcttgacgccaatgtcaaatgaagcaaacattttccacccacactatctgcccacacttcctctcccagttggtgagaacatggactcactgctcccccgagtgaggaacagcaccagacccccaagatcttcacagtggtgccacagtgattggagttctgcggggacccagggaaacatgctttcttcatgggctactcagctctgcagatgctcttgtcagtaaaaaccaaccccaggcccctgaatacatattaggaaaggaatgacagagacataaaaagggggacaagacccccacaataagggggacaaaacagagacacaatcggagaaatgagaccgagacacagtgggggagatgagacagacacacaatgcggggggtgagactgagacacaatgggggggtgagactgagacacaatgggggggacgagacagacacaatgtgggggtgagactgagacacagtggagttgagacagacacaatggggggtgagacagagacacaatggggagttgagacagacacaatggggggtgagacagagacacaatgcggggaggtgagacagagacacaatgaggggtgagactgagacataatggggagttgggacagacacaatggggggtgagactgagacacaatggggagttgggacagacacaatggggggtgagactgagacacaatggggagttgggacagacacaatggggggtgagactgagacacaatggggagttgggacagacacaatggggggtgagactgagacacaatggggagttgagacagacataatggggggtgagactgagacacaatgcggtgggtgagacagagacacaatgagggggtgagactgagacacaatggggggatgagacagacacaatgtggggggtgagacagacacaatggggaggtgagactgagaaacaatgtgggggatgagacagacacaatgtggggggtgagactgagacacagtggggggggatgagacagacacaatgaggggtaagacagagacacaatggggggggtgagactgagacacaatggggggtgagactgagacacaatggggggtgagactgagacacaatggggggtgagactgagacacaatgggggatgagactcacacacaatggggggtgagactgagacacaacggggggtgagactgagacacaatgtggggtgagactgagataaaatggggagttgggacagacacagtgggggagctaaggctgagacacactgtagtgatgacacagacacagtggaggggatgagattgaagcataaaggggacagaagccgagacatgatgggtgaggggttgagagtagacagaatgtggaggagagtgagactgagaaaccatgtcagaaaccacaagctaaatcaaaagggaaacagtaatatggaaaaatatatgcaagaaatacgaaaaggagttagtattcttatggttgagtcagtaagagaaacaccaagagtctaacagatagtaacaaattacataaatttcaatccaaaaaaacacaaataaaaatgtttaagaaatattcaaaaattaccaatccattaaacagcaacactgccacttcctacctacttagtggaactcaaacatctgagagaacacttcggaatacagatgccattctcaaaattgaagtttttctttgttgaccaggagcatactgaagggacctgcctaatttttacagatggctgggtcctgagacagccagatgtcccctgggctggctggaagaggggatcccagagcccacacgggtgtgggagcgaccgggatgaccactgcatgtaacctaggcagggaggccaggcgcaagaccgcacaccaggagtggggagcagagctcccaggtccttgggcagctgacagttttcagtcagcaccagctcaaaaacctcacactggcctgtcgtggaagttgtgacggaacaaaactgtgtacacttggcccgcacagagagactggacgtccactctgaggtctgcagcgatggaagggaaggagtgtatctcatcccgcacaggagactggacgtccactctgaggtctgcagtgatggaagggaaggagtgtattcttgtcccgcacaggagaccggacgtccactctgaggtctgcagtgatggaagggaaggagtgtattctcatcccacaaaggagaccggacgtccactctgaggtctgcagcagtggaagggaaggagtgtatctcatcccacacaggagaccggacgtccactctgaggtctgcagcagtggaagggaaggagtgtatctcatcccatacaggagaccggacatccactctgaggtccgcagcgattgaaaggagggcgtgtattatcccacacaggagaccggacgtccactctgaggtctgcagcgattgaaaggagggcgtgtattatcccacacaggagaccagacgtccactctgcagcgatggaagggaaggcgtgtattctcatcccatacaggagaccggacatccactctaaggtctgcagcgatggaagggagggtgtgtattctcatcccgcacaggagaccggacgcccactctgaggtctgcagcgatggaagggagggcatgtatttgcagggtgccaagcaggagggtggggcagctccgcctaagtcctgaactccccactggctgcagacaagggttctaaaggcagcagcgaatttcaggaaagcagaagctgcaggcaaagtcgtcaatcaggacgttgggcgtacacgtgggttttgacctaaaagggtgggagatctcgaagtgaggacttaggggtcatcagaggattcaaggatttcctgatttgtagttggttaaggaagagcagctttgtttaaaaattggggtcggcagagaaattgttaaccgactcaggggtgtggctccctccaagcccctcaagaagaaatttagacccaaagaacagtgatcagaggtcagcctcccgttccccccatctaaggtctcttcgtgggggtccgcagtggggtctgggcttctgaaaggcagctcggggacatgtgtgaagatgggatctttagagtctacagagaaacaaaaatctctggactcactcccctggctgctgacttgggctgcccgtaaccttcttgcttatcaggttgttcatttacaggcgagctggcttccccttgaaggaactctttgcagggggctggaggactgcaggcctctaacaagggagtccctgctccctctcaaaatgaacccttgatccttggagggactgtgaactttaacgtccacactccgcaggccttgaggtgtctggggacagccccccttcccaccgcccgggggcggcccctcctccgcaccctgggcagaaagaacgcgatgccgtcatcctcaacacgtggctcgggcaacggttgaaatggagagttttcaagaattatttttaataaaataaaccctgaggtcagagactggtcatgtcaggccccggggcagagactgtttacagtctttggtgcctaaagaaaggtcagagaaattccagggctgccctccctccacaggaggtcaagtttcatcaccaaggaggccccttctgcagcctgtgggctctttaaagaccacacccctgcctgtgcctgcgtcagagcccaggttccccgctctgcccccaccagctactgcccttgggcctggcactctccccaggaacgcctgtgaacagcactgtcagctcatctccctgggcttctagtcagaaaggctggtgtgcagaggctcacaggccacctgtgctatttaaacatcgcagtcaactcccctttgcctgttcctatagaaaacaaggtctaacagtcatctccttcctctaaaataagcaaagtgacattgcagaccgtgtatactacctattctgtgtatgcgcacgtgagcatggctggtcaccgaggcacaaagcgtttaagaaggccctgctcaggcccagctcatacttgggtcagagctggggaccctttcatagcagccccctgccaggaagtgccagccaactagtggccagatttctgagtctcacagacataaaaagtctgaaagggttgcctgttgtttcattttggccagtaaagtctcaaaataggaatggccagcttttatcaacaatatttcaaaaggatttttttttaaaggactctctgttgaaaacatttctgtttagagctggagagtctcttgtctagaattatctcatcccctgagaataagaagcagcatctggcggaaaccccagaccccgccaagagtctgccgtgcccaccacagtcgtggcacagcgtgtgcaccaagggcctgccaggcccaccgcaggcatggcacggcatgtgcacttttttccaggtgcctgaagccctctgagcacacgagagggacaaggctgtcaactctgggccacactcaatcagctcaccctcccacagcccacgtacagggagctggcccagtttgggtggccagacctgtctcatctctggatccaccctggccccggtcaattatgttctagagtgtgggggcattcatggatttatagatgtgtagaaatgatcagttctgaccccgagcctccgttcacacaaacccccaagcgcaaagctcccggcagaagtgcgacaggggctctccaggcccacagccgcctttgtgtccacacatgacatcacttcactgtgggtgtcctgaggaacacagcgtccttggagggttccctgaggttggcgtgaccacaaaggacagcttactagcaaggagctgccctcacccccagaaacggtcacaataactcacaaattataaacacctgaaaatccaccagatcagccgttccagagcccagcattaggaggatttggctgggagaaagcatggaggctgtcatggctggcatgggagggggcgccggggacacaggccagggagaagtcccgtccccaaggctgcctcccacacgggcgaagccagcacctgcccatgccctctggcctctacagccttgcagggccacagaagggcccagctgctcacctccacagctcctccttcccaagatacttcccacatatgatgtgactctgacctcggtctcccaaggagtcccatgcattcatctgagcagggcatggggaggcagctggccgtgctgagcaggagggtgtgtggtgccagggcccaggacaggtccgaggtctgcagtgcctgggcagggcctgaacagaacatctcctatggccactgcatgtccgcctccccccacaccaggtttgcccactgaggccagtgccctggacaccagccaagggccctgccatgcccatgacttgtgcagacccaaaagagggacaaggccgagatgatttaatggcccaggggagcctggagacctcatctccagaaattttaccataatgttgcttgaaaaatgccaaaataatacgttttaagtcatttcaaatcagaacataaaaagcaaatgcaaagatacagaagtgcacaagcccctttgaccccaatgcgacctcgaccccaaccccgaccttgaccctgaccccaaccttgaccccccgacccaacctcaaccctgaccctgacccaacccgacccaatccccatcttgaccctgaccccaaccttgacccccaacccgacctcaacctcaaccctgaccccaacccaacccagaccccatcccaaccttgaccctgaccccaaccttgactcccaacccgacctcaacctcaaccctgaccccaacctgaccccaacccaatcctgaccttgaccctgaccccaaacttgaccccaacccaacctcaacctcaaccctgactccaacccgacccgacccagacccaaccccgacctgacctcaaccgtgaccccaaccctgaccccgaccccgacctcgacctcaatcccaatctgaccctgaccccagccccgaccctgacctcgtccctgacccggggaagggtgaggccgttgccctcctcccagcagagcctggggcccccaacggcagcatcctccaaatgctcctggggattaggcaggtcctgccatcatgaggatttgctaaaacccagccagtgcctttcagaagcagcctcacagaaactgcaggaaagagcccattcctcgccttctgccatgagccctctgtggttttaagcatgggagaatttggagaacaatggtgaaagggagttacgtggggctatgttctctaaagaagtccctggctgctgccaccttgcacttgtgaccgatgctggggccaacaggaaatgccagtgcctggagccccagcggggccgggcgctcatccagacgtgctgcagatgaacttggcccttcctcattaaacttcactgtgtggaacataaacctcgtgaattttgattctctactaatttggaaatgggtactacatagacgggcttggctgaagtttgtctttgagttaaagaaaagtttgtttaaaaaataagcagcatcaataaaactgcaagagtaaacctcttaacaatatcacttgtggaggataaggctaatatttactttaagatctgaccattttgctgagtaacagctgtgttccagggataaaatgtggcctgtggtatggtctagtgtgtgtcactggtactgacggagttctgacaagacttgtcagatgtaacttatgtgacctgctaatcagaaatcttttcgatatcatattgaggatatgccccaagaatcacattaatggatcaatttgaatttccacatgagaccgaatgtaatggaagcacattacctaagaaactgtaaacgcagacttccctttattttgcactagactcctttctaagtagtagcaatggtgatgtttttcatgcaagcaaacacaccacagcttgctgatagtaatgaacaatgggcataatttccagagccagaggcgctcccaacacatacatataaatcttaacttaattatgatactaaagaaagtgaattacaaaagagatagctagagatgtttttgaagatgtttcttattttaaccaagcttgagtttcttggggcagagaaagagattgacgatgctcagagatcttgctcattgtgacaaatcagggacgctggacacaggcagcgcgttctggccactatcctttcaaaacagagcttcaagaacaaccttctgcattacagatcctctgcctcttatctttgactgaataccagatctcataggggacacaagacaattttaaaaaggagccatgttctaaaccatttcacaggcagcttcctcctttgaagactctgcatttgacttcctgacactgcatctgcccggtctctaacagcttttgtgtcactcacattgtgttctcctccactaaggagtcttctcctggctcaggagctgtgtgggcaaaggtggtgcactgtgggtgctgagcgcctcctctcctgcattgttcttgccctgggggtggtcgaccactttcctcccaggccatgcagggttaatcctcatctccttcccctgctctgtggtcctggctcttcccgcctctgtgcccagccagcagcactgaccagcagcctctgcatctgccctgttgggattgatcgccctgacctggctgtgggcctctaagcacattccaaaggcctaggccgttccttcctcctcctcctgcagagctgccaagcaagcctcacagcaaacccctgtccacttcctaaacacgcatttgtaacaacagacactcttgggtcccacctctctttgatgtgaccttttacatccctgcggccaccttgtgatttctcctgtgtcttgactgagctgatgcccgtctttcacagcctgtgtgcaattctatctcctaaatacgtaaccccttctctgcctcttccacacagtgccctccacacccagaatgccctcctacgcctcccctgcatttcagccaacgctcctctcctcaaggaagcttttcctaaccaccaactgtgaacattccagtcatctgggctcggcagaggtctgcttatgcatccctttcctgatttgcctctcgctgcggtcaaactgacaacctccactcatcttttgctctttttgtgggactaggttattagaccttcatgtgggacctgggccaggagtgctgagtgttcagggagaaagctttccagccacagaagtccgtcttgttgggcagatatgggaaaagttaaataccaagtcaagaaataagcagtatttttgtcatctttgctgacacacagattgtcttattcatttaatattttttattcctgtgcattaggcacagtcctgggtgtgggagaaacaacaggaacccaaccagacaacggtcccactctctgtagtgggtggtaagagacataattgagtgtgagacacggtcacgcgccaaggaaaacaagaggacccagggaagagacgtgggtgttgggggcgcaactacaggcatggcgtcagggctgggggttgggggccagtggagggggaaggggccaaaagcgtggcaggcgggtgcaggcatctagggccacagcctccactccccactgtgcggtgcagggcaggggtgtgaagtgacctggctcgtggcactcatcatcccagtgctgtcctgaggacggggaagagctgaagcaggcagtcgggtgcaaccccaataatccagtgagaaactgtggcattggtagtgggcgcagcgagatttgatcagagtttggatgtaaaagtgacctaagactttgttaattaaagttttcatgtagagtacagataacacagttgtaaatttggaggacaggaatcactgagctggtggataagacagcctcatggtaagaagcaaaggagaagtcagtctccgagaaggcgtaagtgcagtcaggcgagggaagcaggaggcctcccgggaacctggctggctcactgtcccaaacatgttagcttcgtgctgtgaggacgcttgcaacaaccacatcagcgggcatccacaatggtgtgggataggaaccccactctcgcaaatggggggcatccgcaacagtgtgggataggaaccccgctcccgcaaagcgggggcatccgccatggtgtgggataggaaccccgctcccgcaaaggataattcacggaactggggaagtttcgacaggaaagaaaagatcttcctgtgttttcaggagagagaatgagaagcagcccatattaactattcaagtatgttgaaaagttgttactaggaaaagattattattgtccttgtcctgtaaccagaggaagaattaggacctgtggatacaaaccgcagggagagaggtttcagtgtggaaaatagaagctgtttcttgcaattagagctgaacaaaaataaatgggctgctcaagaagaagtgagctctccatcattggaggtgccaaaactaggtggcagaggcaggtgcaatgctgcattagcccagaaggccaagcatccacgtatcacacggaagacccagatcatctcaagaattcatcctgtccatgatttcgtaaaacagggtcatctcaaccccactgccaattcatgtgacccatccacttggtggcacctctgtaaccacccaagtcttcacacagagcccttgatattgtgagcctacagcttaagaaaacggatacaggtataatataattaccttttctgttgttttttcctcagatatgatggggtcaaggaaatggcaagttcctatggtcagcccttcttctggtaggatttttcctccactaggatgggtctgaatcggggatgaaagcgacttcgcttctgaaaatccagagcctttttaaagtaagccttcataaggaaaactgcgattgtccagtcgatggtttgttaacacacagaccccaggagactgaccaccaggctgggaagtgtaacggcaatgattgagggcgtcagacgtcctctgccaccaggtagggtgaggtcctcacaagatggaagccaagttgtcagcttaacagaaggaaagacgccctccatcttccaccattaaggtctccagcgttcaccttcttggcaggaaataatcattgaaagttatttctgaaatgttggtttgacatttaaagtagtaactggcatcttctgaaatatatttctggtcttttgggacttcatcattttcctgtgaagcttgacctttcaaatccagtgaacaaaagtcctgagatttgtaatgcatcacacctgtgacactttgtcacccatcagcatttttggcgttggcattttccgagtttgcctgatttctgaagaccatgcagataacaggttcagctcccagattctcactgcacgcaaccaaaagaaaaagctgcaatatcatttcagctcctttagatgagacgtgaactgcagttcttcaggctctccaaaccaacagctggctgcacagcagactgatggcgccaacggtgatggagacctcaggcagctgtagggccagatgcccggggaaggacgtcctgggacggtccctcttggtggagttgggaaaagaagcaagagtccatcccctgtgagaacaaagggttagagactgaggaaacctgggacacagcagcccaggggcatagccatctcactgacacactggctccctccagcagacactccgcaagggtgtcctggacacaccggctccctccagcaggcactccgcaagggcgtgcgggtcctggaaacccctccctgtaccaactaacatctcatttcagcaatcagatgtcccagattctaacacaatattaaaatacagaaagtaccacccgcttgccctcttaggagacccctagaaagctctggaaagcccagggctgagcggcaaagccaggaagaagagacaccaacagctggacaaacaaatccgctaacaagcattggaatcgcaggctcagagccagatggccttagagaccttcctagcagttttcaggctgatagaacattttgttcaaataaaattgaatgtggaccccggtttatagaatagacttgttggcctgattgatggaggatgaaagccccctgacttagtccagcccaggagggagaccctcagaaagcaatttgaaacaactgcatctggtccaccctcttttcacctcaaagaaacagccccaggaggccacgggacctgccgagcatgaagtctggcaggccacgtaagcttcacagggcaggtctgcagaccctggccctcagcagccggaaccagcacatctactgttttggtttatgtgctgtcagccaagaaagctgtttcgtgatgattactaattacgagcttggattttctcgccagctggttatggagcaattattacggtgcctgcctcacagggccttataaggattaaagtaagtgtgtgtgagctccagaggggaagccactggaaagaccccctcactgtcagcgcttgacccctctgcacccgccctccctggcatagctgggtcctctgtctctgcacctgccctccctggcatagctgggtcctctgtctctgcacccgccctccctggcatagctgggtcctctgtctctgcacctgccctccctggcaaagctgcgtcctctgtctctgtgcctgcctggccctctcgcctggcccctctgcacccgccctccatggtatagctgggtcctccgtctctgcgcccgccctccccagcgtggctgggtcctctgtctctgcgcccgccggccctttctcttctcaggctcctctttgtctcatctcgtcattcccatcttttgctctttctcacgtctgttgtatgtttgttccatttaccatttatctatctttcactgttaatcaattttaaatacaaataacattttgtttccttaaaacatttaaatttcactgttttacatttaagtgaacacaatctatccacctccatggtcaaggtcgttcagaagcaaactctgtcctcccacctgacccctctgaaggccgtggactcacacgcgagggtccccgtgcctgactgtgcagcgctgactctttcccgggcccctgagcaggccacgtgtgaggctgactcatcaggagccctagacacacgcctggagccctagacacatgccaggagccctacctgccctgagcacctggcccaggccacatagcccacacaggtggcaggactcctttcctccgtttgatctaacttggtttttccatgtctttctgggcaaaatgaagacaaaaccaatcgccaccaacctccctctgtggcgggaaggagtcgcaagtgtgggtgtgatgacactcaaaggagaacctcaggagcccagataaagccagcagcgcgcgggaggccgtcctcttcctgaggaagccggcagcgcgcgggaggccgtcctcttcctgaggaagccggcagcgcgcgggaggccgtcctcttcctgaggaagccggcagcgcgcgggaggccgtcctcttcctgtggaagccggcagcgcgcgggaggccgtcctcttcctgaggaagccggcagcgcgcgggaggccgtcctcttcctgaggaaaccggcagcgcgcgggaggccgtcctcttcctgaggaagcaggcagcgcgcgggaggccgtcctcttcctgaggaaaccggcagcgcgcgggaggccgtcctcttcctgaggaaaccggcagcaggcgggaggccgtcctcttcctgaggaagcaggcagcgcgcgggaggccgtcctcttcctgaggaagccggcagcgcgcgggaggccgtcctcttcctgagaaagcaggcagcgcgcgggaggccgtcctcttcctgaggaaaccggcagcgcgcgggaggccgtcctcttcctgaggaagccggcagcgcgcgggaggccgtcctcttcctgaggaagccggcagcgcccgggaggccgtcctcttcctgaggaagcaggcagcgcgcgggaggccgtcctcttcctgaggaagccggcagcgcgcgggaggccgtcctcttcctgaggaagccggcagcgcgcgggaggccgtcctcttcctgaggaagccggcagcgcgcgggaggccgtcctcttcctgagaaagcaggcagcgcgcgggaggccgtcctcttcctcggccgcaggcaccggagcgcgggaggccgtcctcttatctcaaagccccgctgcctcgtgggtgatgtcaaagtgcacattttcaaatactgtttttgttcattgattggcattcaagggtcacaatacgaccacagttgctcgggttgatttgtgaaagagaacacacacagaacagtcctccacactgggttccaccgggaaggaacatgtgcctctcccgggaaggagcacagggcagctttcagttcttcatcctgcccccaaatcaacagacacttcactggttactcaggttttttaagtcctttttcaatttccaaaggacaggtgggctctttgtggatagaaacagacttcgatcacctacgtgaacctgcatgtttaaaagcccagataactacacctagaaaaacgcaggtcccaggaccagacctactcccaccctgcagccacaagggcctgtggtctccacacaccggaaaaacgattccacatcttgctacaaatcttaactccagccagattttcttctgtgccatggggtagtggagatgaagcttggagggatttatgcaaacgcacaaggaacagtgggaatgaaatggctcagctcaaaccctgctgggggcagatgtcctctccgaggatccagttatgtcacggttcctgcccttagtcaggttttagcttattcatccatgtctgctcccagtgctttttttttttttttttttttttttttgagacagggtctcactctgtgtgcagactggagtgcagtggcacaatctcggttcacggcaaccccccgcctcccgggttcaagtgattctcatgcctcagcctcccaagcaactgagattacagacatccaccatgacatctggctaatttttgtatttttactagagatgggggtttcaccatgttggtcaggttggtcttgaactcctgacctcaagtgatccacccaccttggcctcccaaagtgctcggattacaggtgtgagttacggcgcctggccttgcaatgcatttttaattaaaacaacataaaaattttaataagacatacaagtgctcacagtgccacgctgcagagataaccacacacaccaacaattcggttctttccccaacacgcaccatctggaaattcctatggaggctttgatgtcctttaaaaacatattgttctgaaaacggatttttaatactttttaaacacatggtaagattgacttttttggtgtagagccccatatgttttagcagcgcatgctaacaaacatattcgaagcgaaagtgctacttctgaaatgacacagtggtgcctccacccttaagcacaactctcgtaactggtgcaggaacaatgcgtcagtcgggtggaaaatgaacctagattcacaccctatgcgggaagaaacttcaaatgaattagagacccaaatgtaaaaaaagaaatcgttcaagtcccagaagaaacctgggtgaattcctctttaaccatggggaaatgatgactaactaccgtcaaaatccaagggcgatgaaagaaaatatttacaaatttgcctacaaaaaaatgtttaacaccactaaaaaaacactatatacaaagttaaaaggcagttgacaaatcagaagaaaacaggcacaacacatcccacagatatggagctaataaacccaacatagcaaaaattcttaaaaatggaggctcaaagggccagaaaaaccccaacagaaccatgggagaaggcaaacaattcaccaagaagacattaaaatggtcctcagtcatatttttttttaagtatgatgacagtttctcttggtgatttgaggagagcagcactggtgggaatcggaactgggaccacccttcagccggggctttgtcaacacctgataaaactacacacacttgcttttgacccagcaaccccacttctcagaatattctggaaaacacacctccaagaacacagaaatacgcgtgcatgggatgcatcactgaccccttgtttgaactgcaaaggagctgtgtgcttgcccacatgaggcccaggcagacggaggaatacgggcttggtgtgggaagacgatagctgcaacagacagtgattcctctgatggatctgggcaaagtaagtcgaaaacctcctggaaaggattcaccatcctagatgccattgaaaacagtgattcatgagaagagacaaaatatgaacgttaacaggagtttggaagaagttgattccaactctcatggatgactttgaggggtttaagacttcagtgggaaaaggaagtgcaggtggaagggaaatagtgagagagctgtagttagaagtggagcctgaaaaggggactgaattgctgcagtctcaggatcaaagtttaaggatgagaaattgcttcttatgactaacaaagtggtttcctgagatggaatctactcctggtgaagatgctgcaaacattgttgaaatcacaataagcgatttagaatatcacataaatttagttgataaagcagtagcagggtttgagaggatgactctgactttgaaagacagcctaatgtggtaaaatgctgtcaaacagcatggcatgctacagagaaatcttttatgaaaggagtccatcaatgtggcaaacttcattgttgtcttgttttttaaaattgccacaagcaccccaaccttcagcagcaccaccctgatcagtcagcagccaccaacactgaggcaagaccctccaccagcaagaaaattaggactcactgagggctcagatgatcatcagcatttttagcaatgaagacaggcacattgtcttttagacataatgctgttgcacacttaatagacagacgacagtacagtgtaaatgtaacctccatatgcaacgggaaacccaaaaatgcgtgtgtctggctttgttgcgggggtctggaagtgagcctgtgacatccccgagctgtgtctggatgtggcctatccctagaagtttgtgcctcgtggctgcgctgtctgaaggcctgagcataacttgcacacagacactgcctcacctctttcctatagcagtgaccagcatcccgtggccctgcaggcacctcctagagtgcatctggcagagacacatcactaggccaaaccaaagcaatgggaaccctgtctccaggttcctggttccatgagggaatcaagttacctcagcaacagtgtgtctgcatgtatgtatgaattttaaacaactacaacaaggtggcaaaagcaagccccctctggaggataaagaaaagctgaaatactccacagcacttaccagacggggcgagggaggagccctgctccacgcgcttctccggccacagggtctgaaccttcgggcatgtctgcttagagacgtggaattctgtcaaatgagcccccctagaagcttccggtgagaatctgtctagaactgttgggtgggctgtggtttgacttaaccatctgcatgtagataaattaccatttatatgtgtgaagaaggtatccatccagtgatgatccaacacgtgctcatcacataccattaaattttcaaaagtaactggtgtagctaattatctgtctcaatctggcagcctctgaagtgtcacttacaatttgtgcactcagctcataaacctgaagtatccggatgcccagtgctaggtcagatgcccagtgctaggccagatagcctctcatccttctgaaatgaaagtttggaattgtggtgccacagagaagggaccctcgggtccgtggttgagactgagcctgtggaagctcagaacttggcgatgagcggctgcagtcacgggagcacaggcaccttgagcaggagagacctagggacccctttgtgcagcccctcctcacacggggagacgggcagccctggaggtcgggggttggggctgaggccacccacacacatgggctctcctaccaccttctcctcctgattcaggactgaggaatccaggaaccctggggagctcacaggcaccagggactgagacgctgcaagggcatcagccatcgaccattggacagccaccgcagtgagcaaaccccaaagtctcgtgctaagaagagacttcatttggagaagtgaattctgacaatactccacaaacaaagttaggacagttgttcaggacagcagcagggaggcatcgagtggaagtgaggaggaaccggcctttgtggagctccaagctgcataaagtctcccaaatgcaaagtctcgctccagtcagcaaagttcagagcagctctctccagcatcttcagccactgccaggcaggccggctctcagcacgggtggtaaactttctcattttcctcatgccaaaaatacatcatttccaaaatagaaaaatgatattgttcagaggtgaggcaggtgcgttaggaaagaacaaaaacaggccttattggatatgacaagaagtgcggcccctttcagaagcaaactagagaacctgagccaggaaggacgttgcatttgctgagtggccgaaagctgtcaggggtcggggcatgcagaagacgtcctgttattcacttctcagaacagacctgtgagtaaagaaccacatttcatagattaggaaacagccccggagagcctcggtgacgtgcacgaggtccccagctccgcggcgggtgagccaggctccaagcaccggctttcctggctgcacttcctggagggacaaatttggggatgagaaggaaatgagggcctggcggggtctctagggttttcattttgtgtcttcacataggtggcgaatttgagaaactgtaatggacgattttactctgatgagctctagagggggttgctgagaagcgaggcctgcgttttcctcctgacgaggtctgggcgggcagttgtctcctaccttttgtactaattcacctggtgacactgtcaaaggagagaaaaccacaggttagcaattcccactatattcattccaaacacagcccatcgtaatctaaaaagccatcagggccatcgcagggctccatgtttccctggggatgttacttcctgcacactgaccagcagatgatactgacaacactttgcagcctcaacccgtgataacgggaaaaagataaaataaagaggctggacatggtggctcacacctgtaatcccagcactttgggaggccgaggtgggaggattgcttgagcctagaggtttgagaccagcctgagcaacagaacaagaccctagctcttttaaaataactaaaagagtgtaattatattgcttgtaacacaaaggataaatgcttgaggggacggatgccccattgtccatgatgtgatcagcacgcattgcatgcctgtatcaaagcatctcatgtaccccataaatatatatgcctactatgtacccacaaaatttaaaaataaaacatttttaaagagagaaaagaaaacccacagggggcaggcacagattctggctgcctctggcgcatccacatggatgttggcatttcatcctcttctcagagaaagcagttggtcccacctccacccgctgtctgcggaggggtttggactctcagcatcttggctatgctggcactcttccctgctcagggagccagctgttcctggacccctgctaagaagacctaaccgtggatggcgtcatgatcgccaggccgtcccccgttctttcaccctctcgggacctggaattccatcttgcttccctattgtgtctgtccacggggccagtcacagggcaagtactcaacacgtatttgtcaaatgaaggaatacattgtacgaaggaaaaatcggaaaatgccacctctgaagattggaaatcagaactaacaagaatccaggtgagacattgagctcagcaggaggtttcccagcagtgtgttctctgtgaaaatgtgactcacaaggctgccgggtggggagcggaggggctccgaaatatcatctgggaatgtgaaatgatcagtaacattagcaacgatccgctcaagactgagccaggacaacaaaaagttttggtggctgatgagggtcagtcaggagcgctgggtagaatggagctagccacccgctggtttctggcattggacagaaacgctaagccaggggttctcctcacaggcccagggggaggaacgagcacggggcggtttgaggagggaagatggggcggagccccaggggcagcctcgcgcattgggtccgggtagtgtgatcccagagcctgcgtcttagcactgtgcggcctcgcccttctttttcttccacctgtgcgtggaaatagagcacgaacccaggaatgcagacaccaagtctctccacctcgagacgcgcatagcacacagagctgcagaggccacgcagataaaccaccagcactccagactgggcaggagccgctgtcaggacagaggacacctatccacacccgcctggcctcagaagatcctgggtcaaagaataattctcttttcatgtatatgtgttgaaagaatttcagactagcaagcaagccgttttgactgtttcttaagatacgtagggagtttgttctttctttattagcagaaagccacgttttaaggattaaattttttccataagaacgtaaggaaaatgtggtgaaacaaacaccttctgttggtcagggaaacactgggtaccttcaacgtgaggggctcctgaagacctcctgagaaccacacaatgacccaagacaggacctcagctgcctcttgaaggcagaggggaggctgcacattttacaggtgatctggacgttgttagatgctacaaataagagccaaggaaaagatgttaagcgtctgcgtgagtcaggaaaacctggagcgtgagtggaatcactgcttcctgactcgttccctcgccataggaaatggccctgggcagaaaacaagcccctgaggaggggggagcgtggacgcacgtggggagtgtggaggggggtccccactgaggagaggggagcgtggatgaaggtggggagtgtggaggggggtccacactgaggagaggggagcgtggatgaaggtggggagtgtggaggggggtccccactgaggagaggggagcgtggatgaaggtggggagtgtggaggggggtccccactgaggagaggggagcgtggatgaagatggggagtgtgggggggggtccacacttaggagaggggagcgtggcacacgtgggggtgtggaggggggtcccactgaggagaggggagcgtggatgaaggtggggagtgtggaggggggtccacactgaggagaggggagcatggatgaaggtggggagtgtggaggggggtcccactgaggagaggggagcgtggatgaaggtggggagtgtggaggggggtccccactgaggagaggggagcgtggacgcacgtggggggtgtggaggggggtccccactgaggagaggggagcgtggatgaaggtggggagtgtggaggggggtccccactgaggagaggggagcgtggaccacgtggggggtgtggaggggggtcccactgaggagaggggagcgtggatgaaggtggggagtgtggaggggggtcccactgaggagaggggagcgtggatgaaggtggggagtgtggaggggggtcccactgaggagaggggagcgtggatgaagatgggggtgtggaggggggtcccactgaggagaggggagcgtggacgcacgtggggggtgtggaggggggtccacactgaggagaggggagcgtggatgaagatgggggtgtggaggggggtcccactgaggagaggggagcgtggaccacgtgggggtgtggaggggggtccccactgaggagaggggagcgtggatgaaggtggggagtgtggagggggggtccctctgaggagaggggagcgtggacgcaggtcgggggtgtggagggggccccccacactgggatttccatccgaaatccacattccaagcttcatccaccagccttcgcactcctccggaggtccccaaatccaagtcaccagaggctgatacgcagaagctagaccgtgggtgcgcagctggcggcgagggtgagcagagccctgtcttctggtcagagctaaccccaggaaggccgaggggcatgggccgaggacatgtgcgtgggacccccagcggaggggaccgccgagggacggcctctcttctgtggactgtgtaaggcgccgcgtgtccctcctttctctaagcttaactgctgcaaagcctgccccttagccctctgcccgatgagccttcctggcagatggtggcatattcacccttcatcacggtgcagttacattcgtgccgaggggcgttttgtctgtaaggtcccttgcctatttctgccctgattcccactgagacgggtttccagttaaatctcagagggccagcaaatcccctcgcggggacgctttgtcccagtgacccaaaagtcaacacttgggaggcgtgtgctgtgggagacaaaggggcacaaagcggcaggagccccaggaatcgcggacggaagcgactgtgaaagacccgcgggggctgcccaggggaagaaggaaacgggtcccaggacccgctccagaaggcgcctgcggcaggggctccccggagcgcgtgggaacccgaggaggctgggacgcgcccgggggcagggacgggcgtttgcgaggctgcgaggctgcgagtgaagcgccgggctccggaaaggtgttgcgagtcgaacagtggatcccagggtaaaaagcgaagctgtaaatatgcccaggaccgatccagggagctgctgcttccaccagccacggagtctgggctttatgctgagaaccgtggggggttcgaggcgaggagagtcacagaatcgaaggctgccgcgcggtccgaacggcggcccaggctcagccacgcggtgagaggccgcggggtgtgaagaggctgggccggcaggtcctggcgggtgagggggacgggaagcggccgaggatgggtccttcgcttggattcggacggtggtgctgggagcgtcatccaccatcctcggagcaagagcccaacaccggggaggacccaggctcttcctggacccgctgagcttgggcagcctgggcggccctcagtgaaattctccagcaggtgatggagactctagacctggaacttgggagagccaaggcggtgggagcagcctgcatccaacaaaggagggagaccccaggggcctgtgcaagctacgaccaccggtgcaacccctctgacaagcaatgtgaggccctgtcctcccccgaccctgctctgaaaacggggctgcacttcttatccgctggggacacaggccatcatggaacccgccctggaagcggagaagcaggggagccgcctggccccatatttctcacacccagtccctctggggtcaaatcctaatggacgcgaccggccaggagtcccccagagccacccccgcagccctggctgtctcagccgcccacagggctcaagtggacaatgccagaggcagcaaagtccccatgaagcaaagccaggtgtgagtggcctgctggaccccaccgggctggaggacagaaggtgccccaaggaagccaggccaggcccgcgatggctccgggctgcctcaccttatccaagccccagaaaaaagactgaccaggatgagtctcgtcattgggatgaaacttcaccactagaaatgtgtttttcaaatggaatatcctttaaagtgcagacaccctcggcatctgcagcaaaataagttttattacatttcagtatcttcacctaaaaacattttagggggacacgtcgggaaatatggggaggcgagggtcgcggggggcccgtcaggaaagggggaagcgagggccctacgagacacaccagggtctgtctgacacagaggaaatcacatttttgccaagagtcagcccaaggccaggttctcaactccagcactcgggctgaagcctcgcggccgtgacaatccctcagcaagtgatgctcctgggactgaggatgggaggacgggatgtgctttcaggtgaggctgggacaggtggtcagcagcaggggaatagccgaggaggacgtgggatcagaacgtggaccagggaagccttctgcgtggtggggtccgccacggcctcccgctatgacttcttgcaggaagaaatgcctcaacacctcgctttgtagtaagtgaccctgggccacctggtcctttctgttcactctccccagcaagccctgaaatgtggcgccaccggcagcttacctttgtctactttggcgtggtgtggaaaaccgatgccagagctttaaccaagccttactccagagcacgtgtcccgccaaggcctcttcccgcacctgctgcggtgttggcactggcccccgagggcaggaggaaggaaccatcacgttgcagctccagctccagccctgctgttgttcgtgtttttccatctcccaaaaacctccagggaaatgtgacatcataacaaactgtaattggcataacaagtaatgcccccatcaataataaataaaatgatttcttttaatcctaacatgctttttgactttccgtattttgcatgtttatttttaacttttaggttcagtggtgcatgtgcaggtttgttacgtaaattgcgtgtcacagggtttgctgtacagactgttttgtcacccaggtgataggcatagcaccaggtaagtggtttctcggtccttgctctcctcccgcctccacctgaagtcatgcctgtgtctgttagtccttctttgtgtccatgtgttctcaatgtttagctcccactgtaagtgagaacacgcggtatttggttttctgttcctgaattagtttgctgaggataatgggctctagctccatccatgttgctgcaaaggacgtgatcttgttcttttttatggctgcatagtattccatggtgtatatgaactacgttttatttatctggtctgccactgataggcgtttgggttgattccatgtctttggtattgtgaatagagctgcagtgaacatacacatgcatgtatctttatggtagaatgatttatattcctttgagtataatatacccagggattgcagggtggaatggtagttctgttttgagttctttgagaaaccaccaaactgcatttcacaacggctgaactaatttacactcacaccagcagtgtataagcattcactcttctctacaacctctccagcatctgttattttttgactttttaataatagccatttcgactggcatgagatggtatttcactgtggttttgatttgcatttctctaatgattattgaggttgagcattttttcatgtgcttgttggctgcatgtatgtctctttttgagaagtgtctgttcatgtcctttgccctctttttaatggagtcgtttgggttttttgatgttaatttgtttaagtttcctacagatgctgaatattagacctttgttggatgcacagcttgcaaatatttctcccattctgtaggttgcctattactccatggatagctgcacctgcttttggcatctttgtcatgaaatctttgccagggcctatgttcacaatggcatttcccaggttttttctagggtttttattgttttaggttttacatttaagtctttaatccacattttgtatatggtgtaaggaaggggtccagtttcaatcttctgcatatggctagccagtcttttactatatgtagcagcggtccccaaactttttgacatcagggaccagttttgtggaagaaaatttttccacagactgcaggcaggagcagggtggtttgagatgaaactgttccacctcagatcatcaggcattagattctcataaggagggcacaacctaggtccctcatatgttcacaatagggcttgagctcctatgagaacctaaggccaccgccgccgctaatctgacaggaggcggagctcaggtggtaatgctcgctcacgcgccactcacctcctgatgtgcacccagcaggtgcgcaaagcagacgggatgaacgtctgggtaggtggaagggttgggagcagccaggactcagggaaatgtggcaggagtcccagggagagggcaagaggaacccgagagccccctttgtcctccccccataaagtgtcaacagagccaccttcgtcactgagcacaaagctggcagatagaaagaaagtgctcgataaaatagccactaaataaaatgaatgcacagctttgaaaatcagttgattcttctagttcatagtcttcttacagcaaataaatgatgaagacacatcatttgtgtaaaaaaaaaaaaagcaaagccttaaggaaggtgttcagttgagctgcatgtacagctgaaacagatccaagaaggtcaacgtgatttcgtgcggatgtttcctgtttgtgaaagtataaagacatttctcctactgaaaacaagaggtcttcccccttggtgagcagtgaggattgtggaggagagggtggcatctctggcaagaggacagcagtaaaccaaggcttcctttgactttttttaaaaaaacattcttaatacaccaaattggccagaaaatggaacatgaatatccttgaaattgagatacatcaacttccttacgaccacccagtgcactcaccatcaggaaaggaaggcaggaaggggacggtctgtctccatggcaccgcggccacacttctcagctctcagcactgtccccgcgacggaggagccgccccagcacagctctaagcgctgtccccccgacagaggagctgccccagtctcactctcagcgctgtccccgacaacggaggagccgtcccagtctcagctctcagcgctgtccccgacgacggaggagctgccccagtctcactctcagcgctgtccccccgacagaggagctaccccagtctcagctctcagcgctgtccccgacaacggaggagccgtcccagtctcagctctcagcgctgtcccccacgacggaggagccgccccactctcagctctcagcgctgtcccccacgacggaggagccaccccagtctcactctcagcgctgtcccccacgacggaggagccaccccagtctcactctcagcactgtcccccacgacggaggagccgccccagtctcagctctcagcgctgtccccgacgacggaggagccgccccagcacatccagcgacatggcaggggcaccatcctgggaggctccggacacaggagttacctggacccagcaggggaaacacagctgctgctaagagaacagcttctccgcagaatgcagcccaatccacctctttccaggccactctgttccaggatgtgagactcaaagggccagggtggggcctgcaagacaggctcagctgggggccccgcagggactggaatctgcacaggtgatgtgggtgtgatgcttgagatgtttaccttgtgcaagcaactatttctcctgagaatttaaatagttacgtaatttctagagccagagacatccccagtacacatacacacacatacaactttatataaatcatatcatgttatgaggctgggctttaaaaataaactttatttttggagcagttttaggtgaggaagctacagagacttcctgtgtacccactgtctgcgcacacagccccactcactcccaacacccctcaccagagtggtgcctttgctacaatcagtgaacgcacaatgacacatcattgtcacccagagtccacggtcgacacggggacccacgcctggtgttgtacattctgtggctaacgacacggatccagcatgacagccttgtgcagagcaggtccctgccctggaaatcccctgtgctccagccgcccctccctccctcccctccctcagccccggcagctgctgcatttgttactgtctccacagttttgccgttttcagaatgtcatagaattggaatcatacaacgtgtggccttttcagattggccccttcacctagtaatatacatttaaggttcctccaggtcttttcatggcttgatagctcagttcttctttagcaacaaataatatgccctggtctggaggcaccccagttcacttctccattcacccactgagggacatctcggctgcttccaagttcgggcagttgtgaatggagctgctataaacatgcatgtgcaggcttgtgggacgtaaggtttcaactctttaggtaaatacgaaggagcatgactgctggatgatacggtaatagtaggttaaatttcatgaaaagccaccaagctgtcctccacagtggctgtagcattttgcattcccacaagcaatgaatgatttggtgttgtcagtgttccagactttggccattctttttatttttttattttttattttttgagacaaagtctcattctgttgccgaggctggagtgcagtagcatgatctcggctcactgcaacctgcacctccctggttcaagcaattctcctgcctcagcctcccaagtagttgggactacaggtgcgtgcgaccatgcccagccaattttgtatttttagtagagacggagtttcaccatgttggccaggctggtctcaaactcctgacttcaggtgatccatcctcctcagcctctcaaagtgctgggattgcaggtgtgagccaccgcgcctggccgactttggccattctaataggtgtacaggggcatctcgttgttgttttaatttgtgcttccttgaacatgcaatttggcgcatcttttcatgtgcttgtttgccatctgtagatcctcggcaagtgtctgttagggtctttacatcatttttaatagggttgtttgttttcttatacttaggttttaagagttctttatgtattttggataccagtcctgtatcagatgtgtgttctgcaaataccttctccctgtcatctcattctcttgatgcattctgggtttttacaatgaaaactttgttcctcattttcttcttttttactcaactctgcttcactcttttctaccactgcctcccggcagcctgttcacaaccacgtgtatctttccacattgtatccaccttcacagaattatatacaaacgtgtatctatgtcatatacacagacatatataaaattaaatatatgtatgtggagatattttgtcatttgctttataaaaatagaatcgcacttaaatgcatccttgcatcttatctttcttcttcaataacgcctcatggaaatccctgcaaattaactggtatagacctacttcattttcctaacggtggcttgaaactccagtgtgaatgcaccatcattcactgaatcacttctagatcgtgaggcactggctttgtttctggacatttgctacaaatgctgctgaaacaaacacctcggacaactgtgcttgcacactcgccctcctgtgtccacggcacatattcccaggagtgcagctgatatgttgataatgggtgtattctttatcttggtaggtgttgccatatcacttcaaaagagagtctaacagtgtatcttaccacatggttctccctacttgccgagaccaacagctctttctagccaccctgcctctttgctgataaaaagaactctaaaccctttatgaagccctccaaggccctataattgatttgacatctacctttacctctctcttacctcccactctattttccttttttttttttttttatgaagtcttgctctgtggcccatgctggagtgaaatggtgctatctcagctcactgcaacctccgcctccagcattcaagcgattctcctgcctcggcctcccaagtagctggaactacaggcatgcacaaccatgcctggctaatttttgtatttttagtagagatggggtttcatcatgtaggccaggctggtctcaaactcctgacctctggtgatccgtccacgtcagcctcccaaagtgctgggattacaggcgtgagccactgcgcccacctcacctctggtgatccgcccgcctcagccttccaaagtactgggattacaggcgtgagccgccgcaccccgcctccaaggtgctgggattacaggcgtgagccgccgcgcccagcctatttcccactgttctttgtgaaccctgttccagccacctgccaactctgttcctcaatcaatttctacatattaatgtttctgtgctcacactgctcggtgggaatgtcctgaccccactccccatttcatgagttccatgatccttcagacccaaagtctcatcatcaccaccaagtcctgacctgtgctggggacactctgtgctccacagagtccatcggcctaatccctgcatcatacttggagatgcagggagagtgagtggctgctcagagtcatggataggaggtggtgggaagtggcagagcccacctgacagtgtcttggatttggagacagtgtgactgttccctgctcagtgtaacctgataccaaattctgatgtgaggaagttttcctgagtgcccagcgcctgctttccctttgttgattctaatccgtttctccagagagagtcattcctgtaagaagatccccactgcctgaggttcatggctcaggtgtcttcctgggtggtcttctctgaccacagccaccctattaaattatccattaccttcaccagctggcattttctatatttcacttatttatcttgactcttacataactctaaccactagaggcaaggtccataacagcaggattccatcctcttgggttactgctgcctcagtaaatgtgggatggatggatgcatgcacgcatgaatgatgcacagatgaatgaatggatgcatgaaaggattgatagatgaaaggatgcatggatgacagatgggtaaatgcatggatgatgaatggatggagggatacatgaatggaaggattcatgaatggatgaatggatgggtggctccatggatggatgaatgaatgaatgcatgatgaatggatagttggatggatggatgcatgcatgcatgaatgaatggatggatggatgcatggatggatacatgaatgcatggatgactggatgactgcatggatggatggatggatacctggatggatacttggatggatggatggatgcacggatggatggatgcatggatagatagatgggcacatgagtagatgaacagatgcatagatggatggatgaatggatgcatggatggatggatggatgcatggatggatgcatggatgcatggatggatacatgaatccatggatgagtggatgagtgcatggatggatggatggatggatggatggatggatacttggatggatggatggatggatgggtggatggatggacggatggatggatgcatggatagatggatggatgcatgaatagatgaatggatgcatagatggatggatgaatggatgcatggatggatggatgcatgcatggatggaggcatggatggatgcatggatggatacatggatgagtggatgaatgcatggatggatggatggatagatgatggatacttggatggacagatggatggatggatggatgcatggacggatggatgcatggacggatggatggatgcatgaataggtgaatggatgcatagatggatggatggatggatgcatggatggatggatggatggatggatggatggatgcatgaattgatggatgatgcatggatggatggacagatggatggatggatagctggatggatgcacagatggatggatggatgcatgaatagatggatgatgcatagatggatggatgcatgaattgatggatgatgcatagatggatggatgcacggatggatgcacagatgggtggatgcataaatggatgaatgcatgaatggatggatgaatggatgcacagatggatggatgatgcatagatggatgaaggcatgaatggatggatgaatggatgcatagatagatggatgatgcatacatggatggatgaaatcatgcatggatgcaagaatttatggatggttggattgatgaatgggtgcatggatgaatagatggatggatacgtggatggatgcatggatagtgtatggatgcatagatgcatggatggatgaatgtattgatggatggatggatgcatagatggataggtgcatgaatgaatggatgcattgataatggatggatgcatggataatggatgaatggatgaatggatggatagatgggtgcatggatggatggatggatgggtgcatggatgaatgcctggataatggatggatgcatggatggatggatgaatacatgaatttatggatgaatagatggacagatgcatggataatgaatggactcatgagtggatgcatgagtggattcatagatgcatgtatggattgatgcatgcatggattaatgaaagcatggatggacagatgcatggataatggatgaatgaatggaatgatggatggatgaacatgtggatggatggatgcatgcatgcatgcatggatagatggatggatgaatacatgaatttatggatgaatagatggacagatgcatggataatgaatggactcatgagtggatgcatgagtggattcatagatgcatgtatggattgatgcatgcatggattaatgaatgcatggatggacagatgcatggataatggatgaatgaatggaatgatggatggatgaacatgtggatggatggatgcatgcatgcatgcatggatagatggatgatgcataggtacatggatggatggatggatggaaggatgcatagatggatgcatgcataaattgatggatagatggatggatggatggatggatagatgcatggatgcatggaaggacagaatacttctcttttcctctcctctatggaaattgatggtgttcacttctaatctgccttgtgtactaaaggcaatctctttccccaactacaattcacataatctattagaatatcactagcagtcaaataattttgaactgttgccaacttagtttaagcctagttaatccacttagaaagaccattactcaagtataattacatttcttcttctgaaatgatgccactaaattcaataaatatttgttaaataaatagcaatatgtcatttctgaaaacactggagcagttgaccagcatacaggaagataagatgttgagtatgaccctgaactctgaactacctgttttgtatctcttggagaaaatgtgagtgcacacagcatgcaattctagcagaggctccccaaagcctgcagagcttttctaattgcatgttcattatgaacacataattaaactggtaacattaattttcaattagaggagcacattaaagttttggtgtttgggcattcatgtggacataatccctgagtaattaaaacacagccttcctttcagaggacacagtgcaatctgtgacctgccttgatgaaaaaaatgggaaaggaaagtcctgagagtgacactggcttacaggagaaccaacacatttttgtgtctgcaagagatgtgttctggcttgtagaccttgggagcagctcccagtggggtcgccacccctcacagactcctgaacaaactcatcctctacttggtactgaccacagtagtttttgaggcccaaggcaaatcacacctttttttttgccaatatcatcctttctattacatcaagttaaaattgccctcaagagctaacttgcagaggaaaatgtcagcaaagatcaccagttgcagtctgaagctgatgtggacagggaactgctgttggccacccttgatgaatgagccccgggctatcccagtgggaggaacaaagatgtccaccgttgccaaaggtgtcatgtgcgtttcagtgccacccagtggagttagcaccttcttcctgggaagatctgaccctgcagctggctggcccccaagctaagggctggaatcacaatctgctctgaagagggcatggaccattcatatggcctgggagcagcacaagcactgagtgaggagcccagctttccctggagactaaaacccattcccaagtgtcagctacgagctcactccctgtgcctcatccgtcctctctcgacctattctgggagagctgcagacaaggttcaggaatgaggccatgcagccaaggtggacgagacagaggaaaagagggagacaaaaggtgcttggacccacaccatgtccactcctttgttttttgccgcttcacactttgggttctggaaagattcaccacccagagtctttctccccatgttatccgaagtaaaaatttactgagcagctcttatgtgccaggctctgcatcaggcacttagggtatatctgtgaacaagcaaaacacaaaaaggaccttcatcctgaagggcccacgttgacctccaagcatgcccaccccacagactcatcgatatgatttggctgtatccccaccccaacctcatcttgaattgtagctcccataattcccatgtgttgtgggagggacccagtgggagataacagaatcatgggggcagttccccccatactgttcttgtggtagtgaataagtctcatgagatctgatgggtttataaggggaaacccctttcacttggttccctcattttctctcttgtctgctgccatgtaagacctgcctttcaccttccaccgtgattgtgaggcctcctcagacacgtggaattgtgagtccattaaacctttttttctttataagttacccagtctcgggtatggctttatcagcagcatgaaaacagactcatacaataaattggtaccagtacagtggggtgctgctgtaaagatacccaaaaatgtggaagcgactttggaactgggtaacaggcagaggttggaacagtttggagagctcagaagaagacaggaaaatgtgagacagtttggaacttcctagagacttgttgaatggctttgaccaacatgctgataatgatatggacaataaaatccagactgaggtagtctcagacggaaatgaggaatttgttggcaactggagcgaaggccactcttgtaatgtttcagcaaagagactgccagcattttgcccctgccctagaaatttgtggaactttgaatttgagagagattatttaggatatctggtggaagaaatttctaagtggcaaagcattcaagaggtaacttaggcgctgttaaaggcattcagttttataagggaagcagagcagaagagttcagaaaatgtgcagcctgacaatgcaatagaaaggaaagtcccattttctgaggagaaattcaagccagctgcagaaatttgcataagtaacaaggagctgaatgttaattgccaagacaatggggaaaatgtctccgggacatgtcagaggccttcatggcatcccctcccatcacaggcccagaggcctgggaggaaaaaatggtttcatgggcgaggcccaggatccctgtgctgtgtgcagtctagggacttggtgccctgtgtcccagccactccagccatgactaaaaggggccaaggtacagctcaggtcatggcttcagaggatgcaaaccccaagccctagcagcttccatgtggtgttgagcctgcaggtgcagagaagtcaataattgaggtttgggaacctccacctagatgttagaggatgtatggaaatgcctggatgttcaggcagaagtttgctgcagggtcagggccctcatggggaacccctgctagggcagtgcagaagggaattgtggggtgggcacccccacacacacagagtcaccactgaggtgctacctagtggagctgtgagaaagtcaccatcctccagaccccaggatggtagatccaccaacaacttgctctgtgcacctggtaaagctgcagacactcaacaccagcctgtgaaagctcaggagggagactgtaccttgctaagccacagaggcagagctgcccaagaccatgggaacccacctcttgcatcagtgtgagctggatgtgagacacggagtcaaagatcactttgaagctttaagatttgactgccccgctggattttggacttacgtggggcctgtaactcctttgttttggccaatttctcccatttggaatggatatatttacccaatgcctgtacccccattgtatctagaaagcagctaacttgcctttgattttataggctcataggcagaagggacttgcttgtctcaaatgagactttggactgtggacttttgagttaatgctgaattaagactttggggactgttgggaaggtacgagtggttttaaaatgtgaagacatgagacttgggaggagccaggggtggaattatatggttcagctgtgtctccaccaaaatctcagcttgaattgtagctcccataattattcccatgtgttgtgggagggacccagtgggagataacagaatcacaggggtggtttcccccatattgttctcgtggtagtgaataagtctcacaagatctgatagttttataaagggaaacccctttcacttggttccctcattttttctcgtctgctgccatgtaagatgtgcctttcatcttccaccatgattgtgaggcctccccagccacatggaactgtgagtccattaaacctctttttctttataacaagaaaaaatatcaaaatacccagtctcaggtatgtctttattggcagcatgaaaacagaataatacactcgtgctggagttcatctctttcctcagtgtgggacacttagatcctgtctaatttttcactagtgtaagcagggctgctgttgatatcctggtgtggaactttggtatcagaagctagcctccatttctggcagctggagagccagtgagatggcttcaggctggcctggccccagaggggtgggcatggctcagagttcatcacaaatgtggcttccgtgtccttgctggaaaaatcagcacatcagaccaaagctcaaaactcgcttctaaaacaatattctgtggtcaggactccgcaacagcattttaaatttattcacaaaatctggagtagatggttttggaaagcgttttggtgaatggtctgtattcagccttagatctaccaaatatttcaaagacgttccttgcagaaacccatgagctagccactcgagcaggagtgtttactcccaggctcactcaaacagaagaccttcccactcgagcaggagtgtttactcccaggatcactcgaacagaagaccttcccactcgagcaggagtgtttactcccaggctcactcgaacagaagactttcccactcgagcaggagtgttcactcccaggctcactcgaacagaagactttcccactcgagcaggagtgtttactcccaggctcactcgaacagaagactttcccactcgagcaggagtgtttactcccaggctcactcgaacagaagactttcccactcgagcaggagtgtttactcccaggctcactcgaacagaagactttcccactcgagcaggagtgtttactcccaggctcactcgaacagaagactttcccactcgagcaggagtgtttactcccaggctcactcgaacagaagactttcccactcgagcaggagtgtttactcccaggctcactcgaacagaagactttcccacacaagccctggagccagtctcttggtcaccctttgggtctaggggaagtgagaggtggtcctgttcctggaagagggggtctctaccccaagagaggcttctcagatctcacacaagagggaattcaaggccagtcacagagcacagtgaaagaggcacgtttattagaaatgactctgttacagagcagaaagcaagcagaggacacaccaacttctttttccatttttcttacacagaagtcttgtctgcaaagcgaagtgaagctgtgcatatgtgcagctgagcagagagcatgacaaggtttatcattctattggtttaaagaaaaccatccttgacattttagtgtgtcagtcatcatagcatgactcttactatcttgaaagcatagacctaattatgggtattggacgtctggactttctgttcttggagtttgctttgcaggcatcaccaagctgcttccttagctgtaaacatcttaggaccatggcttgtgtgtggcaaggagtgtgccttgctagtttaagacggtgctgattttaaaatggtgtcaccctggctcctctggactcctgcctcgctaacagtccagtgactgtccatggggctgctaatcaagtccacagcaggaagcagcctcttccctgagcctcccaaccccccagacgacttctcccctggaccttcaggtcctaggatccaaacaccagagacgcacagaggcctagagctccatgctgtgctgtggcctgcttggtctgaccagccaggcccataaatactgatggataatcacaagggtaggaggggagtggacagggagacaggtgtcactcagtctacagattgcccaaaaggagcatcaacgccaaacccgaagtatttacctgaacccagagtcccagagtctcctcccctgtgacttctcctgagacttgggggtggtccggccccctccatctccagcgaacactctgaaaatttgcatctgcccacagcactgccctacgtgaatgtgtttgggggctcccttccttcacaggaccatccggacactgtggccttgcctgcggcccctccccacccggcgcacctcccccacatccccgcagaccttccactgtcaatgtctgccactcctttctgtcaatgtctgccactcctttgcctgggggtctcccacactgcccacccctcatcctggagaccgccagggggcccctaggaagcacgtcttggaccctgaggtgatgtcacaacctctgtggtgcccctgcaccctggaccatcttctccgccagctcccatcacccttgctgcgtctgcctctgtccatcgctttctgtgattacaagtccttggtgagaagtgccactgtgtgtgtggagatgtgggtacagaaaaataccaaaacatctgggtggggtacaaggaacagggtcaggggagaagcctcactcagaggtgagaagggtgaggcccagggtctcaccatcccaaaggggtccagggttgggctccagggtgcagcctggacaccgcttacatgtgcaggtgttggcacctgaggttccacgcaggaattcaggtttgcctggaataacccccggctgccttgtgggaaattagattaattaagagaggccatgtgacctgctgccagtcggcccaaggtgagcttccaggccgtctgcaggtagccctgctcagtgctccccacgtgttgtccacagtgcctgctgccggcaggtcaccaatatgtcattgctgagtgagcaatttgagcacggcactgtcaggtgcagtggggcctgcatctgtcagatctgcaggctggggtgcggccagggtggggagctgtggccgcccgccccagcctgacctccgctccacacacaggagggtttggccttccctgcaggcttcctcctgagctctgctttttttctgctttggagtcttcacgctcaagttcgtgctttcccttacaaccaggaggactccggaggccgggccgcagcaaaacttgcctgtgggttttatgagaatttacataatcatccctggtgtcacccccagggcgactgcactgatgatacaggctgtctccctggcttggcatctagcagcacttaaagagagccacgaagtttacagcagggactgagcaggcggagttctggactgaatcatcgtttccagttaggaagggactcgggagggaccggtcagcaaggcctcagccttggcttctgtttttcttttgatgaaagaaggcaaggccgagcctcacagagacagggtgaaccttgaaccactcagggtggccaaaaaaagtcttcatggaatcctccagtgtataaatgatcctttcaaagccttcccaaggcaagtgaggctggacggggttttctgcaccacagtccaagcctcctcctcccctgctgcctggctctgacggtggctacaagaagctgcaaaggcaccaatggtttgaaagagatttaaaagtaaaaaatcagacaaactgagtgattagttcccagaagtaagcacctggacggagagagggagcgggaggtaagagctccagggaggcaggaggccgctggtggcagcgctctgggtctctgagaatctcagggaaacattggcgctgacccttgttgtgcggaaggcgaagctgcacccccaggtgcatattccactcttattcctaggaaggtggaaaccccatctgtggtggtttagggcgaccggtgcctcggagtctgattttcccttgggtttcagggcagggcgaggttcccagctccatctgcagctgtagctttcctagccagcccgtcccgagagcacgtccaagtgtccatcccgccgtgggttctgcctaattcttgagtaaacggctgccattttgatgaacatcatttgtccttattgctgtgggggtttcctggcttttagcaaatatttactcaatttatcaactgcacaatacaacatcgccatttatgacactattattctgctctcaccaggggtctggcctttgaaatgtaacctccgtgtatccagggatttctcactgagctttaacattttacccccagatagtacttggccctggaaacacgaaaggcggagatttgactgttataaggaggaaaataaatctttttttcaatttaaaagatgaattcaagttaacagacctagaaacgttggctgagcctccaaatggagacatgtgtacccgcagtcccaggccataaataatcacggcatctggctagcgagcacccagtgctataaagaattattggaccataaaaataagccttcagtaatgtataaagaacccccacaactcaacaacaaaaaaacaaacagctcgattcaaaaatggtcaaaggacttgaatagacaatttcttcagagaagacatacaaatggccaataaacccatgaaaagatgctcactatcacaggtcattagggaaattcaaatcaaaaccacaatgaggtgccacctcacagccattagggtaacaagtatcaaaaatagaagacagcaagtgctcgggatgtggagaaactggaggatgtggggacaccgggaccctttacattgcttgaggggatgtaaaacagggcagccgctggggaagacagtgtggcggctcctcaaaaccttaaaaatagaactgccatatgacccagcaatttcacttttgggtatttctcaaaaagaactgagagcagggacttgggcagatgcctgtgcacccgtgtttagggcagccttattcacacccactaaacatggtccaacccaagcgtccgataacggatgaacgcataaacaaaatgtggtctatccatacggtaaagcattattcagcctttaaaaaaggaaggggattctggcacctgccacaatgcggataaaccctgaggacattatgctcggtgaagtaagccagacacaaaaggcccacgatggtatggctgtgctcagagactgacggtcatcagattcacagagacagaaggtagaagggggtgggggatggggagcacttgcttaaagggacagaatttgttggggaaaatgaaggggttctagggtggacgatggtgatggccacacatcatagtgaatgcacttaatgccacacaactgtgtacttaaaaatagttaaaaccactcatttcacgttaaagagccttcggtgacagtctcaggtgagctgatgtcatgagcgggagacagtgagggtgtggacgtgtggttcatggtgcagcagggtcagccttggggacccagaaagctgcaccgaatgtcgcctcggaaaaacgaaacaaagtcagggctcgccctcccttaagtctcctctttctgttcggacttcacctccaccctgcctgtgttcctaactttgaaagctgtggtgggcgtggggagtggaggagctgggtgcccccccgaccacctgccagccccagctgggagcagaggcctgaggacgcgcactgaggggccaggcgtgaccttctgcctgtcattttttcctctcttcctttttttttttttcgcttttcttcaagattttatctttttggagcagctttaagttcacatcaaattacaagaaaggtacagagagctcccacatacctgaccccccagagcctctgcatttccaacaacgcccagcagagggcgaatttgctacagttgatgaacccacagtaacgtcattatcacccagagccacagttttcattagggctccgtctcagtgctgtacgtgacgttctcatgggatctctggggtgccgatttttccggccggaaacctctgtggctgcagcgcctttgccagagctcttgtcctgcgtctaggaagaatgaggtacgcagacaagtgaagcgtgaagaagaaaagttgtatttagtgtcagaacagctcagaggagtgggtggctcctctcgtaggcaggtcgtctctcgagtgttcagctttcagcagagaggaggccccagagagggaagctgctctctgcaggcaggtcattcagatgtctctgcaggtctctgaagctctcagcagagagggtagctcctctctgtggacaggttgtcttgccttctgctgttctcagtggagagggtactcctctctgcagctggtcatcccatccggtcgtctctctgccctctgtcctcttcatcctgtgacccttctctgccctgctctggctgagcccagagcttgtatggacctctgaggggaggaagtgcatgccgattggtccataagtggtcatgggtgggcccagaagaggcatcaagagtccccactcctgtctgcagcactggcagcccagcccccagccttcaggccctcgctggcctgaaggtagggccttcctggggacctgccccttctacccagaaatcaatctgcctcccacggcccttcatggcccctgggcttggccccagccccagctctgagatcggagcaggcgctaggagtggagagaggccaggcagcagaagacacccccgagcctgcagggatgtggtagggggggtccttcctggggcccctgagggtgtaggctgcagaaacacccaggtcctgtacccgggagggcggccacagctgcacctgggagctcccaccaactcggaaggggcagggctcctacttgtccctagctcctgcccgctttttggaataggaggcccaggtttgcagccgtgggtgccgcagctgcagctgtacccgggaaggcagatcttgcctgttcccagctccctcaagtgcacagggaggctcatatccacagctgcagtttgggcagcttcgtagagcaggaggcctgggtctgcagctgtggtttgggcaggtgcagcagcacggggagcacggacagctcccttccgaactcagaaggggcggggcacccactggctccatggagtgtgaggccccagccacgcttccctgctgcaggcggatgatggcagcagccactgctgtcaatgtaggtttggctacatgtgtaatgacacacatcctccattatagtatcatagagttgttccaccgtcccaaaaatatcctgtgctccacccatgcacctcccctccgtgctccacccacgcacctcccctccgtgctccacccacgcacctcccctccgtgctccacccacgcacctcccctccgtgctccacccacgcacctcccctccgtgctccacccacgcacctcccctctttgcccctcctgtcttccttcccagctatgtaaacccctgactttagccgggaaggcgggacggatttgagttttgactcctgtctctccagctgacaccgcctgtaatagaataaagccttcttccccggcaactctctttgtctcagagactgggggctttttgtgcagtgggcagcatgacctggacagaacccctggtgttcgggaacaacttgttggaattgttgttggtattgcattaaatctatggatgaatttgtgagaaattgacctctgatgatagtgagtcttcctgaccatgaatatggaatatctctcctttcatttagtccttctttgacttatttcatcagagttttgtagttttcctcatatagctcttgtacatactttgttagatttatacctcagtatttcattttcagtaaatggtattatgtttttattttcaaattccactcgctcatttgtagtacataggaaagcgactgacttttacatattagccttgtttcctgcgaccttgctataattgcttattagttccagatctgcctttctttcagagtctatgtctatcatggctctggggtgtattcttagcccaactttacaacaagcatcctcagtttctgccttggttcttctgactatggatgagtatcataagaaatgagatattcacagctgggcagcctcttctgttttaaaaatgtccaacagagctaagaagtctactaagttaacatgattccaaacaaaaggccaaagaggtcctcatcccaccccccaggaaacttaaaaataaattcacgggaggctgaggcaggagaatcgcttgaacccgggaggtggaggttgcagtgagccaagatcgcaccactgcactccagcctgggcgacagagcaagattccatcaaaaaaattaaagaaagagaaacctgggtgccgcagagccccgaggccgggcggccttgcctgcctggaacccccttggcggccccgcgggctcctccccacccgcctggcctcccccatctctgcgttcggggcctccccggctgccccaaggcgcgtccgtccccagcaggcagcgcgtttgccttcccgtccctcccaccacccccccattcctggctacctcgcccccctcaagttggggccgccttggggtcttcgttactcccgggacgtctcaggccgtttcagcctccgtttatggatgttaccttgctctggaatgttctccccagggaacgccctctcgcagggcctcggcggcacaccctctggaaccttccagaatcctccggaagagtcagcagagctgactgcgcggagcgagtggctgaaaggagggcggggaggcggccgctctctatctaagaagtggcgggtctggagggtgcgcgagggcagggccgggtgcccaggccccacccacgggccctgcgcgcctggacattctgtcaatgccgggaacgcggagggcgggcgctggcatcggccctgggaggacaggcgctcccggagctccagacgccaggccagaccgccggggacagcgcggggtgcaaggatggggggcacagagaggcgggtggggggcctgggcgagggtggggggcgcggagaggccggtggggggcctgggcgagggtggggggcgcggagaggcgggtgggggcctgggcgagggtggggggcgcggagaggcggggggggggcctgggcgagggtggggggcacggataggccggtggggggcctgggcgagggaccgcgcctgccctgagactgagccgggcctgagcgagggttccgccgggcgcctgcagtcgggcagcctcgtggcgccccagccacggcctctgcacagcacacatctccatctgagatgcagaaacgctaaccgggtccctgccacacgtcaaacgtcggcgcgggaagccacgggcctcctcaggccggaaaggaacagcccgccccctactgtgcagagcgaggctccgcggcctgaggaagggagacctgcctgggcgctgagctcacaggcttctaggcaccttcggaggccgctgaagagcgggggtggcgcgagctggcggtcaccgggaagccccggttaccatggcaaggcgatgggtgcgcggttacccggcgtgcagcctcgcgaggacacctgggccacggacgcgccgcgccctcggggagccggctccggaggcctctctcttcctcctcctcctacccctaatgaccatggattacttttgaaatggaaaaacggtaaaccttttctaacataagtcacacttataaagaaagcatctgctaaagaggacgtgttatgtcatgaaatggggggctctcatcacggcgggggtcagggaaggtccgctagggctttagcagaagagattaacctgctgactaccaggcgccttccaatactaatattctgagattttaatatattttatattaccataaagatcttgagtgctttagaaataatactaggtagctgagcacagtggctcacccctgtaatcccacggctttgggaggccgaggtgggaggcccgaggccaggaggttgagaccagcctggacaacataacatcccatctctacagaaacccaccatttcattaaaacattagcggggcatggtggcagtgttcgtggtctgggaggctaagctgggaggatcacttgagcccaggagtttgtggctgcagtgcaccatggtctcaccactgcactcaagcctgggcaacagagcaaaactcaatcctactgcgtaaagtagaaataccatttctgggccgggcgtggtggctcacgcctgtaatcccagcactttgggaggccgaggcggccggatcacgaggtcaggagatcgagaccttcctggttaacaaggtgaaaccccgtctctactaaaaaaaccacaaaaaattagctgggcgtggtggcaggtgcctgtagtcccagctactccggaggctgaggcaggagaatggtgtgaaccccggggggtggcacttgcagtgagccgagattgcaccactgcactctggcctgggcgaaagagcgagactctgtctcaaaaaaaaaaaaaaaaaaaaaaggaaataccatttctgggtcataaaaaaatatttcagacatcattattccggatatgttaactttttgctaatgggatataaatgcctccaatttgaaattgctcttccaggagtgaattttaattttctcccactgtgaaaatgcgtatttgtcttatggagagagtttttatttattaccaactaacagtaaaagcttcttctcacaattctgacaaagtagagccaatgtaagagttttggtgtcatttttaatgtagaggcttcaattattgttagtcaaagggaacatatcttgaaacattaacaacaaaatcactaacctttgattttctcttttagatttgcacattgttgtctttgttctaggcaaggcattcctgtttcaatttttcctttattccttttcttttttcctgcactgttttcagaatatgttctctgacattttaggccatctaagtcaagaaaccagtatatgaattactttgactggagagaagggatgtttgatcccaaattcctgaattgggctcctctgaaatatcagtaggaactacaagcaaaaaattgatggggcgtggtcctgtggtttggggaatttttatttacttcagtatacatgtcatatgttcatataagttatatattatttattactgtattactccactttcttttggagagtcacactccacgttaacctactaaagtctctgaaaagttctgcaatatatgaaattctctatttttagcacagtatttccaaaactgtattttattgagtttcagaagctattgattgaaagacgtgtacttttgaaagaacaataatcactgctaataaaactatggcatgccatggaattataagacacatcacaactttaaaagcattaagatgttcaaatgagcatcttagaatccattgaaatttgttaatttatcttaagtatgttacccatcactgcaaaacacatgataccaaaaggaaggggcataaaacagccactttatttgctcacaaggctgtgggtcggcagtttgggttgggctcagccatgtggttcatatgctttctagctgggacattctcacagcttcagatgtacccagctggggatggctggtcagggatggtctggtgagtgagacttgcaggtgctggcaggggaaccacagctctccaggcctctgcagcagaggagatcatatctgcttgcatcatggtctcaggtccccacacacagcaaaaggaagacagcctcaatgtgctggcctttttccagcctctgcttttcttgggttaaatttgctaacatcacattaactaaggcaaattgcatggacacatcagattcaagaggtgaagaaataaacttgatgggaagagtggcagagttgcactgcataggggcaagctgacagatgggagaggattattgctgtcatctttccacacacaaacttttaaaaaagaatttcctaagacatctcttaacatttcatacattatagtttagaaaaatctcctggataacaattgaatcttcagtactgagaaacagatgagacaggtatgtatttagagatgctgttgacacctgagacaactggacaaagagatgtcagggccttccattttgcctgctcacaagctcacagtccacttgtcactgttccacagagactggcagaagacacaagactcctggatcagagacaaaggacttcattatgacccacggcacagctagcagcatgagcactgtgttgaccagtttcctaggccccctagtccataggcatggcatgaagggcccacagggatgctgtacacacataggtcagcattgcagctgaggggcagtgggcttggggaatctactgcccttacagggagcagtaagtgggcctgttctttatccagagagagatattatccctatcctcagggttgttccctgcaaacgctacccccaaaaaatggcccaagtaaagattatcagagccttgactccttgacacacccaccaaaaacatgcagactcaggataaggcagagcaaggggattgtgtgtgaaagccctccccatgttgcaaggctagttaggtggaaaccaggccatccagctccctgccctctggcgcccagggcgttgtcatgtgacttgggctgtcaattgaatgcttctgtggggacagttaggaaattgctcaaaggtaggggagaggtggcaagtttccacttataccactggtgtccaggatccaggggcactggcagtggtcagtgtccagaggtagtaagagacgtctgtaaacgtgaagtgagttgcaatgcactaagcaagttttcctgcctcccagcccccttggctcccgcctgtttgcaccactcttgctctacagctctcccatgatgttgcaggctacccaggtcctcagtataaattcatttctgtttgagttaggctgatgatgcttaaaatcaagaaaaatctgtctttagtaggaacttgacattccgatacaatatttttgatgccagaaacatattaaaacctaaaaaaacaagacatgaagttaaaagaagttaagtctagtctctatgatatcctaagccctatatacagtatataatttgtaaattcctgaacaaacagaaaaatgtgacttatacataagagaaaaatcagtcagtagaaggagacccaccaatgaccatgatgttctaattagtagaaaagagctttgaaataacaatgataaatatgttgaagattctacaggaaaagatagttataatgctaagaaattttagaagatatttagaaacttttctttaaaaaggatattctagaaatgaaaaatacagtatctgaaataaaaaaaaattattgaatgaactggccacagtgtctgcttttgtaaaaacctccaaaaagttatgcattgagtatttccatttcagattcatctgctgcccacctctcaccacagatgaatgatcactatgaatggtggtgaccaggtgggcaggggaggggcttgtctgagtgccttagcccaggcttctggcctgccaaccttgaaaacagcaatatcagctgggcgcagtggctcaccactgtaatcccaacactttgggatgccaaggcaggcagatcacttgaagccaggaggtcgagacctgcctggccaacttagtgaaactccgtctctactaaaaatacaaaaattagtctggagtggtggtgcacacctgtagtcccagcgatttgggaggctgaggcacaagaatagcttgaacctgggagacaaaggtagcagtgaactgagatcgcaccactgcacatcagtctgggcgatagagcaagagggtttaaaaaaagaaaagcaatatcacttccttgttagatccagttaccaagccagtgagaagggatgaacgggaaggaagtcacatccttaatgtccaggggggactgttacttctgttatttctttttttatgtatttatttttttgagacagggttttgctatgtctggagcactgcaccctggctggagtgcagtggtgcaattacagctcactgcagcctcaacctcccaggctcaggcagtcctcccaccccagccccgagtagctgggaccacagacgtgcaccaccacacctggctcattttcgatttttttgttgaggggggtctcaccgtgttgcccaggctggtcctgaactcctgggctcaagtgaggagctgttacttctggacagtatgccttggacagtttgcccggccccagtgggtgtgtttctcctctgtcggtgtggctaattttcatctgcctttatccctcagtcatccaagtataacctcagcagtaacggtactgcggccagaacagtgttctcagctggcgcccctcccctctcctcttacgcactttctgaacctttgaagttcaggcacaactgatcagcattcacgttaaaacagagatcctaagactgacagaacagacgctctgtagcaattagataccaaattcccaaccaactcaagtatagcatcacatgacagagggcaggtactgaaggaaatcaaagtattttaccccaaaatatatttcttttacatattttgcttttgttgttgtcgtttttgtttttgtttgtttgtttgtttgtttttgagacagagtcttgttctgtctcccaggctggagtgcactggtacaatctcagctcactgcagcctctacctcctgggtccaaatgattctcaagcctcagcttcccaagtagctgggattacaggtgcatgtcaccatatccaactaatttttgtatttttggagagatggggtttcaccatgttgaccaggctggtctccaactcctgacctgaggtgatccacccaccttggcctcctaaagtgctgggattacaggcatgagccaccatgcctagcccctttgacatattttgaaatggccctgcaaagctgtctctggtggggaaattttacattctgtacagaatccctttccctttcaagatctttttccaaacctgaaatgattgactgagagtctagcaccttttaaggtctgaataggagccatttgccatctgttgcctcctgggtggccacctacgagacatctacataataagaacctgagtcctcacaaccccttatcttgacccagacactcctttctattgaatccaggtccttagataactcttttgaccaacggtcaatcagaaaacctttgaatccatctatgacatggaagtcccttcccttggagttgtcctgcctttgcagtccaaaccaacatatgcctcacatgtattgattgatgtctcatgtctccctaaaatgtataaaaccgagctgtaccctgaacaccttgggcacatgttctcaggacctcctgaggctttgtcatcagtcatgacctataaccttggcaaaataagcttctaattggattgagacctgtctcagatgctctttggtttacaacagatttaagaagctcagaaaaccccaaacaaggacaaatcaacaaaaaataaaacatctaattacatcatggtcaaaccactgaaaattaaaaaaaaaaaaaaaaagcaaaagcttacaattaaccagaaaaaaagatgtatttcgtggagggaaacaataataatgacaaacaggaaacctgttagcaatgcctgttccccagtgccacaaagaaatagcacttgaacataaattctctgagcaaggcaatttttactttctgcagaaagggtactctcgccagcagttttgccacaagagtacaccaaacaaaggaaaagcagacatatgtatcccttacgcatttgggtcatccttactactgtgtcctgcatccattgcctggagccagacctcacagtctaaaatgatacctgatttgctaacaacctaaaactttcttaaataggtaaatgcaaggaagaacaaaggaaaagaggaagttgcttgcgaaagatttaaagaagcaataacatttccaaataaggaagaggcataggccgtgagctggatgtccaacagctacgtaggataggactcaacaaagagtaattagcacaaagcaaggaggcttgaaggaagttagtctttaaaagaaactattacttctaacacttatgatttattatttaacaagggaaactttgaagaggaaactttttactttctacaaaacttcttaccagaaacaatggacactgaagaccacagaagacacctctacagagctgaaaggaaaaacgccaatctagaatttaacgtccaaaggaaatatctcccaaaaatgaaggcaacataaagataattgtagatattagtttccaccctaccctgtccccatattaaatatagtaaggtcacttacacatgcgaaccattatacaaactagtagagtacatccccagaagaagggaaaggcatccacaggaaggaaaggtgcggggtgagccccagtgctcatgggacctccaactccttcacacccccatagcacaggtgagcccaaggcaacgtgcctgccctacctaccctgccaggtcacaatgttcatcccagataatattagtgaaaaacatcttttggcaatgacctttttatcttaaaactgcaaacaaatgtaatgatacttcggttttgaaaaaaatatcatcagtccacaggagatcttacaagacagtctaaagctcagactcttacattatattgaaaatacatggggtgcgtcacccccaccagtgtccccagctgaccatctgcatcttggagaacaatctattttcaataacaaatgctcacaaagtgttttgaggaaataaaagcaaaaaataagatggtggggggcggggagaaagaaaacctacgagtgtttttgcagcattcctggttgttctcacacagcactcttatgagtcctgtatgaaaaatcccagaggaatgaacagcaccacagctcgggtgctgtgaccatttttattcacaacttaggagttgtatatgttcacaagccccaagaccagttatccacaccaatcccctcaggcacagctgccaatctcaagtaagatgagccactattggaggcactgatatcacactgttaccctcactcacttcctcagctgaagccatcagcctgtatattagtctgttctcacattgctataaagaaatacctgagactgggtattgtataaagaaaagagatttaattggcttacacttcctcaggctgtatgggaaggatggctggggaggcctcaggaaatttttgatcatggcagagggcaaagaggaaggaggcatgtcttccatggccagagcaggaagaagaagagagaaaaggaggtgccacacactttcaaacaccaggtctcctgagaactcactcactgatatggtttggctctgtgtccccacccagctctcattccgaattgtaatccccacatgtcaaggacctggtgggaggtgactggatcataggggcggtttcccccatgctgttttcatgatagtgagggagtcctcgtgagagctgatggttttataagtgtttgacagttcctccttcacacgctgtctctcgcctgctgccatgtaagatgtgcctacttccccttctaccatgattgtaagtttcctgaggcctccccagccatgtggaactatgattcaattaaacctctttcctttataaactccccagtctcagggaagctccttatagcagtgtgagatggagtaatacactcactatcacaagaacagcaaaggggaaatccaccccatgatccaatcaccacccaccagacctcacttccaacactgaggattacagttggacatgagatttaggcagggacaaaatccaaaccatatcagcctggttaaagtcaggcccttcagtgaaccaggctgcgtttcagatgggttccctgggagctcccagcaggacccccttcaggtgtcccagggtgacccaggagctggaggagcagccacagctcctctgcccatgtagcttgggcaggtcccccacgttccctgagccttaatgtccttgtacacaagatgattatgagaggaccagctctaagccccaggacaccagatgcagaggcacctaggacagcagcaacaacatgccgagtacatgccaagcacacaccaaggacatgctgagtacataccaagcacatgccaagtacatgtcaagtacacaccaagcacatgccaagtatacatcaagcgcgtgccaggtgcatgccgagtacacaccaagtacatgccaggcacacaccaagtacatatcaagcatacaccaagtacatgccaagcacaaagtcgtttttagttgtattgacagccaccagtgtgagatacaggcttctcttgagcccttgtactacattccagagactacacaaatgtaccaaaaagtaagtggtaaatcccaatttttacaaagtagttcctttagaaatcctacacagaaactatctaaaactcaacctttcctctgaggaaaaatagctactgtttaacccaaaagtagggtatgttattttaatttaggagaacatctggattccttacaaactattaattcttacactaaataactaagacgtttccacaaggtcaagaggtgattaattacaaacggtcaccctggaaacacagaactttccttcatttctgatttttcccgaatagctccttgttctgacatctgatgatgctgtgtccacaaggtctgtgcacaccagctggcgcctgtcaaggccatggccaggccactgacctctgttcattagtttatttaccaagcatccccatgggccagaccctgttctaggcaggaagagcagcagaaggtgaaccataaacaaaccaaaatccaccagcaagcgagtcttcagcatgcagaaggcaacagtgccatggagggcaggcaccaggcaggtggaagaggggccaggtggccggggcaggccgctgggaggaggcgttggctctgtccacgcctggagaaggcaatcaaagatgccttcaaaggacaattttattttagacttttctcctatttctgggtcagtgtttgttgttctgttgctatggaaacactaaaaccctggattcttttttactaataaatggatttcttagccacgaccatcagccaaaacatgctaatggatttggtctttgatttttttcaagttaaacaaccatattcatccataatttaaccatgtacttaacggttcttcagaactgacatagaccaaggcgtgcgtttacttcatccctaccctgagccaggaactgtgcgaaacattttgtgtagattacttcatttacccgacatagcaactctatgaaggaggtatgttgtcccagttcctgaaggagccccaggcacaggggcacagagacattaacacctcacccaggtaacacggccagactagcgtagctgagacttgaacccccttgtctagctccagaccctatgctcttaaccagcattctgtctaaatcccctttaatgccatcatttttagagcaatggagagttggagcaagacaggtattcacaaattcctcaagtgccaccttccctgtagaattccaatagaaatgaatggatgttatgttttcaggaagggtaacaagaagagaagtttatctgtccataaacatcagaaagaaagaaaaatggtggggcttggggtggcttgtaaaaagccctggggctgtatgacatgcccctccaccctgcaaggaggaaaacttgtcagatgcatcataatgaacttaaaactctggacatttccagcagccagtcttgagtaagacatgcacacgcacacataaatgtctgcatgtgcaaatccacacaaaggaaacgctgtgagagttcacacatgagaaaggtagttgagtcctgtcctaggaggtcacctcccacccacactgtctggaaggaaaatctgctaaaagggccagaatatgtaagacagtatcttgttcataaaaactaacagaaatttagtggtcagcaagtttgagcccattaagagtccaactctgcccctaggggacaacaggcttcagaaaaaaacaggaagagtcacatgtgtatgcacacacatacacgggcatacacacatgcaaacatgcatacatgcacacacataatatacatgcatatatatgcatacacacatgcatacatgcacgtatataatacacatacatgcacgtgtgcatacacacacaatacacatacatgcagatgtacatatacacacaggtgcatacatgcatgcacataatacacctacatgcacacaagtgaataaacgcacacacaacacgtgtgcacatatacacatacatgcacacatacatgcacatacacatttacacattcatacatgcatgtacataatacacatatgtgcacatgtacatatatatgcacacatacacacatgcacataatatacacgcacacatacacatacacacgcacacatgcaccaacttctctcttgatgcacaagatatttctcaaaataaccttggccacttagcacctagggacagaattattgacagaacattgggaatgagggtctagactggccacaggtccctcatgctactgtgacaccttcaaatgagtgactgagcaaagtttctcagaggccccaggagtgagctggtagcaccatagcaccacacacagatgactggtgctatgagggggcctaattcttcagacattctgtgcatttagtgatggcaaagcacgttgctcggcttttcccttgttcagttaacagggtgctgttccgggcattacttgagtcttttttggaacccaaagtgctcatgacacgtgacagcaattgtgtttgtcagggatcttcagagaaactgaaccgataggatgaatatagagatttagagggagatttttatttcatatgggctcacacaatgatgaagtcccatgatcttctgtcagcaggctggagatacaggcgagccagtggtgtcgttccagtccaaacccaaaggcccaagaacccggaactcagatgttcaagggcaagagaagaggacgtcccagctcaagaggtgagagcagtctccctttctccacctccatgtcttattccagccctcagcgggttggatgacacccattcccgttccggagtgtgagatgctattcagtccaattcagatgcccatctcatccgtatgccctcagacacaccccagagtcacgtgtgccagctatctgggcacctcttagcccaggcaagctgacacataaaattaaccatcaccacattttgccatgtaaggacctgaaaggcctgatttggccgggctcactgcaagggcctggtttaagcaggtgctcagcctgtcttggcctcattctcatccccatgaactccagcccatatctcccagtcattggggatcctttcctggaggagctgcttggtttgcaaggacaacgctgactggccattatctcaagggccatcagctatgcatgcaccttacatggggggtaagcattttgtttggttttgttttttagctgaaactgtaaaaaaaaattaacatgaactgatgagaatcacccataccatttcatgaaacgcaatcattggtcatgactagggagagcatctgagttggatactgttaaggatacaatttaggctttgcctgcgtgatttggaacagaaatcttgacttagaacttacagaaaaacatttcaacctgagaaagctgattacaaaataaaaaggttaagaaaacatgacacattaaaaaaaaatctaaaaagtgaaaacttcagtcatccaattatttggcattgggctgacaaaataaaagaactttttaagacaccacacacaaaaaatgagtccagcttctcattgagagtgagcatgttattgacacagacagattcaaaatcctaagaaggataatgaggaaaatcggtttttacaaataattattaatagacccaatgctcttagagattcctcctccctgagcaccttttgagcaagaatctttcctcggagctttgggaattccccttctagccctgtgggaacccacaaatcatagaccaggggctctgtaaattctgcagggggaaaatccattttgtggataatttattgcaaaagagttgaaaaggggtgttggttaactagaaaagcttatgcagtaaatagagaaaccaaccattcgactccattgtggctacaaaaatggtggaaaatgacaggtctttcagtcctcgccacctacttcccacacttcaatagtgttaacccttaaggaccatcatcatcaagaggccttaatagagaatccactgaaatgaaaccacaatcaattcaatgctagctacttggcctataaattgaaagcaaagtaaatacaaattactctgcctgctgtgatggtgaatgaggtttttaaacacagctacttgtcagattttggccctttatttttattttccccctcctttatctgagttttacttttctcctagggcgcctttccaaggtgtttgtaaattgcccccaaacgtcaggtctgcagttggcatctcaccacttatgcctgcgcttcactgaggtccgcagggctatggaggctgagctgaaagcacttccaagtgattccccaaggaacgtcctcacaggagcgttcagaggggcccagccagtgctcttgctgctgaaggagcatcccagggaggtgacgagcccagggcccctggcacagggtggccctggaagaaagcactgtgctcccagcacttgaacaggacccttctgcaaacacagcgctcgaacggaacccttctgcaaacacagggctcgaacggaacccttctgcaaacacagggctcgaacggaacccttctgcaaacacagggctccaacggaacccttctgcaaacacagcgctccaacggaacccttctgcaaacacagcgctcgaatggaacccttctgcaaacacagggctcgaacggaacccttctgcaaacacagggctcgaacggaacccttctgcaaacacagcgctcgaacggaacccttctgcaaacacagcgctcgaacggaacccttctgcaaacagcatttgcataaattgcaccgtcatttttgtacttattttacatcagagaggtaggctgggctttttgaaagaggcacactggctgatcttgctcctgatcagctgaatcccttctttgtaaagcagggaagataacagccacctctgagggccaatgtcaagacccaggccttcaaggctcctaagagaacaaggtggctgtgccttgggaggctgaggccagaggattgcttgaggccagaaggtctagaccagcctggacagcatagcaagaccctgtctctacaaaaaaaaaaaaaaaaagtaaaaattagcaaacatggtggctggcatctgtagtcccagctactctggaggctggggcaggaggatggcttgagcccaggaggttgatgctgcagtgaaacatgatcatactactgcactccagcctgggcaatagagcaagaccttgcctcaaaacaaacaaaaacagcacgaggcagccacatggctattgcaggagcaggaggggctctcccagcaaacttctgcttgggctgacgttgggccctgtgggcatggagctttgggcccctccccctggaatgggttgtgctgcagcacaaggcactgtctcctgcggaggagcctggtggagtcggccacagaggaaagggtttaattcatcctcgctacgtttcccacacatgctggggagtaccatacacgccagcattgcactggcttctcctgggttgaccagaagtttctgttgtgcctggtgtgggcagttgtaaattacaggctttgagtcagaagaaaccttaagcccacctgatgctctcggctccgactgtgctcaggctctggctcagcagcctgtgctccccggcgtgtccccgcctcacttcgtctagggggttgctctcctggagggcaaggaggctgcagtgtcacggctgcacctccccgcacgattcggcaggacgtgcccagacggcaggactgaattttcctgttgtggcaaaattaaattctgagcaagtgttataggaggagttttgtctgtaaacgtttattcaagaggcaaaggtagcctttctggctgaaaactagaaactcattctatttaactaatgtgtagtttgcggggaggtgggatttagatgtatttaaaccccacccctgaactattgtgagctttctagtgtgtgaagccaagataccgaacaagtcggcaagagacaaaaacggggagatgccaggacgagcatccgttctgctccgcagtggcccgggggccggtgttcccgcagacaccagcagctgatccgcgcacagaacaggaccccggccacggtgaggaagagagaacgcgcacgcttcagatctctggtcggtaggtggctggaacgccaggtgtggggcgggcgctgcagagcctcccacgcggcacaggcttctgcctgcttttcaaccacggctttgctgctttgacatttctggaatcaaggtgcacctcataagcaacaccaacgaaaatcaagtagggacgaggctgtggctgacgggcaggtgcagaccacaccctctccacgacctggggcgtgtctgtggctgacgggcaggtgcagacctcaccctctccacgacctggggcgtgtctgtggctgacgggcaggtgcagaccacaccctctccacgacctagggtgtggctgtggctgacgggcagatagtgagtaaattgaggcacttcaggtgctccccggagcggaaggccgaggggccctcagggtcccacagacactggggggcagtggctgacacgtcaggaagggctggcctcagcgcgtggcatttaaagcccccaggcagggaggggtcccctgtgtctgatacagcacgtagctaaacagcccttgtgtaaaaagtgacacgcccggcgctgctctttccctaggagcgctgttatggagttggcggtggacctcacgagtgccgagtgcatttgctccccggggaggctcgatgctcagggactccaggcctcggcgtctgcagcagataatcacagaggcttgccaggtggcctccggtgagctccgtgtggctgcagcccagctggaatcgctgagattagggaggaagaggggccggttcctcatagcccgtggcacgtgggaaggatgcgcccccggcgtagagcccgcgctggggaccacaaacacgcccctccagctgctacccactttcatattctgttcaggagctacctggtaggttttttggttaattttcaatgaatgtaagtctatatttctaatgtataggaataaaacaaacaaagaagtagtctatccacaacacacttcccaggggttgggcagcagtgggctcagtcctgccagaatcggccccacaggagcagccccagcagccgggacctggagcctgaggccagcgccgcagaggttggaatccggaaaggaaggcgccctccaaaatgacgtgcagtgagtctggatgacttgaggaaaagaagaagaacatgcattttggatgtctatttacaacaacactgatttcttagatttagagaaggtggcaaaaccctccactgaatgtgattaaagtcgtggagagcaaggtgggacccaggccggcagcagcagcagcgaaatagatcaacagacaacagggtgttcaggacccagttcagggctggagaggacagagatcaacaaagaacttggtcatcgccaaggagcccgatgccgggtgagtgtggacggcagctcacgggagccctgcccgtgcccatcctggtccagccacgcttgctcgaaccacagagagggacagctcagcccacagctacacagaggaagagcctgggcagctgcccacagccccagggatcaggcctaacattttcctaaaacttcctatctgtgttgccttttccactcgcaccgtctcacttacgccacaggctgggaggtaagcagggcacgtatagtccccaccgggcactgggatctgaagcagtcggcgacttgcctggggacagggctgctccacagatctgggcatcaagctgaggttccggctccaagccctgtcgccttctcgcctcttcatttgctttgtggcatcacagcctaagcaaaaaggagaaagcagttcgctcaggatgggagtactctcgtcctggcttccggaaggttcccgacacaggtcccctcctcctacggtgcaggccccggcgcttacggagccacttcccagcctccctggcagcctcagcaccacgctgtctccgctgccgctttaactgtggacgtggtgaccagcagccttccagaggtggcccctccaggaacggtggcatcagctctgtgcagggaggacagctgctcccagacgaagggaggaggcctgggtccccacacagtccctgattcagggtcgtgctggacacgtattctgcacggggcagattatgcggattttagccttgtggatcctacagccgcagccacagaaagatggagacaagggcacgtggctgtgtcccaggaaaactctctctacagctgccggccacttctggccgcctcccggtctagacgctcacatgagagaaaaacagatttaaaaaatcatttaagccattgcctcaaggccatttcacaaatccaatgacaggccaaattgggcaagcacaactatgagtaaggcctattccaatattacagaaaatctgcaagacagtttagaggcgaggttccgccatacctgccagatcgcccaacccctggggcgacaccaggctgaggacacacatttctacttctcgtctctgtgcctttgaccttcccatccctcatgcttctccagctttgtggctccccacagggctgtgtttattctgtgatcactgcccattttggaggtcacaagctctggagcttacaggaactgggcaggacattaaaaagcagcgtgtggggagtgctgtggggaactcacgggccctgtcccatggaaaatattcaagttcaaacgttagtagaacatcacgagggtcaaacaaaatacgcctgcaggcaggacctctgatttatgttagcgctgggtgtctccgcctacccacaaggcagcgccagaaccaccgcctggctcttagcacaaaatctgctcataagtggatgcttagggagtgtttgttgaactgaacagaggtgaattataggctattaccccaagactctgaaatagtagacatgttctgtgaaaacctaggggacctttttccaaatcgggggccgattaattcagctgttggaggctctggcccccatgcagactgaagcctcggttcagggccagcggatggcagaggtgcggcagggtcagagctgcgatccctgcagatccttcagcactggaggagaaacagccaaagggcaggcgtggcccagagccagcggcagctctcctgcaccagcctgggcagagacggcatgaacagagagtaaagtgttaagtccagggatgcttcccccaacattatttgatggtctctttcttaagaaaacaaaggaagcaacaatgtttcaacgtgcctcttcaggtatcctcagtgatttatattaaaaaccagagcctttagaaacatcttgaaatcacactctccagactcaccccaaaatacccaacagtgaaggaggacttactgtcccactagaatgggcactttctctggagggaggagaaggttgtctaaagacactaacagtaggtggggcttgtctaggcctgtgggggacggggcatggtgggggtgtggggagggaccgccttccaccagaaaggatgaaatgagctggattttcatgtcataaacagatttactctccatgtcctgatgtgtataaacctttaaaaagactctagaaatgctataatattcagagcattctctctggatggtgagactataattttttcttacttatttttaatataatttcagttgtcttcaatatataaatattacttttagaatgtaggaaataagtgtaatttttttagagagagagtctcattctgtcgtccaggctggagagcactggcacgagctcagctcactgcaagctccacctcctgggctcaagcaattctcccacctcagcctcccaagtagctgggaccacaggcacacacctccacacccatctaatttttatatttttaggagagatggggtttctctatgtttcccaaaaggctggtcttgagctcctagactcaagtgatctgcccaccttggcctcccaaagtgctgagattacaggtgtgagccactacacccagactaaatgtaattttcaaaagaatatatgtacaatgccctggtcttaataagtatctgatgcatgataggtgcattagtctgttcacacactgcttataaagacatactggagacggggtaattcataaaggaaagaggtgtaatggactcacagttccacatggctggggaggcctcacaatcatggccaaaggcaaaggagaagcaaaggcacgtcttccaaggtgcagacaggagcgtgtgtgcaggggaaccgcccttaataaaaccaccagatcacgtgagacttactcactatctcgagaacagcacaggaaaaacctgcccccacgattcagttacttcccaccagatccctctgaaaacacgtggggattacaggagatacgctgcaagatgacatttgggtggggacacagccacccacagcaataggttttatacaaatatacacgtatgcttgtatgtatatgccattcaccaccttccccaagctaatttttaggcatatatataacatatatgccattcaccaccttccccaagctaatttgtaggcatatatataacatatatataacctatgatgcatcaggtacttattacacatatatgtaacatacacacacatgtaacatatatatacacacacgcatatgtaagctacttttctgcagcctcaacctctccagctcaagccattctcctacctcagccgtcttgagtagctgggactagaggtgtgtgccaccattcgcccccagctatgtttatttgtagagatagggtcttgccatgtttcccagactggtctcaaactctgggctcaagcaaccctctgtcttggcctcccaaagtgctgggattatgataggttatatttgtatcaaagtaccatgtgagcaggaaccttgtactatgatctcactttatagttttaaataataccatcatatttaagagaattactttattctaacctgcataagtaaaataagccaagtgactttagactttagaattcagtgtcactcaattcaggagaggagggcgaggagggtaggaacagccacatgtgggtcagctcagtgtcactcaattcaggagaggagggcgaggagggtaggagcagccacatgtgggtcatctcagtgtcactcaatttaggagaggagggcgaggagggtaggagcagccacatgtgggtcagctcagtgtcactcacttcaggagaggtgggcgaggagggtaggaacagctacaggtgctgagaattcccaccagggtctgtctggccacagggcgccctgtcctgttagcccatgggtgcggatgccacctgggctggcattgcacaggtgggaaggatgggggagcgtgatgctggcagaggttgggcaggagcccgaggcagtgggctctggactcaccaccgtgcacagcaggcgctgaggctcacggagtttcaagggggcgcaccctgggaggtggaaggtgcggtgggtcctgaagggtgctgaggagagtcccctcaggctgagcccacgctgtgcaacatgaaggtgcggattctgtcttacgacgcggctgggggttcaccctccttttctccctgtccacacaatatgaaggtgcggactctgtcttacgatgcggctgggggttcaccctccttttctccctgtccacacaatatgaaggtgcggattctgtcttatgatgcggctgggggttcaccctccttttctccctgtccacagttcaaggctccttcttattcccttaggatatttttttaagtatctagtttacttttctgcttttactgtcaggctgtaaagagcccaccaaaaccacctccttaaagagcaaaaaatccaactggcattggagaccccatctcagccacggaagaggccttctctaacccagacctaggaagcttcttgcacccccttagccatggagaagaaaagctaaaaataccaattcaggtaggcgggatgtccactttttccctggaatccttagcttgcgtgggacacactgggatgggagctgctcctcccgtgctcactggggaacctcctcagaaactgctctggacagaaggtgggaggaacctgcccactttgaggattcccatcccccacaacatgtgactctagcgcatcccacaggtgccagcgtgcgtgtttaccgcgccctcctcggaagcccctctgggctcttcttctgctggtggattcttctgctcctcttcccacgtggatttccaccgcgaccttgccacgtaccatggcttcgggccccatgagtttcacacccaccatgggtgatgcacgcctgtgaggtttggtggtggtgagagctttgcagcaggaaaggcgggtctggaggatgcctcaacccagggcgattctattcccagggggtgtcaacacggaggctccagaacacatccttctaaaactccctggaagccctcacgtcccccgaggcacagcacccagggcaaaggccaggccttggatgcagggaggtaggaggagcttcccatggacacactggagaagaacatgctccttcagctcctcagaaccggtgccagctcagcctcaggcagccccaatgccagctgagccccagagggagttagaggggtgaagactcagaggagcccgggaagaaccaccagttcacttctccgctccctgccctctcacttgacacctctcagtacccaagcactccccaaactgtgtacctcccacacaggtgccaggacagaggccagcatgccctggggctccagcaccttcctgtggctggtaggaacacgcccagtctctccttgtggccctgggcctccagctcacgctcccagcacctgcacctccaggcctggctggggaccccgtctccctgtgcctctcgcagactccctgcccgtctttgcttcactcccacttgtcctttgagacctggcttggtgccatcgcccagggacctgccaggatcccagctgtgcctgatgatgggggcagggtccctgccaagtagaaggaaaggcacagccacatccaggccaaaaaataaaagacaatgatttacctccctctctactgtccataaaacaggtccagcattcaaccaaaatgcttaagtaacacagaaaaaagcaagaataaaaccacattcccaagagacaaaacaaccatcagaacaagacacagatatgactcagatgttggaactatttgaaaggggattttaaataactatgactgacacattaaaggctgtgatggaaaacgtggacaacatgcaagatcagatgggctgggccagattcagtagctcacaactggaatcatatataggccaggcacggtgctcacacctggaatcatatagaggctggtcccggtgcagtatatcacacctggaatcatatagaacacctggaatcgtatagaggctggtcccggtgcagtatatcacacctggaatcatatagaacacctggaatcatatagaggctggtcccggtgcagtatatcacacctggaatcatatagaacacctggaattgtatagaggctggtccccatgcagtatatcacacctggaatcatatagaacacctggaatcatatagaggctggtcccggtgcagtatatcacacctggaatcatatagaacacctggaatcgtatagaggctggtccccgtgcagtatatcacacctggaatcatatagaacacctggaatcgtatagaggctggtccccgtgcagtatatcacacctggaatcatatagaacacctggaatcgtatagaggctggtccccgtgcagtatatcacacctggaatcatatagaacacctggaatcgtatagaggctggtccccgtgcagtatatcacacctggaatcatatagaacacctggaatcatatagaggctggtcctggtgctcacacctggaatcttaggccaggcacagtggctcatacctggaatcccagcacttcgggaggccaaggtgggcagatcacttgaggtcaggagttcaagaaccagcctgcccaacatggcaaaaccccatctctattaaaaatacaaaaatcagccgagtgtggtggtgcacacctgtcatcccaggtacttgggaggctggagcacgagaatcacttgaacctgggaggcagaggctgcagtgagccaagatcacaccagagtgagactctgtctcaaaaataaataaaattttaaaaacgctcgacttggctgggcacggtggctcatgcctgtaatcccagcactttgggaggccgaggcaggcagatcacctgaggttgggagttcgagaccagcctgatcaacatggagaaaccccatctctactaaaaatacaaaattagccaggtgtggtggcacatgcctgtaatcccagatacttgggaggctgaggcagaagaatcgcttgaacctgggaggcggaggttgcggtgaggcgagattgcgccattgcactccagcctagacaaggagagcgaaactccgtctgaaaaaaaaaaaaaaggaaaaatgaaaggtatttccttgattcttaagcatccttacttgtcttgctttgccccattctcccggcctttgatgctcctcccatgctctcctctagctttccgctcctttccctgaagccgcgccgcctgcacctcctgctccgctgaactgcgccgtctgtgcggctcgggcccctccctcctacctttgctgctgtgcgatgattcgttccacggcactgcctctgctgtcatggggccctcaacagacgggctgccaacaagcagggcggggtggaccctggctctcgtctacacactggagtggggacagggccgggtgcacccttcctcttgtctactccctggagtgagtggggactgtctacagtcctgcccaggcagacgcttttccaggacagatcagctcagctaaagcagcatcttctctcccaggcttcactgtctctcagatattctaaataaactattttaaaaagcctacttccgcattcaggactgtgtgtgactatgtgtgactatgtgtgactctgtatcttcagagattatttctggcgaaattacattttggcatttcaccacctgcggggtgttcctggtgttgagtctccaagaggatgggaggccagaggtgctgttggacggtggtgctgtcgcccagggcagcacccactgcttggaggaactgctctctcgggggcagacgtggacctcagggcgcagaggctgcccggttctgtgccatgttctccagtccaggaccagccatctgttcattaggagtcagtccgagattagcggaaccttccttagtaggagggccgtgtctcctctttgcgtcttcctgagcattttagtgagatttggaagagctgatgatgttaaccatctgccattttattgtgaggccccaaaacttaaatttcctacttgtgcttttaccatacctgggaccttgaacatctacatgtcactgggtaaaccacagccgcgactggacccccctcttgactccagtttacgtcaaatggaattttgtaaataattgtggggggtgtggggtcattgagggaccagactgtagcctccaggaataaaagtctgcatatccagttgcgcttgtacattccgatatgactctgccctgcagacacaactgccactcagccatcccccgcgccaccttcactgagcacctcccgtgtccgaagcacccgggggcgatgactgtgagaaaacagaagtgcctccgtcctcaagagctcagtccagcacagcaagcggggtatcctcagcacacaaataaccacacgtgaggtaacgaacgttttaatttacttctactgatgtggcaaatgttgaaagaaaagtgttttctaaataacaaaaacacgactgaaaaagagaaaattaaattaccgctgtctgccttggaagaaagcgctcagcctgcgtgcagcaaggcaccagggtgggcttggagacccccgtgtggaagcggccttgctgtaaagtcagacgggaagcccacggtgggaaggaaccgcctcccgcactgtcggcccacctcttgccacgtctgttttcctaatctgctgtgaaatgtgttttttagaggctgcgattactccatcctgttgcctgtctcaccttcctctggaagatcccggaggcatcgctagcgccagggcaggctgcacctgggaggggcccacaagcaccgggccagctcctccctgggggtgaggctgaggggcagatcgttggcctttttggatgtttctttcttcacgaacttcacatttctatatggctcaaaaaacacccaatgcttaggagcctgggaaggacacattcaaggacatcatggagcagaagctgcacaggccaaccagccacagggcagaagccgcccaatggccacaggtgcttccgaggcagcgcgtcagacacggcagctggggagagctgggcctcctctcgcccctggcctccgtgaggctgcacctcctgggcactggcagagcatgagcaagggcttcagaggggggagtgagcccccagccaggcaggaggctcacgtcaggaggacgtaactgagctgcagatggtcctcccttgggagctccccctaaaggagcagcactgtgaccgggcacataactcaggagccagacagaactccctggattcccttgagctgatttactgagcagtttcagagacctgagcccaagcagactgcctcccaggttacaaaggagtcaggacagagaacgaactgtgaactgcagatgatagctgagaattcgcagacaccaaagcccagaagaggcccaaagaaaaggagggacaaatattcattctaactttaaataagatggaatctgaggctaagaacttacctcagtctcaaaacttctagaccaatcatcaagcgggttgcaaaacagcagcaaggacggtgagtgggtgtggactcgagaggccgtgtgtccactccacttcctttccacgcaactgtgaacctgggttccgcgaagatgcagagagaggccgcgtgcctgtccacttccttccccacgtgactgtgaccctgagttccatgaagatgcagacggaagatgaacaaattccagccatttgttgaatgaggattggctaagactctactgacacagcaggcgctgtagcaggcacctggaattcagcggccaacagtttcaaatcaatgattaaaaagcaaagaaaggctggcggttgctcgggtggctggagaggcccacgcgagacgtgcagccctgccgttggggagggggctgtgtctgcagcgtcacagccggtctggtgggtccctcccagacaccttccaggtctaagacaagtaagcggaggtgatcttgtgccccagtgagaagacctaactgtgcccatccgttcacctcacagacccatcgatggccaacggtgctgggaacattctaggagggtgatgggacagagggatgggtgtttgtgacacattctgggattttcctgtcctcaccccagaactctcctgtctttgtgctatggtttgaatgtgtgtgtccctccaacattcaaagcctggaacttaagcacaagggtgatgctgttaaggggcagggccctttgagacgcagagccctagtgaatggatcggtgctgtcagcagagaggctgaaggccccagggcctttctgtcctccgctactgtgcgagggcacagccatgaggtactgcggaaagcagagggcagctgaccag

SEQ ID NO: 2 - DNA, 20 bp, Homo sapiens
cctggagtctgaaaggactg

SEQ ID NO: 3 - DNA, 20 bp, Homo sapiens
acagacccatcgatggccaa